(19) United States
(12) Patent Application Publication
(10) Pub. No.: US 2023/0265406 A1
(43) Pub. Date: Aug. 24, 2023

(54) RECOMBINANT SIALIDASES WITH REDUCED PROTEASE SENSITIVITY, SIALIDASE FUSION PROTEINS, AND METHODS OF USING THE SAME

(71) Applicant: Palleon Pharmaceuticals Inc., Waltham, MA (US)

(72) Inventors: Li Peng, Lexington, MA (US); Sandip A. Shelke, Belmont, MA (US); Lizhi Cao, Stoneham, MA (US)

(21) Appl. No.: 18/003,234

(22) PCT Filed: Jul. 2, 2021
(86) PCT No.: PCT/US2021/040240

§ 371 Date: Dec. 23, 2022

Related U.S. Application Data
(60) Provisional application No. 63/134,411, filed on Jan. 6, 2021.
(60) Provisional application No. 63/047,989, filed on Jul. 3, 2020.

(51) Int. Cl. C12N 9/24; C07K 16/32; C07K 16/28; C07K 16/40; A61P 35/00

(52) U.S. Cl. CPC C12N 9/2402; C12Y 302/01018; C07K 16/32; C07K 16/2896; C07K 16/40; C07K 16/2887; C07K 16/2827; A61P 35/00; C07K 2319/30

(57) ABSTRACT

The invention relates generally to recombinant sialidases (for example, recombinant sialidases having reduced protease sensitivity), recombinant fusion proteins, and antibody conjugates, and their use in the treatment of cancer.

### CROSS REFERENCE TO RELATED APPLICATIONS

This application claims the benefit of, and priority to, U.S. Provisional Patent Application Ser. No. 63/047,989, filed Jul. 3, 2020, and U.S. Provisional Patent Application Ser. No. 63/134,411, filed Jan. 6, 2021, the entire disclosure of each of which is hereby incorporated by reference in its entirety.

### FIELD OF THE INVENTION

The invention relates generally to recombinant sialidases (for example, recombinant sialidases having reduced protease sensitivity) and recombinant fusion proteins, and their use in the treatment of cancer.

### BACKGROUND

A growing body of evidence supports roles for glycans, and in particular, sialoglycans, at various pathophysiological steps of tumor progression. Glycans regulate tumor proliferation, invasion, hematogenous metastasis and angiogenesis (Fuster et al. (2005) NAT. REV. CANCER 5(7): 526-42). The sialylation of cell surface glycoconjugates is frequently altered in cancers, resulting in the expression of sialylated tumor-associated carbohydrate antigens. The expression of sialylated glycans by tumor cells is often associated with increased aggressiveness and metastatic potential of a tumor (Julien S., Delannoy P. (2015) Sialic Acid and Cancer. In: Taniguchi N., Endo T., Hart G., Seeberger P., Wong CH. (eds) GLYCOSCIENCE: BIOLOGY AND MEDICINE, Springer, Tokyo. https://doi.org/10.1007/978-4-431-54841-6_193).

It has recently become apparent that Siglecs (sialic acid-binding immunoglobulin-like lectins), a family of sialic acid binding lectins, play a role in cancer immune suppression by binding to hypersialylated cancer cells and mediating the suppression of signals from activating NK cell receptors, thereby inhibiting NK cell-mediated killing of tumor cells (Jandus et al. (2014) J. CLIN. INVEST. 124: 1810-1820; Läubli et al. (2014) PROC. NATL. ACAD. SCI. USA 111: 14211-14216; Hudak et al. (2014) NAT. CHEM. BIOL. 10: 69-75). Likewise, enzymatic removal of sialic acids by treatment with sialidase can enhance NK cell-mediated killing of tumor cells (Jandus, supra; Hudak, supra; Xiao et al. (2016) PROC. NATL. ACAD. SCI. USA 113(37): 10304-9).

Cancer immunotherapy with immune checkpoint inhibitors, including antibodies blocking the PD-1/PD-L1 pathway, has improved the outcome of many cancer patients. However, despite advances that have been made to date, many patients do not respond to currently available immune checkpoint inhibitors. Accordingly, there is still a need for effective interventions that overcome the immune suppressive tumor microenvironment and for treating cancers associated with hypersialylated cancer cells.

### SUMMARY OF THE INVENTION

The invention is based, in part, upon the discovery that it is possible to produce recombinant mutant forms of sialidase enzymes, including, for example, sialidase enzymes that are less sensitive to protease degradation than a corresponding sialidase enzyme without the mutation or mutations that render the sialidase less sensitive to protease degradation. The sialidase enzymes can be used in fusion proteins and/or antibody conjugates containing such sialidase enzymes that have suitable substrate specificities and activities to be useful in removing sialic acid and/or sialic acid containing molecules from the surface of cells of interest (e.g., cancer cells) and/or removing sialic acid and/or sialic acid containing molecules from the tumor microenvironment, and/or reducing the concentration of sialic acid and/or sialic acid containing molecules in the tumor microenvironment.

Accordingly, in one aspect, the invention relates to a recombinant mutant sialidase enzyme, e.g., a human mutant sialidase enzyme, wherein the sialidase comprises a mutation that increases resistance (decreases sensitivity) to cleavage by a protease. In certain embodiments, incubation of the recombinant mutant sialidase (e.g., human sialidase) with the protease results in less than 50% (e.g., less than 40%, less than 30%, less than 10%, less than 5%, less than 3%, less than 1%, or less than 0.5%) of the proteolytic cleavage of a corresponding wild-type sialidase (or modified wild-type sialidase lacking the mutations described herein) when incubated with the protease under the same conditions. In certain embodiments, the protease is trypsin.

In certain embodiments, the sialidase is a human sialidase that comprises a substitution of an alanine residue at a position corresponding to position 213 of wild-type human Neu2 (A213); a substitution of a leucine residue at a position corresponding to position 240 of wild-type human Neu2 (L240); a substitution of an arginine residue at a position corresponding to position 241 of wild-type human Neu2 (R241); a substitution of an alanine residue at a position corresponding to position 242 of wild-type human Neu2 (A242); a substitution of an arginine residue at a position corresponding to position 243 of wild-type human Neu2 (R243); a substitution of a valine residue at a position corresponding to position 244 of wild-type human Neu2 (V244); a substitution of a serine residue at a position corresponding to position 258 of wild-type human Neu2 (S258); a substitution of a leucine residue at a position corresponding to position 260 of wild-type human Neu2 (L260); a substitution of a valine residue at a position corresponding to position 265 of wild-type human Neu2 (V265); or a combination of any of the foregoing substitutions.

In certain embodiments, in the sialidase, the alanine residue at a position corresponding to position 213 of wild-type human Neu2 is substituted by cysteine (A213C), asparagine (A213N), serine (A213S), or threonine (A213T); the leucine residue at a position corresponding to position 240 of wild-type human Neu2 (L240) is substituted by aspartic acid (L240D), asparagine (L240N), or tyrosine (L240Y); the arginine residue at a position corresponding to position 241 of wild-type human Neu2 (R241) is substituted by alanine (R241A), aspartic acid (R241D), leucine (R241L), glutamine (R241Q). or tyrosine (R241Y); the alanine residue at a position corresponding to position 242 of wild-type human Neu2 (A242) is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); the arginine residue at a position corresponding to position 243 of wild-type human Neu2 (R243) is substituted by glutamic acid (R243E), histidine (R243H), asparagine (R243N), glutamine (A243Q), or lysine (A243K); the valine residue at a position corresponding to position 244 of wild-type human Neu2 (V244) is substituted by isoleucine (V244I), lysine (V244K), or proline (V244P); the serine residue at a position corresponding to position 258 of wild-type human Neu2 (S258) is substituted by cysteine (S258C); the leucine residue at a position corresponding to position 260 of wild-type human Neu2 (L260) is substituted by aspartic acid (L260D), phenylalanine (L260F), glutamine (L260Q), or threonine (L260T); the valine residue at a position corresponding to position 265 of wild-type human Neu2 (V265) is substituted by phenylalanine (V265F); or the sialidase comprises a combination of any of the foregoing substitutions.

In certain embodiments, the sialidase comprises a combination of substitutions as set forth in TABLE 2, hereinbelow.

In another aspect, the invention provides a recombinant mutant human sialidase enzyme, wherein the sialidase comprises: (a) a substitution of a proline residue at a position corresponding to position 5 of wild-type human Neu2 (P5); (b) a substitution of a lysine residue at a position corresponding to position 9 of wild-type human Neu2 (K9); (c) a substitution of an alanine residue at a position corresponding to position 42 of wild-type human Neu2 (A42); (d) a substitution of a lysine residue at a position corresponding to position 44 of wild-type human Neu2 (K44); (e) a substitution of a lysine residue at a position corresponding to position 45 of wild-type human Neu2 (K45); (f) a substitution of a leucine residue at a position corresponding to position 54 of wild-type human Neu2 (L54); (g) a substitution of a proline residue at a position corresponding to position 62 of wild-type human Neu2 (P62); (h) a substitution of a glutamine residue at a position corresponding to position 69 of wild-type human Neu2 (Q69); (i) a substitution of an arginine residue at a position corresponding to position 78 of wild-type human Neu2 (R78); (j) a substitution of an aspartic acid residue at a position corresponding to position 80 of wild-type human Neu2 (D80); (k) a substitution of an alanine residue at a position corresponding to position 93 of wild-type human Neu2 (A93); (l) a substitution of a glycine residue at a position corresponding to position 107 of wild-type human Neu2 (G107); (m) a substitution of a glutamine residue at a position corresponding to position 108 of wild-type human Neu2 (Q108); (n) a substitution of a glutamine residue at a position corresponding to position 112 of wild-type human Neu2 (Q112); (o) a substitution of a cysteine residue at a position corresponding to position 125 of wild-type human Neu2 (C125); (p) a substitution of a glutamine residue at a position corresponding to position 126 of wild-type human Neu2 (Q126); (q) a substitution of an alanine residue at a position corresponding to position 150 of wild-type human Neu2 (A150); (r) a substitution of a cysteine residue at a position corresponding to position 164 of wild-type human Neu2 (C164); (s) a substitution of an arginine residue at a position corresponding to position 170 of wild-type human Neu2 (R170); (t) a substitution of an alanine residue at a position corresponding to position 171 of wild-type human Neu2 (A171); (u) a substitution of a glutamine residue at a position corresponding to position 188 of wild-type human Neu2 (Q188); (v) a substitution of an arginine residue at a position corresponding to position 189 of wild-type human Neu2 (R189); (w) a substitution of an alanine residue at a position corresponding to position 213 of wild-type human Neu2 (A213); (x) a substitution of a leucine residue at a position corresponding to position 217 of wild-type human Neu2 (L217); (y) a substitution of a glutamic acid residue at a position corresponding to position 225 of wild-type human Neu2 (E225); (z) a substitution of a histidine residue at a position corresponding to position 239 of wild-type human Neu2 (H239); (aa) a substitution of a leucine residue at a position corresponding to position 240 of wild-type human Neu2 (L240); (bb) a substitution of an arginine residue at a position corresponding to position 241 of wild-type human Neu2 (R241); (cc) a substitution of an alanine residue at a position corresponding to position 242 of wild-type human Neu2 (A242); (dd) a substitution of a valine residue at a position corresponding to position 244 of wild-type human Neu2 (V244); (ee) a substitution of a threonine residue at a position corresponding to position 249 of wild-type human Neu2 (T249); (ff) a substitution of an aspartic acid residue at a position corresponding to position 251 of wild-type human Neu2 (D251); (gg) a substitution of a glutamic acid residue at a position corresponding to position 257 of wild-type human Neu2 (E257); (hh) a substitution of a serine residue at a position corresponding to position 258 of wild-type human Neu2 (S258); (ii) a substitution of a leucine residue at a position corresponding to position 260 of wild-type human Neu2 (L260); (jj) a substitution of a valine residue at a position corresponding to position 265 of wild-type human Neu2 (V265); (kk) a substitution of a glutamine residue at a position corresponding to position 270 of wild-type human Neu2 (Q270); (ll) a substitution of a tryptophan residue at a position corresponding to position 292 of wild-type human Neu2 (W292); (mm) a substitution of a serine residue at a position corresponding to position 301 of wild-type human Neu2 (S301); (nn) a substitution of a tryptophan residue at a position corresponding to position 302 of wild-type human Neu2 (W302); (oo) a substitution of a valine residue at a position corresponding to position 363 of wild-type human Neu2 (V363); or (pp) a substitution of a leucine residue at a position corresponding to position 365 of wild-type human Neu2 (L365); or a combination of any of the foregoing substitutions. For example, the sialidase may comprise a substitution of K9, A42, P62, A93, Q216, A242, Q270, S301, W302, V363, or L365, or a combination of any of the foregoing substitutions.

In certain embodiments, in the sialidase: (a) the proline residue at a position corresponding to position 5 of wild-type human Neu2 is substituted by histidine (P5H); (b) the lysine residue at a position corresponding to position 9 of wild-type human Neu2 is substituted by aspartic acid (K9D); (c) the alanine residue at a position corresponding to position 42 of wild-type human Neu2 is substituted by arginine (A42R) or aspartic acid (A42D); (d) the lysine residue at a position corresponding to position 44 of wild-type human Neu2 is substituted by arginine (K44R) or glutamic acid (K44E); (e) the lysine residue at a position corresponding to position 45 of wild-type human Neu2 is substituted by alanine (K45A), arginine (K45R), or glutamic acid (K45E); (f) the leucine residue at a position corresponding to position 54 of wild-type human Neu2 is substituted by methionine (L54M); (g) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T); (h) the glutamine residue at a position corresponding to position 69 of wild-type human Neu2 is substituted by histidine (Q69H); (i) the arginine residue at a position corresponding to position 78 of wild-type human Neu2 is substituted by lysine (R78K); (j) the aspartic acid residue at a position corresponding to position 80 of wild-type human Neu2 is substituted by proline (D80P); (k) the alanine residue at a position corresponding to position 93 of wild-type human Neu2 is substituted by glutamic acid (A93E) or lysine (A93K); (l) the glycine residue at a position corresponding to position 107 of wild-type human Neu2 is substituted by aspartic acid (G107D); (m) the glutamine residue at a position corresponding to position 108 of wild-type human Neu2 is substituted by histidine (Q108H); (n) the glutamine residue at a position corresponding to position 112 of wild-type human Neu2 is substituted by arginine (Q112R) or lysine (Q112K); (o) the cysteine residue at a position corresponding to position 125 of wild-type human Neu2 is substituted by leucine (C125L); (p) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y); (q) the alanine residue at a position corresponding to position 150 of wild-type human Neu2 is substituted by valine (A150V); (r) the cysteine residue at a position corresponding to position 164 of wild-type human Neu2 is substituted by glycine (C164G); (s) the arginine residue at a position corresponding to position 170 of wild-type human Neu2 is substituted by proline (R170P); (t) the alanine residue at a position corresponding to position 171 of wild-type human Neu2 is substituted by glycine (A171G); (u) the glutamine residue at a position corresponding to position 188 of wild-type human Neu2 is substituted by proline (Q188P); (v) the arginine residue at a position corresponding to position 189 of wild-type human Neu2 is substituted by proline (R189P); (w) the alanine residue at a position corresponding to position 213 of wild-type human Neu2 is substituted by cysteine (A213C), asparagine (A213N), serine (A213S), or threonine (A213T); (x) the leucine residue at a position corresponding to position 217 of wild-type human Neu2 is substituted by alanine (L217A) or valine (L217V); (y) the threonine residue at a position corresponding to position 249 of wild-type human Neu2 is substituted by alanine (T249A); (z) the aspartic acid residue at a position corresponding to position 251 of wild-type human Neu2 is substituted by glycine (D251G); (aa) the glutamic acid residue at a position corresponding to position 225 of wild-type human Neu2 is substituted by proline (E225P); (bb) the histidine residue at a position corresponding to position 239 of wild-type human Neu2 is substituted by proline (H239P); (cc) the leucine residue at a position corresponding to position 240 of wild-type human Neu2 is substituted by aspartic acid (L240D), asparagine (L240N), or tyrosine (L240Y); (dd) the arginine residue at a position corresponding to position 241 of wild-type human Neu2 is substituted by alanine (R241A), aspartic acid (R241D), leucine (R241L), glutamine (R241Q). or tyrosine (R241Y); (ee) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ff) the valine residue at a position corresponding to position 244 of wild-type human Neu2 is substituted by isoleucine (V244I), lysine (V244K), or proline (V244P); (gg) the glutamic acid residue at a position corresponding to position 257 of wild-type human Neu2 is substituted by proline (E257P); (hh) the serine residue at a position corresponding to position 258 is substituted by cysteine (S258C); (ii) the leucine residue at a position corresponding to position 260 of wild-type human Neu2 is substituted by aspartic acid (L260D), phenylalanine (L260F), glutamine (L260Q), or threonine (L260T); (jj) the valine residue at a position corresponding to position 265 of wild-type human Neu2 is substituted by phenylalanine (V265F); (kk) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T); (ll) the tryptophan residue at a position corresponding to position 292 of wild-type human Neu2 is substituted by arginine (W292R); (mm) the serine residue at a position corresponding to position 301 of wild-type human Neu2 is substituted by alanine (S301A), aspartic acid (S301D), glutamic acid (S301E), phenylalanine (S301F), histidine (S301H), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y)); (nn) the tryptophan residue at a position corresponding to position 302 of wild-type human Neu2 is substituted by alanine (W302A), aspartic acid (W302D), phenylalanine (W302F), glycine (W302G), histidine (W302H), isoleucine (W302I), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y); (oo) the valine residue at a position corresponding to position 363 of wild-type human Neu2 is substituted by arginine (V363R); or (pp) the leucine residue at a position corresponding to position 365 of wild-type human Neu2 is substituted by glutamine (L365Q), histidine (L365H), isoleucine (L365I), lysine (L365K) or serine (L365S); or the sialidase comprises a combination of any of the foregoing substitutions. For example, the sialidase may comprise a substitution selected from K9D, A42R, P62G, P62N, P62S, P62T, A93E, Q126Y, A242F, A242W, A242Y, Q270A, Q270T, S301A, S301R, W302K, W302R, V363R, and L365I, or a combination of any of the foregoing substitutions.

In certain embodiments of any of the foregoing sialidases, the sialidase further comprises: (a) a substitution or deletion of a methionine residue at a position corresponding to position 1 of wild-type human Neu2 (M1); (b) a substitution of a valine residue at a position corresponding to position 6 of wild-type human Neu2 (V6); (c) a substitution of an isoleucine residue at a position corresponding to position 187 of wild-type human Neu2 (I187); or (d) a substitution of a cysteine residue at a position corresponding to position 332 of wild-type human Neu2 (C332); or a combination of any of the foregoing substitutions.

In certain embodiments, in the sialidase: (a) the methionine residue at a position corresponding to position 1 of wild-type human Neu2 is deleted (ΔM1), is substituted by alanine (M1A), or is substituted by aspartic acid (M1D); (b) the valine residue at a position corresponding to position 6 of wild-type human Neu2 is substituted by tyrosine (V6Y); (c) the isoleucine residue at a position corresponding to position 187 of wild-type human Neu2 is substituted by lysine (I187K); or (d) the cysteine residue at a position corresponding to position 332 of wild-type human Neu2 is substituted by alanine (C332A); or the sialidase comprises a combination of any of the foregoing substitutions.

In certain embodiments of any of the foregoing sialidases, the sialidase comprises: (a) the M1D, V6Y, P62G, A93E, I187K, and C332A substitutions; (b) the M1D, V6Y, K9D, A93E, I187K, C332A, V363R, and L365I substitutions; (c) the M1D, V6Y, P62N, I187K, and C332A substitutions; (d) the M1D, V6Y, I187K, Q270A, S301R, W302K, and C332A substitutions; (e) the M1D, V6Y, P62S, I187K, Q270A, S301R, W302K, and C332A substitutions; (f) the M1D, V6Y, P62T, I187K, Q270A, S301R, W302K, and C332A substitutions; (g) the M1D, V6Y, P62N, I187K, Q270A, S301R, W302K, and C332A substitutions; (h) the M1D, V6Y, P62G, A93E, I187K, S301A, W302R, and C332A substitutions; (i) the M1D, V6Y, P62G, A93E, Q126Y, I187K, Q270T, and C332A substitutions; (j) the M1D, V6Y, P62G, A93E, Q126Y, I187K, and C332A substitutions; (k) the M1D, V6Y, P62G, A93E, Q126Y, I187K, A242F, Q270T, and C332A substitutions; or (l) the M1D, V6Y, A42R, P62G, A93E, Q126Y, I187K, A242F, Q270T, and C332A substitutions.

In certain embodiments of any of the foregoing sialidases, the sialidase has a different substrate specificity than the corresponding wild-type sialidase. For example, in certain embodiments the sialidase can cleave α2,3, α2,6, and/or α2,8 linkages. In certain embodiments the sialidase can cleave α2,3 and α2,8 linkages.

In certain embodiments of any of the foregoing sialidases, the sialidase comprises any one of SEQ ID NOs: 48-54, 149, 154, 159, 191, or 198, or an amino acid sequence that has at least 85%, 90%, 95%, 96%, 97%, 98%, or 99% sequence identity to any one of SEQ ID NOs: 48-54, 149, 154, 159, 191, or 198.

In addition, the invention provides a recombinant mutant human sialidase comprising a mutation or combination of mutations set forth in any one of TABLES 1, 2, 7-9, 11-13, 15-30, 34, or 35, hereinbelow. In certain embodiments, the sialidase further comprises a mutation or combination of mutations set forth in any one of TABLES 3-6, hereinbelow.

In addition, the invention provides a fusion protein comprising (or consisting essentially of): (a) a recombinant mutant human sialidase disclosed herein; and (b) an immunoglobulin Fc domain and/or an immunoglobulin antigen-binding domain; wherein the sialidase and the Fc domain and/or the antigen-binding domain are linked by a peptide bond or an amino acid linker. In certain embodiments, the fusion protein further comprises a linker, for example, an amino acid linker, connecting the sialidase enzyme and the Fc domain and/or the antigen-binding domain. In certain embodiments, the immunoglobulin antigen-binding domain is associated (for example, covalently or non-covalently associated) with a second immunoglobulin antigen-binding domain to produce an antigen-binding site.

In certain embodiments, the immunoglobulin Fc domain is derived from a human IgG1, IgG2, IgG3, IgG4, IgA1, IgA2, IgD, IgE, or IgM Fc domain, e.g., the immunoglobulin Fc domain is derived from a human IgG1, IgG2, IgG3, or IgG4 Fc domain, e.g., the immunoglobulin Fc domain is derived from a human IgG1 Fc domain.

In certain embodiments, the immunoglobulin antigen-binding domain is derived from an antibody selected from trastuzumab, daratumumab, girentuximab, ofatumumab, avelumab and rituximab.

In certain embodiments, the fusion protein comprises any one of SEQ ID NOs: 203-210, 218, 220, 222, 224, 226, 228, 230, 232, 234, 236, 238, 240, or 242, or an amino acid sequence that has at least 85%, 90%, 95%, 96%, 97%, 98%, or 99% sequence identity to any one of SEQ ID NOs: 203-210, 218, 220, 222, 224, 226, 228, 230, 232, 234, 236, 238, 240, or 242.

In addition, the invention provides an antibody conjugate comprising any of the foregoing fusion proteins. In certain embodiments, the antibody conjugate comprises a single sialidase. In other embodiments, the antibody conjugate comprises two sialidases, which can be the same or different. In certain embodiments the antibody conjugate comprises two identical sialidases. In certain embodiments, the antibody conjugate comprises a single antigen-binding site. In other embodiments, the antibody conjugate comprises two antigen-binding sites, which can be the same or different. In certain embodiments, the antibody conjugate comprises two identical antigen-binding sites.

In certain embodiments, the antibody conjugate has a molecular weight from about 135 kDa to about 165 kDa, or the antibody conjugate has a molecular weight from about 215 kDa to about 245 kDa.

In certain embodiments, the antibody conjugate comprises: (a) a first polypeptide comprising an immunoglobulin light chain; (b) a second polypeptide comprising an immunoglobulin heavy chain; and (c) a third polypeptide comprising an immunoglobulin Fc domain and a sialidase; wherein the first and second polypeptides are covalently linked together and the second and third polypeptides are covalently linked together, and wherein the first polypeptide and the second polypeptide together define an antigen-binding site. The third polypeptide may, for example, comprise the sialidase and the immunoglobulin Fc domain in an N- to C-terminal orientation.

In certain embodiments, the antibody conjugate comprises: (a) a first polypeptide comprising a first immunoglobulin light chain; (b) a second polypeptide comprising a first immunoglobulin heavy chain and a first sialidase; (c) a third polypeptide comprising a second immunoglobulin heavy chain and a second sialidase; and (d) a fourth polypeptide comprising a second immunoglobulin light chain; wherein the first and second polypeptides are covalently linked together, the third and fourth polypeptides are covalently linked together, and the second and third polypeptides are covalently linked together, and wherein the first polypeptide and the second polypeptide together define a first antigen-binding site, and the third polypeptide and the fourth polypeptide together define a second antigen-binding site. The second and third polypeptides may, for example, comprise the first and second immunoglobulin heavy chain and the first and second sialidase, respectively, in an N- to C-terminal orientation.

In certain embodiments, the antibody conjugate comprises: (a) a first polypeptide comprising a first sialidase, a first immunoglobulin Fc domain, and a first single chain variable fragment (scFv); and (b) a second polypeptide comprising a second sialidase, a second immunoglobulin Fc domain, and an optional second single chain variable fragment (scFv); wherein the first and second polypeptides are covalently linked together, and wherein the first scFv defines a first antigen-binding site, and the second scFv, when present, defines a second antigen-binding site. The first polypeptide may, for example comprise the first sialidase, the first immunoglobulin Fc domain, and the first scFv in an N- to C-terminal orientation. The second polypeptide may, for example, comprise the second sialidase, the second immunoglobulin Fc domain, and the optional second scFv in an N- to C-terminal orientation.

In certain embodiments, the antibody conjugate comprises: (a) a first polypeptide comprising an immunoglobulin light chain; (b) a second polypeptide comprising an immunoglobulin heavy chain and a single chain variable fragment (scFv); and (c) a third polypeptide comprising an immunoglobulin Fc domain and a sialidase, wherein the first and second polypeptides are covalently linked together and the second and third polypeptides are covalently linked together, and wherein the immunoglobulin light chain and immunoglobulin heavy chain together define a first antigen-binding site and the scFv defines a second antigen-binding site. The second polypeptide may, for example comprise the immunoglobulin heavy chain and the scFv in an N- to C-terminal orientation. The third polypeptide may, for example, comprise the sialidase and the immunoglobulin Fc domain in an N- to C-terminal orientation.

In another aspect, the invention provides an isolated nucleic acid comprising a nucleotide sequence encoding any of the foregoing recombinant mutant human sialidases, any of the foregoing fusion proteins, or at least a portion of any of the foregoing antibody conjugates. In another aspect, the invention provides an expression vector comprising any of the foregoing nucleic acids. In another aspect, the invention provides a host cell comprising any of the foregoing expression vectors.

In another aspect, the invention provides a pharmaceutical composition comprising any of the foregoing recombinant mutant human sialidases, any of the foregoing fusion proteins, or any of the foregoing antibody conjugates.

In another aspect, the invention provides a method of treating cancer in a subject in need thereof. The method comprises administering to the subject an effective amount of any of the foregoing sialidases, any of the foregoing fusion proteins, any of the foregoing antibody conjugates, or any of the foregoing pharmaceutical compositions. In certain embodiments, the cancer is an epithelial cancer. In certain embodiments, the cancer is a solid tumor, soft tissue tumor, hematopoietic tumor or metastatic lesion. In certain embodiments, the solid tumor is a sarcoma, adenocarcinoma, or carcinoma. In certain embodiments, the solid tumor is a head and neck (e.g., pharynx), thyroid, lung (e.g., small cell or non-small cell lung carcinoma (NSCLC)), breast, lymphoid, gastrointestinal (e.g., oral, esophageal, stomach, liver, pancreas, small intestine, colon and rectum, anal canal), genital or genitourinary tract (e.g., renal, urothelial, bladder, ovarian, uterine, cervical, endometrial, prostate, testicular), CNS (e.g., neural or glial cell, e.g., neuroblastoma or glioma), or skin (e.g., melanoma) tumor. In certain embodiments, the hematopoietic tumor is a leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphoma (DLBCL), follicular lymphoma, hairy cell leukemia, myelodyplastic syndrome (MDS), lymphoma, Hodgkin's disease, malignant lymphoma, non-Hodgkin's lymphoma, Burkitt's lymphoma, multiple myeloma, or Richter's Syndrome (Richter's Transformation). In certain embodiments, the cancer is selected from an endometrial cancer, ovarian cancer, cervical cancer, vulvar cancer, uterine cancer, fallopian tube cancer, breast cancer, prostate cancer, lung cancer, pancreatic cancer, urinary cancer, bladder cancer, head and neck cancer, oral cancer and liver cancer.

In another aspect, the invention provides a method of increasing expression of HLA-DR, CD86, CD83, IFNγ, IL-1b, IL-6, TNFα, IL-17A, IL-2, or IL-6 in a cell, tissue, or subject. The method comprises contacting the cell, tissue, or subject with an effective amount of any of the foregoing sialidases, any of the foregoing fusion proteins, any of the foregoing antibody conjugates, or any of the foregoing pharmaceutical compositions. In certain embodiments, the cell is selected from a dendritic cell and a peripheral blood mononuclear cell (PBMC).

These and other aspects and features of the invention are described in the following detailed description and claims.

### DESCRIPTION OF THE DRAWINGS

The invention can be more completely understood with reference to the following drawings.

FIG. 1 depicts an SDS-PAGE gel showing recombinant human Neu1, Neu2, Neu3, and Salmonella typhimurium (St-sialidase) under non-reducing and reducing conditions. Monomer and dimer species are indicated.

FIG. 2 is a bar graph showing the enzymatic activity of recombinant human Neu1, Neu2, and Neu3.

FIG. 3 is a line graph showing enzymatic activity as a function of substrate concentration for recombinant human Neu2 and Neu3 at the indicated pH.

FIG. 4 depicts a schematic representation of an exemplary sialic acid biotinylated probe that can be used in phage display or yeast display screening for Neu2 variants.

FIG. 5 depicts an exemplary protocol that facilitates phage display screening of Neu2 variants.

FIG. 6 depicts an exemplary protocol that facilitates yeast display screening of Neu2 variants.

FIG. 7A depicts an SDS-PAGE gel showing recombinant Neu2-Fc (wildtype) and Neu2-M106-Fc under non-reducing and reducing conditions. FIG. 7B is an SEC-HPLC trace of Neu2-Fc (wildtype) and Neu2-M106-Fc. The monomer species has a retention time of 21 minutes.

FIG. 8 is a line graph depicting the enzymatic activity of Neu2 variant M106.

FIGS. 9A-9I depict schematic representations of certain antibody conjugate constructs containing a sialidase enzyme, e.g., a human sialidase enzyme, and an antigen binding site. For each antibody conjugate construct that contains more than one (e.g., two) sialidase, each sialidase may be the same or different. For each antibody conjugate construct that contains more than one (e.g., two) antigen binding site, each antigen binding site may be the same or different. For each antibody conjugate construct that contains an Fc domain, it is understood that the Fc domain can be a wild type Fc domain or can be an engineered Fc domain. For example, the Fc domain may be engineered to contain either a “knob” mutation, e.g., T366Y, or a “hole” mutation, e.g., Y407T, or both, to promote heterodimerization, or the Fc domain may be engineered to contain one or more modifications, e.g., point mutations, to provide any other modified Fc domain functionality.

FIG. 10 depicts schematic representations of certain antibody conjugate constructs containing a sialidase enzyme, e.g., a human sialidase enzyme, and an antigen binding site. For each antibody conjugate construct that contains more than one (e.g., two) antigen binding site, each antigen binding site may be the same or different. For each antibody conjugate construct that contains an Fc domain, it is understood that the Fc domain can be a wild type Fc domain or can be an engineered Fc domain. For example, the Fc domain may be engineered to contain either a “knob” mutation, e.g., T366Y, or a “hole” mutation, e.g., Y407T, or both, to promote heterodimerization, or the Fc domain may be engineered to contain one or more modifications, e.g., point mutations, to provide any other modified Fc domain functionality.

FIGS. 11A-11E are schematic representations of exemplary fusion protein conjugates referred to as a Raptor antibody sialidase conjugate (FIG. 11A), a Janus antibody sialidase conjugate (FIG. 11B), a Lobster antibody sialidase conjugate (FIG. 11C), a Bunk antibody sialidase conjugate (FIG. 11D), and a Lobster-Fab antibody sialidase conjugate (FIG. 11E).

FIG. 12 depicts an SDS-PAGE gel showing purified recombinant human Janus Trastuzumab under non-reducing and reducing conditions.

FIG. 13 depicts an SEC-HPLC trace of purified Janus Trastuzumab, showing approximately 90% monomer purity.

FIG. 14 depicts the enzyme activity of Janus Trastuzumab assayed using 4-MU-Neu5Ac as a substrate.

FIG. 15 depicts binding to HER2 antigen as determined by ForteBio Octet for Janus Trastuzumab (top), and trastuzumab (bottom). Equilibrium dissociation constants (KD) are indicated.

FIGS. 16A-D depict the testing of various configurations of antibody sialidase conjugates in a mouse syngeneic tumor model utilizing EMT6 mouse breast cancer cells engineered to express human HER2. Mice are treated via intraperitoneal injection of 10 mg/kg of each test article on the days marked with black triangles and tumor volume (mm³) recorded. Each line represents an individual mouse. Mice are treated with either trastuzumab (FIG. 16A), Raptor (FIG. 16B), Janus (FIG. 16C) or Lobster (FIG. 16D).

FIGS. 17A-D depict the testing of the Janus antibody sialidase conjugate in a mouse syngeneic tumor model utilizing EMT6 mouse breast cancer cells engineered to express human HER2. Mice were treated via intraperitoneal injection of 10 mg/kg of Janus on the days marked with black triangles and tumor volume (mm³) recorded. Mice were also treated on the same days as Janus with either anti-mouse NK1.1 (10 mg/kg) to deplete natural killer cells (FIG. 17A), liposomal clodronate (0.5 mg/mouse, three times a week for two weeks) to deplete macrophages (FIG. 17B), or anti-mouse CD8a (10 mg/kg) to deplete CD8+ T cells (FIG. 17C). Each line represents an individual mouse. FIG. 17D depicts the mean tumor volume with error bars of the indicated treatment groups from Example 5.

FIGS. 18A-B depict the testing of the Janus antibody sialidase conjugate in a mouse syngeneic orthotopic tumor model utilizing a second source of EMT6 mouse breast cancer cells engineered to express human HER2. Mice are treated via intraperitoneal injection of 10 mg/kg of each test article on the days marked with black triangles and tumor volume (mm³) recorded. Each line represents an individual mouse. Mice are treated (Y) with either vehicle, trastuzumab, Janus or Janus Loss of Function (FIG. 18A). FIG. 18B depicts the rechallenge experiment of either the three mice treated with Janus from FIG. 18A with complete regressions of the original EMT6-HER2 tumors (cured mice) or naïve mice. Cured mice were inoculated with either EMT6-HER2 cells or parental EMT6 cells on the left and right lower flank region. Naïve mice were inoculated with EMT6-HER2 cells.

FIGS. 19A-B depict the testing of the Janus antibody sialidase conjugate in a mouse syngeneic orthotopic tumor model in combination with anti-mouse PD1. Mice are treated via intraperitoneal injection of 10 mg/kg of either anti-mouse PD1 alone (FIG. 19A) or Janus and anti-mouse PD1 (10 mg/kg of each, FIG. 19B) on the days marked with black triangles (▾) and tumor volume (mm³) recorded. Each line represents an individual mouse.

FIG. 20 depicts the testing of various test articles in a mouse syngeneic tumor model injected with a B16 melanoma cell line expressing human HER2. Mice are treated via intraperitoneal injection of 10 mg/kg of either Janus, trastuzumab or a combination of anti-mouse PD1 and anti-mouse CTLA4 (10 mg/kg of each) on the days marked with black triangles (▾) and tumor volume (mm³) recorded. Each line represents an individual mouse.

FIG. 21 depicts the testing of Janus Trastuzumab in a mouse syngeneic tumor model utilizing EMT6 mouse breast cancer cells engineered to express human HER2. Each line represents an individual mouse. Tick marks indicate dosing frequency (total of 5 doses, biweekly). Mice are treated with either Janus Trastuzumab or isotype control.

FIG. 22A depicts an SDS-PAGE gel showing Neu2-M173-Fc under non-reducing and reducing conditions. FIG. 22B is an SEC-HPLC trace of Neu2-M173-Fc. The monomer species has a retention time of 6.367 minutes. The monomer species has a purity of approximately 90% after purification by Protein A and CHT chromatography.

FIG. 23 depicts the enzyme activity of Neu2-M173-Fc, using 4-MU-Neu5Ac as the substrate, and fixing enzyme concentration to 2 μg/well.

FIG. 24A depicts an SDS-PAGE gel showing Neu2-M106 under non-reducing (NR) and reducing (R) conditions. FIG. 24B depicts a schematic representation of the Neu2 structure with the position of the R243 cleavage site indicated.

FIG. 25 depicts a reducing SDS-PAGE gel showing Neu2-M106 produced by a large or small scale expression with (+) or without (−) trypsin treatment.

FIG. 26 depicts an SDS-PAGE gel showing Neu2-M106 following incubation with trypsin and one of the protease inhibitors iron citrate (Fe Cit), aprotinin, AEBSF, leupeptin, or E-64 at the indicated concentrations.

FIG. 27A is a table depicting a sequence alignment of different sialidase sequences, showing conservation of the P1 arginine, which is a protease cleavage site.

FIG. 27B is a chart showing different mutations and combinations of mutations surrounding the trypsin cleavage site in Neu2.

FIG. 28A depicts a reducing SDS-PAGE analysis of Neu2 variants with the indicated mutation at position A242 with or without trypsin treatment. Trypsin digestion was for 5 minutes at 4° C. using a 5,000% dilution of trypsin. The digestion was quenched by addition of SDS, and 2 μg of protein was loaded on the gel. FIG. 28B depicts the enzymatic activity of Neu2 variants with the indicated mutation at position A242. FIG. 28C is an SEC-HPLC trace of Neu2 variants with the indicated mutation at position A242. Neu2-M106 (the mutational background in which the mutations at position A242 were tested) is shown as a control.

FIG. 29 depicts a reducing SDS-PAGE analysis of the indicated Neu2 variants with or without trypsin treatment. Neu2-M106 is shown as a control. For example, Neu2-M255 was shown to have a greater than 10 fold improved trypsin resistance relative to Neu2-M106.

FIG. 30A depicts an SDS-PAGE gel showing recombinant Neu2-M259-Fc under non-reducing and reducing conditions. FIG. 30B is an SEC-HPLC trace of Neu2-M259-Fc.

FIG. 31A is a line graph showing enzymatic activity of the indicated Neu2-Fc variants as a function of substrate (4-MU-Neu5Ac) concentration. FIG. 31B is a line graph showing enzymatic activity of the indicated Neu2-Fc variants as a function of enzyme concentration.

FIG. 32 is a line graph depicting thermal stability of the indicated Neu2-Fc variants.

FIG. 33 depicts testing of a Neu2-Fc fusion protein in a mouse syngeneic subcutaneous tumor model. Mean tumor volumes over 21 days for the indicated treatments are indicated in FIG. 33A. Triangles indicate dosing. Individual tumor volumes on day 21 are depicted in FIG. 33B.

FIG. 34 is a bar graph depicting enzymatic activity of the indicated Neu2-Fc variants following incubation at 37° C. for the indicated length of time.

FIG. 35 is an SEC-HPLC trace of Janus Trastuzumab 2.

FIG. 36 depicts binding to HER2 antigen as determined by ForteBio Octet for Janus Trastuzumab 2.

FIG. 37 depicts the testing of Janus Trastuzumab 2 in a mouse syngeneic tumor model utilizing EMT6 mouse breast cancer cells engineered to express human HER2, where tumor volume was measured after treatment with an isotype control (FIG. 37A), 1 mg/kg Trastuzumab (FIG. 37B), 10 mg/kg Trastuzumab (FIG. 37C), 1 mg/kg Janus Trastuzumab 2 (FIG. 37D), or 10 mg/kg Janus Trastuzumab 2 (FIG. 37E), where each line represents an individual mouse, and in FIG. 37F, each line represents the mean tumor volume for the indicated treatment group. Triangles indicate dosing frequency. In FIGS. 37A-E, Complete Responses (CR) and Partial Responses (PR) are indicated.

Various features and aspects of the invention are discussed in more detail below.

### DETAILED DESCRIPTION

The invention relates to a recombinant sialidase (e.g., a recombinant human sialidase) that comprises at least one mutation relative to a wild-type sialidase, e.g., a substitution, deletion, or addition (insertion) of at least one amino acid. The mutation may, for example, enhance resistance (decrease sensitivity) to protease (e.g., trypsin) degradation. Alternatively or in addition, the sialidase may include a mutation, or a combination of mutations, that can improve the expression, activity or both the expression and activity of the sialidase.

The invention further provides fusion proteins and/or antibody conjugates comprising a mutant sialidase enzyme and an antibody or portion thereof, e.g., an immunoglobulin Fc domain and/or an antigen-binding domain. The sialidase enzyme portion of the fusion protein and/or antibody conjugate comprises at least one mutation relative to a wild-type sialidase (for example, a mutation that increases resistance to protease (e.g., trypsin) degradation). The sialidase can also include one or more of the other mutations described herein that improve expression and/or activity of the sialidase.

The invention further relates to pharmaceutical compositions and methods of using fusion proteins and/or antibody conjugates to treat cancer, e.g., a solid tumor, soft tissue tumor, hematopoietic tumor, metastatic lesion, or an epithelial cell cancer.

### I. Recombinant Human Sialidases

As used herein, the term “sialidase” refers to any enzyme, or a functional fragment thereof, that cleaves a terminal sialic acid residue from a substrate, for example, a glycoprotein or a glycolipid. The term sialidase includes variants having one or more amino acid substitutions, deletions, or insertions relative to a wild-type sialidase sequence, and/or fusion proteins or conjugates including a sialidase. Sialidases are also called neuraminidases, and, unless indicated otherwise, the two terms are used interchangeably herein. As used herein, the term “functional fragment” of a sialidase refers to fragment of a full-length sialidase that retains, for example, at least 10%, at least 20%, at least 30%, at least 40%, at least 50%, at least 60%, at least 70%, at least 80%, at least 90%, at least 95%, or 100% of the enzymatic activity of the corresponding full-length, naturally occurring sialidase. Sialidase enzymatic activity may be assayed by any method known in the art, including, for example, by measuring the release of sialic acid from the fluorogenic substrate 4-methylumbelliferyl-N-acetylneuraminic acid (4MU-NeuAc). In certain embodiments, the functional fragment comprises at least 100, 150, 200, 250, 300, 310, 320, 330, 340, 350, 360, or 370 consecutive amino acids present in a full-length, naturally occurring sialidase.

Four sialidases have been found in the human genome and are referred to as Neu1, Neu2, Neu3 and Neu4.

Human Neu1 is a lysosomal neuraminidase enzyme which functions in a complex with beta-galactosidase and cathepsin A. The amino acid sequence of human Neu1 is depicted in SEQ ID NO: 7, and a nucleotide sequence encoding human Neu1 is depicted in SEQ ID NO: 23.

Human Neu2 is a cytosolic sialidase enzyme. The amino acid sequence of human Neu2 is depicted in SEQ ID NO: 1, and a nucleotide sequence encoding human Neu2 is depicted in SEQ ID NO: 24. Unless stated otherwise, as used herein, wild-type human Neu2 refers to human Neu2 having the amino acid sequence of SEQ ID NO: 1.

Human Neu3 is a plasma membrane sialidase with an activity specific for gangliosides. Human Neu3 has two isoforms: isoform 1 and isoform 2. The amino acid sequence of human Neu3, isoform 1 is depicted in SEQ ID NO: 8, and a nucleotide sequence encoding human Neu3, isoform 1 is depicted in SEQ ID NO: 25. The amino acid sequence of human Neu3, isoform 2 is depicted in SEQ ID NO: 9, and a nucleotide sequence encoding human Neu3, isoform 2 is depicted in SEQ ID NO: 34.

Human Neu4 has two isoforms: isoform 1 is a peripheral membrane protein and isoform 2 localizes to the lysosome lumen. The amino acid sequence of human Neu4, isoform 1 is depicted in SEQ ID NO: 10, and a nucleotide sequence encoding human Neu4, isoform 1 is depicted in SEQ ID NO: 26. The amino acid sequence of human Neu4, isoform 2 is depicted in SEQ ID NO: 11, and a nucleotide sequence encoding human Neu4, isoform 2 is depicted in SEQ ID NO: 35.

Four sialidases have also been found in the mouse genome and are referred to as Neu1, Neu2, Neu3 and Neu4. The amino acid sequence of mouse Neu1 is depicted in SEQ ID NO: 38, and a nucleotide sequence encoding mouse Neu1 is depicted in SEQ ID NO: 42. The amino acid sequence of mouse Neu2 is depicted in SEQ ID NO: 39 and a nucleotide sequence encoding mouse Neu2 is depicted in SEQ ID NO: 43. The amino acid sequence of mouse Neu3 is depicted in SEQ ID NO: 40, and a nucleotide sequence encoding mouse Neu3 is depicted in SEQ ID NO: 44. The amino acid sequence of mouse Neu4 is depicted in SEQ ID NO: 41, and a nucleotide sequence encoding mouse Neu4 is depicted in SEQ ID NO: 45.

Exemplary prokaryotic sialidases include sialidases from Salmonella typhimurium and Vibrio cholera. The amino acid sequence of Salmonella typhimurium sialidase (St-sialidase) is depicted in SEQ ID NO: 30, and a nucleotide sequence encoding Salmonella typhimurium sialidase is depicted in SEQ ID NO: 6. The amino acid sequence of Vibrio cholera sialidase is depicted in SEQ ID NO: 36, and a nucleotide sequence encoding Vibrio cholera sialidase is depicted in SEQ ID NO: 37.

In certain embodiments, a recombinant mutant sialidase (e.g., human sialidase) has at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, about 100%, or more than 100% of the enzymatic activity of a corresponding (or template) wild-type sialidase (e.g., human sialidase).

In certain embodiments, a recombinant mutant sialidase (e.g., human sialidase) has about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more than 100% of the enzymatic activity of a corresponding (or template) wild-type sialidase (e.g., human sialidase).

In certain embodiments, the recombinant mutant sialidase (e.g., human sialidase) has the same substrate specificity as the corresponding wild-type sialidase (e.g., human sialidase). In other embodiments, the recombinant mutant sialidase (e.g., human sialidase) has a different substrate specificity than the corresponding wild-type sialidase (e.g., human sialidase). For example, in certain embodiments the recombinant mutant human sialidase can cleave α2,3, α2,6, and/or α2,8 linkages. In certain embodiments the sialidase can cleave α2,3 and α2,8 linkages.

In certain embodiments, the expression yield of the recombinant mutant sialidase (e.g., human sialidase) sialidase in mammalian cells, e.g., HEK293 cells, CHO cells, murine myeloma cells (e.g., NS0, Sp2/0), or human fibrosarcoma cells (e.g., HT-1080), is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of the corresponding wild-type sialidase (e.g., human sialidase).

In certain embodiments, the recombinant mutant sialidase (e.g., human sialidase) has about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more than 100% of the enzymatic activity of a corresponding wild-type sialidase (e.g., human sialidase), and the expression yield of the recombinant mutant sialidase (e.g., human sialidase) in mammalian cells, e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of a corresponding sialidase (e.g., human sialidase) human sialidase.

In certain embodiments, the amino acid sequence of the recombinant mutant sialidase (e.g., human sialidase) has at least 50%, 55%, 60%, 65%, 70%, 75%, 80%, 85%, 90%, 95%, 96%, 97%, 98%, or 99% sequence identity to the amino acid sequence of a corresponding wild-type sialidase (e.g., human sialidase).

a. Substitutions of Residues to Decrease Proteolytic Cleavage

It has been discovered that certain sialidases (e.g., human Neu2) are susceptible to cleavage by a protease (e.g., trypsin). As a result, proteolytic cleavage of the sialidase may occur during recombinant protein production, harvesting, purification, or formulation, during administration to a subject, or after administration to a subject. Accordingly, in certain embodiments, the recombinant mutant sialidase (e.g., human sialidase) comprises a substitution of at least one wild-type amino acid residue, wherein the substitution decreases cleavage of the sialidase by a protease (e.g., trypsin) relative to a sialidase without the substitution.

In certain embodiments, the protease is a trypsin (e.g., a mammalian trypsin, a bovine trypsin, a human trypsin such as trypsin 1, trypsin 2, or mesotrypsin, a cod trypsin, a Streptomyces griseus trypsin, a Saccharopolyspora erythraeus trypsin, a Streptomyces exfoliatus trypsin, and a Streptomyces albidoflavus trypsin), α-lytic protease, or a serine protease such as kallikreins, elastase and chymotrypsin.

In certain embodiments, incubation of the recombinant mutant sialidase (e.g., human sialidase) with a protease (e.g., trypsin) results in from about 1% to about 50%, from about 1% to about 40%, from about 1%, to about 30%, from about 1% to about 20%, from about 1% to about 10%, from about 1% to about 5%, from about 5% to about 50%, from about 5% to about 40%, from about 5% to about 30%, from about 5% to about 20%, from about 5% to about 10%, from about 10% to about 50%, from about 10% to about 40%, from about 10% to about 30%, from about 10% to about 20%, from about 20% to about 50%, from about 20% to about 40%, from about 20% to about 30%, from about 30% to about 50%, from about 30% to about 40%, or from about 40% to about 50% of the proteolytic cleavage of a corresponding sialidase (e.g., wild-type sialidase) without the mutation when incubated with the protease under the same conditions. In certain embodiments, incubation of the recombinant mutant sialidase (e.g., human sialidase) with a protease (e.g., trypsin) results in less than 50%, less than 40%, less than 30%, less than 10%, less than 5%, less than 3%, less than 1%, or less than 0.5% of the proteolytic cleavage of a corresponding sialidase (e.g., wild-type sialidase) without the mutation(s) when incubated with the protease under the same conditions.

Proteolytic cleavage can be assayed by any method known in the art, including for example, by SDS-PAGE as described in Example 5 herein.

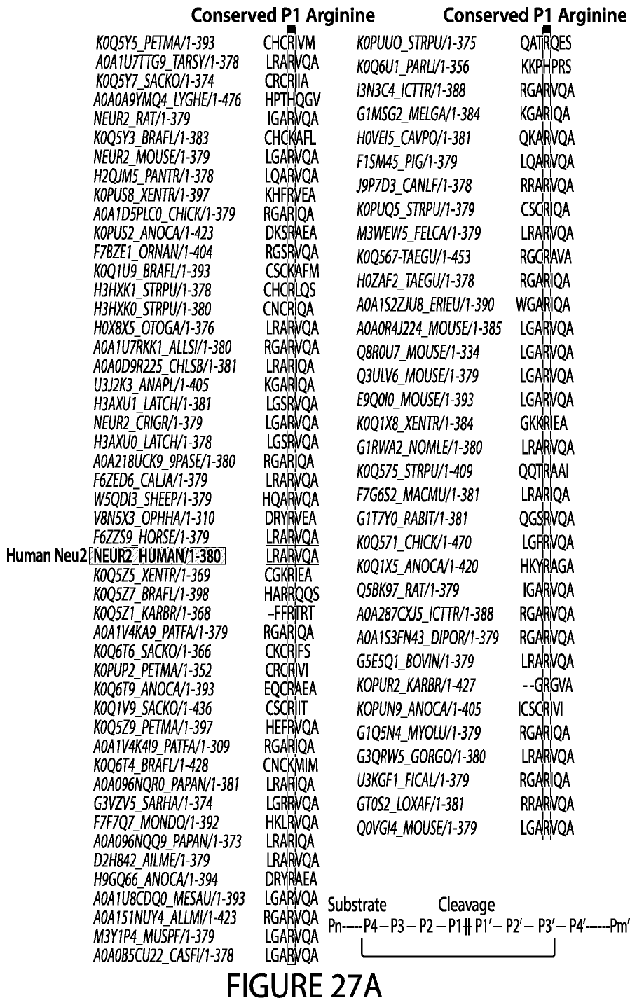

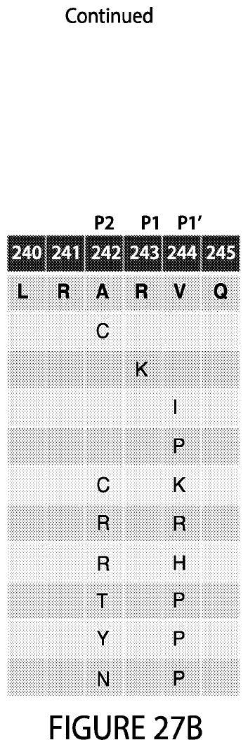

As shown in FIG. 27A, the arginine residue at a position corresponding to position 243 of wild-type human Neu2 (SEQ ID NO: 1) is conserved among many different sialidases. Accordingly, it is believed that mutating amino acids nearby this conserved arginine residue can reduce proteolytic cleavage of the sialidase. Thus, in certain embodiments, mutations within about 5 amino acid, about 4 amino acids, about 3 amino acids, or about 2 amino acids of the arginine residue at a position corresponding to position 243 of wild-type human Neu2 (SEQ ID NO: 1) can be substituted to increase resistance to proteolytic cleavage.

Exemplary substitutions that increase resistance to proteolytic cleavage include: (i) a substitution of an alanine residue at a position corresponding to position 242 of wild-type human Neu2 (SEQ ID NO: 1), e.g., a substitution by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ii) a substitution of an arginine residue at a position corresponding to position 243 of wild-type human Neu2 (SEQ ID NO: 1), e.g., a substitution by glutamic acid (R243E), histidine (R243H), asparagine (R243N), glutamine (R243Q), or lysine (R243K); (iii) a substitution of a valine residue at a position corresponding to position 244 of wild-type human Neu2 (SEQ ID NO: 1), e.g., a substitution by isoleucine (V244I), lysine (V244K), or proline (V244P); or (iv) a combination of any of the foregoing. In certain embodiments, the alanine at a position corresponding to position 242 of wild-type human Neu2 is substituted by an aromatic amino acid, e.g., tryptophan (A242W), tyrosine (A242Y), or phenylalanine (A242F). In certain embodiments, the alanine at a position corresponding to position 242 of wild-type human Neu2 is substituted by cysteine (A242C). In certain embodiments, the recombinant mutant human sialidase or the recombinant non-human sialidase comprises a substitution or combination of substitutions corresponding to a substitution or combination of substitutions listed in TABLE 1 (amino acid positions corresponding to wild-type human Neu2 (SEQ ID NO: 1)).

TABLE 1

| Wild Type | |
|---|---|
| Human Neu2 | |
| (SEQ ID NO: 1) | |
| Amino Acid | Exemplary Substitution(s) at Specified Position(s) |
| A242 | C, F, G, H, I, K, L, M, N, P, Q, R, S, V, W, Y |
| R243 | E, H, N, Q, K |
| V244 | I, K, P |

Additional exemplary substitutions that increase resistance to proteolytic cleavage (and/or increase expression yield and/or enzymatic activity) include: (i) a substitution of a leucine residue at a position corresponding to position 240 of wild-type human Neu2 (SEQ ID NO: 1), e.g., a substitution by aspartic acid (L240D), asparagine (L240N), or tyrosine (L240Y); (ii) a substitution of an alanine residue at a position corresponding to position 213 of wild-type human Neu2 (SEQ ID NO: 1), e.g., a substitution by cysteine (A213C), asparagine (A213N), serine (A213S), or threonine (A213T); (iii) a substitution of an arginine residue at a position corresponding to position 241 of wild-type human Neu2 (SEQ ID NO: 1), e.g., a substitution by alanine (R241A), aspartic acid (R241D), leucine (R241L), glutamine (R241Q). or tyrosine (R241Y); (iv) a substitution of a serine residue at a position corresponding to position 258 of wild-type human Neu2 (SEQ ID NO: 1), e.g., a substitution by cysteine (S258C); (v) a substitution of a leucine residue at a position corresponding to position 260 of wild-type human Neu2 (SEQ ID NO: 1), e.g., a substitution by aspartic acid (L260D), phenylalanine (L260F), glutamine (L260Q), or threonine (L260T); (vi) a substitution of a valine residue at a position corresponding to position 265 of wild-type human Neu2 (SEQ ID NO: 1), e.g., a substitution by phenylalanine (V265F); or (vii) a combination of any of the foregoing. It is contemplated that, in certain embodiments, a substitution or a combination of substitutions at these positions may improve hydrophobic and/or aromatic interaction between secondary structure elements in the sialidase (e.g., between an α-helix and the nearest β-sheet) thereby stabilizing the structure and improving resistance to proteolytic cleavage.

In certain embodiments, the recombinant mutant sialidase or the recombinant non-human sialidase comprises a mutation at position L240. In certain embodiments, the recombinant mutant sialidase comprises a combination of mutations at positions (i) A213 and A242, (ii) A213, A242, and S258, (iii) L240 and L260, (iv) R241 and A242, (v) A242 and L260, (vi) A242 and V265, or (vii) L240 and A242. In certain embodiments, the recombinant mutant human sialidase comprises a combination of substitutions selected from (i) A213C, A242F, and S258C, (ii) A213C and A242F, (iii) A213T and A242F, (iv) R241Y and A242F, and (v) L240Y and A242F. In certain embodiments, the recombinant mutant human sialidase or the recombinant non-human sialidase comprises a substitution or combination of substitutions corresponding to a substitution or combination of substitutions listed in TABLE 2 (amino acid positions corresponding to wild-type human Neu2 (SEQ ID NO: 1)).

TABLE 2
Substitution(s)

A242C, V244P
A242R, V244R
A242R, V244H
A242Y, V244P
A242T, V244P
A242N, V244P
A213C, A242F
A213S, A242F
A213T, A242F
A213N, A242F
A213C, A242F, S258C
A242F, L260F
A242F, V265F
L240Y
L240Y, L260F
L240D, L260T
L240N, L260T
L240N, L260D
L240N, L260Q
L240Y, A242F
R241A, A242F
R241Y, A242F

b. Substitution of Cysteine Residues

In certain embodiments, the recombinant mutant human sialidase further comprises a substitution of at least one cysteine (cys, C) residue. It has been discovered that certain cysteine residues in sialidases may inhibit expression of functional protein as a result of protein aggregation. Accordingly, in certain embodiments, the recombinant mutant human sialidase contains at least one mutation to remove a free cysteine (e.g., for Neu1 (SEQ ID NO: 7), a mutation of, for example, one or more of C111, C117, C171, C183, C218, C240, C242, and C252; for Neu2 (SEQ ID NO: 1), a mutation of, for example, one or more of C125, C196, C219, C272, C332, and C352; for Neu3 (SEQ ID NO: 8), a mutation of, for example, one or more of C7, C90, C99, C106, C127, C136, C189, C194, C226, C242, C250, C273, C279, C295, C356, C365, C368, C384, C383, C394, and C415; and for Neu4 (SEQ ID NO: 10), a mutation of, for example, one or more of C88, C125, C126, C186, C191, C211, C223, C239, C276, C437, C453, C480, and C481). Free cysteines can be substituted with any amino acid. In certain embodiments, the free cysteine is substituted with serine (ser, S), isoleucine (iso, I), valine (val, V), phenylalanine (phe, F), leucine (leu, L), or alanine (ala, A). Exemplary cysteine substitutions in Neu2 include C125A, C125I, C125S, C125V, C196A, C196L, C196V, C272S, C272V, C332A, C332S, C332V, C352L, and C352V. In certain embodiments, the cysteine at a position corresponding to position 332 of wild-type human Neu2 is substituted by a hydrophobic amino acid, e.g., alanine (C332A), valine (C332V), isoleucine (C332I), or leucine (C332L). In certain embodiments, the cysteine at a position corresponding to position 332 of wild-type human Neu2 is substituted by alanine (C332A).

In certain embodiments, the recombinant mutant human sialidase comprises two or more cysteine substitutions. Exemplary double or triple cysteine substitutions in Neu2 include: C125S and C332S; C272V and C332A; C272V and C332S; C332A and C352L; C125S and C196L; C196L and C352L; C196L and C332A; C332A and C352L; and C196L, C332A and C352L.

In certain embodiments, the recombinant mutant human sialidase is a Neu2 sialidase and comprises the substitutions C322A and C352L.

In certain embodiments, the sialidase contains an amino acid substitution at 2, 3, 4, 5, or 6 cysteines typically present in a human sialidase, e.g., Neu2 or Neu3.

In certain embodiments, the recombinant mutant human sialidase comprises a substitution or combination of substitutions corresponding to a substitution or combination of substitutions listed in TABLE 3 (amino acid positions corresponding to wild-type human Neu2 (SEQ ID NO: 1)).

TABLE 3
Substitution(s)

C125A
C125I
C125S
C125V
C196A
C196L
C196V
C272S
C272V
C332A
C332S
C332V
C352L
C352V
C125S + C332S
C272V + C332A
C272V + C332S
C332A + C352L
C125S + C196L
C196L + C352L
C196L + C332A
C196L + C332A + C352L

c. Substitutions of Residues to Increase pI and/or Decrease Hydrophobicity

In addition the sialidase (e.g., human sialidase) may further comprise one or more substitutions that modify pI (e.g., increase pI) and/or hydrophobicity (e.g., decrease hydrophobicity) of the sialidase.

The isoelectric point (pI) of a protein is the pH at which the net charge is zero. The pI also generally indicates the pH at which the protein is least soluble, which may affect the ability to express and purify the protein. Generally, a protein has good solubility if its pI is greater than 2 units above the pH of the solution. Human Neu2 has a predicted pI of 7.5. Thus, human Neu2 is least soluble around neutral pH, which is undesirable because expression and physiological systems are at neutral pH. In contrast, the sialidase from Salmonella typhimurium (St-sialidase), which exhibits good solubility and recombinant expression, has a pI of 9.6. Accordingly, to increase expression of human Neu2 or the other human sialidases, a recombinant mutant human sialidase may be designed to contain one or more amino acid substitution(s) wherein the substitution(s) increase(s) the pI of the sialidase relative to a sialidase without the substitution. Additionally, decreasing the number of hydrophobic amino acids on the surface of a sialidase may improve expression of sialidase by, for example, reducing aggregation. Accordingly, to increase expression of human Neu2 or the other human sialidases, a recombinant mutant human sialidase may be designed to contain one or more amino acid substitution(s) wherein the substitution(s) decrease(s) the hydrophobicity of a surface of the sialidase relative to a sialidase without the substitution(s).

Accordingly, in certain embodiments, the recombinant mutant human sialidase comprises at least one amino acid substitution, wherein the substitution increases the isoelectric point (pI) of the sialidase and/or decreases the hydrophobicity of the sialidase relative to a sialidase without the substitution. This may be achieved by introducing one or more charged amino acids, for example, positively or negatively charged amino acids, into the recombinant sialidase. In certain embodiments, the amino acid substitution is to a charged amino acid, for example, a positively charged amino acid such as lysine (lys, K), histidine (his, H), or arginine (arg, R), or a negatively charged amino acid such as aspartic acid (asp, D) or glutamic acid (glu, E). In certain embodiments, the amino acid substitution is to a lysine residue. In certain embodiments, the substitution increases the pI of the sialidase to about 7.75, about 8, about 8.25, about 8.5, about 8.75, about 9, about 9.25, about 9.5, or about 9.75.

In certain embodiments, the amino acid substitution occurs at a surface exposed D or E amino acid, in a helix or loop, or in a position that has a K or R in the corresponding position of St-sialidase. In certain embodiments, the amino acid substitution occurs at an amino acid that is remote from the catalytic site or otherwise not involved in catalysis, an amino acid that is not conserved with the other human Neu proteins or with St-Sialidase or Clostridium NanH, or an amino acid that is not located in a domain important for function (e.g., an Asp-box or beta strand).

Exemplary amino acid substitutions in Neu2 that increase the isoelectric point (pI) of the sialidase and/or decrease the hydrophobicity of the sialidase relative to a sialidase without the substitution include A2E, A2K, D215K, V325E, V325K, E257K, and E319K. In certain embodiments, the recombinant mutant human sialidase comprises two or more amino acid substitutions, including, for example, A2K and V325E, A2K and V325K, E257K and V325K, A2K and E257K, and E257K and A2K and V325K.

In certain embodiments, the recombinant mutant human sialidase comprises a substitution or combination of substitutions corresponding to a substitution or combination of substitutions listed in TABLE 4 (amino acid positions corresponding to wild-type human Neu2 (SEQ ID NO: 1)).

TABLE 4
Substitution(s)

A2K
E72K
D215K
E257K
V325K
A2K + E257K
A2K + V325E
A2K + V325K
E257K + V325K

d. Addition of N-terminal Peptides and N- or C-terminal Substitutions

In addition, the sialidase (e.g., human sialidase) may further comprise an N-terminal peptide and/or N- or C-terminal substitutions.

It has been discovered that the addition of a peptide sequence of two or more amino acids to the N-terminus of a human sialidase can improve expression and/or activity of the sialidase. In certain embodiments, the peptide is at least 2 amino acids in length, for example, from 2 to 20, from 2 to 10, from 2 to 5, or 2, 3, 4, 5, 6, 7, 8, 9, 10, 11, 12, 13, 14, 15, 16, 17, 18, 19, or 20 amino acids in length. In certain embodiments, the peptide may form, or have a propensity to form, an α-helix.

In mice, a Neu2 isoform (type B) found in thymus contains six amino acids not present in the canonical isoform of Neu2 found in skeletal muscle. In certain embodiments herein, the N-terminal six amino acids of the mouse thymus Neu2 isoform, MEDLRP (SEQ ID NO: 4), or variations thereof, can be added onto a human Neu, e.g., human Neu2. In certain embodiments, the recombinant mutant human sialidase comprises a peptide at least two amino acid residues in length covalently associated with an N-terminal amino acid of the sialidase. In certain embodiments the recombinant mutant human sialidase comprises the peptide MEDLRP (SEQ ID NO: 4) or EDLRP (SEQ ID NO: 3) covalently associated with an N-terminal amino acid of the sialidase. In certain embodiments, the sialidase may further comprise a cleavage site, e.g., a proteolytic cleavage site, located between the peptide, e.g., MEDLRP (SEQ ID NO: 4) or EDLRP (SEQ ID NO: 3), and the remainder of the sialidase. In certain embodiments, the peptide, e.g., MEDLRP (SEQ ID NO: 4) or EDLRP (SEQ ID NO: 3), may be post-translationally cleaved from the remainder of the sialidase.

Alternatively to, or in combination with, the N-terminal addition, 1-5 amino acids of the 12 amino acid N-terminal region of the recombinant mutant human sialidase may be removed, e.g., the N-terminal methionine can be removed. In certain embodiments, if the recombinant mutant human sialidase is Neu2, the N-terminal methionine can be removed, the first five amino acids (MASLP; SEQ ID NO: 12) can be removed, or the second through fourth amino acids (ASLP; SEQ ID NO: 13) can be removed.

In certain embodiments, 1-5 amino acids of the 12 amino acid N-terminal region of the recombinant mutant human sialidase are substituted with MEDLRP (SEQ ID NO: 4), EDLRP (SEQ ID NO: 3), or TVEKSVVF (SEQ ID NO: 14). For example, in certain embodiments, if the recombinant mutant human sialidase is Neu2, the amino acids MASLP (SEQ ID NO: 12), ASLP (SEQ ID NO: 13) or M are substituted with MEDLRP (SEQ ID NO: 4), EDLRP (SEQ ID NO: 3) or TVEKSVVF (SEQ ID NO: 14).

Human sialidases have a β-propeller structure, characterized by 6 blade-shaped β-sheets arranged toroidally around a central axis. Generally, hydrophobic interactions between the blades of a β-propeller, including between the N- and C-terminal blades, enhance stability. Accordingly, in order to increase expression of human Neu2 or the other human sialidases, a recombinant mutant human sialidase can be designed comprising an amino acid substitution that increases hydrophobic interactions and/or hydrogen bonding between the N- and C-terminal β-propeller blades of the sialidase.

Accordingly, in certain embodiments, the recombinant mutant human sialidase comprises a substitution of at least one wild-type amino acid residue, wherein the substitution increases hydrophobic interactions and/or hydrogen bonding between the N- and C-termini of the sialidase relative to a sialidase without the substitution. In certain embodiments, the wild-type amino acid is substituted with asparagine (asn, N), lysine (lys, K), tyrosine (tyr, Y), phenylalanine (phe, F), or tryptophan (trp, W). Exemplary substitutions in Neu2 that increase hydrophobic interactions and/or hydrogen bonding between the N- and C-termini include L4N, L4K, V6Y, L7N, L4N and L7N, L4N and V6Y and L7N, V12N, V12Y, V12L, V6Y, V6F, or V6W. In certain embodiments, the valine at a position corresponding to position 6 of wild-type human Neu2 is substituted by an aromatic amino acid, e.g., tryptophan (V6W), tyrosine (V6Y), or phenylalanine (V6F). In certain embodiments, the sialidase comprises the V6Y substitution.

In certain embodiments, the recombinant mutant human sialidase comprises a combination of the above substitutions. For example, a recombinant mutant human Neu2 sialidase can comprise the additional amino acids MEDLRP (SEQ ID NO: 4), EDLRP (SEQ ID NO: 3), or TVEKSVVF (SEQ ID NO: 14) at the N-terminus and, in combination, can comprise at least one L4N, L4K, V6Y, L7N, L4N and L7N, L4N and V6Y and L7N, V12N, V12Y, V12L, V6Y, V6F, or V6W substitution. In certain embodiments, the amino acids MASLP (SEQ ID NO: 12), ASLP (SEQ ID NO: 13) or M of a recombinant mutant human Neu2 sialidase are replaced with MEDLRP (SEQ ID NO: 4), EDLRP (SEQ ID NO: 3) or TVEKSVVF (SEQ ID NO: 14) and the recombinant mutant human Neu2 sialidase also comprises at least one L4N, L4K, V6Y, L7N, L4N and L7N, L4N and V6Y and L7N, V12N, V12Y, V12L, V6Y, V6F, or V6W substitution.

In certain embodiments, the recombinant mutant human sialidase comprises a mutation or combination of mutations corresponding to a mutation or combination of mutations listed in TABLE 5 (amino acid positions corresponding to wild-type human Neu2 (SEQ ID NO: 1)).

TABLE 5
Mutation(s)
Substitute M at the N-terminus with EDLRP (SEQ ID NO: 3)
Substitute M at the N-terminus with MEDLRP (SEQ ID NO: 4)
Insert MEDLRP (SEQ ID NO: 4) at the N-terminus
Substitute MASLP (SEQ ID NO: 12) at the N-terminus with
MEDLRP (SEQ ID NO: 4)
L4N
V6Y
L7N
V6F
V6W

Additionally, in certain embodiments, the sialidase comprises a substitution or deletion of an N-terminal methionine at the N-terminus of the sialidase. For example, in certain embodiments, the sialidase comprises a substitution of a methionine residue at a position corresponding to position 1 of wild-type human Neu2 (SEQ ID NO: 1). In certain embodiments, the methionine at a position corresponding to position 1 of wild-type human Neu2 is substituted by alanine (M1A). In certain embodiments, the methionine at a position corresponding to position 1 of wild-type human Neu2 is substituted by a negatively charged amino acid, e.g., glutamic acid (M1E) or aspartic acid (M1D). In certain embodiments, the methionine at a position corresponding to position 1 of wild-type human Neu2 is substituted by aspartic acid (M1D). In other embodiments, the sialidase comprises a deletion of a methionine residue at a position corresponding to position 1 (ΔM1) of wild-type human Neu2 (SEQ ID NO: 1).

In certain embodiments, the recombinant mutant human sialidase comprises a substitution or combination of substitutions corresponding to a substitution or combination of substitutions listed in TABLE 6 (amino acid positions corresponding to wild-type human Neu2 (SEQ ID NO: 1)).

TABLE 6
Mutation(s)
Deletion of M1, V6Y, I187K
M1R, V6Y, I187K
M1H, V6Y, I187K
M1K, V6Y, I187K
M1D, V6Y, I187K
M1T, V6Y, I187K
M1N, V6Y, I187K
M1Q, V6Y, I187K
M1G, V6Y, I187K
M1A, V6Y, I187K
M1V, V6Y, I187K
M1L, V6Y, I187K
M1F, V6Y, I187K
M1Y, V6Y, I187K

e. Other Substitutions

In addition, the sialidase may further comprise at least one of the following substitutions: A328E, K370N, or H210N. In certain embodiments, the isoleucine at a position corresponding to position 187 of wild-type human Neu2 is substituted by a positively charged amino acid, e.g., lysine (I187K) or arginine (I187R). In certain embodiments, the isoleucine at a position corresponding to position 187 of wild-type human Neu2 is substituted by lysine (I187K). In certain embodiments, a recombinant mutant human Neu2 comprises the substitution of the amino acids GDYDAPTHQVQW (SEQ ID NO: 15) with the amino acids SMDQGSTW (SEQ ID NO: 16) or STDGGKTW (SEQ ID NO: 17). In certain embodiments, a recombinant mutant human Neu2 comprises the substitution of the amino acids PRPPAPEA (SEQ ID NO: 18) with the amino acids QTPLEAAC (SEQ ID NO: 19). In certain embodiments, a recombinant mutant human Neu2 comprises the substitution of the amino acids NPRPPAPEA (SEQ ID NO: 20) with the amino acids SQNDGES (SEQ ID NO: 21).

The invention further provides a recombinant mutant human sialidase comprising at least one substitution at a position corresponding to V212, A213, Q214, D215, T216, L217, E218, C219, Q220, V221, A222, E223, V224, E225, or T225.

The invention further provides a recombinant mutant human sialidase comprising an amino acid substitution at a position identified in TABLE 7 (amino acid positions corresponding to wild-type human Neu2 (SEQ ID NO: 1). In certain embodiments, the sialidase comprises an amino acid substitution identified in TABLE 7. In certain embodiments, the sialidase comprises a combination of any amino acid substitutions identified in TABLE 7.

TABLE 7

| Wild Type | |
|---|---|
| Human Neu2 | |
| (SEQ ID NO: 1) | Exemplary Substitution(s) at Specified |
| Amino Acid | Position(s) |
| M1 | D |
| L4 | S, T, Y, L, F, A, P, V, I, N, D, or H |
| P5 | G |
| V6 | Y |
| L7 | F, Y, S, I, T, or N |
| K9 | D |
| V12 | L, A, P, V, N, D, or H |
| F13 | S, N, R, K, T, G, D, E, or A |
| I22 | S, N, R, K, T, G, D, E, A, Y, L, F, P, V, I, or H |
| A24 | S, N, R, K, T, G, D, E, A, Y, L, F, P, V, I, or H |
| L34 | S, T, Y, L, F, A, P, V, I, N, D, or H |
| A36 | S, T, Y, L, F, A, P, V, I, N, D, or H |
| A42 | R or D |
| K44 | R or E |
| K45 | A, E, or R |
| L54 | M |
| P62 | H, G, N, T, S, F, I, D, or E |
| H64 | F, Y, S, I, T, or N |
| Q69 | H |
| R78 | K |
| D80 | P |
| P89 | S, T, Y, L, F, A, P, V, I, N, D, H, or M |
| A93 | E or K |
| G107 | D |
| Q108 | H |
| Q112 | E, R, or K |
| C125 | Y, F, or L |
| Q126 | E, F, H, I, L, or Y |
| A150 | V |
| T156 | R, N, D, C, G, H, I, L, F, S, Y, V, A, P, or T |
| F157 | R, N, D, C, G, H, I, L, F, S, Y, V, A, or P |
| A158 | R, N, D, C, G, H, I, L, F, S, Y, V, A, P, or T |
| V159 | R, N, D, C, G, H, I, L, F, S, Y, V, A, or P |
| G160 | R, N, D, C, G, H, I, L, F, S, Y, V, A, P, or T |
| P161 | R, N, D, C, G, H, I, L, F, S, Y, V, A, or P |
| G162 | R, N, D, C, G, H, I, L, F, S, Y, V, A, P, or T |
| H163 | R, N, D, C, G, H, I, L, F, S, Y, V, A, or P |
| C164 | R, N, D, C, G, H, I, L, F, S, Y, V, A, P, or T |
| L165 | R, N, D, C, G, H, I, L, F, S, Y, V, A, or P |
| R170 | P |
| A171 | G |
| V176 | R, N, D, C, G, H, I, L, F, S, Y, V, P, or A |
| P177 | S, T, Y, L, F, A, P, V, I, N, D, or H |
| A178 | S, T, Y, L, F, A, P, V, I, N, D, or H |
| L184 | S, N, R, K, T, G, D, E, A, F, H, I, L, P, V, or Y |
| H185 | S, N, R, K, T, G, D, E, or A |
| P186 | S, N, R, K, T, G, D, E, A, F, H, I, L, P, V, or Y |
| I187 | S, N, R, K, T, G, D, E, or A |
| Q188 | P, S, N, R, K, T, G, D, E, or A |
| R189 | P |
| P190 | F, M, A, D, G, H, N, P, R, S, or T |
| I191 | M, A, D, F, H, I, L, N, P, S, T, V, Y, E, G, K, or R |
| A194 | S, T, Y, L, F, A, P, V, I, N, D, or H |
| A213 | C, N, S, or T |
| L217 | R, N, D, C, G, H, I, L, F, S, Y, or V |
| C219 | R, N, D, C, G, H, I, L, F, S, Y, or V |
| A222 | D |
| E225 | P or C |
| H239 | P |
| L240 | D, N, or Y |
| R241 | A, D, L, Q, or Y |
| A242 | C, F, G, H, I, K, L, M, N, Q, R, S, V, W, or Y |
| V244 | I or P |
| T249 | A |
| D251 | G |
| E257 | P |
| S258 | C |
| L260 | D, F, Q, or T |
| V265 | F |
| Q270 | S, T, A, H, P, or F |
| G271 | S, N, R, K, T, G, D, E, or A |
| C272 | S, N, R, K, T, G, D, E, A, C, H, Y, F, H, L, P, or V |
| A290 | C |
| W292 | R |
| S301 | A, D, E, F, G, H, I, K, L, M, N, P, Q, T, V, W, Y, |
| | C, or R |
| W302 | A, D, E, F, G, H, I, L, M, N, P, Q, R, S, T, V, Y, |
| | or K |
| E319 | D |
| V325 | F, Y, S, I, T, N, A, D, H, L, P, or V |
| L326 | F, Y, S, I, T, N, A, D, H, L, P, or V |
| L327 | F, Y, S, I, T, N, A, D, H, L, P, or V |
| C332 | A, D, G, H, N, P, R, S, or T |
| Y359 | A or S |
| V363 | R, S, T, Y, L, F, A, P, V, I, N, D, or H |
| L365 | K, Q, F, Y, S, I, T, N, A, D, H, L, P, or V |

For example, in certain embodiments, the recombinant mutant human sialidase comprises: (a) a substitution of a proline residue at a position corresponding to position 5 of wild-type human Neu2 (P5); (b) a substitution of a lysine residue at a position corresponding to position 9 of wild-type human Neu2 (K9); (c) a substitution of an alanine residue at a position corresponding to position 42 of wild-type human Neu2 (A42); (d) a substitution of a lysine residue at a position corresponding to position 44 of wild-type human Neu2 (K44); (e) a substitution of a lysine residue at a position corresponding to position 45 of wild-type human Neu2 (K45); (f) a substitution of a leucine residue at a position corresponding to position 54 of wild-type human Neu2 (L54); (g) a substitution of a proline residue at a position corresponding to position 62 of wild-type human Neu2 (P62); (h) a substitution of a glutamine residue at a position corresponding to position 69 of wild-type human Neu2 (Q69); (i) a substitution of an arginine residue at a position corresponding to position 78 of wild-type human Neu2 (R78); (j) a substitution of an aspartic acid residue at a position corresponding to position 80 of wild-type human Neu2 (D80); (k) a substitution of an alanine residue at a position corresponding to position 93 of wild-type human Neu2 (A93); (l) a substitution of a glycine residue at a position corresponding to position 107 of wild-type human Neu2 (G107); (m) a substitution of a glutamine residue at a position corresponding to position 108 of wild-type human Neu2 (Q108); (n) a substitution of a glutamine residue at a position corresponding to position 112 of wild-type human Neu2 (Q112); (o) a substitution of a cysteine residue at a position corresponding to position 125 of wild-type human Neu2 (C125); (p) a substitution of a glutamine residue at a position corresponding to position 126 of wild-type human Neu2 (Q126); (q) a substitution of an alanine residue at a position corresponding to position 150 of wild-type human Neu2 (A150); (r) a substitution of a cysteine residue at a position corresponding to position 164 of wild-type human Neu2 (C164); (s) a substitution of an arginine residue at a position corresponding to position 170 of wild-type human Neu2 (R170); (t) a substitution of an alanine residue at a position corresponding to position 171 of wild-type human Neu2 (A171); (u) a substitution of a glutamine residue at a position corresponding to position 188 of wild-type human Neu2 (Q188); (v) a substitution of an arginine residue at a position corresponding to position 189 of wild-type human Neu2 (R189); (w) a substitution of an alanine residue at a position corresponding to position 213 of wild-type human Neu2 (A213); (x) a substitution of a leucine residue at a position corresponding to position 217 of wild-type human Neu2 (L217); (y) a substitution of a glutamic acid residue at a position corresponding to position 225 of wild-type human Neu2 (E225); (z) a substitution of a histidine residue at a position corresponding to position 239 of wild-type human Neu2 (H239); (aa) a substitution of a leucine residue at a position corresponding to position 240 of wild-type human Neu2 (L240); (bb) a substitution of an arginine residue at a position corresponding to position 241 of wild-type human Neu2 (R241); (cc) a substitution of an alanine residue at a position corresponding to position 242 of wild-type human Neu2 (A242); (dd) a substitution of a valine residue at a position corresponding to position 244 of wild-type human Neu2 (V244); (ee) a substitution of a threonine residue at a position corresponding to position 249 of wild-type human Neu2 (T249); (ff) a substitution of an aspartic acid residue at a position corresponding to position 251 of wild-type human Neu2 (D251); (gg) a substitution of a glutamic acid residue at a position corresponding to position 257 of wild-type human Neu2 (E257); (hh) a substitution of a serine residue at a position corresponding to position 258 of wild-type human Neu2 (S258); (ii) a substitution of a leucine residue at a position corresponding to position 260 of wild-type human Neu2 (L260); (jj) a substitution of a valine residue at a position corresponding to position 265 of wild-type human Neu2 (V265); (kk) a substitution of a glutamine residue at a position corresponding to position 270 of wild-type human Neu2 (Q270); (ll) a substitution of an alanine residue at a position corresponding to position 290 of wild-type human Neu2 (A290); (mm) a substitution of a tryptophan residue at a position corresponding to position 292 of wild-type human Neu2 (W292); (nn) a substitution of a serine residue at a position corresponding to position 301 of wild-type human Neu2 (S301); (oo) a substitution of a tryptophan residue at a position corresponding to position 302 of wild-type human Neu2 (W302); (pp) a substitution of a valine residue at a position corresponding to position 363 of wild-type human Neu2 (V363); or (qq) a substitution of a leucine residue at a position corresponding to position 365 of wild-type human Neu2 (L365); or a combination of any of the foregoing substitutions. For example, the sialidase may comprise a substitution of K9, A42, P62, A93, Q216, A242, Q270, S301, W302, V363, or L365, or a combination of any of the foregoing substitutions.

In certain embodiments, in the sialidase: (a) the proline residue at a position corresponding to position 5 of wild-type human Neu2 is substituted by histidine (P5H); (b) the lysine residue at a position corresponding to position 9 of wild-type human Neu2 is substituted by aspartic acid (K9D); (c) the alanine residue at a position corresponding to position 42 of wild-type human Neu2 is substituted by a positively charged amino acid, e.g., arginine (A42R) or lysine (A42K), or is substituted by aspartic acid (A42D); (d) the lysine residue at a position corresponding to position 44 of wild-type human Neu2 is substituted by arginine (K44R) or glutamic acid (K44E); (e) the lysine residue at a position corresponding to position 45 of wild-type human Neu2 is substituted by alanine (K45A), arginine (K45R), or glutamic acid (K45E); (f) the leucine residue at a position corresponding to position 54 of wild-type human Neu2 is substituted by methionine (L54M); (g) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T); (h) the glutamine residue at a position corresponding to position 69 of wild-type human Neu2 is substituted by histidine (Q69H); (i) the arginine residue at a position corresponding to position 78 of wild-type human Neu2 is substituted by lysine (R78K); (j) the aspartic acid residue at a position corresponding to position 80 of wild-type human Neu2 is substituted by proline (D80P); (k) the alanine residue at a position corresponding to position 93 of wild-type human Neu2 is substituted by a negatively charged amino acid, e.g., aspartic acid (A93D) or glutamic acid (A93E), or is substituted by lysine (A93K); (l) the glycine residue at a position corresponding to position 107 of wild-type human Neu2 is substituted by aspartic acid (G107D); (m) the glutamine residue at a position corresponding to position 108 of wild-type human Neu2 is substituted by histidine (Q108H); (n) the glutamine residue at a position corresponding to position 112 of wild-type human Neu2 is substituted by glutamic acid (Q112E), arginine (Q112R), or lysine (Q112K); (o) the cysteine residue at a position corresponding to position 125 of wild-type human Neu2 is substituted by leucine (C125L); (p) the glutamine residue at a position corresponding to position 126 of wild-type human Neu2 is substituted by an aromatic amino acid, e.g., phenylalanine (Q126F), tyrosine (Q126Y), or tryptophan (Q126W), or is substituted by leucine (Q126L), glutamic acid (Q126E), histidine (Q126H), or isoleucine (Q126I); (q) the alanine residue at a position corresponding to position 150 of wild-type human Neu2 is substituted by valine (A150V); (r) the cysteine residue at a position corresponding to position 164 of wild-type human Neu2 is substituted by glycine (C164G); (s) the arginine residue at a position corresponding to position 170 of wild-type human Neu2 is substituted by proline (R170P); (t) the alanine residue at a position corresponding to position 171 of wild-type human Neu2 is substituted by glycine (A171G); (u) the glutamine residue at a position corresponding to position 188 of wild-type human Neu2 is substituted by proline (Q188P); (v) the arginine residue at a position corresponding to position 189 of wild-type human Neu2 is substituted by proline (R189P); (w) the alanine residue at a position corresponding to position 213 of wild-type human Neu2 is substituted by cysteine (A213C), asparagine (A213N), serine (A213S), or threonine (A213T); (x) the leucine residue at a position corresponding to position 217 of wild-type human Neu2 is substituted by alanine (L217A) or valine (L217V); (y) the threonine residue at a position corresponding to position 249 of wild-type human Neu2 is substituted by alanine (T249A); (z) the aspartic acid residue at a position corresponding to position 251 of wild-type human Neu2 is substituted by glycine (D251G); (aa) the glutamic acid residue at a position corresponding to position 225 of wild-type human Neu2 is substituted by cysteine (E225C) or proline (E225P); (bb) the histidine residue at a position corresponding to position 239 of wild-type human Neu2 is substituted by proline (H239P); (cc) the leucine residue at a position corresponding to position 240 of wild-type human Neu2 is substituted by aspartic acid (L240D), asparagine (L240N), or tyrosine (L240Y); (dd) the arginine residue at a position corresponding to position 241 of wild-type human Neu2 is substituted by alanine (R241A), aspartic acid (R241D), leucine (R241L), glutamine (R241Q). or tyrosine (R241Y); (ee) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ff) the valine residue at a position corresponding to position 244 of wild-type human Neu2 is substituted by isoleucine (V244I), lysine (V244K), or proline (V244P); (gg) the glutamic acid residue at a position corresponding to position 257 of wild-type human Neu2 is substituted by proline (E257P); (hh) the serine residue at a position corresponding to position 258 is substituted by cysteine (S258C); (ii) the leucine residue at a position corresponding to position 260 of wild-type human Neu2 is substituted by aspartic acid (L260D), phenylalanine (L260F), glutamine (L260Q), or threonine (L260T); (jj) the valine residue at a position corresponding to position 265 of wild-type human Neu2 is substituted by phenylalanine (V265F); (kk) the glutamine residue at a position corresponding to position 270 of wild-type human Neu2 is substituted by a polar, uncharged amino acid, e.g., serine (Q270S) or threonine (Q270T), or is substituted by alanine (Q270A), histidine (Q270H), phenylalanine (Q270F), or proline (Q270P); (ll) the alanine residue at a position corresponding to position 290 of wild-type human Neu2 is substituted by cysteine (A290C); (mm) the tryptophan residue at a position corresponding to position 292 of wild-type human Neu2 is substituted by arginine (W292R); (nn) the serine residue at a position corresponding to position 301 of wild-type human Neu2 is substituted by alanine (S301A), aspartic acid (S301D), glutamic acid (S301E), phenylalanine (S301F), histidine (S301H), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y)); (oo) the tryptophan residue at a position corresponding to position 302 of wild-type human Neu2 is substituted by alanine (W302A), aspartic acid (W302D), phenylalanine (W302F), glycine (W302G), histidine (W302H), isoleucine (W302I), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y); (pp) the valine residue at a position corresponding to position 363 of wild-type human Neu2 is substituted by arginine (V363R); or (qq) the leucine residue at a position corresponding to position 365 of wild-type human Neu2 is substituted by glutamine (L365Q), histidine (L365H), isoleucine (L365I), lysine (L365K) or serine (L365S); or the sialidase comprises a combination of any of the foregoing substitutions. For example, the sialidase may comprise a substitution selected from K9D, A42R, P62G, P62N, P62S, P62T, A93E, Q126Y, A242F, A242W, A242Y, Q270A, Q270T, S301A, S301R, W302K, W302R, V363R, and L365I, or a combination of any of the foregoing substitutions.

In certain embodiments, the recombinant mutant human sialidase comprises a deletion of a leucine residue at a position corresponding to position 184 of wild-type human Neu2 (ΔL184), a deletion of a histidine residue at a position corresponding to position 185 of wild-type human Neu2 (ΔH185), a deletion of a proline residue at a position corresponding to position 186 of wild-type human Neu2 (ΔP186), a deletion of an isoleucine residue at a position corresponding to position 187 of wild-type human Neu2 (ΔI187), and a deletion of a glutamine residue at a position corresponding to position 184 of wild-type human Neu2 (ΔQ188), or a combination of any of the foregoing deletions.

In certain embodiments, the recombinant mutant human sialidase comprises an insertion between a threonine residue at a position corresponding to position 216 of wild-type human Neu2 and a leucine residue at a position corresponding to position 217 of wild-type human Neu2, for example, an insertion of an amino acid selected from S, T, Y, L, F, A, P, V, I, N, D, and H.

Additional exemplary sialidase mutations, and combinations of sialidase mutations, are described in International (PCT) Patent Application Publication No. WO 2019/136167, including in the Detailed Description in the section entitled “I. Recombinant Human Sialidases,” and in the Examples in Examples 1, 2, 3, 4, 5, and 6, and International (PCT) Patent Application Publication No. WO 2021/003469, including in the Detailed Description in the section entitled “I. Recombinant Human Sialidases,” and in the Examples in Examples 2, 3, 4, and 5.

f. Combinations of Substitutions

The invention further provides a recombinant mutant human sialidase comprising a combination of any of the mutations contemplated herein. For example, the recombinant mutant sialidase enzyme may comprise a combination of 2, 3, 4, 5, 6, 7, 8, 9, 10, 11, 12, 13, 14, 15 or more of the mutations contemplated herein. It is contemplated that the recombinant mutant sialidase enzyme may comprise 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 3-4 of the mutations contemplated herein.

For example, alternatively or in addition to the modifications to decrease sensitivity to protease (e.g., trypsin) degradation, the recombinant mutant sialidase enzyme may comprise one or more of the following modifications described herein.

In certain embodiments, the recombinant mutant sialidase enzyme further comprises an M1 deletion (ΔM1), M1A substitution, M1D substitution, V6Y substitution, K9D substitution, P62G substitution, P62N substitution, P62S substitution, P62T substitution, A93E substitution, I187K substitution, Q270A substitution, S301R substitution, W302K substitution, C332A substitution, V363R substitution, L365I substitution, or a combination of any of the foregoing.

In certain embodiments, the recombinant mutant sialidase enzyme further comprises a M1 deletion (ΔM1), M1A substitution, M1D substitution, V6Y substitution, I187K substitution, C332A substitution, or a combination of any of the foregoing. For example, the recombinant mutant sialidase enzyme may comprise a combination of mutations selected from: M1A and V6Y; M1A and I187K; M1A and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; M1A, V6Y, and I187K; M1A, V6Y, and C332A; M1A, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A.

In certain embodiments, the recombinant mutant sialidase enzyme further comprises (i) an amino acid substitution identified in TABLE 7, or a combination of any amino acid substitutions identified in TABLE 7, and (ii) an M1 deletion (ΔM1), M1A substitution, M1D substitution, V6Y substitution, I187K substitution, C332A substitution, or a combination of any of the foregoing. For example, the recombinant mutant sialidase enzyme may comprise (i) an amino acid substitution identified in TABLE 7, or a combination of any amino acid substitutions identified in TABLE 7, and (ii) a combination of mutations selected from: M1A and V6Y; M1A and I187K; M1A and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; M1A, V6Y, and I187K; M1A, V6Y, and C332A; M1A, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A.

In certain embodiments, the recombinant mutant sialidase enzyme comprises: (a) the M1D, V6Y, P62G, A93E, I187K, and C332A substitutions; (b) the M1D, V6Y, K9D, A93E, I187K, C332A, V363R, and L365I substitutions; (c) the M1D, V6Y, P62N, I187K, and C332A substitutions; (d) the M1D, V6Y, I187K, Q270A, S301R, W302K, and C332A substitutions; (e) the M1D, V6Y, P62S, I187K, Q270A, S301R, W302K, and C332A substitutions; (f) the M1D, V6Y, P62T, I187K, Q270A, S301R, W302K, and C332A substitutions; (g) the M1D, V6Y, P62N, I187K, Q270A, S301R, W302K, and C332A substitutions; (h) the M1D, V6Y, P62G, A93E, I187K, S301A, W302R, and C332A substitutions; (i) the M1D, V6Y, P62G, A93E, Q126Y, I187K, Q270T, and C332A substitutions; (j) the M1D, V6Y, P62G, A93E, Q126Y, I187K, and C332A substitutions; (k) the M1D, V6Y, P62G, A93E, Q126Y, I187K, A242F, Q270T, and C332A substitutions; (l) the M1D, V6Y, A42R, P62G, A93E, Q126Y, I187K, A242F, Q270T, and C332A substitutions; (m) the M1D, V6Y, P62G, A93E, Q112E, Q126Y, I187K, Q270T, A242F, and C332A; or (n) the M1D, V6Y, P62G, A93E, Q126Y, I187K, E225C, Q270T, A290C, A242F, C332A substitutions.

In certain embodiments, the recombinant mutant human sialidase further comprises a substitution of a serine residue at a position corresponding to position 301 of wild-type human Neu2 (S301) in combination with a substitution of a tryptophan residue at a position corresponding to position 302 of wild-type human Neu2 (W302). For example, the recombinant mutant human sialidase may comprise a combination of substitutions corresponding to a combination of substitutions listed in a row of TABLE 8 (amino acid positions corresponding to wild-type human Neu2 (SEQ ID NO: 1)). For example, the recombinant mutant human sialidase may comprise: the S301K and W302R substitutions; the S301K and W302K substitutions; or the S301A and W302S substitutions.

TABLE 8
Substitutions

S301A, W302R
S301A, W302S
S301A, W302T
S301K, W302S
S301N, W302S
S301T, W302S
S301T, W302T
S301T, W302R
S301A, W302A
S301K, W302R
S301K, W302T
S301N, W302T
S301K, W302K
S301P, W302R
S301P, W302S
S301P, W302T

In certain embodiments, the recombinant mutant human sialidase further comprises a combination of substitutions corresponding to a combination of substitutions listed in a row of TABLE 9 (amino acid positions corresponding to wild-type human Neu2 (SEQ ID NO: 1)).

TABLE 9
Substitutions

M1D, V6Y, P62G, I187K, C332A
M1D, V6Y, K9D, I187K, C332A, V363R, L365I
M1D, V6Y, P62G, A93E, I187K, C332A
M1D, V6Y, K9D, I187K, C332A, V363R, L365K
M1D, V6Y, K9D, I187K, C332A, V363R, L365S
M1D, V6Y, K9D, I187K, C332A, V363R, L365Q
M1D, V6Y, K9D, I187K, C332A, V363R, L365H
M1D, V6Y, A93K, I187K, C332A
M1D, V6Y, A93E, I187K, C332A
V6Y, I187K, W292R
V6Y, G107D, I187K
V6Y, C125L
C125L, I187K
V6Y, C125L, I187K
M1D, V6Y, K45A, I187K, C332A
M1D, V6Y, Q270A, I187K, C332A
M1D, V6Y, K44R, K45R, I187K, C332A
M1D, V6Y, Q112R, I187K, C332A
M1D, V6Y, Q270F, I187K, C332A
M1D, V6Y, I187K, S301R, W302K, C332A
M1D, V6Y, K44E, K45E, I187K, C332A
M1D, V6Y, I187K, L217V, C332A
M1D, V6Y, I187K, L217A, C332A
M1D, V6Y, K44E, K45E, I187K, S301R, W302K, C332A
M1D, V6Y, Q112R, I187K, S301R, W302K, C332A
M1D, V6Y, I187K, Q270A, S301R, W302K, C332A
M1D, V6Y, K44E, K45E, Q112R, I187K, C332A
M1D, V6Y, K44E, K45E, I187K, Q270A, C332A
M1D, V6Y, K45A, I187K, Q270A, C332A
M1D, V6Y, I187K, Q270H, C332A
M1D, V6Y, I187K, Q270P, C332A
M1D, V6Y, Q112K, I187K, C332A
M1D, V6Y, P62S, I187K, Q270A, S301R, W302K, C332A
M1D, V6Y, P62T, I187K, Q270A, S301R, W302K, C332A
M1D, V6Y, P62N, I187K, Q270A, S301R, W302K, C332A
V6Y, P62H, I187K
V6Y, Q108H, I187K
M1D, V6Y, P62H, I187K, C332A
M1D, V6Y, P62G, I187K, C332A
V6Y, P62G, I187K
M1D, V6Y, P62H, I187K
M1D, V6Y, Q108H, I187K
M1D, V6Y, P62N, I187K, C332A
M1D, V6Y, P62D, I187K, C332A
M1D, V6Y, P62E, I187K, C332A
V6Y, C164G, I187K, T249A
V6Y, C164G, I187K
V6Y, Q126L, I187K D251G
V6Y, L54M, Q69H, R78K, A171G, I187K
V6Y, P62T, I187K
V6Y, A150V, I187K
P5H, V6Y, P62S, I187K
V6Y, C164G, I187K
Q126Y, Q170T
Q126Y, A242F, Q270T
M1D, V6Y, P62G, A93E, Q126E, I187K, C332A
M1D, V6Y, P62G, A93E, Q126I, I187K, C332A
M1D, V6Y, P62G, A93E, Q126L, I187K, C332A
M1D, V6Y, P62G, A93E, Q126Y, I187K, C332A
M1D, V6Y, P62G, A93E, Q126F, I187K, C332A
M1D, V6Y, P62G, A93E, Q126H, I187K, C332A
M1D, V6Y, P62G, A93E, I187K, Q270S, C332A
M1D, V6Y, P62G, A93E, I187K, Q270T, C332A
M1D, V6Y, P62G, A93E, Q126Y, I187K, Q270T, C332A
M1D, V6Y, P62G, A93E, Q126Y, I187K, A242F, Q270T, C332A
M1D, V6Y, P62G, D80P, A93E, I187K, C332A
M1D, V6Y, P62G, A93E, R170P, I187K, C332A
M1D, V6Y, P62G, A93E, I187K, Q188P, C332A
M1D, V6Y, P62G, A93E, I187K, R189P, C332A
M1D, V6Y, P62G, A93E, I187K, E225P, C332A
M1D, V6Y, P62G, A93E, I187K, H239P, C332A
M1D, V6Y, P62G, A93E, I187K, E257P, C332A
M1D, V6Y, P62G, A93E, I187K, S301A, C332A
M1D, V6Y, P62G, A93E, I187K, S301D, C332A
M1D, V6Y, P62G, A93E, I187K, S301E, C332A
M1D, V6Y, P62G, A93E, I187K, S301F, C332A
M1D, V6Y, P62G, A93E, I187K, S301H, C332A
M1D, V6Y, P62G, A93E, I187K, S301K, C332A
M1D, V6Y, P62G, A93E, I187K, S301L, C332A
M1D, V6Y, P62G, A93E, I187K, S301M, C332A
M1D, V6Y, P62G, A93E, I187K, S301N, C332A
M1D, V6Y, P62G, A93E, I187K, S301P, C332A
M1D, V6Y, P62G, A93E, I187K, S301Q, C332A
M1D, V6Y, P62G, A93E, I187K, S301R, C332A
M1D, V6Y, P62G, A93E, I187K, S301T, C332A
M1D, V6Y, P62G, A93E, I187K, S301V, C332A
M1D, V6Y, P62G, A93E, I187K, S301W, C332A
M1D, V6Y, P62G, A93E, I187K, S301Y, C332A
M1D, V6Y, P62G, A93E, I187K, W302A, C332A
M1D, V6Y, P62G, A93E, I187K, W302D, C332A
M1D, V6Y, P62G, A93E, I187K, W302F, C332A
M1D, V6Y, P62G, A93E, I187K, W302G, C332A
M1D, V6Y, P62G, A93E, I187K, W302H, C332A
M1D, V6Y, P62G, A93E, I187K, W302I, C332A
M1D, V6Y, P62G, A93E, I187K, W302L, C332A
M1D, V6Y, P62G, A93E, I187K, W302M, C332A
M1D, V6Y, P62G, A93E, I187K, W302N, C332A
M1D, V6Y, P62G, A93E, I187K, W302P, C332A
M1D, V6Y, P62G, A93E, I187K, W302Q, C332A
M1D, V6Y, P62G, A93E, I187K, W302R, C332A
M1D, V6Y, P62G, A93E, I187K, W302S, C332A
M1D, V6Y, P62G, A93E, I187K, W302T, C332A
M1D, V6Y, P62G, A93E, I187K, W302V, C332A
M1D, V6Y, P62G, A93E, I187K, W302Y, C332A
M1D, V6Y, P62G, A93E, I187K, S301A, W302A, C332A
M1D, V6Y, P62G, A93E, I187K, S301A, W302R, C332A
M1D, V6Y, P62G, A93E, I187K, S301A, W302S, C332A
M1D, V6Y, P62G, A93E, I187K, S301A, W302T, C332A
M1D, V6Y, P62G, A93E, I187K, S301K, W302S, C332A
M1D, V6Y, P62G, A93E, I187K, S301K, W302R, C332A
M1D, V6Y, P62G, A93E, I187K, S301K, W302T, C332A
M1D, V6Y, P62G, A93E, I187K, S301N, W302S, C332A
M1D, V6Y, P62G, A93E, I187K, S301N, W302T, C332A
M1D, V6Y, P62G, A93E, I187K, S301T, W302R, C332A
Q126Y, Q270T
Q126Y, A242F, Q270T
M1D, V6Y, A42R, P62G, A93E, Q126Y, I187K, A242F, Q270T, C332A
M1D, V6Y, P62G, A93E, Q112E, Q126Y, I187K, Q270T, A242F, C332A
M1D, V6Y, P62G, A93E, Q126Y, I187K, E225C, Q270T, A290C, A242F, C332A
V6Y, A42R, P62G, A93E, Q126Y, I187K, Q270T, A242F, C332A
M1D, A42R, P62G, A93E, Q126Y, I187K, Q270T, A242F, C332A
M1D, V6Y, P62G, A93E, Q126Y, I187K, Q270T, A242F, C332A
M1D, V6Y, A42R, A93E, Q126Y, I187K, Q270T, A242F, C332A
M1D, V6Y, A42R, P62G, Q126Y, I187K, Q270T, A242F, C332A
M1D, V6Y, A42R, P62G, A93E, I187K, Q270T, A242F, C332A
M1D, V6Y, A42R, P62G, A93E, Q126Y, Q270T, A242F, C332A
M1D, V6Y, A42R, P62G, A93E, Q126Y, I187K, Q270T, C332A
M1D, V6Y, A42R, P62G, A93E, Q126Y, I187K, A242F, C332A
M1D, V6Y, A42R, P62G, A93E, Q126Y, I187K, Q270T, A242F

In certain embodiments, the sialidase comprises a substitution of M1, A42, and Q270, or a combination of any of the foregoing substitutions. These substitutions were found to, for example, improve the activity of the enzyme (see, Example 11). In certain embodiments, the sialidase comprises a substitution at each of M1, A42, and Q270. In certain embodiments, the sialidase comprises a substitution selected from M1D, A42R, and Q270T, or a combination of any of the foregoing substitutions. In certain embodiments, the sialidase comprises each of the M1D, A42R, and Q270T substitutions.

In certain embodiments, the sialidase comprises a substitution of V6, P62, A93, Q126, and I187, or a combination of any of the foregoing substitutions. These substitutions were found to, for example, improve the expression/yield of the enzyme (see, Example 11). In certain embodiments, the sialidase comprises a substitution at each of V6, P62, A93, Q126, and I187. In certain embodiments, the sialidase comprises a substitution selected from V6Y, P62G, A93E, Q126Y, and I187K, or a combination of any of the foregoing substitutions. In certain embodiments, the sialidase comprises each of the V6Y, P62G, A93E, Q126Y, and I187K substitutions.

In certain embodiments, the sialidase comprises a substitution of M1, A42, A242, and Q270, or a combination of any of the foregoing substitutions. These substitutions were found to, for example, improve the stability of the enzyme (see, Example 11). In certain embodiments, the sialidase comprises a substitution at each of M1, A42, A242, and Q270. In certain embodiments, the sialidase comprises a substitution selected from M1D, A42R, A242F, and Q270T, or a combination of any of the foregoing substitutions. In certain embodiments, the sialidase comprises each of the M1D, A42R, A242F, and Q270T substitutions.

In certain embodiments, the recombinant mutant sialidase enzyme comprises: (a) the V6Y, A42R, P62G, A93E, Q126Y, I187K, Q270T, A242F, and C332A substitutions; (b) the ΔM1 deletion and the V6Y, A42R, P62G, A93E, Q126Y, I187K, Q270T, A242F, and C332A substitutions; (c) the M1D, A42R, P62G, A93E, Q126Y, I187K, Q270T, A242F, and C332A substitutions; (d) the M1D, V6Y, P62G, A93E, Q126Y, I187K, Q270T, A242F, and C332A substitutions; (e) the M1D, V6Y, A42R, A93E, Q126Y, I187K, Q270T, A242F, and C332A substitutions; (f) the M1D, V6Y, A42R, P62G, Q126Y, I187K, Q270T, A242F, and C332A substitutions; (g) the M1D, V6Y, A42R, P62G, A93E, I187K, Q270T, A242F, and C332A substitutions; (h) the M1D, V6Y, A42R, P62G, A93E, Q126Y, Q270T, A242F, and C332A substitutions; (i) the M1D, V6Y, A42R, P62G, A93E, Q126Y, I187K, Q270T, and C332A substitutions; (j) the M1D, V6Y, A42R, P62G, A93E, Q126Y, I187K, A242F, and C332A substitutions; or (k) the M1D, V6Y, A42R, P62G, A93E, Q126Y, I187K, Q270T, and A242F substitutions.

In certain embodiments, the sialidase comprises a substitution of M1, V6, A42, P62, A93, Q126, I187, A242, Q270, C332A, or a combination of any of the foregoing substitutions. In certain embodiments, the sialidase comprises a substitution at each of M1, V6, A42, P62, A93, Q126, I187, A242, Q270, and C332. In certain embodiments, the sialidase comprises a substitution selected from M1D, V6Y, A42R, P62G, A93E, Q126Y, I187K, A242F, Q270T, and C332A, or a combination of any of the foregoing substitutions. In certain embodiments, the sialidase comprises each of the M1D, V6Y, A42R, P62G, A93E, Q126Y, I187K, A242F, Q270T, and C332A substitutions.

In certain embodiments, the recombinant mutant human sialidase comprises the amino acid sequence of any one of SEQ ID NOs: 48-54, 149, 154, 159, 191, or 198, or an amino acid sequence that has at least 85%, 90%, 95%, 96%, 97%, 98%, or 99% sequence identity to any one of SEQ ID NOs: 48-54, 149, 154, 159, 191, or 198. In certain embodiments, the recombinant mutant human sialidase is then modified, or has been modified, to include one or more modifications to decrease protease sensitivity.

In certain embodiments, the recombinant mutant human sialidase comprises the amino acid sequence of

(SEQ ID NO: 199)
X₁X₂SX₃X₄X₅LQX₆ESVFQSGAHAYRIPALLYLPGQQSLLAFAEQRX₇S
X₈X₉DEHAELIVX₁₀RRGDYDAX₁₁THQVQWX₁₂AQEVVAQAX₁₃LX₁₄GHR
SMNPCPLYDX₁₅QTGTLFLFFIAIPX₁₆X₁₇VTEX₁₈QQLQTRANVTRLX₁₉
X₂₀VTSTDHGRTWSSPRDLTDAAIGPX₂₁YREWSTFAVGPGHX₂₂LQLHD
X₂₃X₂₄RSLVVPAYAYRKLHPX₂₅X₂₆X₂₇PIPSAFX₂₈FLSHDHGRTWARG
HFVX₂₉QDTX₃₀ECQVAEVX₃₁TGEQRVVTLNARSX₃₂X₃₃X₃₄X₃₅RX₃₆QA
QSX₃₇NX₃₈GLDFQX₃₉X₄₀QX₄₁VKKLX₄₂EPPPX₄₃GX₄₄QGSVISFPSPR
SGPGSPAQX₄₅LLYTHPTHX₄₆X₄₇QRADLGAYLNPRPPAPEAWSEPX₄₈L
LAKGSX₄₉AYSDLQSMGTGPDGSPLFGX₅₀LYEANDYEEIX₅₁FX₅₂MFTL
KQAFPAEYLPQ,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Ala or Arg, X₈ is Lys, Arg, or Glu, X₉ is Lys, Ala, Arg, or Glu, X₁₀ is Leu or Met, X₁₁ is Pro, Asn, Asp, His, Glu, Gly, Ser or Thr, X₁₂ is Gln or His, X₁₃ is Arg or Lys, X₁₄ is Asp or Pro, X₁₅ is Ala, Glu or Lys, X₁₆ is Gly or Asp, X₁₇ is Gln or His, X₁₈ is Gln, Arg, or Lys, X₁₉ is Ala, Cys, Ile, Ser, Val, or Leu, X₂₀ is Gln, Leu, Glu, Phe, His, Ile, Leu, or Tyr, X₂₁ is Ala or Val, X₂₂ is Cys or Gly, X₂₃ is Arg or Pro, X₂₄ is Ala or Gly, X₂s is Arg, Ile, or Lys, X₂₆ is Gln or Pro, X₂₇ is Arg or Pro, X₂₈ is Ala, Cys, Leu, or Val, X₂₉ is Ala, Cys, Asn, Ser, or Thr, X₃₀ is Leu, Ala, or Val, X₃i is Glu or Pro, X₃₂ is His or Pro, X₃₃ is Leu, Asp, Asn, or Tyr, X₃₄ is Arg, Ala, Asp, Leu, Gln, or Tyr, X₃₅ is Ala, Cys, Phe, Gly, His, Ile, Lys, Leu, Met, Asn, Gln, Arg, Ser, Val, Trp, or Tyr, X₃₆ is Val, Ile, or Lys, X₃₇ is Thr or Ala, X₃₈ is Asp or Gly, X₃₉ is Glu, Lys, or Pro, X₄₀ is Ser or Cys, X₄i is Leu, Asp, Phe, Gln, or Thr, X₄₂ is Val or Phe, X₄₃ is Gln, Ala, His, Phe, Pro, Ser, or Thr, X₄₄ is Cys or Val, X₄₅ is Trp or Arg, X₄₆ is Ser, Arg, Ala, Asp, Glu, Phe, Gly, His, Ile, Lys, Leu, Met, Asn, Pro, Gln, Thr, Val, Trp, or Tyr, X₄₇ is Trp, Lys, Ala, Asp, Glu, Phe, Gly, His, Ile, Lys, Leu, Met, Asn, Pro, Gln, Arg, Ser, Thr, Val, or Tyr, X₄₈ is Lys or Val, X₄₉ is Ala, Cys, Ser, or Val, X₅₀ is Cys, Leu, or Val, X₅₁ is Val or Arg, and X₅₂ is Leu, Gln, His, Ile, Lys, or Ser, and the sialidase comprises at least one mutation relative to wild-type human Neu2 (SEQ ID NO: 1). In certain embodiments, the recombinant mutant human sialidase is then modified, or has been modified, to include one or more modifications to decrease protease sensitivity.

In certain embodiments, the recombinant mutant human sialidase comprises the amino acid sequence of

(SEQ ID NO: 200)
X₁ASLPX₂LQX₃ESVFQSGAHAYRIPALLYLPGQQSLLAFAEQRX₄SKKD
EHAELIVLRRGDYDAX₅THQVQWQAQEVVAQARLDGHRSMNPCPLYDX₆
QTGTLFLFFIAIPGQVTEQQQLQTRANVTRLCX₇VTSTDHGRTWSSPRD
LTDAAIGPAYREWSTFAVGPGHCLQLHDRARSLVVPAYAYRKLHPX₈QR
PIPSAFCFLSHDHGRTWARGHFVAQDTLECQVAEVETGEQRVVTLNARS
HLRX₉RVQAQSTNDGLDFQESQLVKKLVEPPPX₁₀GCQGSVISFPSPRSG
PGSPAQWLLYTHPTHX₁₁X₁₂QRADLGAYLNPRPPAPEAWSEPVLLAKGS
X₁₃AYSDLQSMGTGPDGSPLFGCLYEANDYEEIX₁₄FX₁₅MFTLKQAFPAE
YLPQ,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Arg or Ala, X₅ is Pro, Asn, Asp, His, Glu, Gly, Ser or Thr, X₆ is Ala, Glu, or Lys, X₇ is Gln, Leu, Glu, Phe, His, Ile, Leu, or Tyr, X₈ is Arg, Ile, or Lys, X₉ is Ala, Cys, Phe, Gly, His, Ile, Lys, Leu, Met, Asn, Gln, Arg, Ser, Val, Trp, or Tyr, X₁₀ is Gln, Ala, His, Phe, Pro, Ser, or Thr, X₁₁ is Ser, Arg, Ala, Asp, Glu, Phe, Gly, His, Ile, Lys, Leu, Met, Asn, Pro, Gln, Thr, Val, Trp, or Tyr, X₁₂ is Trp, Lys, Ala, Asp, Glu, Phe, Gly, His, Ile, Lys, Leu, Met, Asn, Pro, Gln, Arg, Ser, Thr, Val, or Tyr, X₁₃ is Ala, Cys, Ser, or Val, X₁₄ is Val or Arg, and X₁₅ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Arg or Ala, X₅ is Pro, Asn, Gly, Ser or Thr, X₆ is Ala or Glu, X₇ is Gln or Tyr, X₈ is Ile or Lys, X₉ is Ala or Thr, X₁₀ is Gln, Ala, or Thr, X₁₁ is Ser, Arg, or Ala, X₁₂ is Trp, Lys, or Arg, X₁₃ is Ala or Cys, X₁₄ is Val or Arg, and X₁₅ is Leu or Ile. In certain embodiments, the recombinant mutant human sialidase is then modified, or has been modified, to include one or more modifications to decrease protease sensitivity.

In certain embodiments, the recombinant mutant human sialidase comprises the amino acid sequence of

(SEQ ID NO: 47)
X₁X₂SX₃X₄X₅LQX₆ESVFQSGAHAYRIPALLYLPGQQSLLAFAEQRASX₇
X₈DEHAELIVX₉RRGDYDAX₁₀THQVQWX₁₁AQEVVAQAX₁₂LDGHRSMNPC
PLYDX₁₃QTGTLFLFFIAIPX₁₄X₁₅VTEX₁₆QQLQTRANVTRLX₁₇X₁₈VTS
TDHGRTWSSPRDLTDAAIGPX₁₉YREWSTFAVGPGHX₂₀LQLHDRX₂₁RSL
VVPAYAYRKLHPX₂₂QRPIPSAFX₂₃FLSHDHGRTWARGHFVAQDTX₂₄EC
QVAEVETGEQRVVTLNARSHLRARVQAQSX₂₅NX₂₆GLDFQX₂₇SQLVKKL
VEPPPX₂₈GX₂₉QGSVISFPSPRSGPGSPAQX₃₀LLYTHPTHX₃₁X₃₂QRAD
LGAYLNPRPPAPEAWSEPX₃₃LLAKGSX₃₄AYSDLQSMGTGPDGSPLFG
X₃₅LYEANDYEEIX₃₆FX₃₇MFTLKQAFPAEYLPQ,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp; X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₆ is Val or Arg, and X₃₇ is Leu, Gln, His, Ile, Lys, or Ser, and the sialidase comprises at least one mutation relative to wild-type human Neu2 (SEQ ID NO: 1). In certain embodiments, the recombinant mutant human sialidase is then modified, or has been modified, to include one or more modifications to decrease protease sensitivity.

In certain embodiments, the recombinant mutant human sialidase comprises the amino acid sequence of

(SEQ ID NO: 46)
X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅Q
TGTLFLFFIAIPGQVTEQQQLQTRANVTRLCQVTSTDHGRTWSSPRDLT
DAAIGPAYREWSTFAVGPGHCLQLHDRARSLVVPAYAYRKLHPX₆QRPI
PSAFCFLSHDHGRTWARGHFVAQDTLECQVAEVETGEQRVVTLNARSHL
RARVQAQSTNDGLDFQESQLVKKLVEPPPX₇GCQGSVISFPSPRSGPGS
PAQWLLYTHPTHX₈X₉QRADLGAYLNPRPPAPEAWSEPVLLAKGSX₁₀AY
SDLQSMGTGPDGSPLFGCLYEANDYEEIX₁₁FX₁₂MFTLKQAFPAEYLPQ,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, and X₁₂ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile. In certain embodiments, the recombinant mutant human sialidase is then modified, or has been modified, to include one or more modifications to decrease protease sensitivity.

In certain embodiments, the recombinant mutant human sialidase comprises the amino acid sequence of

(SEQ ID NO: 182)
X₁X₂SX₃X₄X₅LQX₆ESVFQSGAHAYRIPALLYLPGQQSLLAFAEQRASX₇X₈
DEHAELIVX₉RRGDYDAX₁₀THQVQWX₁₁AQEVVAQAX₁₂LX₁₃GHRSMNPCP
LYDX₁₄QTGTLFLFFIAIPX₁₅X₁₆VTEX₁₇QQLQTRANVTRLX₁₈X₁₉VTSTD
HGRTWSSPRDLTDAAIGPX₂₀YREWSTFAVGPGHX₂₁LQLHDX₂₂X₂₃RSLVV
PAYAYRKLHPX₂₄X₂₅X₂₆PIPSAFX₂₇FLSHDHGRTWARGHFVX₂₈QDTX₂₉E
CQVAEVX₃₀TGEQRVVTLNARSX₃₁X₃₂X₃₃X₃₄RX₃₅QAQSX₃₆NX₃₇GLDFQ
X₃₈X₃₉QX₄₀VKKLX₄₁EPPPX₄₂GX₄₃QGSVISFPSPRSGPGSPAQX₄₄LLYTH
PTHX₄₅X₄₆QRADLGAYLNPRPPAPEAWSEPX₄₇LLAKGSX₄₈AYSDLQSMGT
GPDGSPLFGX₄₉LYEANDYEEIX₅₀FX₅₁MFTLKQAFPAEYLPQ,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Lys, Arg, or Glu, X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Asp or Pro, X₁₄ is Ala, Glu or Lys, X₁₅ is Gly or Asp, X₁₆ is Gln or His, X₁₇ is Gln, Arg, or Lys, X₁₈ is Ala, Cys, Ile, Ser, Val, or Leu, X₁₉ is Gln, Leu, Glu, Phe, His, Ile, Leu, or Tyr, X₂₀ is Ala or Val, X₂₁ is Cys or Gly, X₂₂ is Arg or Pro, X₂₃ is Ala or Gly, X₂₄ is Arg, Ile, or Lys, X₂₅ is Gln or Pro, X₂₆ is Arg or Pro, X₂₇ is Ala, Cys, Leu, or Val, X₂₈ is Ala, Cys, Asn, Ser, or Thr, X₂₉ is Leu, Ala, or Val, X₃₀ is Glu or Pro, X₃₁ is His or Pro, X₃₂ is Leu, Asp, Asn, or Tyr, X₃₃ is Arg, Ala, Asp, Leu, Gln, or Tyr, X₃₄ is Ala, Cys, Phe, Gly, His, Ile, Lys, Leu, Met, Asn, Gln, Arg, Ser, Val, Trp, or Tyr, X₃₅ is Val, Ile, or Lys, X₃₆ is Thr or Ala, X₃₇ is Asp or Gly, X₃₈ is Glu, Lys, or Pro, X₃₉ is Ser or Cys, X₄₀ is Leu, Asp, Phe, Gln, or Thr, X₄₁ is Val or Phe, X₄₂ is Gln, Ala, His, Phe, Pro, Ser, or Thr, X₄₃ is Cys or Val, X₄₄ is Trp or Arg, X₄₅ is Ser, Arg, Ala, Asp, Glu, Phe, Gly, His, Ile, Lys, Leu, Met, Asn, Pro, Gln, Thr, Val, Trp, or Tyr, X₄₆ is Trp, Lys, Ala, Asp, Glu, Phe, Gly, His, Ile, Lys, Leu, Met, Asn, Pro, Gln, Arg, Ser, Thr, Val, or Tyr, X₄₇ is Lys or Val, X₄₈ is Ala, Cys, Ser, or Val, X₄₉ is Cys, Leu, or Val, X₅₀ is Val or Arg, and X₅₁ is Leu, Gln, His, Ile, Lys, or Ser, and the sialidase comprises at least one mutation relative to wild-type human Neu2 (SEQ ID NO: 1). In certain embodiments, the recombinant mutant human sialidase is then modified, or has been modified, to include one or more modifications to decrease protease sensitivity.

In certain embodiments, the recombinant mutant human sialidase comprises the amino acid sequence of

(SEQ ID NO: 183)
X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅QT
GTLFLFFIAIPGQVTEQQQLQTRANVTRLCX₆VTSTDHGRTWSSPRDLT
DAAIGPAYREWSTFAVGPGHCLQLHDRARSLVVPAYAYRKLHPX₇QRPI
PSAFCFLSHDHGRTWARGHFVAQDTLECQVAEVETGEQRVVTLNARSHL
RX₈RVQAQSTNDGLDFQESQLVKKLVEPPPX₉GCQGSVISFPSPRSGPGS
PAQWLLYTHPTHX₁₀X₁₁QRADLGAYLNPRPPAPEAWSEPVLLAKGSX₁₂A
YSDLQSMGTGPDGSPLFGCLYEANDYEEIX₁₃FX₁₄MFTLKQAFPAEYLPQ,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Gln, Leu, Glu, Phe, His, Ile, Leu, or Tyr, X₇ is Arg, Ile, or Lys, X₈ is Ala, Cys, Phe, Gly, His, Ile, Lys, Leu, Met, Asn, Gln, Arg, Ser, Val, Trp, or Tyr, X₉ is Gln, Ala, His, Phe, Pro, Ser, or Thr, X₁₀ is Ser, Arg, Ala, Asp, Glu, Phe, Gly, His, Ile, Lys, Leu, Met, Asn, Pro, Gln, Thr, Val, Trp, or Tyr, X₁₁ is Trp, Lys, Ala, Asp, Glu, Phe, Gly, His, Ile, Lys, Leu, Met, Asn, Pro, Gln, Arg, Ser, Thr, Val, or Tyr, X₁₂ is Ala, Cys, Ser, or Val, X₁₃ is Val or Arg, and X₁₄ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Gln or Tyr, X₇ is Ile or Lys, X₈ is Ala or Thr, X₉ is Gln, Ala, or Thr, X₁₀ is Ser, Arg, or Ala, X₁₁ is Trp, Lys, or Arg, X₁₂ is Ala or Cys, X₁₃ is Val or Arg, and X₁₄ is Leu or Ile. In certain embodiments, the recombinant mutant human sialidase is then modified, or has been modified, to include one or more modifications to decrease protease sensitivity.

In certain embodiments, the recombinant mutant human sialidase comprises a conservative substitution relative to a recombinant mutant human sialidase sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) Proc. Natl. Acad. Sci. USA 87:2264-2268; Altschul, (1993) J. Mol. Evol. 36:290-300; Altschul et al., (1997) Nucleic Acids Res. 25:3389-3402, incorporated by reference herein) are tailored for sequence similarity searching. For a discussion of basic issues in searching sequence databases see Altschul et al., (1994) Nature Genetics 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) Proc. Natl. Acad. Sci. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; —W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

### II. Fusion Proteins/Antibody Conjugates

To promote the selective removal of sialic acids on hypersialylated cancer cells and/or in the tumor microenvironment, it may be helpful to target a sialidase as described herein to such a cell or to such a tumor microenvironment. Additionally, in order to promote the removal of sialic acid by a sialidase in a subject, it may be helpful to extend the plasma half-life of the sialidase in the subject. These can be achieved by including the sialidase in a fusion protein and/or antibody conjugate (e.g., a chemically conjugated conjugate).

Accordingly, the invention further provides fusion proteins comprising a sialidase enzyme, or a functional fragment thereof, and a portion or fragment of an antibody, such as an immunoglobulin Fc domain (also referred to herein as an Fc domain), or an immunoglobulin antigen-binding domain (also referred to herein as an antigen-binding domain). In certain embodiments, the sialidase and antibody or portion thereof (e.g., immunoglobulin Fc domain or antigen-binding domain) are linked by a peptide bond or an amino acid linker.

As used herein, unless otherwise indicated, the term “fusion protein” is understood to refer to a single polypeptide chain comprising amino acid sequences based upon two or more separate proteins or polypeptide chains, where the two amino acid sequences may be fused together directly or via an intervening linker sequence, e.g., via an intervening amino acid linker. A nucleotide sequence encoding a fusion protein can, for example, be created using conventional recombinant DNA technologies.

In certain embodiments, the fusion protein comprises a tag, such as a Strep tag (e.g., a Strep II tag), a His tag (e.g., a 10×His tag), a myc tag, or a FLAG tag. The tag can be located on the C-terminus or the N-terminus of the fusion protein. In certain embodiments, a fusion protein comprises a sialidase portion joined to a polypeptide comprising an immunoglobulin heavy chain in an N- to C-terminal orientation, wherein the sialidase portion comprises an N-terminal addition of MEDLRP (SEQ ID NO: 4), and a Strep II Tag is located on the C-terminus of the immunoglobulin heavy chain or the N-terminus of the sialidase portion.

a. Sialidase Portion

The sialidase portion of the fusion protein described herein can be any sialidase, e.g., a fungal, bacterial, non-human mammalian or human sialidase. In certain embodiments, the sialidase portion is a recombinant human sialidase comprising at least one mutation relative to a wild-type human sialidase, e.g., a substitution, deletion, or addition of at least one amino acid, as described above.

In certain embodiments, the sialidase is any recombinant mutant human sialidase disclosed herein, or a functional fragment thereof.

In certain embodiments, the sialidase (e.g., human sialidase) is engineered to include one or more modifications, for example, an amino acid deletion, amino acid addition, or amino acid substitution, to decrease protease sensitivity, and may then, alternatively or in addition, include one or more of the following substitutions.

In certain embodiments, the sialidase portion comprises a C332A and C352L mutation. In certain embodiments, the sialidase comprises an N-terminal addition of MEDLRP (SEQ ID NO: 4) or EDLRP (SEQ ID NO: 3). In certain embodiments, the sialidase portion comprises a LSHSLST (SEQ ID NO: 22) peptide on the N-terminus. In certain embodiments, the sialidase portion comprises an N-terminal addition of MEDLRP (SEQ ID NO: 4) and an A2K substitution. In certain embodiments, the sialidase portion comprises an N-terminal addition of MEDLRP (SEQ ID NO: 4) and a C332A substitution. In certain embodiments, the sialidase portion comprises an N-terminal addition of MEDLRP (SEQ ID NO: 4), a C332A substitution, and a C352L substitution.

In certain embodiments, the sialidase portion comprises an M1 deletion (ΔM1), M1A substitution, M1D substitution, V6Y substitution, K9D substitution, A42R substitution, P62G substitution, P62N substitution, P62S substitution, P62T substitution, A93E substitution, Q126Y substitution, I187K substitution, A242F substitution, A242T substitution, Q270A substitution, Q270T substitution, S301R substitution, S301R substitution, W302K substitution, W302R substitution, C332A substitution, V363R substitution, L365I substitution, or a combination of any of the foregoing.

In certain embodiments, the sialidase portion comprises the amino acid sequence of any one of SEQ ID NOs: 48-54, 149, 154, 159, 191, or 198, or an amino acid sequence that has at least 85%, 90%, 95%, 96%, 97%, 98%, or 99% sequence identity to any one of SEQ ID NOs: 48-54, 149, 154, 159, 191, or 198.

b. Antibody Portion

As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

In certain embodiments, the fusion protein comprises an immunoglobulin Fc domain. As used herein, unless otherwise indicated, the term “immunoglobulin Fc domain” refers to a fragment of an immunoglobulin heavy chain constant region which, either alone or in combination with a second immunoglobulin Fc domain, is capable of binding to an Fc receptor. An immunoglobulin Fc domain may include, e.g., immunoglobulin CH2 and CH3 domains. An immunoglobulin Fc domain may include, e.g., immunoglobulin CH2 and CH3 domains and an immunoglobulin hinge region. Boundaries between immunoglobulin hinge regions, CH2, and CH3 domains are well known in the art, and can be found, e.g., in the PROSITE database (available on the world wide web at prosite.expasy.org).

In certain embodiments, the immunoglobulin Fc domain is derived from a human IgG1, IgG2, IgG3, IgG4, IgA1, IgA2, IgD, IgE, and IgM Fc domain. A single amino acid substitution (S228P according to Kabat numbering; designated IgG4Pro) may be introduced to abolish the heterogeneity observed in recombinant IgG4 antibody. See Angal, S. et al. (1993) MOL. IMMUNOL. 30:105-108.

In certain embodiments, the immunoglobulin Fc domain is derived from a human IgG1 isotype or another isotype that elicits antibody-dependent cell-mediated cytotoxicity (ADCC) and/or complement mediated cytotoxicity (CDC). In certain embodiments, the immunoglobulin Fc domain is derived from a human IgG1 isotype (e.g., SEQ ID NO: 31, SEQ ID NO: 5, or SEQ ID NO: 211).

In certain embodiments, the immunoglobulin Fc domain is derived from a human IgG4 isotype or another isotype that elicits little or no antibody-dependent cell-mediated cytotoxicity (ADCC) and/or complement mediated cytotoxicity (CDC). In certain embodiments, the immunoglobulin Fc domain is derived from a human IgG4 isotype.

In certain embodiments, the immunoglobulin Fc domain comprises either a “knob” mutation, e.g., T366Y, or a “hole” mutation, e.g., Y407T, for heterodimerization with a second polypeptide (residue numbers according to EU numbering, Kabat, E. A., et al. (1991) SEQUENCES OF PROTEINS OF IMMUNOLOGICAL INTEREST, FIFTH EDITION, U.S. Department of Health and Human Services, NIH Publication No. 91-3242). For example, in certain embodiments, the immunoglobulin Fc domain is derived from a human IgG1 Fc domain and comprises a Y407T mutation (e.g., the fusion protein comprises SEQ ID NO: 32, SEQ ID NO: 147, SEQ ID NO: 213, or SEQ ID NO: 215). In certain embodiments, the immunoglobulin Fc domain is derived from a human IgG1 Fc domain and comprises a T366Y mutation (e.g., the fusion protein comprises SEQ ID NO: 33, SEQ ID NO: 148, SEQ ID NO: 214, or SEQ ID NO: 216).

In certain embodiments, the immunoglobulin Fc domain is modified to prevent glycosylation of the Fc domain. For example, in certain embodiments, the immunoglobulin Fc domain is derived from a human IgG1 Fc domain and comprises a mutation at position N297, for example, an N297A mutation (residue numbers according to EU numbering, Kabat, E. A., et al., supra). For example, in certain embodiments, the fusion protein comprises SEQ ID NO: 212, SEQ ID NO: 215, or SEQ ID NO: 216.

In certain embodiments, the fusion protein comprises an immunoglobulin antigen-binding domain. The inclusion of such a domain may improve targeting of a fusion protein to a sialylated cancer cell and/or to the tumor microenvironment. As used herein, unless otherwise indicated, the term “immunoglobulin antigen-binding domain” refers to a polypeptide that, alone or in combination with another immunoglobulin antigen-binding domain, defines an antigen-binding site. Exemplary immunoglobulin antigen-binding domains include, for example, immunoglobulin heavy chain variable region and an immunoglobulin light chain variable region, where the variable regions together define an antigen binding site.

The immunoglobulin antigen-binding domain and/or antigen binding site can be derived from an antibody selected from, for example, adecatumumab, ascrinvacumab, cixutumumab, conatumumab, daratumumab, drozitumab, duligotumab, durvalumab, dusigitumab, enfortumab, enoticumab, epratuxumab, figitumumab, ganitumab, glembatumumab, intetumumab, ipilimumab, iratumumab, icrucumab, lexatumumab, lucatumumab, mapatumumab, narnatumab, necitumumab, nesvacumab, ofatumumab, olaratumab, panitumumab, patritumab, pritumumab, radretumab, ramucirumab, rilotumumab, robatumumab, seribantumab, tarextumab, teprotumumab, tovetumab, vantictumab, vesencumab, votumumab, zalutumumab, flanvotumab, altumomab, anatumomab, arcitumomab, bectumomab, blinatumomab, detumomab, ibritumomab, minretumomab, mitumomab, moxetumomab, naptumomab, nofetumomab, pemtumomab, pintumomab, racotumomab, satumomab, solitomab, taplitumomab, tenatumomab, tositumomab, tremelimumab, abagovomab, atezolizumab, durvalumab, avelumab, igovomab, oregovomab, capromab, edrecolomab, nacolomab, amatuximab, bavituximab, brentuximab, cetuximab, derlotuximab, dinutuximab, ensituximab, futuximab, girentuximab, indatuximab, isatuximab, margetuximab, rituximab, siltuximab, ublituximab, ecromeximab, abituzumab, alemtuzumab, bevacizumab, bivatuzumab, brontictuzumab, cantuzumab, cantuzumab, citatuzumab, clivatuzumab, dacetuzumab, demcizumab, dalotuzumab, denintuzumab, elotuzumab, emactuzumab, emibetuzumab, enoblituzumab, etaracizumab, farletuzumab, ficlatuzumab, gemtuzumab, imgatuzumab, inotuzumab, labetuzumab, lifastuzumab, lintuzumab, lirilumab, lorvotuzumab, lumretuzumab, matuzumab, milatuzumab, moxetumomab, nimotuzumab, obinutuzumab, ocaratuzumab, otlertuzumab, onartuzumab, oportuzumab, parsatuzumab, pertuzumab, pidilizumab, pinatuzumab, polatuzumab, sibrotuzumab, simtuzumab, tacatuzumab, tigatuzumab, trastuzumab, tucotuzumab, urelumab, vandortuzumab, vanucizumab, veltuzumab, vorsetuzumab, sofituzumab, catumaxomab, ertumaxomab, depatuxizumab, ontuxizumab, blontuvetmab, tamtuvetmab, nivolumab, pembrolizumab, epratuzumab, MEDI9447, urelumab, utomilumab, hu3F8, hu14.18-IL-2, 3F8/OKT3BsAb, lirilumab, BMS-986016 pidilizumab, AMP-224, AMP-514, BMS-936559, atezolizumab, and avelumab. In certain embodiments, the immunoglobulin antigen-binding domain can be derived from an antibody selected from trastuzumab, daratumumab, girentuximab, ofatumumab, avelumab, and rituximab.

In certain embodiments, the immunoglobulin antigen-binding domain is derived from trastuzumab. The trastuzumab heavy chain amino acid sequence is depicted in SEQ ID NO: 63, and the trastuzumab light chain amino acid sequence is depicted in SEQ ID NO: 64. The amino acid sequence of an exemplary scFv derived from trastuzumab is depicted in SEQ ID NO: 65.

The immunoglobulin antigen-binding domain and/or antigen binding site can be derived from an antibody that binds a cancer antigen selected from, for example, adenosine A2a receptor (A2aR), A kinase anchor protein 4 (AKAP4), B melanoma antigen (BAGE), brother of the regulator of imprinted sites (BORIS), breakpoint cluster region Abelson tyrosine kinase (BCR/ABL), CA125, CAIX, CD19, CD20, CD22, CD30, CD33, CD52, CD73, CD137, carcinoembryonic antigen (CEA), a claudin (e.g. a claudin 18, e.g., claudin 18.2), CS1, cytotoxic T-lymphocyte-associated antigen 4 (CTLA-4), estrogen receptor binding site associated antigen 9 (EBAG9), epidermal growth factor (EGF), epidermal growth factor receptor (EGFR), EGF-like module receptor 2 (EMR2), epithelial cell adhesion molecule (EpCAM) (17-1A), FR-alpha, G antigen (GAGE), disialoganglioside GD2 (GD2), glycoprotein 100 (gp100), human epidermal growth factor receptor 2 (HER2), hepatocyte growth factor (HGF), human papillomavirus 16 (HPV-16), heat-shock protein 105 (HSP105), isocitrate dehydrogenase type 1 (IDH1), idiotype (NeuGcGM3), indoleamine-2,3-dioxygenase 1 (IDO1), IGF-1, IGF1R, IGG1K, killer cell immunoglobulin-like receptor (KIR), lymphocyte activation gene 3 (LAG-3), lymphocyte antigen 6 complex K (LY6K), Matrix-metalloproteinase-16 (MMP16), melanotransferrin (MFI2), melanoma antigen 3 (MAGE-A3), melanoma antigen C2 (MAGE-C2), melanoma antigen D4 (MAGE-D4), melanoma antigen recognized by T-cells 1 (Melan-A/MART-1), N-methyl-N′-nitroso-guanidine human osteosarcoma transforming gene (MET), mucin 1 (MUC1), mucin 4 (MUC4), mucin 16 (MUC16), New York esophageal squamous cell carcinoma 1 (NY-ESO-1), prostatic acid phosphatase (PAP), programmed cell death receptor 1 (PD-1), programmed cell death receptor ligand 1 (PD-L1), phosphatidylserine, preferentially expressed antigen of melanoma (PRAME), prostate specific antigen (PSA), protein tyrosine kinase 7 (PTK7, also known as colon carcinoma kinase 4 (CCK4)), receptor tyrosine kinase orphan receptor 1 (ROR1), scatter factor receptor kinase, sialyl-Tn, sperm-associated antigen 9 (SPAG-9), synovial sarcoma X-chromosome breakpoint 1 (SSX1), survivin, telomerase, T-cell immunoglobulin domain and mucin domain-3 (TIM-3), vascular endothelial growth factor (VEGF) (e.g., VEGF-A), vascular endothelial growth factor Receptor 2 (VEGFR2), V-domain immunoglobulin-containing suppressor of T-cell activation (VISTA), Wilms' Tumor-1 (WT1), X chromosome antigen 1b (XAGE-1b), 5T4, Mesothelin, Glypican 3 (GPC3), Folate Receptor α (FRα), Prostate Specific Membrane Antigen (PSMA), cMET, CD38, B Cell Maturation Antigen (BCMA), CD123, CLDN6, CLDN9, LRRC15, PRLR (Prolactin Receptor), RING finger protein 43 (RNF43), Uroplakin-1 B (UPK1 B), tumor necrosis factor superfamily member 9 (TNFSF9), tumor necrosis factor receptor superfamily member 21 (TNFSRF21), bone morphogenetic protein receptor type-1B (BMPR1B), Kringle domain-containing transmembrane protein 2 (KREMEN2), Delta-like protein 3 (DLL3), Siglec7 and Siglec9. Additional exemplary cancer antigens include those found on cancer stem cells, e.g., SSEA3, SSEA4, TRA-1-60, TRA-1-81, SSEA1, CD133 (AC133), CD90 (Thy-1), CD326 (EpCAM), Cripto-1 (TDGF1), PODXL-1 (Podocalyxin-like protein 1), ABCG2, CD24, CD49f (Integrin a6), Notch2, CD146 (MCAM), CD10 (Neprilysin), CD117 (c-KIT), CD26 (DPP-4), CXCR4, CD34, CD271, CD13 (Alanine aminopeptidase), CD56 (NCAM), CD105 (Endoglin), LGR5, CD114 (CSF3R), CD54 (ICAM-1), CXCR1, 2, TIM-3 (HAVCR2), CD55 (DAF), DLL4 (Delta-like ligand 4), CD20 (MS4A1), and CD96.

The invention further provides antibody conjugates containing one or more of the fusion proteins disclosed herein. As used herein, unless otherwise indicated, the term “antibody conjugate” is understood to refer to an antibody, or a functional fragment thereof, that comprises antigen-binding activity and/or Fc receptor-binding activity, conjugated (e.g., covalently coupled) to an additional functional moiety. In certain embodiments, the antibody or functional antibody fragment is conjugated to a sialidase enzyme, e.g., a recombinant mutant human sialidase enzyme disclosed herein. In certain embodiments, an antibody conjugate comprises a single polypeptide chain. In certain embodiments, an antibody conjugate comprises two, three, four, or more polypeptide chains that are covalently or non-covalently associated together to produce a multimeric complex, e.g., a dimeric, trimeric or tetrameric complex.

TABLE 10 shows antibodies and antibody-drug conjugates suitable for use in accordance with the present invention, the antigen bound by the antibody or antibody-drug conjugate, and for certain antibodies, the type of cancer targeted by the antibody or antibody-drug conjugate.

TABLE 10

| Antibody or antibody- | | |
|---|---|---|
| drug conjugate | Cancer Antigen | Cancer Type |
| oregovomab | CA125 | |
| girentuximab | CAIX | |
| obinutuzumab | CD20 | |
| ofatumumab | CD20 | |
| rituximab | CD20 | |
| alemtuzumab | CD52 | |
| Ipilimumab | cytotoxic T-lymphocyte-associated | |
| | antigen 4 (CTLA-4) | |
| tremelimumab | CTLA-4 | |
| Cetuximab | epidermal growth factor receptor | |
| | (EGFR) | |
| necitumumab | EGFR | |
| panitumumab | EGFR | |
| zalutumumab | EGFR | |
| edrecolomab | epithelial cell adhesion molecule | |
| | (EpCAM) (17-1A) | |
| farletuzumab | FR-alpha | |
| Pertuzumab | human epidermal growth factor | |
| | receptor 2 (HER2) | |
| trastuzumab | HER2 | |
| rilotumumab | HGF | |
| figitumumab | IGF-1 | |
| Ganitumab | IGF1R | |
| durvalumab | IGG1K | |
| bavituximab | Phosphatidylserine | |
| onartuzumab | scatter factor receptor kinase | |
| bevacizumab | vascular endothelial growth factor-A | |
| | (VEGF-A) | |
| ramucirumab | vascular endothelial growth factor | |
| | Receptor 2 (VEGFR2) | |
| blinatumomab | CD19 | acute lymphoblastic leukemia |
| | | (ALL) |
| Rituximab; | CD20 | non-Hodgkin's lymphoma |
| ofatumumab; | | (NHL), chronic lymphocytic |
| ibritumomab (e.g., ⁹⁰Y- | | leukemia (CLL) |
| ibritumomab; | | B-cell NHL |
| tositumomab (e.g., | | pre-B ALL |
| ¹³¹I- | | |
| tositumomab | | |
| brentuximab (e.g., | CD30 | Hodgkin's lymphoma |
| brentuximab vedotin | | |
| gemtuzumab (e.g., | CD33 | acute myelogenous leukemia |
| gemtuzumab ozogamicin | | (AML) |
| Alemtuzumab | CD52 | CLL |
| Ipilimumab | cytotoxic T-lymphocyte-associated | Unresectable or metastatic |
| | antigen 4 (CTLA-4) | melanoma |
| cetuximab; | epidermal growth factor receptor | colorectal cancer (CRC) |
| panitumumab | (EGFR) | Head and Neck |
| Catumaxomab | epithelial cell adhesion molecule | Malignant ascites |
| | (EpCAM) | |
| trastuzumab; | human epidermal growth factor | Breast |
| pertuzumab | receptor 2 (HER2) | |
| nivolumab; | programmed cell death receptor 1 | Metastatic melanoma, non- |
| pembrolizumab | (PD-1) | small cell lung cancer |
| | | (NSCLC) |
| Bevacizumab | vascular endothelial growth factor | Breast, Cervical |
| | (VEGF) | CRC, NSCLC |
| | | renal cell carcinoma (RCC), |
| | | Ovarian |
| | | Glioblastoma |
| Ramucirumab | vascular endothelial growth factor | Gastric |
| | receptor 2 (VEGF-R2) | NSCLC |
| Epratuzumab; | CD22 | acute lymphoblastic leukemia |
| moxetumomab; | | (ALL) |
| inotuzumab (e.g., | | |
| inotuzumab ozogamicin) | | |
| MEDI9447 | CD73 | Advanced solid tumors |
| Urelumab; | CD137 | Advanced solid tumors |
| utomilumab (PF- | | |
| 05082566) | | |
| Elotuzumab | CD2 subset 1 (CS1) | Multiple myeloma |
| Tremelimumab | cytotoxic T-lymphocyte-associated | Malignant mesothelioma |
| | antigen 4 (CTLA-4) | |
| Necitumumab | epidermal growth factor receptor | non-small cell lung cancer |
| | (EGFR) | (NSCLC) |
| dinutuximab; hu3F8; | disialoganglioside GD2 (GD2) | Neuroblastoma |
| hu14. 18-IL-2; | | Retinoblastoma |
| 3F8/OKT3BsAb | | Melanoma other solid tumors |
| Racotumomab | Idiotype (NeuGcGM3) | NSCLC, Breast |
| | | Melanoma |
| Lirilumab | killer cell immunoglobulin-like | Lymphoma |
| | receptor (KIR) | |
| BMS-986016 | lymphocyte activation gene 3 | Breast, Hematological, |
| | (LAG-3 | Advanced solid tumors |
| Onartuzumab | N-methyl-N′-nitroso-guanidine | NSCLC |
| | human osteosarcoma transforming | |
| | gene (MET) | |
| abagovomab; | mucin 16 (MUC16) | Ovarian |
| oregovomab | | |
| pidilizumab; | programmed cell death receptor 1 | B-cell lymphoma |
| AMP-224; AMP-514 | (PD-1) | Melanoma, CRC |
| BMS-936559; | programmed cell death receptor | NSCLC, renal cell carcinoma |
| atezolizumab; | ligand 1 (PD-L1) | (RCC) |
| durvalumab; avelumab | | Bladder, Breast |
| | | Melanoma, squamous cell |
| | | carcinoma of the head and |
| | | neck (SCCHN) |
| naptumomab (e.g., | 5T4 | RCC, CRC |
| naptumomab estafenatox) | | Prostate |

c. Linker

In certain embodiments, the sialidase portion of the fusion protein can be linked or fused directly to the antibody portion (e.g., immunoglobulin Fc domain and/or immunoglobulin antigen-binding domain) of the fusion protein. In other embodiments, the sialidase portion can be covalently bound to the antibody portion by a linker.

The linker may couple, with one or more natural amino acids, the sialidase, or functional fragment thereof, and the antibody portions or fragments, where the amino acid (for example, a cysteine amino acid) may be introduced by site-directed mutagenesis. The linker may include one or more unnatural amino acids. It is contemplated that, in certain circumstances, a linker containing for example, one or more sulfhydryl reactive groups (e.g., a maleimide) may covalently link a cysteine in the sialidase portion or the antibody portion that is a naturally occurring cysteine residue or is the product of site-specific mutagenesis.

The linker may be a cleavable linker or a non-cleavable linker. Optionally or in addition, the linker may be a flexible linker or an inflexible linker.

The linker should be a length sufficiently long to allow the sialidase and the antibody portions to be linked without steric hindrance from one another and sufficiently short to retain the intended activity of the fusion protein. The linker preferably is sufficiently hydrophilic to avoid or minimize instability of the fusion protein. The linker preferably is sufficiently hydrophilic to avoid or minimize insolubility of the fusion protein. The linker should be sufficiently stable in vivo (e.g., it is not cleaved by serum, enzymes, etc.) to permit the fusion protein to be operative in vivo.

The linker may be from about 1 angstroms (Å) to about 150 Å in length, or from about 1 Å to about 120 Å in length, or from about 5 Å to about 110 Å in length, or from about 10 Å to about 100 Å in length. The linker may be greater than about 2, 3, 4, 5, 6, 7, 8, 9, 10, 11, 12, 13, 14, 15, 16, 17, 18, 19, 20, 25, 27, 30 or greater angstroms in length and/or less than about 110, 100, 90, 85, 80, 75, 70, 65, 60, 55, 50, 45, 43, 42, 41, 40, 39, 38, 37, 36, 35, 34, 33, 32, 31, or fewer A in length. Furthermore, the linker may be about 5, 10, 15, 20, 25, 30, 35, 40, 45, 50, 55, 60, 65, 70, 75, 80, 85, 90, 95, 100, 110, and 120 Å in length.

In certain embodiments, the linker comprises a polypeptide linker that connects or fuses the sialidase portion of the fusion protein to the antibody portion (e.g., immunoglobulin Fc domain and/or immunoglobulin antigen-binding domain) of the fusion protein. For example, it is contemplated that a gene encoding a sialidase portion linked directly or indirectly (for example, via an amino acid containing linker) to an antibody portion can be created and expressed using conventional recombinant DNA technologies. For example, the amino terminus of a sialidase portion can be linked to the carboxy terminus of either the light or the heavy chain of an antibody portion. For example, for a Fab fragment, the amino terminus or carboxy terminus of the sialidase can be linked to the first constant domain of the heavy antibody chain (CH1). When a linker is employed, the linker may comprise hydrophilic amino acid residues, such as Gln, Ser, Gly, Glu, Pro, His and Arg. In certain embodiments, the linker is a peptide containing 1-25 amino acid residues, 1-20 amino acid residues, 2-15 amino acid residues, 3-10 amino acid residues, 3-7 amino acid residues, 4-25 amino acid residues, 4-20 amino acid residues, 4-15 amino acid residues, 4-10 amino acid residues, 5-25 amino acid residues, 5-20 amino acid residues, 5-15 amino acid residues, or 5-amino acid residues. Exemplary linkers include glycine and serine-rich linkers, e.g., (GlyGlyPro)ₙ, or (GlyGlyGlyGlySer)ₙ, where n is 1-5. In certain embodiments, the linker comprises, consists, or consists essentially of GGGGS (SEQ ID NO: 184). In certain embodiments, the linker comprises, consists, or consists essentially of GGGGSGGGGS (SEQ ID NO: 145). In certain embodiments, the linker comprises, consists, or consists essentially of EPKSS (SEQ ID NO: 146). Additional exemplary linker sequences are disclosed, e.g., in George et al. (2003) PROTEIN ENGINEERING 15:871-879, and U.S. Pat. Nos. 5,482,858 and 5,525,491.

In certain embodiments, the fusion protein comprises the amino acid sequence of any one of SEQ ID NOs: 66-85, 98-142, 150-153, 155-158, 160-163, 166-178, 185, 187, 189, 192-197, 203-210, 218, 220, 222, 224, 226, 228, 230, 232, 234, 236, 238, 240, 242, 244, or 249, or an amino acid sequence that has at least 85%, 90%, 95%, 96%, 97%, 98%, or 99% sequence identity to any one of SEQ ID NOs: 66-85, 98-142, 150-153, 155-158, 160-163, 166-178, 185, 187, 189, 192-197, 203-210, 218, 220, 222, 224, 226, 228, 230, 232, 234, 236, 238, 240, 242, 244, or 249.

d. Antibody Conjugates

The invention further provides antibody conjugates comprising a fusion protein disclosed herein. The antibody conjugate may comprise a single polypeptide chain (i.e., a fusion protein disclosed herein) or, the antibody conjugate may comprise additional polypeptide chains (e.g., one, two, or three additional polypeptide chains). For example, an antibody conjugate may comprise a first polypeptide (fusion protein) comprising a recombinant mutant human sialidase enzyme and an immunoglobulin heavy chain, and a second polypeptide comprising an immunoglobulin light chain, where, for example, the immunoglobulin heavy and light chains together define a single antigen-binding site.

In certain embodiments, the antibody conjugate can include a single sialidase. In other embodiments, the antibody conjugate can include more than one (e.g., two) sialidases. If more than one sialidase is included, the sialidases can be the same or different. In certain embodiments, the antibody conjugate can include a single antigen-binding site. In other embodiments, the antibody conjugate can include more than one (e.g., two) antigen-binding sites. If two antigen-binding sites are used, they can be the same or different. In certain embodiments, the antibody conjugate comprises an immunoglobulin Fc fragment.

In certain embodiments, the antibody conjugate comprises one or two immunoglobulin heavy chains, or a functional fragment thereof. In certain embodiments, the antibody conjugate comprises one or two immunoglobulin light chains, or a functional fragment thereof. In certain embodiments, the antibody conjugate comprises a sialidase fused to the N- or C-terminus of an immunoglobulin heavy chain or an immunoglobulin light chain.

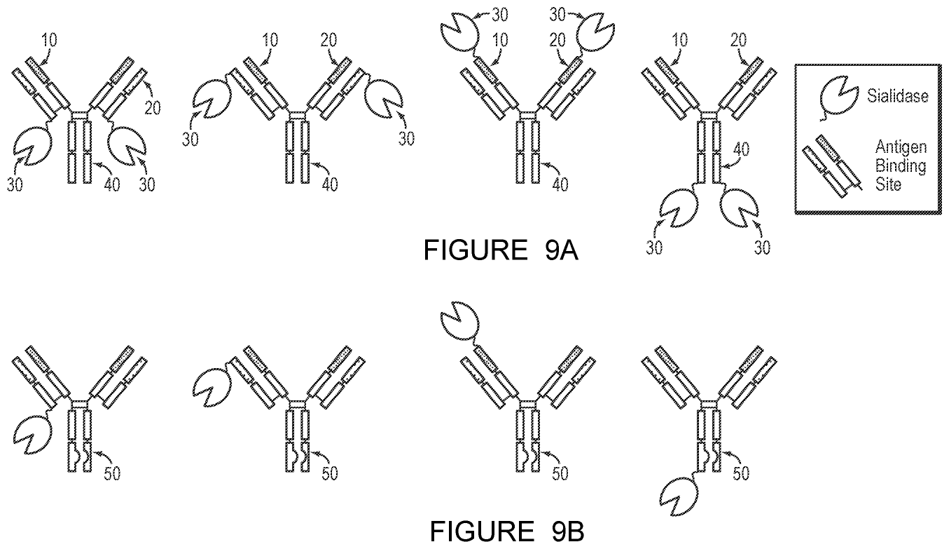

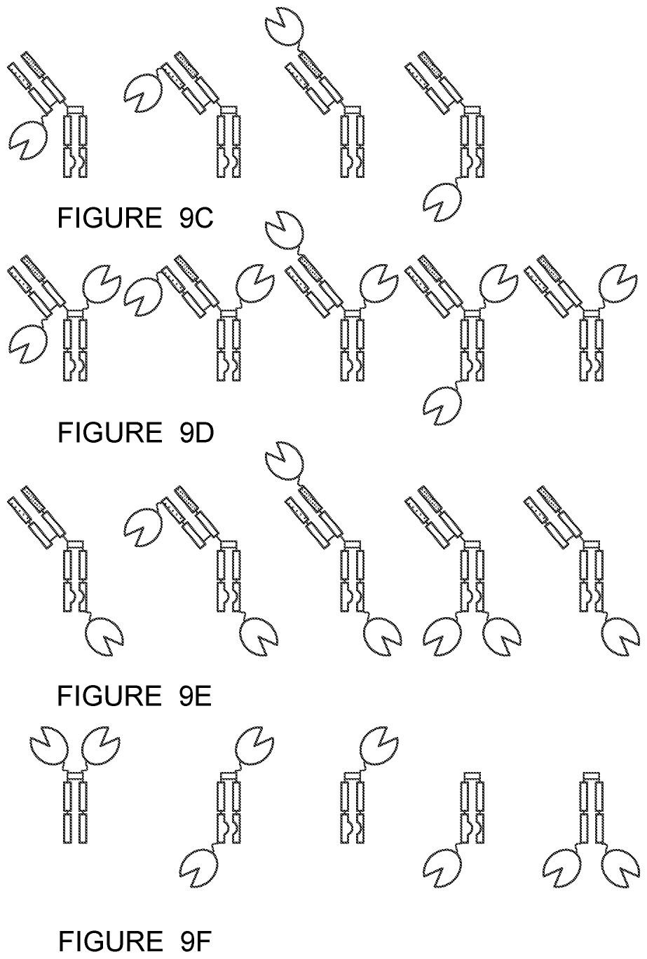

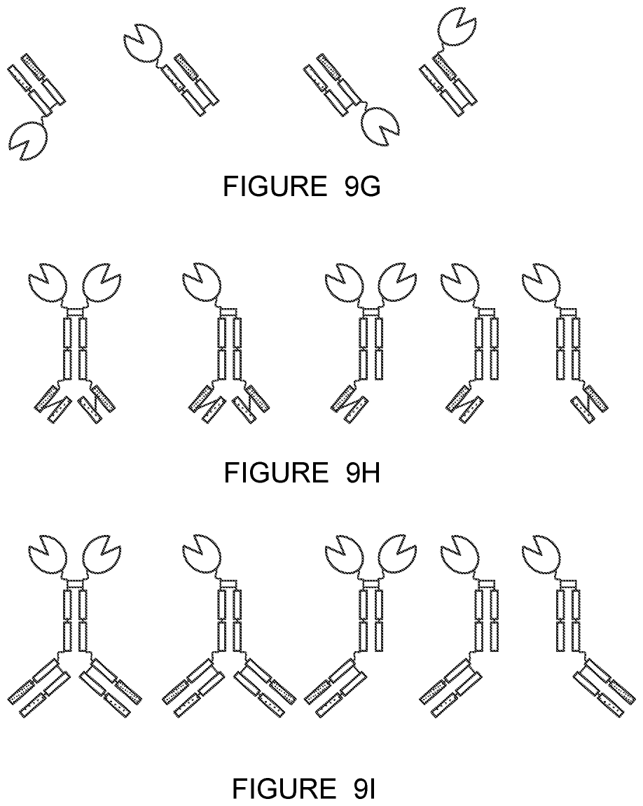

FIG. 9 depicts exemplary antibody conjugate constructs containing one or more sialidase enzymes. For example, in FIG. 9A, a first antigen-binding site is depicted as 10, a second antigen-binding site is depicted as 20, a sialidase is depicted as 30, and a Fab is depicted as 40. In each of the constructs depicted in FIGS. 9A-9I it is understood that the Fc may optionally be modified in some manner, e.g. using Knobs-into-Holes type technology, e.g., as depicted by 50 in FIG. 9B. Throughout FIG. 9 similar structures are depicted by similar schematic representations.

FIG. 9A depicts antibody conjugate constructs comprising a first polypeptide comprising a first immunoglobulin light chain; a second polypeptide comprising a first immunoglobulin heavy chain; a third polypeptide comprising a second immunoglobulin heavy chain; and a fourth polypeptide comprising a second immunoglobulin light chain. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first antigen-binding site as depicted as 10, and the third polypeptide and the fourth polypeptide together define a second antigen-binding site as depicted as 20. A sialidase enzyme as depicted as 30 can be conjugated to the N- or C-terminus of the first and second immunoglobulin light chain or the first and second immunoglobulin heavy chain.

FIG. 9B depicts antibody conjugate constructs comprising a first polypeptide comprising a first immunoglobulin light chain; a second polypeptide comprising a first immunoglobulin heavy chain; a third polypeptide comprising a second immunoglobulin heavy chain; and a fourth polypeptide comprising a second immunoglobulin light chain. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first antigen-binding site, and the third polypeptide and the fourth polypeptide together define a second antigen-binding site. A sialidase enzyme can be conjugated to the N- or C-terminus of the first immunoglobulin light chain or the first immunoglobulin heavy chain.

FIG. 9C depicts antibody conjugate constructs comprising a first polypeptide comprising an immunoglobulin light chain; a second polypeptide comprising an immunoglobulin heavy chain; and a third polypeptide comprising an immunoglobulin Fc domain. The first and second polypeptides can be covalently linked together and the second and third polypeptides can be covalently linked together. The covalent linkages can be disulfide bonds. In certain embodiments, the first polypeptide and the second polypeptide together define an antigen-binding site. A sialidase enzyme can be conjugated to the N- or C-terminus of the first immunoglobulin light chain or the first immunoglobulin heavy chain.

FIG. 9D depicts antibody conjugate constructs comprising a first polypeptide comprising an immunoglobulin light chain; a second polypeptide comprising an immunoglobulin heavy chain; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The third polypeptide comprises the sialidase and the immunoglobulin Fc domain in an N- to C-terminal orientation. In certain embodiments, the first polypeptide and the second polypeptide together define an antigen-binding site. An optional second sialidase enzyme can be conjugated to the N- or C-terminus of the first immunoglobulin light chain or the first immunoglobulin heavy chain.

FIG. 9E depicts antibody conjugate constructs comprising a first polypeptide comprising an immunoglobulin light chain; a second polypeptide comprising an immunoglobulin heavy chain; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The third polypeptide comprises the immunoglobulin Fc domain and the sialidase in an N- to C-terminal orientation. In certain embodiments, the first polypeptide and the second polypeptide together define an antigen-binding site. An optional second sialidase enzyme can be conjugated to the N- or C-terminus of the first immunoglobulin light chain or the first immunoglobulin heavy chain.

FIG. 9F depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N- or C-terminus of the first immunoglobulin Fc domain or to the N- or C-terminus of the second immunoglobulin Fc domain. An optional second sialidase enzyme can be conjugated to the N- or C-terminus of the first immunoglobulin Fc domain or to the N- or C-terminus of the second immunoglobulin Fc domain.

FIG. 9G depicts antibody conjugate constructs comprising a first polypeptide comprising an immunoglobulin light chain; and a second polypeptide comprising an immunoglobulin heavy chain variable region. The first and second polypeptides can be covalently linked together. The covalent linkages can be disulfide bonds. In certain embodiments, the first polypeptide and the second polypeptide together define an antigen-binding site. The sialidase enzyme can be conjugated to the N- or C-terminus of the immunoglobulin light chain or the immunoglobulin heavy chain variable region.

FIG. 9H depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N-terminus of the first immunoglobulin Fc domain or the second immunoglobulin Fc domain. An optional second sialidase enzyme can be conjugated to the N-terminus of the second immunoglobulin Fc domain or the first immunoglobulin Fc domain, respectively. A single chain variable fragment (scFv) can be conjugated to the C-terminus of the first immunoglobulin Fc domain or the second immunoglobulin Fc domain. An optional second single chain variable fragment (scFv) can be conjugated to the C-terminus of the first immunoglobulin Fc domain or the second immunoglobulin Fc domain, respectively.

FIG. 9I depicts antibody conjugate constructs similar to those depicted in FIG. 9H except that each scFv is replaced with an immunoglobulin antigen binding fragment, e.g., a Fab. For example, FIG. 9I depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N-terminus of the first immunoglobulin Fc domain or the second immunoglobulin Fc domain. An optional second sialidase enzyme can be conjugated to the N-terminus of the second immunoglobulin Fc domain or the first immunoglobulin Fc domain, respectively. An antibody fragment (Fab) can be conjugated or fused to the C-terminus of the first immunoglobulin Fc domain or the second immunoglobulin Fc domain. An optional second antibody fragment (Fab) can be conjugated or fused to the C-terminus of the second immunoglobulin Fc domain or the first immunoglobulin Fc domain, respectively. In the case of a fusion, the C terminus of the Fc domain is linked (either by a bond or an amino acid linker) to a first polypeptide chain defining an immunoglobulin antigen binding fragment. In the case of antibodies that have an antigen binding site defined by a single variable region, then this may be sufficient to impart binding affinity to a target antigen. In other instances, e.g., in the case of a human antibody, the first polypeptide chain defining an immunoglobulin antigen binding fragment can be conjugated (e.g., covalently conjugated, e.g., via a disulfide bond) to a second polypeptide chain defining an immunoglobulin antigen binding fragment, there the two antigen binding fragments together define an antigen binding site for binding the target antigen.

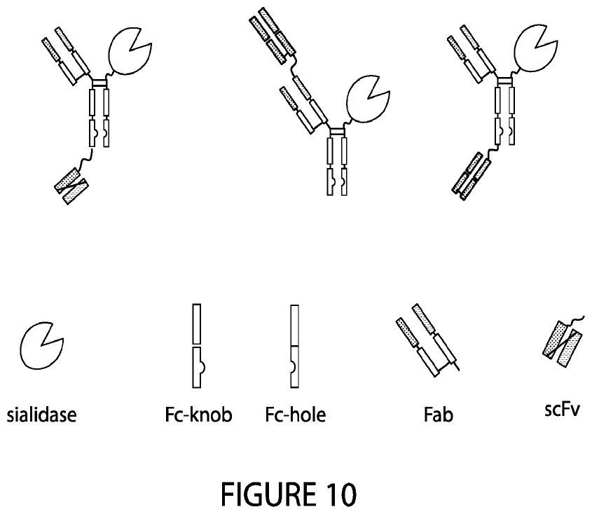

FIG. 10 depicts additional antibody conjugate constructs. For example, FIG. 10 depicts an antibody conjugate construct comprising a first polypeptide comprising an immunoglobulin light chain; a second polypeptide comprising an immunoglobulin heavy chain and an scFv; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The second polypeptide comprises the heavy chain and the scFv in an N- to C-terminal orientation. The third polypeptide comprises the sialidase and the immunoglobulin Fc domain in an N- to C-terminal orientation. In certain embodiments, the first polypeptide and the second polypeptide together define a first antigen-binding site. In certain embodiments, the scFv defines a second antigen-binding site. FIG. 10 depicts an additional antibody construct comprising a first polypeptide comprising an immunoglobulin light chain; a second polypeptide comprising an immunoglobulin heavy chain; and a third polypeptide comprising an immunoglobulin Fc domain and a first sialidase enzyme, wherein a Fab fragment is conjugated to the N-terminus of the immunoglobulin heavy chain. The first and second polypeptides can be covalently linked together and the second and third polypeptides can be covalently linked together. The covalent linkages can be disulfide bonds. The third polypeptide comprises the sialidase and the immunoglobulin Fc domain in an N- to C-terminal orientation. In certain embodiments, the first polypeptide and the second polypeptide together define a first antigen-binding site. In certain embodiments, the Fab fragment defines a second antigen-binding site. In each of the constructs depicted in FIG. 10 it is understood that an scFv, when present, may be replaced with a Fab fragment, or a Fab fragment, when present, may be replaced with an scFv. In each of the constructs depicted in FIG. 10, it is understood that the Fc may optionally be modified in some manner.

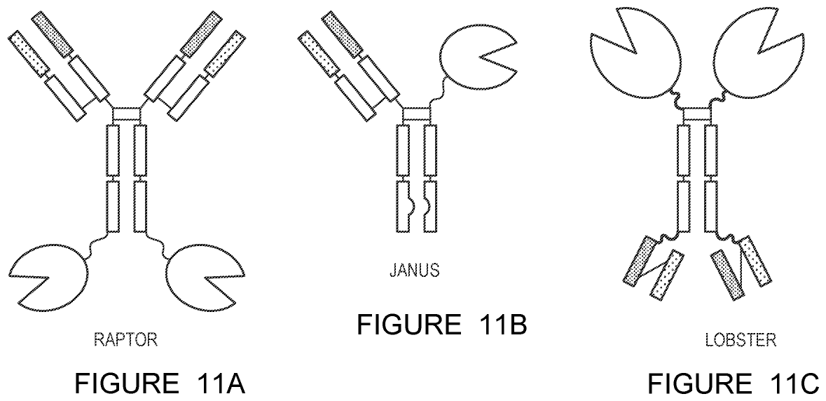

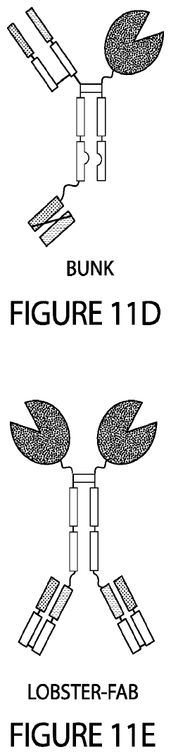

In certain embodiments, the antibody conjugate comprises a first polypeptide comprising a first immunoglobulin light chain; a second polypeptide comprising a first immunoglobulin heavy chain and a first sialidase; a third polypeptide comprising a second immunoglobulin heavy chain and a second sialidase; and a fourth polypeptide comprising a second immunoglobulin light chain. An example of this embodiment is shown in FIG. 11A. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first antigen-binding site, and the third polypeptide and the fourth polypeptide together define a second antigen-binding site. In certain embodiments, the second and third polypeptides comprise the first and second immunoglobulin heavy chain and the first and second sialidase, respectively, in an N- to C-terminal orientation. In certain embodiments, the second and third polypeptides comprise the first and second sialidase and the first and second immunoglobulin heavy chain, respectively, in an N- to C-terminal orientation.

In certain embodiments, the antibody conjugate comprises a first polypeptide comprising an immunoglobulin light chain; a second polypeptide comprising an immunoglobulin heavy chain; and a third polypeptide comprising an immunoglobulin Fc domain and a sialidase. An example of this embodiment is shown in FIG. 11B. The first and second polypeptides can be covalently linked together and the second and third polypeptides can be covalently linked together. The covalent linkages can be disulfide bonds. In certain embodiments, the first polypeptide and the second polypeptide together define an antigen-binding site. In certain embodiments, the third polypeptide comprises the sialidase and the immunoglobulin Fc domain in an N- to C-terminal orientation, or the immunoglobulin Fc domain and the sialidase in an N- to C-terminal orientation.

In certain embodiments, the first polypeptide comprises the amino acid sequence of SEQ ID NO: 66, or an amino acid sequence that has at least 85%, 90%, 95%, 96%, 97%, 98%, or 99% sequence identity to SEQ ID NO: 66. In certain embodiments, the second polypeptide comprises the amino acid sequence of SEQ ID NO: 67 or 189 or an amino acid sequence that has at least 85%, 90%, 95%, 96%, 97%, 98%, or 99% sequence identity to SEQ ID NO: 67 or 189. In certain embodiments, the third polypeptide comprises the amino acid sequence of any one of SEQ ID NOs: 68-74, 98-112, 150, 151, 155, 156, 160, 161, 185, 187, 192, 195, 203-208, or an amino acid sequence that has at least 85%, 90%, 95%, 96%, 97%, 98%, or 99% sequence identity to any one of SEQ ID NOs: 68-74, 98-112, 150, 151, 155, 156, 160, 161, 185, 187, 192, 195, or 203-208.

In certain embodiments, the third polypeptide comprises the amino acid sequence of

(SEQ ID NO: 201)
X₁X₂SX₃X₄X₅LQX₆ESVFQSGAHAYRIPALLYLPGQQSLLAFAEQRX₇SX₈
X₉DEHAELIVX₁₀RRGDYDAX₁₁THQVQWX₁₂AQEVVAQAX₁₃LX₁₄GHRSMN
PCPLYDX₁₅QTGTLFLFFIAIPX₁₆X₁₇VTEX₁₈QQLQTRANVTRLX₁₉X₂₀V
TSTDHGRTWSSPRDLTDAAIGPX₂₁YREWSTFAVGPGHX₂₂LQLHDX₂₃X₂₄
RSLVVPAYAYRKLHPX₂₅X₂₆X₂₇PIPSAFX₂₈FLSHDHGRTWARGHFVX₂₉
QDTX₃₀ECQVAEVX₃₁TGEQRVVTLNARSX₃₂X₃₃X₃₄X₃₅RX₃₆QAQSX₃₇NX₃₈
GLDFQX₃₉X₄₀QX₄₁VKKLX₄₂EPPPX₄₃GX₄₄QGSVISFPSPRSGPGSPAQX₄₅
LLYTHPTHX₄₆X₄₇QRADLGAYLNPRPPAPEAWSEPX₄₈LLAKGSX₄₉AYSDL
QSMGTGPDGSPLFGX₅₀LYEANDYEEIX₅₁FX₅₂MFTLKQAFPAEYLPQX₅₃D
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKG
FYPSDIAVEWESNGQPENNYKTTPPVLDSDGSFFLTSKLTVDKSRWQQGN
VFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Ala or Arg, X₈ is Lys, Arg, or Glu, X₉ is Lys, Ala, Arg, or Glu, X₁₀ is Leu or Met, X₁₁ is Pro, Asn, Asp, His, Glu, Gly, Ser or Thr, X₁₂ is Gln or His, X₁₃ is Arg or Lys, X₁₄ is Asp or Pro, X₁₅ is Ala, Glu or Lys, X₁₆ is Gly or Asp, X₁₇ is Gln or His, X₁₈ is Gln, Arg, or Lys, X₁₉ is Ala, Cys, Ile, Ser, Val, or Leu, X₂₀ is Gln, Leu, Glu, Phe, His, Ile, Leu, or Tyr, X₂₁ is Ala or Val, X₂₂ is Cys or Gly, X₂₃ is Arg or Pro, X₂₄ is Ala or Gly, X₂₅ is Arg, Ile, or Lys, X₂₆ is Gln or Pro, X₂₇ is Arg or Pro, X₂₈ is Ala, Cys, Leu, or Val, X₂₉ is Ala, Cys, Asn, Ser, or Thr, X₃₀ is Leu, Ala, or Val, X₃₁ is Glu or Pro, X₃₂ is His or Pro, X₃₃ is Leu, Asp, Asn, or Tyr, X₃₄ is Arg, Ala, Asp, Leu, Gln, or Tyr, X₃₅ is Ala, Cys, Phe, Gly, His, Ile, Lys, Leu, Met, Asn, Gln, Arg, Ser, Val, Trp, or Tyr, X₃₆ is Val, Ile, or Lys, X₃₇ is Thr or Ala, X₃₈ is Asp or Gly, X₃₉ is Glu, Lys, or Pro, X₄₀ is Ser or Cys, X₄₁ is Leu, Asp, Phe, Gln, or Thr, X₄₂ is Val or Phe, X₄₃ is Gln, Ala, His, Phe, Pro, Ser, or Thr, X₄₄ is Cys or Val, X₄₅ is Trp or Arg, X₄₆ is Ser, Arg, Ala, Asp, Glu, Phe, Gly, His, Ile, Lys, Leu, Met, Asn, Pro, Gln, Thr, Val, Trp, or Tyr, X₄₇ is Trp, Lys, Ala, Asp, Glu, Phe, Gly, His, Ile, Lys, Leu, Met, Asn, Pro, Gln, Arg, Ser, Thr, Val, or Tyr, X₄₈ is Lys or Val, X₄₉ is Ala, Cys, Ser, or Val, X₅₀ is Cys, Leu, or Val, X₅₁ is Val or Arg, X₅₂ is Leu, Gln, His, Ile, Lys, or Ser, and X₅₃ is GGGGS (SEQ ID NO: 184), GGGGSGGGGS (SEQ ID NO: 145), or EPKSS (SEQ ID NO: 146), and the sialidase comprises at least one mutation relative to wild-type human Neu2 (SEQ ID NO: 1).

In certain embodiments, the third polypeptide comprises the amino acid sequence of

(SEQ ID NO: 202)
X₁ASLPX₂LQX₃ESVFQSGAHAYRIPALLYLPGQQSLLAFAEQRX₄SKKDE
HAELIVLRRGDYDAX₅THQVQWQAQEVVAQARLDGHRSMNPCPLYDX₆QT
GTLFLFFIAIPGQVTEQQQLQTRANVTRLCX₇VTSTDHGRTWSSPRDLTD
AAIGPAYREWSTFAVGPGHCLQLHDRARSLVVPAYAYRKLHPX₈QRPIPS
AFCFLSHDHGRTWARGHFVAQDTLECQVAEVETGEQRVVTLNARSHLRX₉
RVQAQSTNDGLDFQESQLVKKLVEPPPX₁₀GCQGSVISFPSPRSGPGSPA
QWLLYTHPTHX₁₁X₁₂QRADLGAYLNPRPPAPEAWSEPVLLAKGSX₁₃AYS
DLQSMGTGPDGSPLFGCLYEANDYEEIX₁₄FX₁₅MFTLKQAFPAEYLPQX₁₆
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLTSKLTVDKSR
WQQGNVFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Arg or Ala, X₅ is Pro, Asn, Asp, His, Glu, Gly, Ser or Thr, X₆ is Ala, Glu, or Lys, X₇ is Gln, Leu, Glu, Phe, His, Ile, Leu, or Tyr, X₈ is Arg, Ile, or Lys, X₉ is Ala, Cys, Phe, Gly, His, Ile, Lys, Leu, Met, Asn, Gln, Arg, Ser, Val, Trp, or Tyr, X₁₀ is Gln, Ala, His, Phe, Pro, Ser, or Thr, X₁₁ is Ser, Arg, Ala, Asp, Glu, Phe, Gly, His, Ile, Lys, Leu, Met, Asn, Pro, Gln, Thr, Val, Trp, or Tyr, X₁₂ is Trp, Lys, Ala, Asp, Glu, Phe, Gly, His, Ile, Lys, Leu, Met, Asn, Pro, Gln, Arg, Ser, Thr, Val, or Tyr, X₁₃ is Ala, Cys, Ser, or Val, X₁₄ is Val or Arg, X₁₅ is Leu, Gln, His, Ile, Lys, or Ser, and X₁₆ is GGGGS (SEQ ID NO: 184), GGGGSGGGGS (SEQ ID NO: 145), or EPKSS (SEQ ID NO: 146), and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Arg or Ala, X₅ is Pro, Asn, Gly, Ser or Thr, X₆ is Ala or Glu, X₇ is Gln or Tyr, X₈ is Ile or Lys, X₉ is Ala or Thr, X₁₀ is Gln, Ala, or Thr, X₁₁ is Ser, Arg, or Ala, X₁₂ is Trp, Lys, or Arg, X₁₃ is Ala or Cys, X₁₄ is Val or Arg, and X₁₅ is Leu or Ile.

In certain embodiments, the third polypeptide comprises the amino acid sequence of

(SEQ ID NO: 76)
X₁X₂SX₃X₄X₅LQX₆ESVFQSGAHAYRIPALLYLPGQQSLLAFAEQRASX₇
X₈DEHAELIVX₉RRGDYDAX₁₀THQVQWX₁₁AQEVVAQAX₁₂LDGHRSMNPC
PLYDX₁₃QTGTLFLFFIAIPX₁₄X₁₅VTEX₁₆QQLQTRANVTRLX₁₇X₁₈VTS
TDHGRTWSSPRDLTDAAIGPX₁₉YREWSTFAVGPGHX₂₀LQLHDRX₂₁RSL
VVPAYAYRKLHPX₂₂QRPIPSAFX₂₃FLSHDHGRTWARGHFVAQDTX₂₄E
CQVAEVETGEQRVVTLNARSHLRARVQAQSX₂₅NX₂₆GLDFQX₂₇SQLVK
KLVEPPPX₂₈GX₂₉QGSVISFPSPRSGPGSPAQX₃₀LLYTHPTHX₃₁X₃₂Q
RADLGAYLNPRPPAPEAWSEPX₃₃LLAKGSX₃₄AYSDLQSMGTGPDGSP
LFGX₃₅LYEANDYEEIX₃₆FX₃₇MFTLKQAFPAEYLPQGGGGSGGGGSDKT
HTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVK
GFYPSDIAVEWESNGQPENNYKTTPPVLDSDGSFFLTSKLTVDKSRWQQ
GNVFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp; X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂s is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₆ is Val or Arg, and X₃₇ is Leu, Gln, His, Ile, Lys, or Ser, and the sialidase comprises at least one mutation relative to wild-type human Neu2 (SEQ ID NO: 1).

In certain embodiments, the third polypeptide comprises the amino acid sequence of

(SEQ ID NO: 75)
X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅QT
GTLFLFFIAIPGQVTEQQQLQTRANVTRLCQVTSTDHGRTWSSPRDLTD
AAIGPAYREWSTFAVGPGHCLQLHDRARSLVVPAYAYRKLHPX₆QRPIP
SAFCFLSHDHGRTWARGHFVAQDTLECQVAEVETGEQRVVTLNARSHLR
ARVQAQSTNDGLDFQESQLVKKLVEPPPX₇GCQGSVISFPSPRSGPGSP
AQWLLYTHPTHX₈X₉QRADLGAYLNPRPPAPEAWSEPVLLAKGSX₁₀AYS
DLQSMGTGPDGSPLFGCLYEANDYEEIX₁₁FX₁₂MFTLKQAFPAEYLPQG
GGGSGGGGSDKTHTCPPCPAPELLGGPSVFLFPPKPKDTLMISRTPEV
TCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGS
FFLTSKLTVDKSRWQQGNVFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, and X₁₂ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the third polypeptide comprises the amino acid sequence of

(SEQ ID NO: 144)
X₁X₂SX₃X₄X₅LQX₆ESVFQSGAHAYRIPALLYLPGQQSLLAFAEQRASX₇
X₈DEHAELIVX₉RRGDYDAX₁₀THQVQWX₁₁AQEVVAQAX₁₂LDGHRSMNPC
PLYDX₁₃QTGTLFLFFIAIPX₁₄X₁₅VTEX₁₆QQLQTRANVTRLX₁₇X₁₈VTS
TDHGRTWSSPRDLTDAAIGPX₁₉YREWSTFAVGPGHX₂₀LQLHDRX₂₁RSL
VVPAYAYRKLHPX₂₂QRPIPSAFX₂₃FLSHDHGRTWARGHFVAQDTX₂₄EC
QVAEVETGEQRVVTLNARSHLRARVQAQSX₂₅NX₂₆GLDFQX₂₇SQLVKKL
VEPPPX₂₈GX₂₉QGSVISFPSPRSGPGSPAQX₃₀LLYTHPTHX₃₁X₃₂QRADL
GAYLNPRPPAPEAWSEPX₃₃LLAKGSX₃₄AYSDLQSMGTGPDGSPLFGX₃₅
LYEANDYEEIX₃₆FX₃₇MFTLKQAFPAEYLPQX₃₈DKTHTCPPCPAPELLG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWES
NGQPENNYKTTPPVLDSDGSFFLTSKLTVDKSRWQQGNVFSCSVMHEAL
HNHYTQKSLSLSPGK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp; X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₆ is Val or Arg, X₃₇ is Leu, Gln, His, Ile, Lys, or Ser, X₃₈ is GGGGSGGGGS (SEQ ID NO: 145) or EPKSS (SEQ ID NO: 146), and the sialidase comprises at least one mutation relative to wild-type human Neu2 (SEQ ID NO: 1).

In certain embodiments, the third polypeptide comprises the amino acid sequence of

(SEQ ID NO: 143)
X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅QT
GTLFLFFIAIPGQVTEQQQLQTRANVTRLCQVTSTDHGRTWSSPRDLTD
AAIGPAYREWSTFAVGPGHCLQLHDRARSLVVPAYAYRKLHPX₆QRPIP
SAFCFLSHDHGRTWARGHFVAQDTLECQVAEVETGEQRVVTLNARSHLR
ARVQAQSTNDGLDFQESQLVKKLVEPPPX₇GCQGSVISFPSPRSGPGSP
AQWLLYTHPTHX₈X₉QRADLGAYLNPRPPAPEAWSEPVLLAKGSX₁₀AYS
DLQSMGTGPDGSPLFGCLYEANDYEEIX₁₁FX₁₂MFTLKQAFPAEYLPQ
X₁₃DKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLTSKLT
VDKSRWQQGNVFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, X₁₂ is Leu, Gln, His, Ile, Lys, or Ser, and X₁₃ is GGGGSGGGGS (SEQ ID NO: 145) or EPKSS (SEQ ID NO: 146), and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the third polypeptide comprises the amino acid sequence of

(SEQ ID NO: 165)
X₁X₂SX₃X₄X₅LQX₆ESVFQSGAHAYRIPALLYLPGQQSLLAFAEQRASX₇
X₈DEHAELIVX₉RRGDYDAX₁₀THQVQWX₁₁AQEVVAQAX₁₂LX₁₃GHRSMNP
CPLYDX₁₄QTGTLFLFFIAIPX₁₅X₁₆VTEX₁₇QQLQTRANVTRLX₁₈X₁₉VTS
TDHGRTWSSPRDLTDAAIGPX₂₀YREWSTFAVGPGHX₂₁LQLHDX₂₂X₂₃RS
LVVPAYAYRKLHPX₂₄X₂₅X₂₆PIPSAFX₂₇FLSHDHGRTWARGHFVX₂₈QDT
X₂₉ECQVAEVX₃₀TGEQRVVTLNARSX₃₁X₃₂X₃₃X₃₄RX₃₅QAQSX₃₆NX₃₇GLD
FQX₃₈X₃₉QX₄₀VKKLX₄₁EPPPX₄₂GX₄₃QGSVISFPSPRSGPGSPAQX₄₄LLY
THPTHX₄₅X₄₆QRADLGAYLNPRPPAPEAWSEPX₄₇LLAKGSX₄₈AYSDLQSM
GTGPDGSPLFGX₄₉LYEANDYEEIX₅₀FX₅₁MFTLKQAFPAEYLPQX₅₂DKTH
TCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYP
SDIAVEWESNGQPENNYKTTPPVLDSDGSFFLTSKLTVDKSRWQQGNVFS
CSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Lys, Arg, or Glu, X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Asp or Pro, X₁₄ is Ala, Glu or Lys, X₁₅ is Gly or Asp, X₁₆ is Gln or His, X₁₇ is Gln, Arg, or Lys, X₁₈ is Ala, Cys, Ile, Ser, Val, or Leu, X₁₉ is Gln, Leu, Glu, Phe, His, Ile, Leu, or Tyr, X₂₀ is Ala or Val, X₂₁ is Cys or Gly, X₂₂ is Arg or Pro, X₂₃ is Ala or Gly, X₂₄ is Arg, Ile, or Lys, X₂₅ is Gln or Pro, X₂₆ is Arg or Pro, X₂₇ is Ala, Cys, Leu, or Val, X₂₈ is Ala, Cys, Asn, Ser, or Thr, X₂₉ is Leu, Ala, or Val, X₃₀ is Glu or Pro, X₃₁ is His or Pro, X₃₂ is Leu, Asp, Asn, or Tyr, X₃₃ is Arg, Ala, Asp, Leu, Gln, or Tyr, X₃₄ is Ala, Cys, Phe, Gly, His, Ile, Lys, Leu, Met, Asn, Gln, Arg, Ser, Val, Trp, or Tyr, X₃₅ is Val, Ile, or Lys, X₃₆ is Thr or Ala, X₃₇ is Asp or Gly, X₃₈ is Glu, Lys, or Pro, X₃₉ is Ser or Cys, X₄₀ is Leu, Asp, Phe, Gln, or Thr, X₄₁ is Val or Phe, X₄₂ is Gln, Ala, His, Phe, Pro, Ser, or Thr, X₄₃ is Cys or Val, X₄₄ is Trp or Arg, X₄₅ is Ser, Arg, Ala, Asp, Glu, Phe, Gly, His, Ile, Lys, Leu, Met, Asn, Pro, Gln, Thr, Val, Trp, or Tyr, X₄₆ is Trp, Lys, Ala, Asp, Glu, Phe, Gly, His, Ile, Lys, Leu, Met, Asn, Pro, Gln, Arg, Ser, Thr, Val, or Tyr, X₄₇ is Lys or Val, X₄₈ is Ala, Cys, Ser, or Val, X₄₉ is Cys, Leu, or Val, X₅₀ is Val or Arg, X₅₁ is Leu, Gln, His, Ile, Lys, or Ser, X₅₂ is GGGGS (SEQ ID NO: 184), GGGGSGGGGS (SEQ ID NO: 145), or EPKSS (SEQ ID NO: 146), and the sialidase comprises at least one mutation relative to wild-type human Neu2 (SEQ ID NO: 1).

In certain embodiments, the third polypeptide comprises the amino acid sequence of

(SEQ ID NO: 164)
X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅QT
GTLFLFFIAIPGQVTEQQQLQTRANVTRLCX₆VTSTDHGRTWSSPRDLTD
AAIGPAYREWSTFAVGPGHCLQLHDRARSLVVPAYAYRKLHPX₇QRPIPS
AFCFLSHDHGRTWARGHFVAQDTLECQVAEVETGEQRVVTLNARSHLRX₈
RVQAQSTNDGLDFQESQLVKKLVEPPPX₉GCQGSVISFPSPRSGPGSPA
QWLLYTHPTHX₁₀X₁₁QRADLGAYLNPRPPAPEAWSEPVLLAKGSX₁₂AYSD
LQSMGTGPDGSPLFGCLYEANDYEEIX₁₃FX₁₄MFTLKQAFPAEYLPQX₁₅D
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKG
FYPSDIAVEWESNGQPENNYKTTPPVLDSDGSFFLTSKLTVDKSRWQQGN
VFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Gln, Leu, Glu, Phe, His, Ile, Leu, or Tyr, X₇ is Arg, Ile, or Lys, X₈ is Ala, Cys, Phe, Gly, His, Ile, Lys, Leu, Met, Asn, Gln, Arg, Ser, Val, Trp, or Tyr, X₉ is Gln, Ala, His, Phe, Pro, Ser, or Thr, X₁₀ is Ser, Arg, Ala, Asp, Glu, Phe, Gly, His, Ile, Lys, Leu, Met, Asn, Pro, Gln, Thr, Val, Trp, or Tyr, X₁₁ is Trp, Lys, Ala, Asp, Glu, Phe, Gly, His, Ile, Lys, Leu, Met, Asn, Pro, Gln, Arg, Ser, Thr, Val, or Tyr, X₁₂ is Ala, Cys, Ser, or Val, X₁₃ is Val or Arg, X₁₄ is Leu, Gln, His, Ile, Lys, or Ser, X₁₅ is GGGGS (SEQ ID NO: 184), GGGGSGGGGS (SEQ ID NO: 145), or EPKSS (SEQ ID NO: 146), and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Gln or Tyr, X₇ is Ile or Lys, X₈ is Ala or Thr, X₉ is Gln, Ala, or Thr, X₁₀ is Ser, Arg, or Ala, X₁₁ is Trp, Lys, or Arg, X₁₂ is Ala or Cys, X₁₃ is Val or Arg, and X₁₄ is Leu or Ile.

In certain embodiments, the first polypeptide comprises SEQ ID NO: 66, the second polypeptide comprises SEQ ID NO: 67, and the third polypeptide comprises SEQ ID NO: 68. In certain embodiments, the first polypeptide comprises SEQ ID NO: 66, the second polypeptide comprises SEQ ID NO: 67, and the third polypeptide comprises SEQ ID NO: 69. In certain embodiments, the first polypeptide comprises SEQ ID NO: 66, the second polypeptide comprises SEQ ID NO: 67, and the third polypeptide comprises SEQ ID NO: 70. In certain embodiments, the first polypeptide comprises SEQ ID NO: 66, the second polypeptide comprises SEQ ID NO: 67, and the third polypeptide comprises SEQ ID NO: 71. In certain embodiments, the first polypeptide comprises SEQ ID NO: 66, the second polypeptide comprises SEQ ID NO: 67, and the third polypeptide comprises SEQ ID NO: 72. In certain embodiments, the first polypeptide comprises SEQ ID NO: 66, the second polypeptide comprises SEQ ID NO: 67, and the third polypeptide comprises SEQ ID NO: 73. In certain embodiments, the first polypeptide comprises SEQ ID NO: 66, the second polypeptide comprises SEQ ID NO: 67, and the third polypeptide comprises SEQ ID NO: 74. In certain embodiments, the first polypeptide comprises SEQ ID NO: 66, the second polypeptide comprises SEQ ID NO: 67, and the third polypeptide comprises SEQ ID NO: 98. In certain embodiments, the first polypeptide comprises SEQ ID NO: 66, the second polypeptide comprises SEQ ID NO: 67, and the third polypeptide comprises SEQ ID NO: 99. In certain embodiments, the first polypeptide comprises SEQ ID NO: 66, the second polypeptide comprises SEQ ID NO: 67, and the third polypeptide comprises SEQ ID NO: 100. In certain embodiments, the first polypeptide comprises SEQ ID NO: 66, the second polypeptide comprises SEQ ID NO: 67, and the third polypeptide comprises SEQ ID NO: 101. In certain embodiments, the first polypeptide comprises SEQ ID NO: 66, the second polypeptide comprises SEQ ID NO: 67, and the third polypeptide comprises SEQ ID NO: 102. In certain embodiments, the first polypeptide comprises SEQ ID NO: 66, the second polypeptide comprises SEQ ID NO: 67, and the third polypeptide comprises SEQ ID NO: 103. In certain embodiments, the first polypeptide comprises SEQ ID NO: 66, the second polypeptide comprises SEQ ID NO: 67, and the third polypeptide comprises SEQ ID NO: 104. In certain embodiments, the first polypeptide comprises SEQ ID NO: 66, the second polypeptide comprises SEQ ID NO: 67, and the third polypeptide comprises SEQ ID NO: 105. In certain embodiments, the first polypeptide comprises SEQ ID NO: 66, the second polypeptide comprises SEQ ID NO: 67, and the third polypeptide comprises SEQ ID NO: 106. In certain embodiments, the first polypeptide comprises SEQ ID NO: 66, the second polypeptide comprises SEQ ID NO: 67, and the third polypeptide comprises SEQ ID NO: 107. In certain embodiments, the first polypeptide comprises SEQ ID NO: 66, the second polypeptide comprises SEQ ID NO: 67, and the third polypeptide comprises SEQ ID NO: 108. In certain embodiments, the first polypeptide comprises SEQ ID NO: 66, the second polypeptide comprises SEQ ID NO: 67, and the third polypeptide comprises SEQ ID NO: 109. In certain embodiments, the first polypeptide comprises SEQ ID NO: 66, the second polypeptide comprises SEQ ID NO: 67, and the third polypeptide comprises SEQ ID NO: 110. In certain embodiments, the first polypeptide comprises SEQ ID NO: 66, the second polypeptide comprises SEQ ID NO: 67, and the third polypeptide comprises SEQ ID NO: 111. In certain embodiments, the first polypeptide comprises SEQ ID NO: 66, the second polypeptide comprises SEQ ID NO: 67, and the third polypeptide comprises SEQ ID NO: 112. In certain embodiments, the first polypeptide comprises SEQ ID NO: 66, the second polypeptide comprises SEQ ID NO: 67, and the third polypeptide comprises SEQ ID NO: 150. In certain embodiments, the first polypeptide comprises SEQ ID NO: 66, the second polypeptide comprises SEQ ID NO: 67, and the third polypeptide comprises SEQ ID NO: 151. In certain embodiments, the first polypeptide comprises SEQ ID NO: 66, the second polypeptide comprises SEQ ID NO: 67, and the third polypeptide comprises SEQ ID NO: 155. In certain embodiments, the first polypeptide comprises SEQ ID NO: 66, the second polypeptide comprises SEQ ID NO: 67, and the third polypeptide comprises SEQ ID NO: 156. In certain embodiments, the first polypeptide comprises SEQ ID NO: 66, the second polypeptide comprises SEQ ID NO: 67, and the third polypeptide comprises SEQ ID NO: 160. In certain embodiments, the first polypeptide comprises SEQ ID NO: 66, the second polypeptide comprises SEQ ID NO: 67, and the third polypeptide comprises SEQ ID NO: 161. In certain embodiments, the first polypeptide comprises SEQ ID NO: 66, the second polypeptide comprises SEQ ID NO: 67, and the third polypeptide comprises SEQ ID NO: 192. In certain embodiments, the first polypeptide comprises SEQ ID NO: 66, the second polypeptide comprises SEQ ID NO: 67, and the third polypeptide comprises SEQ ID NO: 195. In certain embodiments, the first polypeptide comprises SEQ ID NO: 66, the second polypeptide comprises SEQ ID NO: 189, and the third polypeptide comprises SEQ ID NO: 185. In certain embodiments, the first polypeptide comprises SEQ ID NO: 66, the second polypeptide comprises SEQ ID NO: 189, and the third polypeptide comprises SEQ ID NO: 187. In certain embodiments, the first polypeptide comprises SEQ ID NO: 66, the second polypeptide comprises SEQ ID NO: 189, and the third polypeptide comprises SEQ ID NO: 205.

In certain embodiments, the antibody conjugate comprises a first polypeptide comprising a first sialidase, a first immunoglobulin Fc domain, and a first single chain variable fragment (scFv) (it is also understood that the scFv may be replaced by a first polypeptide chain of an immunoglobulin antigen binding fragment, e.g., Fab fragment); and a second polypeptide comprising a second sialidase, a second immunoglobulin Fc domain, and a second single chain variable fragment (scFv) (it is also understood that the scFv may be replaced by a second polypeptide chain of an immunoglobulin antigen binding fragment, e.g., Fab fragment). An example of this embodiment is shown in FIG. 11C (in the construct depicted in FIG. 11C it is understood that an scFv, when present, may be replaced with a Fab fragment, or a Fab fragment, when present, may be replaced with an scFv). The first and second polypeptides can be covalently linked together. The covalent linkages can be disulfide bonds. In certain embodiments, the first scFv defines a first antigen-binding site, and the second scFv defines a second antigen-binding site. In certain embodiments, the first polypeptide comprises the first sialidase, the first immunoglobulin Fc domain, and the first scFv in an N- to C-terminal orientation. In certain embodiments, the first polypeptide comprises the first scFv, the first immunoglobulin Fc domain, and the first sialidase in an N- to C-terminal orientation. In certain embodiments, the second polypeptide comprises the second sialidase, the second immunoglobulin Fc domain, and the second scFv in an N- to C-terminal orientation. In certain embodiments, the second polypeptide comprises the second scFv, the second immunoglobulin Fc domain, and the second sialidase in an N- to C-terminal orientation.

In certain embodiments, the first polypeptide comprises the amino acid sequence of any one of SEQ ID NOs: 77-83, 166-178, 194, 197, 244, or 249, or an amino acid sequence that has at least 85%, 90%, 95%, 96%, 97%, 98%, or 99% sequence identity to any one of SEQ ID NOs: 77-83, 166-178, 194, 197, 244, or 249. In certain embodiments, the second polypeptide comprises the amino acid sequence of any one of SEQ ID NOs: 77-83, 166-178, 194, 197, 244, or 249, or an amino acid sequence that has at least 85%, 90%, 95%, 96%, 97%, 98%, or 99% sequence identity to any one of SEQ ID NOs: 77-83, 166-178, 194, 197, 244, or 249.

In certain embodiments, the first and/or second polypeptide comprises the amino acid sequence of

(SEQ ID NO: 248)
X₁X₂SX₃X₄X₅LQX₆ESVFQSGAHAYRIPALLYLPGQQSLLAFAEQRX₇SX₈
X₉DEHAELIVX₁₀RRGDYDAX₁₁THQVQWX₁₂AQEVVAQAX₁₃LX₁₄GHRSMN
PCPLYDX₁₅QTGTLFLFFIAIPX₁₆X₁₇VTEX₁₈QQLQTRANVTRLX₁₉X₂₀V
TSTDHGRTWSSPRDLTDAAIGPX₂₁YREWSTFAVGPGHX₂₂LQLHDX₂₃X₂₄
RSLVVPAYAYRKLHPX₂₅X₂₆X₂₇PIPSAFX₂₈FLSHDHGRTWARGHFVX₂₉
QDTX₃₀ECQVAEVX₃₁TGEQRVVTLNARSX₃₂X₃₃X₃₄X₃₅RX₃₆QAQSX₃₇NX₃₈
GLDFQX₃₉X₄₀QX₄₁VKKLX₄₂EPPPX₄₃GX₄₄QGSVISFPSPRSGPGSPAQX₄₅
LLYTHPTHX₄₆X₄₇QRADLGAYLNPRPPAPEAWSEPX₄₈LLAKGSX₄₉AYSDL
QSMGTGPDGSPLFGX₅₀LYEANDYEEIX₅₁FX₅₂MFTLKQAFPAEYLPQX₅₃
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGKGGGGSGGGGSGGGGSEVQLVESG
GGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMD
YWGQGTLVTVSSGGGGSGGGGSGGGGSDIQMTQSPSSLSASVGDRVTITC
RASQDVNTAVAWYQQKPGKAPKLLIYSASFLYSGVPSRFSGSRSGTDFTL
TISSLQPEDFATYYCQQHYTTPPTFGQGTKVEIK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Ala or Arg, X₈ is Lys, Arg, or Glu, X₉ is Lys, Ala, Arg, or Glu, X₁₀ is Leu or Met, X₁₁ is Pro, Asn, Asp, His, Glu, Gly, Ser or Thr, X₁₂ is Gln or His, X₁₃ is Arg or Lys, X₁₄ is Asp or Pro, X₁₅ is Ala, Glu or Lys, X₁₆ is Gly or Asp, X₁₇ is Gln or His, X₁₈ is Gln, Arg, or Lys, X₁₉ is Ala, Cys, Ile, Ser, Val, or Leu, X₂₀ is Gln, Leu, Glu, Phe, His, Ile, Leu, or Tyr, X₂₁ is Ala or Val, X₂₂ is Cys or Gly, X₂₃ is Arg or Pro, X₂₄ is Ala or Gly, X₂₅ is Arg, Ile, or Lys, X₂₆ is Gln or Pro, X₂₇ is Arg or Pro, X₂₈ is Ala, Cys, Leu, or Val, X₂₉ is Ala, Cys, Asn, Ser, or Thr, X₃₀ is Leu, Ala, or Val, X₃₁ is Glu or Pro, X₃₂ is His or Pro, X₃₃ is Leu, Asp, Asn, or Tyr, X₃₄ is Arg, Ala, Asp, Leu, Gln, or Tyr, X₃₅ is Ala, Cys, Phe, Gly, His, Ile, Lys, Leu, Met, Asn, Gln, Arg, Ser, Val, Trp, or Tyr, X₃₆ is Val, Ile, or Lys, X₃₇ is Thr or Ala, X₃₈ is Asp or Gly, X₃₉ is Glu, Lys, or Pro, X₄₀ is Ser or Cys, X₄₁ is Leu, Asp, Phe, Gln, or Thr, X₄₂ is Val or Phe, X₄₃ is Gln, Ala, His, Phe, Pro, Ser, or Thr, X₄₄ is Cys or Val, X₄₅ is Trp or Arg, X₄₆ is Ser, Arg, Ala, Asp, Glu, Phe, Gly, His, Ile, Lys, Leu, Met, Asn, Pro, Gln, Thr, Val, Trp, or Tyr, X₄₇ is Trp, Lys, Ala, Asp, Glu, Phe, Gly, His, Ile, Lys, Leu, Met, Asn, Pro, Gln, Arg, Ser, Thr, Val, or Tyr, X₄₈ is Lys or Val, X₄₉ is Ala, Cys, Ser, or Val, X₅₀ is Cys, Leu, or Val, X₅₁ is Val or Arg, X₅₂ is Leu, Gln, His, Ile, Lys, or Ser, and X₅₃ is GGGGS (SEQ ID NO: 184), GGGGSGGGGS (SEQ ID NO: 145), or EPKSS (SEQ ID NO: 146), and the sialidase comprises at least one mutation relative to wild-type human Neu2 (SEQ ID NO: 1).

In certain embodiments, the first and/or second polypeptide comprises the amino acid sequence of

(SEQ ID NO: 247)
X₁ASLPX₂LQX₃ESVFQSGAHAYRIPALLYLPGQQSLLAFAEQRX₄SKKD
EHAELIVLRRGDYDAX₅THQVQWQAQEVVAQARLDGHRSMNPCPLYDX₆Q
TGTLFLFFIAIPGQVTEQQQLQTRANVTRLCX₇VTSTDHGRTWSSPRDLT
DAAIGPAYREWSTFAVGPGHCLQLHDRARSLVVPAYAYRKLHPX₈QRPIP
SAFCFLSHDHGRTWARGHFVAQDTLECQVAEVETGEQRVVTLNARSHLR
X₉RVQAQSTNDGLDFQESQLVKKLVEPPPX₁₀GCQGSVISFPSPRSGPGS
PAQWLLYTHPTHX₁₁X₁₂QRADLGAYLNPRPPAPEAWSEPVLLAKGSX₁₃A
YSDLQSMGTGPDGSPLFGCLYEANDYEEIX₁₄FX₁₅MFTLKQAFPAEYLP
QX₁₆DKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTV
DKSRWQQGNVFSCSVMHEALHNHYTQKSLSLSPGKGGGGSGGGGSGGGG
SEVQLVESGGGLVQPGGSLRLSCAASGFNIKDTYIHWVRQAPGKGLEWV
ARIYPTNGYTRYADSVKGRFTISADTSKNTAYLQMNSLRAEDTAVYYCS
RWGGDGFYAMDYWGQGTLVTVSSGGGGSGGGGSGGGGSDIQMTQSPSSL
SASVGDRVTITCRASQDVNTAVAWYQQKPGKAPKLLIYSASFLYSGVPS
RFSGSRSGTDFTLTISSLQPEDFATYYCQQHYTTPPTFGQGTKVEIK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Arg or Ala, X₅ is Pro, Asn, Asp, His, Glu, Gly, Ser or Thr, X₆ is Ala, Glu, or Lys, X₇ is Gln, Leu, Glu, Phe, His, Ile, Leu, or Tyr, X₈ is Arg, Ile, or Lys, X₉ is Ala, Cys, Phe, Gly, His, Ile, Lys, Leu, Met, Asn, Gln, Arg, Ser, Val, Trp, or Tyr, X₁₀ is Gln, Ala, His, Phe, Pro, Ser, or Thr, X₁₁ is Ser, Arg, Ala, Asp, Glu, Phe, Gly, His, Ile, Lys, Leu, Met, Asn, Pro, Gln, Thr, Val, Trp, or Tyr, X₁₂ is Trp, Lys, Ala, Asp, Glu, Phe, Gly, His, Ile, Lys, Leu, Met, Asn, Pro, Gln, Arg, Ser, Thr, Val, or Tyr, X₁₃ is Ala, Cys, Ser, or Val, X₁₄ is Val or Arg, X₁₅ is Leu, Gln, His, Ile, Lys, or Ser, and X₁₆ is GGGGS (SEQ ID NO: 184), GGGGSGGGGS (SEQ ID NO: 145), or EPKSS (SEQ ID NO: 146), and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Arg or Ala, X₅ is Pro, Asn, Gly, Ser or Thr, X₆ is Ala or Glu, X₇ is Gln or Tyr, X₈ is Ile or Lys, X₉ is Ala or Thr, X₁₀ is Gln, Ala, or Thr, X₁₁ is Ser, Arg, or Ala, X₁₂ is Trp, Lys, or Arg, X₁₃ is Ala or Cys, X₁₄ is Val or Arg, and X₁₅ is Leu or Ile.

In certain embodiments, the first and/or second polypeptide comprises the amino acid sequence of

(SEQ ID NO: 85)
X₁X₂SX₃X₄X₅LQX₆ESVFQSGAHAYRIPALLYLPGQQSLLAFAEQRASX₇
X₈DEHAELIVX₉RRGDYDAX₁₀THQVQWX₁₁AQEVVAQAX₁₂LDGHRSMNPC
PLYDX₁₃QTGTLFLFFIAIPX₁₄X₁₅VTEX₁₆QQLQTRANVTRLX₁₇X₁₈VTS
TDHGRTWSSPRDLTDAAIGPX₁₉YREWSTFAVGPGHX₂₀LQLHDRX₂₁RSL
VVPAYAYRKLHPX₂₂QRPIPSAFX₂₃FLSHDHGRTWARGHFVAQDTX₂₄EC
QVAEVETGEQRVVTLNARSHLRARVQAQSX₂₅NX₂₆GLDFQX₂₇SQLVKKL
VEPPPX₂₈GX₂₉QGSVISFPSPRSGPGSPAQX₃₀LLYTHPTHX₃₁X₃₂QRAD
LGAYLNPRPPAPEAWSEPX₃₃LLAKGSX₃₄AYSDLQSMGTGPDGSPLFG
X₃₅LYEANDYEEIX₃₆FX₃₇MFTLKQAFPAEYLPQGGGGSGGGGSDKTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNV
FSCSVMHEALHNHYTQKSLSLSPGKGGGGSGGGGSGGGGSEVQLVESGG
GLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAM
DYWGQGTLVTVSSGGGGSGGGGSGGGGSDIQMTQSPSSLSASVGDRVTI
TCRASQDVNTAVAWYQQKPGKAPKLLIYSASFLYSGVPSRFSGSRSGTD
FTLTISSLQPEDFATYYCQQHYTTPPTFGQGTKVEIK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp; X₇ is Lys, Arg, or Glu. X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Ala, Glu or Lys, X₁₄ is Gly or Asp, X₁₅ is Gln or His, X₁₆ is Gln, Arg, or Lys, X₁₇ is Ala, Cys, Ile, Ser, Val, or Leu, X₁₈ is Gln or Leu, X₁₉ is Ala or Val, X₂₀ is Cys or Gly, X₂₁ is Ala or Gly, X₂₂ is Arg, Ile, or Lys, X₂₃ is Ala, Cys, Leu, or Val, X₂₄ is Leu, Ala, or Val, X₂₅ is Thr or Ala, X₂₆ is Asp or Gly, X₂₇ is Glu or Lys, X₂₈ is Gln, Ala, His, Phe, or Pro, X₂₉ is Cys or Val, X₃₀ is Trp or Arg, X₃₁ is Ser or Arg, X₃₂ is Trp or Lys, X₃₃ is Lys or Val, X₃₄ is Ala, Cys, Ser, or Val, X₃₅ is Cys, Leu, or Val, X₃₆ is Val or Arg, and X₃₇ is Leu, Gln, His, Ile, Lys, or Ser, and the sialidase comprises at least one mutation relative to wild-type human Neu2 (SEQ ID NO: 1).

In certain embodiments, the first and/or second polypeptide comprises the amino acid sequence of

(SEQ ID NO: 84)
X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅QT
GTLFLFFIAIPGQVTEQQQLQTRANVTRLCQVTSTDHGRTWSSPRDLTD
AAIGPAYREWSTFAVGPGHCLQLHDRARSLVVPAYAYRKLHPX₆QRPIP
SAFCFLSHDHGRTWARGHFVAQDTLECQVAEVETGEQRVVTLNARSHLR
ARVQAQSTNDGLDFQESQLVKKLVEPPPX₇GCQGSVISFPSPRSGPGSP
AQWLLYTHPTHX₈X₉QRADLGAYLNPRPPAPEAWSEPVLLAKGSX₁₀AYS
DLQSMGTGPDGSPLFGCLYEANDYEEIX₁₁FX₁₂MFTLKQAFPAEYLPQG
GGGSGGGGSDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLY
SKLTVDKSRWQQGNVFSCSVMHEALHNHYTQKSLSLSPGKGGGGSGGGG
SGGGGSEVQLVESGGGLVQPGGSLRLSCAASGFNIKDTYIHWVRQAPGK
GLEWVARIYPTNGYTRYADSVKGRFTISADTSKNTAYLQMNSLRAEDTA
VYYCSRWGGDGFYAMDYWGQGTLVTVSSGGGGSGGGGSGGGGSDIQMTQ
SPSSLSASVGDRVTITCRASQDVNTAVAWYQQKPGKAPKLLIYSASFLY
SGVPSRFSGSRSGTDFTLTISSLQPEDFATYYCQQHYTTPPTFGQGTKV
EIK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Arg, Ile, or Lys, X₇ is Gln, Ala, His, Phe, or Pro, X₈ is Ser or Arg, X₉ is Trp or Lys, X₁₀ is Ala, Cys, Ser, or Val, X₁₁ is Val or Arg, and X₁₂ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Ile or Lys, X₇ is Gln or Ala, X₈ is Ser or Arg, X₉ is Trp or Lys, X₁₀ is Ala or Cys, X₁₁ is Val or Arg, and X₁₂ is Leu or Ile.

In certain embodiments, the first and/or second polypeptide comprises the amino acid sequence of

(SEQ ID NO: 180)
X₁X₂SX₃X₄X₅LQX₆ESVFQSGAHAYRIPALLYLPGQQSLLAFAEQRASX₇
X₈DEHAELIVX₉RRGDYDAX₁₀THQVQWX₁₁AQEVVAQAX₁₂LX₁₃GHRSMNP
CPLYDX₁₄QTGTLFLFFIAIPX₁₅X₁₆VTEX₁₇QQLQTRANVTRLX₁₈X₁₉VTS
TDHGRTWSSPRDLTDAAIGPX₂₀YREWSTFAVGPGHX₂₁LQLHDX₂₂X₂₃RS
LVVPAYAYRKLHPX₂₄X₂₅X₂₆PIPSAFX₂₇FLSHDHGRTWARGHFVX₂₈QDT
X₂₉ECQVAEVX₃₀TGEQRVVTLNARSX₃₁X₃₂X₃₃X₃₄RX₃₅QAQSX₃₆NX₃₇GLD
FQX₃₈X₃₉QX₄₀VKKLX₄₁EPPPX₄₂GX₄₃QGSVISFPSPRSGPGSPAQX₄₄LL
YTHPTHX₄₅X₄₆QRADLGAYLNPRPPAPEAWSEPX₄₇LLAKGSX₄₈AYSDLQ
SMGTGPDGSPLFGX₄₉LYEANDYEEIX₅₀FX₅₁MFTLKQAFPAEYLPQX₅₂D
KTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRW
QQGNVFSCSVMHEALHNHYTQKSLSLSPGKGGGGSGGGGSGGGGSEVQL
VESGGGLVQPGGSLRLSCAASGFNIKDTYIHWVRQAPGKGLEWVARIYP
TNGYTRYADSVKGRFTISADTSKNTAYLQMNSLRAEDTAVYYCSRWGGD
GFYAMDYWGQGTLVTVSSGGGGSGGGGSGGGGSDIQMTQSPSSLSASVG
DRVTITCRASQDVNTAVAWYQQKPGKAPKLLIYSASFLYSGVPSRFSGS
RSGTDFTLTISSLQPEDFATYYCQQHYTTPPTFGQGTKVEIK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Lys, Arg, or Glu, X₈ is Lys, Ala, Arg, or Glu, X₉ is Leu or Met, X₁₀ is Pro, Asn, Asp, His, Glu, Gly, Ser or Thr, X₁₁ is Gln or His, X₁₂ is Arg or Lys, X₁₃ is Asp or Pro, X₁₄ is Ala, Glu or Lys, X₁₅ is Gly or Asp, X₁₆ is Gln or His, X₁₇ is Gln, Arg, or Lys, X₁₈ is Ala, Cys, Ile, Ser, Val, or Leu, X₁₉ is Gln, Leu, Glu, Phe, His, Ile, Leu, or Tyr, X₂₀ is Ala or Val, X₂₁ is Cys or Gly, X₂₂ is Arg or Pro, X₂₃ is Ala or Gly, X₂₄ is Arg, Ile, or Lys, X₂₅ is Gln or Pro, X₂₆ is Arg or Pro, X₂₇ is Ala, Cys, Leu, or Val, X₂₈ is Ala, Cys, Asn, Ser, or Thr, X₂₉ is Leu, Ala, or Val, X₃₀ is Glu or Pro, X₃₁ is His or Pro, X₃₂ is Leu, Asp, Asn, or Tyr, X₃₃ is Arg, Ala, Asp, Leu, Gln, or Tyr, X₃₄ is Ala, Cys, Phe, Gly, His, Ile, Lys, Leu, Met, Asn, Gln, Arg, Ser, Val, Trp, or Tyr, X₃₅ is Val, Ile, or Lys, X₃₆ is Thr or Ala, X₃₇ is Asp or Gly, X₃₈ is Glu, Lys, or Pro, X₃₉ is Ser or Cys, X₄₀ is Leu, Asp, Phe, Gln, or Thr, X₄₁ is Val or Phe, X₄₂ is Gln, Ala, His, Phe, Pro, Ser, or Thr, X₄₃ is Cys or Val, X₄₄ is Trp or Arg, X₄₅ is Ser, Arg, Ala, Asp, Glu, Phe, Gly, His, Ile, Lys, Leu, Met, Asn, Pro, Gln, Thr, Val, Trp, or Tyr, X₄₆ is Trp, Lys, Ala, Asp, Glu, Phe, Gly, His, Ile, Lys, Leu, Met, Asn, Pro, Gln, Arg, Ser, Thr, Val, or Tyr, X₄₇ is Lys or Val, X₄₈ is Ala, Cys, Ser, or Val, X₄₉ is Cys, Leu, or Val, X₅₀ is Val or Arg, X₅₁ is Leu, Gln, His, Ile, Lys, or Ser, X₅₂ is GGGGS (SEQ ID NO: 184), GGGGSGGGGS (SEQ ID NO: 145), or EPKSS (SEQ ID NO: 146), and the sialidase comprises at least one mutation relative to wild-type human Neu2 (SEQ ID NO: 1).

In certain embodiments, the first and/or second polypeptide comprises the amino acid sequence of

(SEQ ID NO: 179)
X₁ASLPX₂LQX₃ESVFQSGAHAYRIPALLYLPGQQSLLAFAEQRASKKDE
HAELIVLRRGDYDAX₄THQVQWQAQEVVAQARLDGHRSMNPCPLYDX₅QT
GTLFLFFIAIPGQVTEQQQLQTRANVTRLCX₆VTSTDHGRTWSSPRDLTD
AAIGPAYREWSTFAVGPGHCLQLHDRARSLVVPAYAYRKLHPX₇QRPIPS
AFCFLSHDHGRTWARGHFVAQDTLECQVAEVETGEQRVVTLNARSHLRX₈
RVQAQSTNDGLDFQESQLVKKLVEPPPX₉GCQGSVISFPSPRSGPGSPAQ
WLLYTHPTHX₁₀X₁₁QRADLGAYLNPRPPAPEAWSEPVLLAKGSX₁₂AYSDL
QSMGTGPDGSPLFGCLYEANDYEEIX₁₃FX₁₄MFTLKQAFPAEYLPQX₁₅DK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNV
FSCSVMHEALHNHYTQKSLSLSPGKGGGGSGGGGSGGGGSEVQLVESGGG
LVQPGGSLRLSCAASGFNIKDTYIHWVRQAPGKGLEWVARIYPTNGYTRY
ADSVKGRFTISADTSKNTAYLQMNSLRAEDTAVYYCSRWGGDGFYAMDYW
GQGTLVTVSSGGGGSGGGGSGGGGSDIQMTQSPSSLSASVGDRVTITCRA
SQDVNTAVAWYQQKPGKAPKLLIYSASFLYSGVPSRFSGSRSGTDFTLTI
SSLQPEDFATYYCQQHYTTPPTFGQGTKVEIK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Pro, Asn, Asp, His, Glu, Gly, Ser or Thr, X₅ is Ala, Glu, or Lys, X₆ is Gln, Leu, Glu, Phe, His, Ile, Leu, or Tyr, X₇ is Arg, Ile, or Lys, X₈ is Ala, Cys, Phe, Gly, His, Ile, Lys, Leu, Met, Asn, Gln, Arg, Ser, Val, Trp, or Tyr, X₉ is Gln, Ala, His, Phe, Pro, Ser, or Thr, X₁₀ is Ser, Arg, Ala, Asp, Glu, Phe, Gly, His, Ile, Lys, Leu, Met, Asn, Pro, Gln, Thr, Val, Trp, or Tyr, X₁₁ is Trp, Lys, Ala, Asp, Glu, Phe, Gly, His, Ile, Lys, Leu, Met, Asn, Pro, Gln, Arg, Ser, Thr, Val, or Tyr, X₁₂ is Ala, Cys, Ser, or Val, X₁₃ is Val or Arg, X₁₄ is Leu, Gln, His, Ile, Lys, or Ser, X₁₅ is GGGGS (SEQ ID NO: 184), GGGGSGGGGS (SEQ ID NO: 145), or EPKSS (SEQ ID NO: 146), and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Pro, Asn, Gly, Ser or Thr, X₅ is Ala or Glu, X₆ is Gln or Tyr, X₇ is Ile or Lys, X₈ is Ala or Thr, X₉ is Gln, Ala, or Thr, X₁₀ is Ser, Arg, or Ala, X₁₁ is Trp, Lys, or Arg, X₁₂ is Ala or Cys, X₁₃ is Val or Arg, and X₁₄ is Leu or Ile.

In certain embodiments, the first and second polypeptide comprise SEQ ID NO: 77. In certain embodiments, the first and second polypeptide comprise SEQ ID NO: 78. In certain embodiments, the first and second polypeptide comprise SEQ ID NO: 79. In certain embodiments, the first and second polypeptide comprise SEQ ID NO: 80. In certain embodiments, the first and second polypeptide comprise SEQ ID NO: 81. In certain embodiments, the first and second polypeptide comprise SEQ ID NO: 82. In certain embodiments, the first and second polypeptide comprise SEQ ID NO: 83. In certain embodiments, the first and second polypeptide comprise SEQ ID NO: 166. In certain embodiments, the first and second polypeptide comprise SEQ ID NO: 167. In certain embodiments, the first and second polypeptide comprise SEQ ID NO: 168. In certain embodiments, the first and second polypeptide comprise SEQ ID NO: 169. In certain embodiments, the first and second polypeptide comprise SEQ ID NO: 170. In certain embodiments, the first and second polypeptide comprise SEQ ID NO: 171. In certain embodiments, the first and second polypeptide comprise SEQ ID NO: 172. In certain embodiments, the first and second polypeptide comprise SEQ ID NO: 173. In certain embodiments, the first and second polypeptide comprise SEQ ID NO: 174. In certain embodiments, the first and second polypeptide comprise SEQ ID NO: 175. In certain embodiments, the first and second polypeptide comprise SEQ ID NO: 176. In certain embodiments, the first and second polypeptide comprise SEQ ID NO: 177. In certain embodiments, the first and second polypeptide comprise SEQ ID NO: 178. In certain embodiments, the first and second polypeptide comprise SEQ ID NO: 194. In certain embodiments, the first and second polypeptide comprise SEQ ID NO: 197. In certain embodiments, the first and second polypeptide comprise SEQ ID NO: 244. In certain embodiments, the first and second polypeptide comprise SEQ ID NO: 249.

In certain embodiments, the antibody conjugate comprises: a first polypeptide comprising an immunoglobulin light chain; a second polypeptide comprising an immunoglobulin heavy chain and a single chain variable fragment (scFv) (it is also understood that the scFv may be replaced by a first polypeptide chain of an immunoglobulin antigen binding fragment, e.g., Fab fragment); and a third polypeptide comprising an immunoglobulin Fc domain and a sialidase. An example of this embodiment is shown in FIG. 11D. The first and second polypeptides can be covalently linked together and the second and third polypeptides can be covalently linked together. The covalent linkages can be disulfide bonds. In certain embodiments, the first polypeptide and the second polypeptide together define a first antigen-binding site (i.e., the immunoglobulin light chain and immunoglobulin heavy chain together define a first antigen-binding site). In certain embodiments, the scFv defines a second antigen-binding site. In certain embodiments, the second polypeptide comprises the immunoglobulin heavy chain and the scFv in an N- to C-terminal orientation, or the scFv and the immunoglobulin heavy chain in an N- to C-terminal orientation. In certain embodiments, the third polypeptide comprises the sialidase and the immunoglobulin Fc domain in an N- to C-terminal orientation, or the sialidase and the immunoglobulin Fc domain in an N- to C-terminal orientation.

In certain embodiments, the antibody conjugate comprises a first polypeptide comprising a first immunoglobulin light chain; a second polypeptide comprising a first sialidase, a first immunoglobulin Fc domain, and a first immunoglobulin heavy chain variable region; a third polypeptide comprising a second sialidase, a second immunoglobulin Fc domain, and a second immunoglobulin heavy chain variable region; and a fourth polypeptide comprising a second immunoglobulin light chain. It is also understood that an immunoglobulin light chain may be replaced by an immunoglobulin heavy chain variable region and an immunoglobulin heavy chain variable region may be replaced by an immunoglobulin light chain (e.g., the antibody conjugate may comprise a first polypeptide comprising a first immunoglobulin heavy chain variable region; a second polypeptide comprising a first sialidase, a first immunoglobulin Fc domain, and a first immunoglobulin light chain; a third polypeptide comprising a second sialidase, a second immunoglobulin Fc domain, and a second immunoglobulin light chain; and a fourth polypeptide comprising a second immunoglobulin heavy chain variable region). An example of this embodiment is shown in FIG. 11E. The second and third polypeptides can be covalently linked together. The covalent linkages can be disulfide bonds. In certain embodiments, the first and second polypeptides define a first antigen-binding site, and the third and fourth polypeptides define a second antigen-binding site. In certain embodiments, the second polypeptide comprises the first sialidase, the first immunoglobulin Fc domain, and the first immunoglobulin heavy chain variable region in an N- to C-terminal orientation. In certain embodiments, the third polypeptide comprises the second sialidase, the second immunoglobulin Fc domain, and the second immunoglobulin heavy chain variable region in an N- to C-terminal orientation.

In certain embodiments, the antibody conjugate has a molecular weight from about 135 kDa to about 165 kDa, e.g., about 140 kDa. In other embodiments, the antibody conjugate has a molecular weight from about 215 kDa to about 245 kDa, e.g., about 230 kDa.

In certain embodiments, the antibody conjugate comprises two polypeptides that each comprise an immunoglobulin Fc domain, and the first polypeptide has either a “knob” mutation, e.g., T366Y, or a “hole” mutation, e.g., Y407T, for heterodimerization with the second polypeptide, and the second polypeptide has either a respective “knob” mutation, e.g., T366Y, or a “hole” mutation, e.g., Y407T, for heterodimerization with the first polypeptide (residue numbers according to EU numbering, Kabat, E. A., et al. (1991) supra). For example, in certain embodiments, the antibody comprises two polypeptides that each comprise an immunoglobulin Fc domain derived from human IgG1 Fc domain, and the first polypeptide comprises a Y407T mutation (e.g., the first polypeptide comprises SEQ ID NO: 32, SEQ ID NO: 147, SEQ ID NO: 213, or SEQ ID NO: 215), and the second polypeptide comprises a T366Y mutation (e.g., the second polypeptide comprises SEQ ID NO: 33, SEQ ID NO: 148, SEQ ID NO: 214, or SEQ ID NO: 216).

As used herein, the term “multispecific antibody” is understood to mean an antibody that specifically binds to at least two different antigens, i.e., an antibody that comprises at least two antigen-binding sites that bind to at least two different antigens. As used herein, the term “bispecific antibody” is understood to mean an antibody that specifically binds to two different antigens, i.e., an antibody that comprises two antigen-binding sites each of which bind to separate and distinct antigens. In other words, a first binding site binds a first antigen and a second binding site binds a second, different antigen. A multispecific or bispecific antibody may, for example, be a human or humanized antibody, and/or be a full length antibody or an antibody fragment (e.g., a F(ab′)2 bispecific antibody).

The present invention encompasses antibody conjugates comprising antibody fragments, which may be generated by traditional means, such as enzymatic digestion, or by recombinant techniques. For a review of certain antibody fragments, see Hudson et al. (2003) supra.

In certain embodiments, the antibody conjugate or fusion protein can be covalently or non-covalently associated with a biological modifier, wherein the biological modifier can be used to enhance the solubility of the antibody, increase binding specificity, decrease immunogenicity or toxicity or modify the pharmacokinetic profile of the antibody. For example, the biological modifier can be used to increase the molecular weight of the antibody to increase its circulating half-life.

It is contemplated that the antibody conjugate or fusion protein may be covalently bound to one or more (for example, 2, 3, 4, 5, 6, 8, 9, 10 or more) biological modifiers that may comprise linear or branched polymers. Exemplary biological modifiers may include, for example, a variety of polymers, such as those described in U.S. Pat. No. 7,842,789.

Particularly useful are polyalkylene ethers such as polyethylene glycol (PEG) and derivatives thereof (for example, alkoxy polyethylene glycol, for example, methoxypolyethylene glycol, ethoxypolyethylene glycol and the like); block copolymers of polyoxyethylene and polyoxypropylene (Pluronics); polymethacrylates; carbomers; and branched or unbranched polysaccharides which comprise the saccharide monomers such as D-mannose, D- and L-galactose, fucose, fructose, D-xylose, L-arabinose, and D-glucuronic acid.

In other embodiments, the biological modifier can be a hydrophilic polyvinyl polymer such as polyvinyl alcohol and polyvinylpyrrolidone (PVP)-type polymers. The biological modifier can be a functionalized polyvinylpyrrolidone, for example, carboxy or amine functionalized on one (or both) ends of the polymer (as available from PolymerSource). Alternatively, the biological modifier can include Poly N-(2-hydroxypropyl)methacrylamide (HPMA), or functionalized HPMA (amine, carboxy, etc.), Poly(N-isopropylacrylamide) or functionalized poly(N-isopropylacrylamide). Alternatively, the biological modifier can include Poly N-(2-hydroxypropyl)methacrylamide (HPMA), or functionalized HPMA (amine, carboxy, etc.), Poly(N-isopropylacrylamide) or functionalized poly(N-isopropylacrylamide). The modifier prior to conjugation need not be, but preferably is, water soluble, but the final conjugate should be water soluble.

In general, the biological modifier may have a molecular weight from about 2 kDa to about 5 kDa, from about 2 kDa to about 10 kDa, from about 2 kDa to about 20 kDa, from about 2 kDa to about 30 kDa, from about 2 kDa to about 40 kDa, from about 2 kDa to about 50 kDa, from about 2 kDa to about 60 kDa, from about 2 kDa to about 70 kDa, from about 2 kDa to about 80 kDa, from about 2 kDa to about 90 kDa, from about 2 kDa to about 100 kDa, from about 2 kDa to about 150 kDa, from about 5 kDa to about 10 kDa, from about 5 kDa to about 20 kDa, from about 5 kDa to about 30 kDa, from about 5 kDa to about 40 kDa, from about 5 kDa to about 50 kDa, from about 5 kDa to about 60 kDa, from about 5 kDa to about 70 kDa, from about 5 kDa to about 80 kDa, from about 5 kDa to about 90 kDa, from about 5 kDa to about 100 kDa, from about 5 kDa to about 150 kDa, from about 10 kDa to about 20 kDa, from about 10 kDa to about 30 kDa, from about 10 kDa to about 40 kDa, from about 10 kDa to about 50 kDa, from about 10 kDa to about 60 kDa, from about 10 kDa to about 70 kDa, from about 10 kDa to about 80 kDa, from about 10 kDa to about 90 kDa, from about 10 kDa to about 100 kDa, from about 10 kDa to about 150 kDa, from about 20 kDa to about 30 kDa, from about 20 kDa to about 40 kDa, from about 20 kDa to about 50 kDa, from about 20 kDa to about 60 kDa, from about 20 kDa to about 70 kDa, from about 20 kDa to about 80 kDa, from about 20 kDa to about 90 kDa, from about 20 kDa to about 100 kDa, from about kDa to about 150 kDa, from about 30 kDa to about 40 kDa, from about 30 kDa to about 50 kDa, from about 30 kDa to about 60 kDa, from about 30 kDa to about 70 kDa, from about kDa to about 80 kDa, from about 30 kDa to about 90 kDa, from about 30 kDa to about 100 kDa, from about 30 kDa to about 150 kDa, from about 40 kDa to about 50 kDa, from about 40 kDa to about 60 kDa, from about 40 kDa to about 70 kDa, from about 40 kDa to about 80 kDa, from about 40 kDa to about 90 kDa, from about 40 kDa to about 100 kDa, from about 40 kDa to about 150 kDa, from about 50 kDa to about 60 kDa, from about 50 kDa to about 70 kDa, from about 50 kDa to about 80 kDa, from about 50 kDa to about 90 kDa, from about 50 kDa to about 100 kDa, from about 50 kDa to about 150 kDa, from about 60 kDa to about 70 kDa, from about 60 kDa to about 80 kDa, from about 60 kDa to about 90 kDa, from about 60 kDa to about 100 kDa, from about 60 kDa to about 150 kDa, from about 70 kDa to about 80 kDa, from about 70 kDa to about 90 kDa, from about 70 kDa to about 100 kDa, from about 70 kDa to about 150 kDa, from about 80 kDa to about 90 kDa, from about 80 kDa to about 100 kDa, from about 80 kDa to about 150 kDa, from about 90 kDa to about 100 kDa, from about 90 kDa to about 150 kDa, or from about 100 kDa to about 150 kDa.

It is contemplated that the antibody conjugate or fusion protein is attached to about or fewer polymer molecules (e.g., 9, 8, 7, 6, 5, 4, 3, 2, or 1), each polymer molecule having a molecular weight of at least about 20,000 D, or at least about 30,000 D, or at least about 40,000 D.

Although a variety of polymers can be used as biological modifiers, it is contemplated that the antibody conjugates or fusion proteins described herein may be attached to polyethylene glycol (PEG) polymers. In one embodiment, the antibody conjugate or fusion protein described herein is covalently attached to at least one PEG having an actual MW of at least about 20,000 D. In another embodiment, the antibody conjugate or fusion protein described herein is covalently attached to at least one PEG having an actual MW of at least about 30,000 D. In another embodiment, the antibody conjugate or fusion protein described herein is covalently attached to at least one PEG having an actual MW of at least about 40,000 D. In certain embodiments, the PEG is methoxyPEG(5000)-succinimidylpropionate (mPEG-SPA), methoxyPEG(5000)-succinimidylsuccinate (mPEG-SS). Such PEGS are commercially available from Nektar Therapeutics or SunBiowest.

Attachment sites on an antibody conjugate or fusion protein for a biological modifier include the N-terminal amino group and epsilon amino groups found on lysine residues, as well as other amino, imino, carboxyl, sulfhydryl, hydroxyl or other hydrophilic groups. The polymer may be covalently bonded directly to the antibody conjugate or fusion protein with or without the known use of a multifunctional (ordinarily bifunctional) crosslinking agent using chemistries and used in the art. For example, sulfhydryl groups can be derivatized by coupling to maleimido-substituted PEG (e.g. alkoxy-PEG amine plus sulfosuccinimidyl 4-(N-maleimidomethyl)cyclohexane-1-carboxylate), or PEG-maleimide commercially available from Shearwater Polymers, Inc., Huntsville, Ala.).

### III. Methods of Making a Recombinant Sialidase, Fusion Protein, or Antibody Conjugate

Methods for producing recombinant sialidases (e.g., human sialidases), fusion proteins, e.g., those disclosed herein, antibodies, or antibody conjugates, e.g., those disclosed herein, are known in the art. For example, DNA molecules encoding light chain variable regions and/or heavy chain variable regions can be synthesized chemically or by recombinant DNA methodologies. For example, the sequences of the antibodies can be cloned from hybridomas by conventional hybridization techniques or polymerase chain reaction (PCR) techniques, using the appropriate synthetic nucleic acid primers. The resulting DNA molecules encoding the variable regions of interest can be ligated to other appropriate nucleotide sequences, including, for example, constant region coding sequences, and expression control sequences, to produce conventional gene expression constructs (i.e., expression vectors) encoding the desired antibodies. Production of defined gene constructs is within routine skill in the art.

Nucleic acids encoding desired recombinant human sialidases, fusion proteins, and/or antibody conjugates can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving fusion proteins comprising an antibody or portion thereof, the expression vector optionally contains sequences encoding all or part of a constant region, enabling an entire, or a part of, a heavy or light chain to be expressed. The gene construct can be introduced into eukaryotic host cells using conventional techniques.

The host cells express a recombinant human sialidase or a fusion protein and/or antibody conjugate comprising a sialidase and V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments involving fusion proteins and/or antibody conjugates, a host cell is transfected with a single vector expressing a polypeptide expressing a sialidase and an entire, or part of, a heavy chain (e.g., a heavy chain variable region) or a sialidase and a light chain (e.g., a light chain variable region), or a polypeptide expressing an entire, or part of, a heavy chain (e.g., a heavy chain variable region) or a light chain (e.g., a light chain variable region). In some embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain, wherein in (a) or in (b), the polypeptide may also comprise a sialidase. In some embodiments, a host cell is co-transfected with more than one expression vector (e.g., one expression vector expressing a polypeptide comprising an entire, or part of, a heavy chain or heavy chain variable region, optionally comprising a sialidase fused thereto, and another expression vector expressing a polypeptide comprising an entire, or part of, a light chain or light chain variable region, optionally comprising a sialidase fused thereto).

A polypeptide comprising a sialidase or a fusion protein, e.g., a fusion protein comprising an immunoglobulin heavy chain variable region or light chain variable region, can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

In embodiments in which a fusion protein and/or antibody conjugate is produced, a sialidase fused to a monoclonal antibody, Fc domain, or an antigen-binding domain of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The sialidase will be fused to one or more of the chains. The intact fusion protein and/or antibody conjugate can be harvested and purified or isolated using techniques known in the art, e.g., Protein A, Protein G, affinity tags such as glutathione-S-transferase (GST) or histidine tags. It is within ordinary skill in the art to express the heavy chain and the light chain from a single expression vector or from two separate expression vectors.

In certain embodiments, in order to express a protein, e.g., a recombinant human sialidase, as a secreted protein, a native N-terminal signal sequence of the protein is replaced, e.g., with MDMRVPAQLLGLLLLWLPGARC (SEQ ID NO: 28). In certain embodiments, to express a protein, e.g., a recombinant human sialidase, as a secreted protein, an N-terminal signal sequence, e.g., MDMRVPAQLLGLLLLWLPGARC (SEQ ID NO: 28), is added. Additional exemplary N-terminal signal sequences include signal sequences from interleukin-2, CD-5, IgG kappa light chain, trypsinogen, serum albumin, and prolactin. In certain embodiments, in order to express a protein, e.g., a recombinant human sialidase, as a secreted protein, a C terminal lysosomal signal motif, e.g., YGTL (SEQ ID NO: 29) is removed.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, N.Y.), which involves a vaccinia virus-based vector to express antibodies in mammalian cells. High levels of combinatorial diversity of IgG heavy and light chains can be produced. See, e.g., U.S. Pat. No. 6,706,477 (Zauderer); U.S. Pat. No. 6,800,442 (Zauderer); and U.S. Pat. No. 6,872,518 (Zauderer). Another approach for converting a mouse antibody into a form suitable for use in humans is technology practiced commercially by KaloBios Pharmaceuticals, Inc. (Palo Alto, Calif.). This technology involves the use of a proprietary human “acceptor” library to produce an “epitope focused” library for antibody selection. Another approach for modifying a mouse antibody into a form suitable for medical use in humans is HUMAN ENGINEERING™ technology, which is practiced commercially by XOMA (US) LLC. See, e.g., International (PCT) Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

Any suitable approach, including any of the above approaches, can be used to reduce or eliminate human immunogenicity of an antibody.

In addition, it is possible to create fully human antibodies in mice. Fully human mAbs lacking any non-human sequences can be prepared from human immunoglobulin transgenic mice by techniques referenced in, e.g., Lonberg et al., NATURE 368:856-859, 1994; Fishwild et al., NATURE BIOTECHNOLOGY 14:845-851, 1996; and Mendez et al., NATURE GENETICS 15:146-156, 1997. Fully human monoclonal antibodies can also be prepared and optimized from phage display libraries by techniques referenced in, e.g., Knappik et al., J. MOL. BIOL. 296:57-86, 2000; and Krebs et al., J. IMMUNOL. METH. 254:67-84 2001).

The present invention encompasses fusion proteins comprising antibody fragments, which may be generated by traditional means, such as enzymatic digestion, or by recombinant techniques. For a review of certain antibody fragments, see Hudson et al. (2003) NAT. MED. 9:129-134.

Various techniques have been developed for the production of antibody fragments. Traditionally, these fragments were derived via proteolytic digestion of intact antibodies (see, e.g., Morimoto et al. (1992) JOURNAL OF BIOCHEMICAL AND BIOPHYSICAL METHODS 24:107-117; and Brennan et al. (1985) SCIENCE 229:81). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)₂ fragments (Carter et al. (1992) BIO/TECHNOLOGY 10:163-167). According to another approach, F(ab′)₂ fragments can be isolated directly from recombinant host cell culture. Fab and F(ab′)₂ fragments with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See U.S. Pat. Nos. 5,571,894 and 5,587,458.

Methods for making bispecific antibodies are known in the art. See Milstein and Cuello (1983) NATURE 305:537, International (PCT) Publication No. WO93/08829, and Traunecker et al. (1991) EMBO J., 10:3655. For further details of generating bispecific antibodies see, for example, Suresh et al. (1986) METHODS ENZYMOL. 121:210. Bispecific antibodies include cross-linked or “heteroconjugate” or “heterodimer” antibodies. For example, one of the antibodies in the heterodimer can be coupled to avidin, the other to biotin. Heterodimer antibodies may be made using any convenient cross-linking method. Suitable cross-linking agents are well known in the art, and are disclosed in U.S. Pat. No. 4,676,980, along with a number of cross-linking techniques.

Examples of heterodimeric or asymmetric IgG-like molecules include but are not limited to those obtained with the following technologies or using the following formats: Triomab/Quadroma, Knobs-into-Holes, CrossMabs, electrostatically-matched antibodies, LUZ-Y, Strand Exchange Engineered Domain body, Biclonic and DuoBody.

Advantages of using antibody fragments (e.g., F(ab) and F(ab′)₂ fragments) include the elimination of non-specific binding between Fc portions of antibodies and Fc receptors on cells (such as macrophages, dendritic cells, neutrophils, NK cells and B cells). In addition, they may be able to penetrate tissues more efficiently due to their smaller size.

Heterodimeric antibodies, or asymmetric antibodies, allow for greater flexibility and new formats for attaching a variety of drugs to the antibody arms. One of the general formats for creating a heterodimeric antibody is the “knobs-into-holes” format. This format is specific to the heavy chain part of the constant region in antibodies. The “knobs” part is engineered by replacing a small amino acid with a larger one, which fits into a “hole”, which is engineered by replacing a large amino acid with a smaller one. What connects the “knobs” to the “holes” are the disulfide bonds between each chain. The “knobs-into-holes” shape facilitates antibody dependent cell mediated cytotoxicity. Single chain variable fragments (scFv) are connected to the variable domain of the heavy and light chain via a short linker peptide. The linker is rich in glycine, which gives it more flexibility, and serine/threonine, which gives it specificity. Two different scFv fragments can be connected together, via a hinge region, to the constant domain of the heavy chain or the constant domain of the light chain. This gives the antibody bispecificity, allowing for the binding specificities of two different antigens. The “knobs-into-holes” format enhances heterodimer formation but doesn't suppress homodimer formation.

Several approaches to support heterodimerization have been described, for example in International (PCT) Publication Nos. WO96/27011, WO98/050431, WO2007/110205, WO2007/147901, WO2009/089004, WO2010/129304, WO2011/90754, WO2011/143545, WO2012/058768, WO2013/157954, and WO2013/096291, and European Patent Publication No. EP1870459. Typically, in the approaches known in the art, the CH₃ domain of the first heavy chain and the CH₃ domain of the second heavy chain are both engineered in a complementary manner so that the heavy chain comprising one engineered CH₃ domain can no longer homodimerize with another heavy chain of the same structure (e.g. a CH3-engineered first heavy chain can no longer homodimerize with another CH₃-engineered first heavy chain; and a CH₃-engineered second heavy chain can no longer homodimerize with another CH₃-engineered second heavy chain). Thereby the heavy chain comprising one engineered CH₃ domain is forced to heterodimerize with another heavy chain comprising the CH₃ domain, which is engineered in a complementary manner. As a result, the CH₃ domain of the first heavy chain and the CH₃ domain of the second heavy chain are engineered in a complementary manner by amino acid substitutions, such that the first heavy chain and the second heavy chain are forced to heterodimerize, whereas the first heavy chain and the second heavy chain can no longer homodimerize (e.g., for steric reasons).

### IV. Pharmaceutical Compositions

For therapeutic use, a recombinant sialidase (e.g., human sialidase) or a fusion protein and/or antibody conjugate thereof preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-methacrylate), ethylene vinyl acetate, or poly-D(−)-3-hydroxybutyric acid. Sustained release compositions may also include liposomes that can be prepared by any of several methods known in the art.

Pharmaceutical compositions containing a recombinant human sialidase, a recombinant human sialidase fusion protein, or an antibody conjugate disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a recombinant human sialidase, a recombinant human sialidase fusion protein, or an antibody conjugate disclosed herein is administered by IV infusion. In certain embodiments, a recombinant human sialidase, a recombinant human sialidase fusion protein, or an antibody conjugate disclosed herein is administered by intratumoral injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

In certain embodiments, a pharmaceutical composition may contain a stabilizing agent. In certain embodiments, the stabilizing agent is a cation, such as a divalent cation. In certain embodiments, the cation is calcium or magnesium. The cation can be in the form of a salt, such as calcium chloride (CaCl₂) or magnesium chloride (MgCl₂).

In certain embodiments, the stabilizing agent is present in an amount from about 0.05 mM to about 5 mM. For example, the stabilizing agent may be present in an amount of from about 0.05 mM to about 4 mM, from about 0.05 mM to about 3 mM, from about 0.05 mM to about 2 mM, from about 0.05 mM to about 1 mM, from about 0.05 mM to about 0.5 mM, from about 0.5 mM to about 4 mM, from about 0.5 mM to about 3 mM, from about 0.5 mM to about 2 mM, from about 0.5 mM to about 1 mM, from about 1 mM to about 4 mM, from about 1 mM to about 3 mM, of from about 1 mM to about 2 mM.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

Generally, a therapeutically effective amount of active component, for example, a recombinant human sialidase or fusion protein and/or antibody conjugate thereof, is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the recombinant human sialidase or fusion protein and/or antibody conjugate thereof, and the disease being treated. Exemplary dosing frequencies are once per day, once per week and once every two weeks. A preferred route of administration is parenteral, e.g., intravenous infusion. In certain embodiments, a recombinant human sialidase or a fusion protein and/or antibody conjugate thereof is lyophilized, and then reconstituted in buffered saline, at the time of administration.

### V. Therapeutic Uses

The compositions and methods disclosed herein can be used to treat various forms of cancer in a subject or inhibit cancer growth in a subject. The invention provides a method of treating a cancer in a subject. The method comprises administering to the subject an effective amount of a recombinant human sialidase or a fusion protein and/or antibody conjugate thereof, e.g., a recombinant human sialidase, fusion protein, or antibody conjugate disclosed herein, either alone or in a combination with another therapeutic agent to treat the cancer in the subject. The term “effective amount” as used herein refers to the amount of an active agent (e.g., recombinant human sialidase or fusion protein thereof according to the present invention) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CIVIL), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma).

In certain embodiments the cancer is an epithelial cancer, e.g., an epithelial cancer that upregulates the expression of sialylated glycans. Exemplary epithelial cancers include, but are not limited to, endometrial cancer, colon cancer, ovarian cancer, cervical cancer, vulvar cancer, uterine cancer or fallopian tube cancer, breast cancer, prostate cancer, lung cancer, pancreatic cancer, urinary cancer, bladder cancer, head and neck cancer, oral cancer and liver cancer. Epithelial cancers also include carcinomas, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, baso squamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossifi cans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

In certain embodiments, the cancer is breast cancer. In certain embodiments, the cancer is an adenocarcinoma. In certain embodiments, the cancer is a metastatic cancer. In certain embodiments, the cancer is a refractory cancer.

In certain embodiments, the cancer is resistant to or non-responsive to treatment with an antibody, e.g., an antibody with ADCC activity, e.g., trastuzumab.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more additional therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodilator, a statin, an anti-inflammatory agent (e.g. methotrexate), or an NSAID. In certain embodiments, the additional therapy may include a combination of therapeutics of different classes.

In certain embodiments, a method or composition described herein is administered in combination with a checkpoint inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist, and TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), durvalumab (AstraZeneca), MEDI4736, avelumab, and BMS 936559 (Bristol Myers Squibb Co.).

In certain embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, a method or composition described herein is administered in combination with (i) a PD-1 or PD-L1 inhibitor, e.g., a PD-1 or PD-L1 inhibitor disclosed herein, and (ii) CTLA-4 inhibitor, e.g., a CTLA-4 inhibitor disclosed herein.

In certain embodiments, a method or composition described herein is administered in combination with an DO inhibitor. Exemplary IDO inhibitors include 1-methyl-D-tryptophan (known as indoximod), epacadostat (INCB24360), navoximod (GDC-0919), and BMS-986205.

Exemplary cytotoxic agents that can be administered in combination with a method or composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a method or composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

The invention also provides a method of increasing the expression of HLA-DR, CD86, CD83, IFNγ, IL-1b, IL-6, TNFα, IL-17A, IL-2, or IL-6 in a cell, tissue, or subject. The method comprises contacting the cell, tissue, or subject with an effective amount of a sialidase, fusion protein, and/or antibody conjugate, e.g., a sialidase, fusion protein, or antibody conjugate disclosed herein. In certain embodiments, the cell is selected from a dendritic cell and a peripheral blood mononuclear cell (PBMC).

In certain embodiments, expression of HLA-DR, CD86, CD83, IFNγ, IL-1b, IL-6, TNFα, IL-17A, IL-2, or IL-6 in the cell, tissue, or subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with the sialidase, fusion protein, or antibody conjugate. Gene expression may be measured by any suitable method known in the art, for example, by ELISA, or by Luminex multiplex assays.

The invention also provides a method of promoting infiltration of immune cells into a tumor in a subject in need thereof. The method comprises administering to the subject an effective amount of a sialidase, fusion protein, and/or antibody conjugate, e.g., a sialidase, fusion protein, or antibody conjugate disclosed herein. In certain embodiments, the immune cells are T-cells, e.g., CD4+ and/or CD8+ T-cells, e.g., CD69⁺CD8⁺ and/or GzmB⁺CD8⁺ T-cells. In certain embodiments, the immune cells are natural killer (NK) cells.

In certain embodiments, the infiltration of immune cells into the tumor in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical tumor and/or subject that has not been administered the sialidase, fusion protein, or antibody conjugate. Infiltration of immune cells into a tumor may be measured by any suitable method known in the art, for example, antibody staining.

The invention also provides a method of increasing the amount of circulating natural killer (NK) cells in a subject in need thereof. The method comprises administering to the subject an effective amount of a sialidase, fusion protein, and/or antibody conjugate, e.g., a sialidase, fusion protein, or antibody conjugate disclosed herein, so as to increase the number of circulating NK cells relative to prior to administration of the sialidase, fusion protein, antibody conjugate, or pharmaceutical composition.

In certain embodiments, the amount of circulating NK cells in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical subject that has not been administered the sialidase, fusion protein, or antibody conjugate. Circulating NK cells in a subject may be measured by any suitable method known in the art, for example, antibody staining.

The invention also provides a method of increasing the amount of T-cells in the draining lymph node in a subject in need thereof. The method comprises administering to the subject an effective amount of a sialidase, fusion protein, antibody conjugate, and/or pharmaceutical composition, e.g., a sialidase, fusion protein, antibody conjugate, and/or pharmaceutical composition disclosed herein, so as to increase the number of T-cells in the draining lymph node relative to prior to administration of the sialidase, fusion protein, antibody conjugate, or pharmaceutical composition. In certain embodiments, the immune cells are T-cells, e.g., CD4+ and/or CD8+ T-cells.

In certain embodiments, the amount of T-cells in the draining lymph node in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical subject that has not been administered the sialidase, fusion protein, antibody conjugate, or pharmaceutical composition. T-cells in the draining lymph node in a subject may be measured by any suitable method known in the art, for example, antibody staining.

The invention also provides a method of increasing expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 in a cell, tissue, or subject. The method comprises contacting the cell, tissue, or subject with an effective amount of a sialidase, fusion protein, antibody conjugate, and/or pharmaceutical composition, e.g., a sialidase, fusion protein, antibody conjugate, and/or pharmaceutical composition disclosed herein, so as to increase the expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 relative to the cell, tissue or subject prior to contact with the sialidase, fusion protein, antibody conjugate, or pharmaceutical composition.

In certain embodiments, expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 in the cell, tissue, or subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell, tissue, or subject that has not been contacted with the sialidase, fusion protein, antibody conjugate, or pharmaceutical composition. Gene expression may be measured by any suitable method known in the art, for example, by ELISA, Luminex multiplex assays, or Nanostring technology.

The invention also provides a method of removing sialic acid from a cell or tissue. The method comprises contacting the cell or tissue with an effective amount of a sialidase, fusion protein, and/or antibody conjugate, e.g., a sialidase, fusion protein, or antibody conjugate disclosed herein. The invention also provides a method of removing sialic acid from a cell in a subject, the method comprising administering to the subject an effective amount of a pharmaceutical composition comprising a sialidase, fusion protein, and/or antibody conjugate, e.g., a sialidase, fusion protein, or antibody conjugate disclosed herein, thereby to remove sialic acid from the cell.

In certain embodiments, the cell is tumor cell, dendritic cell (DC) or monocyte. In certain embodiments, the cell is a monocyte, and the method results in increased expression of an MHC-II molecule (e.g., HLA-DR) on the monocyte. In certain embodiments, expression of an MHC-II molecule in the cell or tissue is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with the sialidase, fusion protein, and/or antibody conjugate. Gene expression may be measured by any suitable method known in the art, for example, by ELISA, by Luminex multiplex assays, or by flow cytometry.

The invention also provides a method of enhancing phagocytosis of a tumor cell. The method comprises contacting the tumor cell with a sialidase, fusion protein, and/or antibody conjugate, e.g., a sialidase, fusion protein, or antibody conjugate disclosed herein, in an amount effective to remove sialic acid from the tumor cell, thereby enhancing phagocytosis of the tumor cell. In certain embodiments, the disclosure relates to a method of increasing phagocytosis of a tumor cell in a subject, the method comprising administering to the subject an effective amount of a pharmaceutical composition comprising a sialidase, fusion protein, and/or antibody conjugate, e.g., a sialidase, fusion protein, or antibody conjugate disclosed herein, in an amount effective to remove sialic acid from the tumor cell, thereby to increase phagocytosis of the tumor cell.

In certain embodiments, phagocytosis is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical tumor cell or population of tumor cells that has not or have not been contacted with the sialidase, fusion protein, and/or antibody conjugate. Phagocytosis may be measured by any suitable method known in the art.

The invention also provides a method of activating a dendritic cell (DC). The method comprises contacting the DC with a tumor cell that has been treated with a sialidase, fusion protein, and/or antibody conjugate, e.g., a sialidase, fusion protein, or antibody conjugate disclosed herein. In certain embodiments, the disclosure relates to a method of activating a dendritic cell (DC) or a population of DCs in a subject, the method comprising administering to the subject an amount of a pharmaceutical composition comprising a sialidase, fusion protein, and/or antibody conjugate, e.g., a sialidase, fusion protein or antibody conjugate disclosed herein, effective to remove sialic acid from a tumor cell in the subject, thereby to activate the DC or the population of DCs in the subject.

In certain embodiments, activation of the DC or a population of DCs is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical DC or population of DCs that has not or have not been contacted with a tumor cell that has been treated with the sialidase, fusion protein, and/or antibody conjugate. Activation may be measured by any suitable method known in the art.

The invention also provides a method of reducing Siglec-15 binding activity, thereby to increase anti-tumor activity in a tumor microenvironment, the method comprising contacting a T cell with a sialidase, fusion protein, and/or antibody conjugate, e.g., a sialidase, fusion protein, or antibody conjugate disclosed herein. In certain embodiments, the disclosure relates to a method of reducing Siglec-15 binding activity, thereby to increase anti-tumor activity in a tumor microenvironment of a patient, the method comprising administering to the subject an effective amount of a pharmaceutical composition comprising a sialidase, fusion protein, and/or antibody conjugate, e.g., a sialidase, fusion protein, or antibody conjugate disclosed herein, thereby to increase anti-tumor activity (e.g., T cell activity) in the subject.

In certain embodiments, Siglec-15 binding activity is reduced by at least about 10%, at least about 20%, at least about 50%, at least about 75%, or about 100%, relative to Siglec-15 that has not or have not been contacted with the sialidase, fusion protein, and/or antibody conjugate. Binding may be measured by any suitable method known in the art.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This example describes the construction of recombinant human sialidases (Neu1, Neu2, and Neu3).

The human sialidases Neu1, Neu2, Neu3 (isoform 1), and Neu4 (isoform 1) were expressed as secreted proteins with a 10×His tag. To express Neu1 as a secreted protein, the native N terminal signal peptide (MTGERPSTALPDRRWGPRILGFWGGCRVWVFAAIFLLLSLAASWSKA; SEQ ID NO: 27) was replaced by MDMRVPAQLLGLLLLWLPGARC (SEQ ID NO: 28), and the C terminal lysosomal signal motif (YGTL; SEQ ID NO: 29) was removed. To express Neu2, Neu3, and Neu4 as secreted proteins, the N terminal signal peptide MDMRVPAQLLGLLLLWLPGARC (SEQ ID NO: 28) was added to each.

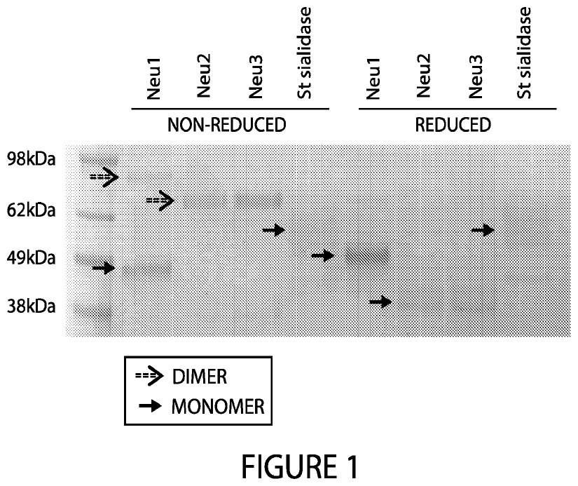

Sialidases were expressed in a 200 mL transfection of HEK293F human cells in 24-well plates using the pCEP4 mammalian expression vector with an N-terminal 6×His tag. Sialidases were purified using Ni-NTA columns, quantified with a UV-Vis spectrophotometer (NanoDrop), and examined by SDS-PAGE as shown in FIG. 1. Neu1 expressed well, with a yield of ˜3 μg/ml, and was present primarily in a monomeric form. Neu2 and Neu3 expression each gave yields of ˜0.15 μg/mL and each were present primarily in a dimeric form. Neu4 had no detectable expression yield as measured by NanoDrop. Bacterial sialidase from Salmonella typhimurium (St-sialidase; SEQ ID NO: 30), which was used as a positive control for expression, gave a comparable yield to Neu1, and was present primarily in a monomeric form.

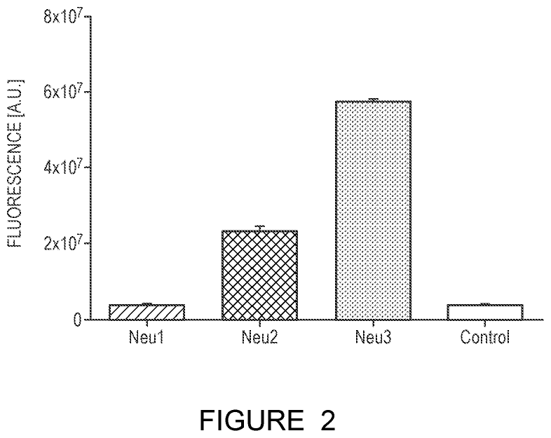

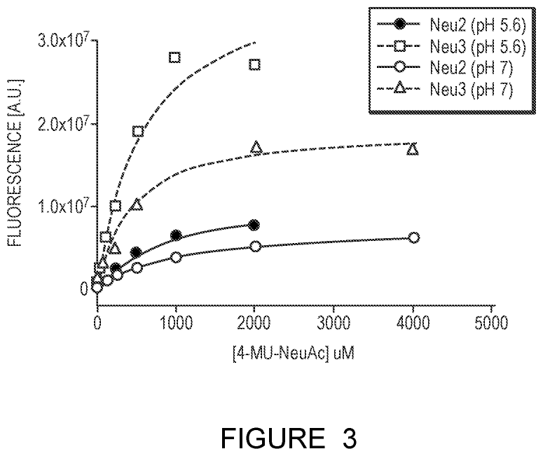

The activity of the recombinantly expressed sialidases was assayed by measuring the release of sialic acid from the fluorogenic substrate 4-methylumbelliferyl-N-acetylneuraminic acid (4MU-NeuAc). As shown in FIG. 2, Neu1 has no detectable activity above a no-enzyme control, which is consistent with previous reports indicating that Neu1 is inactive unless it is in complex with beta-galactosidase and protective protein/cathepsin A (PPCA). Neu2 and Neu3 were active. An enzyme kinetics assay was performed with Neu2 and Neu3. A fixed concentration of enzyme at 1 nM was incubated with fluorogenic substrate 4MU-NeuAc at concentrations ranging from 4000 μM to 7.8 μM. Assays were conducted at both acidic (pH 5.6) and neutral (pH 7) conditions. As shown in FIG. 3, both Neu2 and Neu3 were active at acidic and neutral conditions and showed enzyme kinetics that were comparable to those previously reported.

Most of the recombinantly expressed sialidases ran as aggregates or dimers on a non-reducing SDS-PAGE gel. Subsequent treatment with the reducing agent dithiothreitol (DTT) resulted in a monomeric form of the enzyme that ran at 42 kDa on a reducing SDS-PAGE gel (FIG. 1).

### Example 2

This example describes the construction of recombinant human sialidases with mutations that increase expression and/or activity of the sialidase.

### A. Rational Design

Structural and sequence analysis identified residues A93 and P62 of Neu2 as candidates for substitutions to increase solubility and/or expression. In particular, a comparison of homologous sialidase sequences showed a preference for D or E amino acid residues at positions corresponding to position 93 of Neu2, and a preference for G amino acid residues at positions corresponding to position 62 of Neu2.

The beta-propeller family of proteins are usually stabilized by extensive hydrogen bonding interactions at the N- and C-termini of the protein. A structural analysis revealed that Neu2, which is a member of the beta-propeller family, appears to lack these stabilizing interactions. In contrast, sialidases from Salmonella typhimurium and Micromonospora viridifaciens (the bacterial sialidase most homologous to human Neu2) have extensive hydrogen bonding interactions at their N- and C-termini. Accordingly, residues K9, V363, and L365 of Neu2 were mutated to promote hydrogen bonding between the N- and C-termini of Neu2.

### B. Phage Display

Neu2 was expressed in a phage display system allowing for screening of Neu2 variants for both expression level and resistance to heat denaturation. Neu2 with V6Y and I187K substitutions was used as a template for library preparation. Designed phage display libraries 1, 2, and 3 are depicted in TABLES 11-13, respectively. Each library included all of the possible combinations of the mutations depicted. A fourth library included random mutations generated by error prone PCR.

TABLE 11
Phage Library 1

| Wild-Type | | | |
|---|---|---|---|
| Neu2 Amino | | Substituting | Codon |
| Acid(s) | Design | Amino Acids | Usage |
| 184-188 | Adjust length of 5 wild-type | S, N, R, K, T, G, | RVM |
| LHPIQ | residues from 2 to 5 and | D, E, A | |
| | substitute each residue with | | |
| | one of 9 polar amino acids | | |
| P190, I191 | Substitute with one of 5 non- | F, I, L, M, V | NTK |
| | polar amino acids | | |
| C219 | Substitute with one of 12 polar | R, N, D, C, G, | NDT |
| | or nonpolar amino acids | H, I, L, F, S, Y, | |
| | | V | |
| L217 | Substitute with one of 12 polar | R, N, D, C, G, | NDT |
| | or nonpolar amino acids | H, I, L, F, S, Y, | |
| | | V | |
| T216 and L217 | Insert one of 12 polar or | S, T, Y, L, F, A, | NHT |
| | nonpolar amino acids between | P, V, I, N, D, H | |
| | the wild-type amino acids | | |
| G271 | Substitute with one of 9 polar | S, N, R, K, T, G, | RVM |
| | amino acids | D, E, A | |
| C272 | Substitute with one of 9 polar | S, N, R, K, T, G, | RVM |
| | amino acids | D, E, A | |

TABLE 12
Phage Library 2

| Wild-Type | | | |
|---|---|---|---|
| Neu2 Amino | | Substituting | Codon |
| Acid(s) | Design | Amino Acids | Usage |
| 156-165 | Substitute with one of 12 | R, N, D, C, G, H, | NDT |
| TFAVGPGHCL | hydrophobic or polar amino | I, L, F, S, Y, V | |
| | acids | | |
| V176 | Substitute with one of 12 | R, N, D, C, G, H, | NDT |
| | hydrophobic or polar amino | I, L, F, S, Y, V | |
| | acids | | |
| P177 | Substitute with one of 12 | S, T, Y, L, F, A, | NHT |
| | hydrophobic or polar amino | P, V, I, N, D, H | |
| | acids | | |
| A178 | Substitute with one of 12 | S, T, Y, L, F, A, | NHT |
| | hydrophobic or polar amino | P, V, I, N, D, H | |
| | acids | | |
| A194 | Substitute with one of 12 | S, T, Y, L, F, A, | NHT |
| | hydrophobic or polar amino | P, V, I, N, D, H | |
| | acids | | |

TABLE 13
Phage Library 3

| Wild-Type Neu2 | | Substituting | Codon |
|---|---|---|---|
| Amino Acid(s) | Design | Amino Acids | Usage |
| F13 | Substitute with one of 9 polar | S, N, R, K, T, G, | RMV |
| | amino acids chain AAs | D, E, A | |
| L4 | Substitute with one of 12 | S, T, Y, L, F, A, | NHT |
| | hydrophobic or polar amino | P, V, I, N, D, H | |
| | acids | | |
| L7, V12 | Substitute with one of 6 polar | F, Y, S, I, T, N | WHT |
| | or aromatic amino acids | | |
| I22, A24 | Substitute with one of 9 polar | S, N, R, K, T, G, | RMV |
| | amino acids | D, E, A | |
| V325, L326, and | Substitute with one of 6 polar | F, Y, S, I, T, N | WHT |
| L327 | or aromatic amino acids | | |
| L365 | Substitute with one of 6 polar | F, Y, S, I, T, N | WHT |
| | or aromatic amino acids | | |
| P89 | Substitute with one of 5 non- | F, I, L, M, V | NTK |
| | polar amino acids | | |
| L34, A36, and | Substitute with one of 12 | S, T, Y, L, F, A, | NHT |
| V363 | hydrophobic or polar amino | P, V, I, N, D, H | |
| | acids | | |

The codon usage columns in TABLES 11-13 represent degenerate codon codes used in the design of the library, where the first, second, and third positions of a given codon encoding an amino acid are as shown in TABLE 14 and as described in Mena et al. (2005) PROTEIN ENG DES SEL. 18(12):559-61.

TABLE 14

| Degenerate Codon Code | Mixed bases |
|---|---|
| R | A, G |
| Y | C, T/U |
| M | A, C |
| K | G, T/U |
| S | G, C |
| W | A, T |
| H | A, C, T/U |
| B | G, C, T (or |
| | U) |
| V | A, C, G |
| D | A, G, T/U |
| N | A, C, G, T/U |

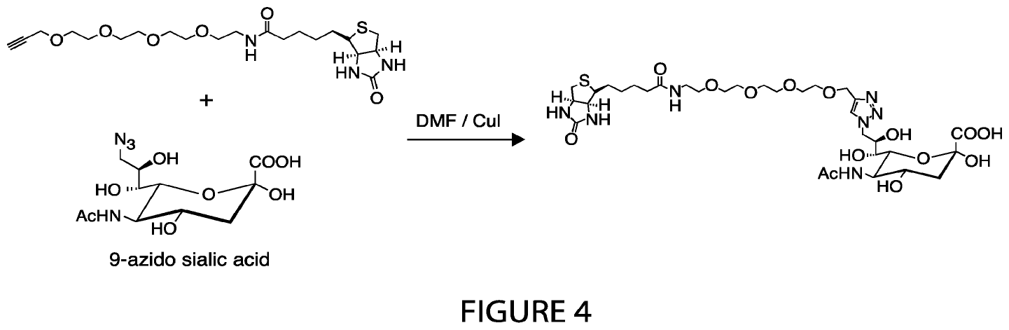

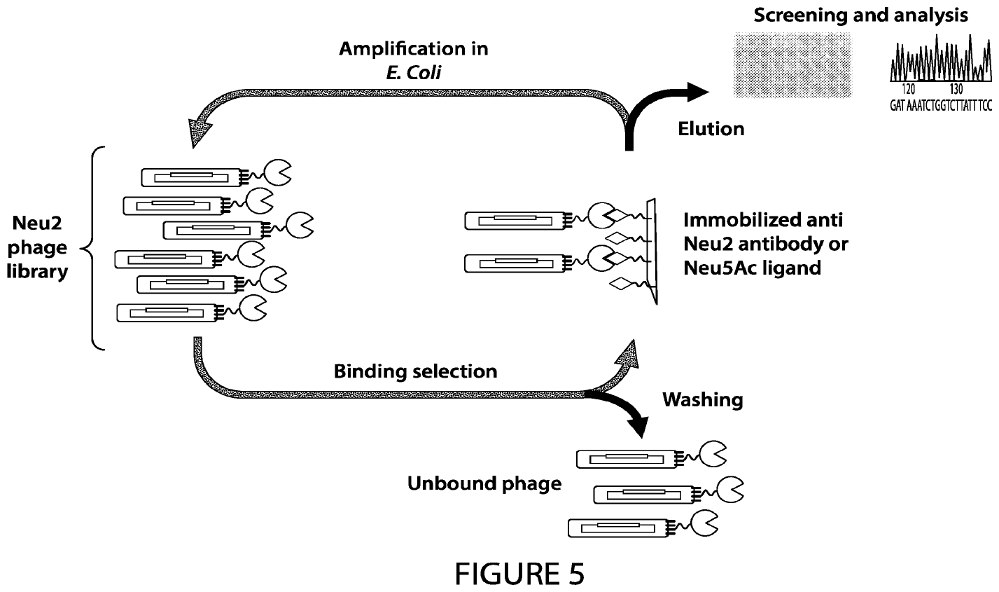

The phage display libraries were screened for binding to a conformation-specific antibody and/or a sialic acid biotinylated probe after heating to enrich for thermal stability and expression. The sialic acid biotinylated probe and its synthesis is depicted in FIG. 4. An exemplary phage display screening procedure is depicted in FIG. 5. Briefly, phage libraries expressing the desired Neu2 variants were generated. Phage were screened for binding to immobilized anti-Neu2 antibody and/or sialic acid biotinylated probe. Following washing to remove unbound phage, bound phage were eluted from the antibody or probe and analyzed as appropriate.

### C. Yeast Display

Neu2 was also expressed in a yeast display system allowing for screening of Neu2 variants for both expression level and resistance to heat denaturation. Neu2 with V6Y and I187K substitutions was used as a template for library preparation. Designed yeast display libraries 1a, 1b, 1c, 1d, 2a, 2b, 2c, 3a, 3b, and 3c are depicted in TABLES 15-24, respectively. Each library included all of the possible combinations of the mutations depicted. Five additional sublibraries were generated by error prone PCR, at an approximate average rate of 1, 2, 3, 4, and 5 substitutions per enzyme.

TABLE 15
Yeast Library 1a

| Wild-Type Neu2 | | Codon |
|---|---|---|
| Amino Acid(s) | Substituting Amino Acids | Usage |
| L184 | All | NNK |
| H185 | All | NNK |
| P186 | None (Wild-type) | |
| K187 | None (Wild-type) | |
| Q188 | None (Wild-type) | |
| P190 | A, D, G, H, I, L, N, P, R, S, T, V | VNT |
| I191 | A, D, F, H, I, L, N, P, S, T, V, Y | NHT |
| C219 | None (Wild-type) | |
| G271 | All | NNK |
| C272 | All | NNK |

Total Diversity: 2.30E+07

TABLE 16
Yeast Library 1b

| Wild-Type Neu2 | | Codon |
|---|---|---|
| Amino Acid(s) | Substituting Amino Acids | Usage |
| L184 | A, D, F, H, I, L, N, P, S, T, V, Y | NHT |
| H185 | All | NNK |
| P186 | A, D, F, H, I, L, N, P, S, T, V, Y | NHT |
| K187 | None (Wild-type) | |
| Q188 | None (Wild-type) | |
| P190 | A, D, G, H, I, L, N, P, R, S, T, V | VNT |
| I191 | A, D, F, H, I, L, N, P, S, T, V, Y | NHT |
| C219 | None (Wild-type) | |
| G271 | A, D, E, G, K, N, R, S, T | RVM |
| C272 | A, C, D, G, H, N, P, R, S, T | NVT |

Total Diversity: 4.11E+07

TABLE 17
Yeast Library 1c

| Wild-Type Neu2 | | Codon |
|---|---|---|
| Amino Acid(s) | Substituting Amino Acids | Usage |
| L184 | A, D, F, H, I, L, N, P, S, T, V, Y | NHT |
| H185 | A, D, E, G, K, N, R, S, T | RVM |
| P186 | A, D, F, H, I, L, N, P, S, T, V, Y | NHT |
| K187 | A, D, E, G, K, N, R, S, T | RVM |
| Q188 | None (Wild-type) | |
| P190 | F, L, I, V | NTT |
| I191 | A, D, F, H, I, L, N, P, S, T, V, Y | NHT |
| C219 | None (Wild-type) | |
| G271 | A, D, E, G, K, N, R, S, T | RVM |
| C272 | A, D, F, H, L, P, S, V, Y | BHT |

Total Diversity: 4.53E+07

TABLE 18
Yeast Library 1d

| Wild-Type Neu2 | | Codon |
|---|---|---|
| Amino Acid(s) | Substituting Amino Acids | Usage |
| L184 | A, D, F, H, L, P, S, V, Y | BHT |
| H185 | A, D, E, G, K, N, R, S, T | RVM |
| P186 | A, D, F, H, L, P, S, V, Y | BHT |
| K187 | A, D, E, G, K, N, R, S, T | RVM |
| Q188 | A, D, F, H, L, P, S, V, Y | BHT |
| P190 | None (Wild-type) | |
| I191 | A, D, E, G, K, N, R, S, T | RVM |
| C219 | None (Wild-type) | |
| G271 | A, D, E, G, K, N, R, S, T | RVM |
| C272 | A, D, F, H, L, P, S, V, Y | BHT |

Total Diversity: 4.30E+07

TABLE 19
Yeast Library 2a

| Wild-Type Neu2 | | Codon |
|---|---|---|
| Amino Acid(s) | Substituting Amino Acids | Usage |
| T156 | R, N, D, C, G, H, I, L, F, S, Y, V | NDT |
| F157 | None (Wild-type) | |
| A158 | R, N, D, C, G, H, I, L, F, S, Y, V | NDT |
| V159 | None (Wild-type) | |
| G160 | R, N, D, C, G, H, I, L, F, S, Y, V | NDT |
| P161 | None (Wild-type) | |
| G162 | R, N, D, C, G, H, I, L, F, S, Y, V | NDT |
| H163 | None (Wild-type) | |
| C164 | R, N, D, C, G, H, I, L, F, S, Y, V | NDT |
| L165 | None (Wild-type) | |
| V176 | L, V, P, A, H, D | SHT |
| P177 | L, V, P, A, H, D | SHT |
| A178 | L, V, P, A, H, D | SHT |
| A194 | L, V, P, A, H, D | SHT |

Total Diversity: 3.22E+08

TABLE 20
Yeast Library 2b

| Wild-Type Neu2 | | Codon |
|---|---|---|
| Amino Acid(s) | Substituting Amino Acids | Usage |
| T156 | None (Wild-type) | |
| F157 | R, N, D, C, G, H, I, L, F, S, Y, V | NDT |
| A158 | None (Wild-type) | |
| V159 | R, N, D, C, G, H, I, L, F, S, Y, V | NDT |
| G160 | None (Wild-type) | |
| P161 | R, N, D, C, G, H, I, L, F, S, Y, V | NDT |
| G162 | None (Wild-type) | |
| H163 | R, N, D, C, G, H, I, L, F, S, Y, V | NDT |
| C164 | None (Wild-type) | |
| L165 | R, N, D, C, G, H, I, L, F, S, Y, V | NDT |
| V176 | L, V, P, A, H, D | SHT |
| P177 | L, V, P, A, H, D | SHT |
| A178 | L, V, P, A, H, D | SHT |
| A194 | L, V, P, A, H, D | SHT |

Total Diversity: 3.22E+08

TABLE 21
Yeast Library 2c

| Wild-Type Neu2 | | Codon |
|---|---|---|
| Amino Acid(s) | Substituting Amino Acids | Usage |
| T156 | A, D, G, H, N, P, R, S, T | VVC |
| F157 | A, D, F, H, L, P, S, V, Y | BHT |
| A158 | A, D, G, H, N, P, R, S, T | VVC |
| V159 | A, D, F, H, L, P, S, V, Y | BHT |
| G160 | A, D, G, H, N, P, R, S, T | VVC |
| P161 | A, D, F, H, L, P, S, V, Y | BHT |
| G162 | A, D, G, H, N, P, R, S, T | VVC |
| H163 | A, D, F, H, L, P, S, V, Y | BHT |
| C164 | A, D, G, H, N, P, R, S, T | VVC |
| L165 | A, D, F, H, L, P, S, V, Y | BHT |
| V176 | None (Wild-type) | |
| P177 | None (Wild-type) | |
| A178 | None (Wild-type) | |
| A194 | None (Wild-type) | |

Total Diversity: 3.49E+09

TABLE 22
Yeast Library 3a

| Wild-Type Neu2 | | Codon |
|---|---|---|
| Amino Acid(s) | Substituting Amino Acids | Usage |
| L4 | S, T, Y, L, F, A, P, V, I, N, D, H | NHT |
| L7 | F, Y, S, I, T, N | WHT |
| V12 | None (Wild-type) | |
| F13 | None (Wild-type) | |
| I22 | S, N, R, K, T, G, D, E, A | RMV |
| A24 | S, N, R, K, T, G, D, E, A | RMV |
| L34 | None (Wild-type) | |
| A36 | None (Wild-type) | |
| H64 | F, Y, S, I, T, N | WHT |
| P89 | F, I, L, V | NTT |
| C164 | None (Wild-type) | |
| V325 | F, Y, S, I, T, N | WHT |
| L326 | F, Y, S, I, T, N | WHT |
| L327 | F, Y, S, I, T, N | WHT |
| C332 | None (Wild-type) | |
| V363 | S, T, Y, L, F, A, P, V, I, N, D, H | NHT |

Total Diversity: 3.63E+08

TABLE 23
Yeast Library 3b

| Wild-Type Neu2 | | Codon |
|---|---|---|
| Amino Acid(s) | Substituting Amino Acids | Usage |
| L4 | None (Wild-type) | |
| L7 | F, Y, S, I, T, N | WHT |
| V12 | S, T, Y, L, F, A, P, V, I, N, D, H | NHT |
| F13 | S, N, R, K, T, G, D, E, A | RMV |
| I22 | S, N, R, K, T, G, D, E, A | RMV |
| A24 | S, N, R, K, T, G, D, E, A | RMV |
| L34 | S, T, Y, L, F, A, P, V, I, N, D, H | NHT |
| A36 | S, T, Y, L, F, A, P, V, I, N, D, H | NHT |
| H64 | None (Wild-type) | |
| P89 | None (Wild-type) | |
| C164 | A, G, S, T | RST |
| V325 | None (Wild-type) | |
| L326 | None (Wild-type) | |
| L327 | None (Wild-type) | |
| C332 | A, D, G, H, N, P, R, S, T | VVC |
| V363 | None (Wild-type) | |

Total Diversity: 2.72E+08

TABLE 24
Yeast Library 3c

| Wild-Type Neu2 | | Codon |
|---|---|---|
| Amino Acid(s) | Substituting Amino Acids | Usage |
| L4 | None (Wild-type) | |
| L7 | None (Wild-type) | |
| V12 | None (Wild-type) | |
| F13 | None (Wild-type) | |
| I22 | S, T, Y, L, F, A, P, V, I, N, D, H | NHT |
| A24 | S, T, Y, L, F, A, P, V, I, N, D, H | NHT |
| L34 | None (Wild-type) | |
| A36 | None (Wild-type) | |
| H64 | F, Y, S, I, T, N | WHT |
| P89 | S, T, Y, L, F, A, P, V, I, N, D, H | NHT |
| C164 | A, G, S, T | RST |
| V325 | A, D, F, H, L, P, S, V, Y | BHT |
| L326 | A, D, F, H, L, P, S, V, Y | BHT |
| L327 | A, D, F, H, L, P, S, V, Y | BHT |
| C332 | A, D, G, H, N, P, R, S, T | VVC |
| V363 | None (Wild-type) | |

Total Diversity: 2.72E+08

The codon usage columns in TABLES 15-24 represent degenerate codon codes used in the design of the library, where the first, second, and third positions of a given codon encoding an amino acid are as shown herein above in TABLE 14 and as described in Mena et al. (2005) PROTEIN ENG DES SEL. 18(12):559-61.

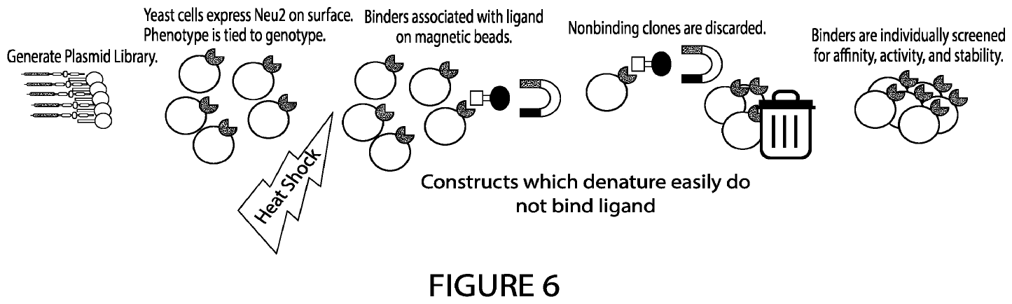

The yeast display libraries were screened for binding to a conformation-specific antibody and/or a sialic acid biotinylated probe after heating to enrich for thermal stability and expression. An exemplary yeast display screening procedure is depicted in FIG. 6. Briefly, a plasmid library encoding for the desired Neu2 variants, and yeast cells expressing the desired Neu2 variants on the surface, were generated. Yeast cells were heat shocked and then screened for binding to anti-Neu2 antibody and/or sialic acid biotinylated probe on magnetic beads. The magnetic beads were isolated to remove unbound cells, and bound cells were further analyzed for Neu2 affinity, activity, and stability as appropriate.

### D. Results

Mutant sialidases including mutations identified using the rational design, phage display, and yeast display approaches described in this Example were expressed as secreted proteins with a C-terminal human Fc tag in Expi293F cells using the pCEP4 mammalian expression vector. Expression was assayed using a ForteBio Octet with anti-human Fc sensors and Western blot and enzymatic activity was assayed using the fluorogenic substrate 4MU-NeuAc as described above.

Expression and activity levels for the mutant sialidases are shown in TABLE 25. In TABLE 25, enzymatic activity is indicated as “+++,” which denotes activity >2 fold higher than wild-type Neu2, “++,” which denotes activity comparable to wild-type Neu2, “+,” which denotes activity lower than wild-type Neu2, or “−,” which denotes no detectable activity, and expression is indicated as “++++,” which denotes expression >15 fold higher than wildtype-Neu2, “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 25

| Identifier | Mutation(s) | Activity | Expression |
|---|---|---|---|
| Neu2-M104 | M1D, V6Y, P62G, I187K, C332A | + | ++++ |
| Neu2-M105 | M1D, V6Y, K9D, I187K, C332A, | + | +++ |
| | V363R, L365I | | |
| Neu2-M106 | M1D, V6Y, P62G, A93E, I187K, | ++ | ++++ |
| | C332A | | |
| Neu2-M107 | M1D, V6Y, K9D, I187K, C332A, | + | ++++ |
| | V363R, L365K | | |
| Neu2-M108 | M1D, V6Y, K9D, I187K, C332A, | + | ++++ |
| | V363R, L365S | | |
| Neu2-M109 | M1D, V6Y, K9D, I187K, C332A, | + | ++++ |
| | V363R, L365Q | | |
| Neu2-M110 | M1D, V6Y, K9D, I187K, C332A, | + | +++ |
| | V363R, L365H | | |
| Neu2-M111 | M1D, V6Y, A93K, I187K, C332A | ++ | ++ |
| Neu2-M112 | M1D, V6Y, A93E, I187K, C332A | ++ | ++ |
| Neu2-M113 | V6Y, I22N, C125Y, I187K, | − | +++ |
| | S301C, E319D | | |
| Neu2-M114 | V6Y, P62T, C125F, I187K, | − | +++ |
| | A222D | | |
| Neu2-M115 | V6Y, I187K, W292R | + | +++ |
| Neu2-M116 | V6Y, G107D, I187K | + | +++ |
| Neu2-M117 | C125L | + | + |
| Neu2-M118 | V6Y, C125L | + | ++ |
| Neu2-M119 | C125L, I187K | ++ | ++ |
| Neu2-M120 | V6Y, C125L, I187K | + | +++ |
| Neu2-M121 | M1D, V6Y, K45A, I187K, C332A | ++ | ++ |
| Neu2-M122 | M1D, V6Y, Q270A, I187K, | ++ | +++ |
| | C332A | | |
| Neu2-M123 | M1D, V6Y, K44R, K45R, I187K, | + | ++ |
| | C332A | | |
| Neu2-M124 | M1D, V6Y, Q112R, I187K, | + | ++ |
| | C332A | | |
| Neu2-M125 | M1D, V6Y, Q270F, I187K, | + | ++ |
| | C332A | | |
| Neu2-M126 | M1D, V6Y, I187K, S301R, | ++ | +++ |
| | W302K, C332A | | |
| Neu2-M127 | M1D, V6Y, K44E, K45E, I187K, | ++ | + |
| | C332A | | |
| Neu2-M128 | M1D, V6Y, I187K, L217V, | + | ++ |
| | C332A | | |
| Neu2-M129 | M1D, V6Y, I187K, L217A, | + | ++ |
| | C332A | | |
| Neu2-M130 | M1D, V6Y, I187K, C332A, | − | +++ |
| | Y359A | | |
| Neu2-M131 | M1D, V6Y, I187K, C332A, | − | +++ |
| | Y359S | | |
| Neu2-M132 | M1D, V6Y, K44E, K45E, I187K, | ++ | ++ |
| | S301R, W302K, C332A | | |
| Neu2-M133 | M1D, V6Y, Q112R, I187K, | ++ | +++ |
| | S301R, W302K, C332A | | |
| Neu2-M134 | M1D, V6Y, I187K, Q270A, | ++ | +++ |
| | S301R, W302K, C332A | | |
| Neu2-M135 | M1D, V6Y, K44E, K45E, Q112R, | + | + |
| | I187K, C332A | | |
| Neu2-M136 | M1D, V6Y, K44E, K45E, I187K, | ++ | ++ |
| | Q270A, C332A | | |
| Neu2-M137 | M1D, V6Y, K45A, I187K, | ++ | +++ |
| | Q270A, C332A | | |
| Neu2-M138 | M1D, V6Y, I187K, Q270H, | ++ | +++ |
| | C332A | | |
| Neu2-M139 | M1D, V6Y, I187K, Q270P, | + | +++ |
| | C332A | | |
| Neu2-M140 | M1D, V6Y, Q112K, I187K, | ++ | ++ |
| | C332A | | |
| Neu2-M141 | M1D, V6Y, P62S, I187K, Q270A, | + | +++ |
| | S301R, W302K, C332A | | |
| Neu2-M142 | M1D, V6Y, P62T, I187K, Q270A, | + | +++ |
| | S301R, W302K, C332A | | |
| Neu2-M143 | M1D, V6Y, P62N, I187K, Q270A, | + | +++ |
| | S301R, W302K, C332A | | |
| Neu2-M144 | V6Y, P62H, I187K | + | +++ |
| Neu2-M145 | V6Y, Q108H, I187K | + | ++ |
| Neu2-M146 | M1D, V6Y, P62H, I187K, C332A | ++ | +++ |
| Neu2-M147 | M1D, V6Y, P62G, I187K, C332A | ++ | ++ |
| Neu2-M148 | V6Y, P62G, I187K | + | ++ |
| Neu2-M149 | M1D, V6Y, P62H, I187K | ++ | ++ |
| Neu2-M150 | M1D, V6Y, Q108H, I187K | ++ | ++ |
| Neu2-M151 | M1D, V6Y, P62F, I187K, C332A | + | − |
| Neu2-M152 | M1D, V6Y, P62I, I187K, C332A | + | − |
| Neu2-M153 | M1D, V6Y, P62N, I187K, C332A | ++ | +++ |
| Neu2-M154 | M1D, V6Y, P62D, I187K, C332A | ++ | + |
| Neu2-M155 | M1D, V6Y, P62E, I187K, C332A | ++ | +++ |
| Neu2-M156 | V6Y, C164G, I187K, T249A | + | + |
| Neu2-M157 | V6Y, C164G, I187K | + | + |
| Neu2-M158 | V6Y, Q126L, I187K D251G | + | ++ |
| Neu2-M159 | V6Y, L54M, Q69H, R78K, | + | + |
| | A171G, I187K | | |
| Neu2-M160 | V6Y, P62T, I187K | + | ++ |
| Neu2-M161 | V6Y, A150V, I187K | + | ++ |
| Neu2-M162 | P5H, V6Y, P62S, I187K | + | ++ |
| Neu2-M163 | V6Y, C164G, I187K | + | + |

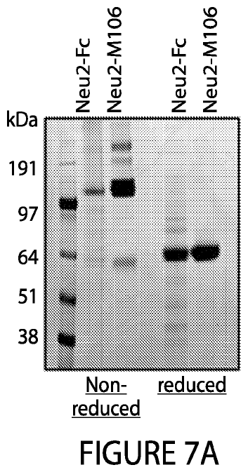

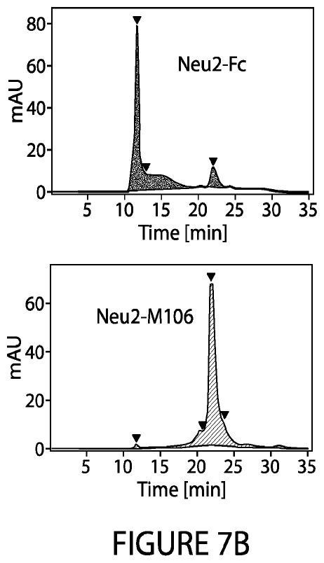

To confirm these results, Neu2-M106 (with amino acid sequence SEQ ID NO: 48, encoded by nucleotide sequence SEQ ID NO: 89) was expressed and purified with a protein A column. FIG. 7A is an image of an SDS-PAGE gel showing recombinant wildtype human Neu2 and Neu2 variant M106 (each with a C-terminal human Fc tag) under non-reducing and reducing conditions. FIG. 7B is an SEC-HPLC trace for recombinant wildtype human Neu2 and Neu2 variant M106 (each with a C-terminal human Fc tag). While Neu2-Fc had a yield of 0.3 mg/liter following protein-A purification, and monomer content of 7% as determined by SEC, Neu2-M106 had a yield of 20 mg/liter, and a monomer content of 85%.

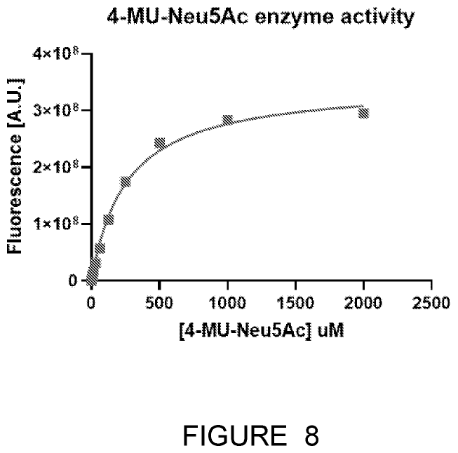

The enzyme kinetics of Neu2-M106 were assayed by measuring the release of sialic acid from the fluorogenic substrate 4-methylumbelliferyl-N-acetylneuraminic acid (4MU-NeuAc) as described above. A fixed concentration of enzyme at 2 μg/well was incubated with fluorogenic substrate 4MU-NeuAc at concentrations ranging from 4 mM to 0.03 μM. FIG. 8 depicts the enzyme activity of Neu2 variant M106. Enzymatic activity of Neu2-M106 was comparable to wildtype Neu2, with a K_{M} determined to be 230 μM.

Together, these results show that the mutations identified by rational design, phage display, and/or yeast display approaches described herein can increase stability and/or expression of a sialidase.

### Example 3

This Example describes the construction and expression of antibody-sialidase genetic fusion proteins, and antibody sialidase conjugates (ASCs) containing the fusion proteins, with mutated human sialidases.

The architecture for five types of exemplary ASCs is depicted in FIG. 11. The first type of ASC, referred to as “Raptor,” includes an antibody (with two heavy chains and two light chains) with a sialidase fused at the C-terminus of each heavy chain of the antibody (FIG. 11A). The second type of ASC, referred to as “Janus,” contains one antibody arm (with one heavy chain and one light chain), and one sialidase-Fc fusion with a sialidase fused at the N-terminus of one arm of the Fc. Each Fc domain polypeptide in the Janus ASC contains either the “knob” (T366Y) or “hole” (Y407T) mutation for heterodimerization (residue numbers according to EU numbering, Kabat, E. A., et al. (1991) supra) (FIG. 11B). The third type of ASC, referred to as “Lobster,” contains two Fc domain polypeptides each with a sialidase fused at the N-terminus of the Fc and a scFv fused at the C-terminus of the Fc (FIG. 11C). The fourth type of ASC, referred to as “Bunk,” contains one antibody arm (with one heavy chain and one light chain) with an scFv fused at the C-terminus of one arm of the Fc and one sialidase-Fc fusion with a sialidase fused at the N-terminus of the other arm of the Fc. Each Fc domain polypeptide in the Bunk ASC contains either the “knob” (T366Y) or “hole” (Y407T) mutation for heterodimerization (residue numbers according to EU numbering, Kabat, E. A., et al. (1991) supra) (FIG. 11D). The fifth type of ASC, referred to as “Lobster-Fab,” contains two Fc domain polypeptides each with (i) a sialidase fused at the N-terminus of the Fc and (ii) a Fab fused at the C-terminus of the Fc (FIG. 11E).

Janus ASCs including Neu2 variants described in Example 2 and trastuzumab were made and tested for activity and expression. Expression was assayed using a ForteBio Octet with anti-human Fc sensors and Western blot and enzymatic activity was assayed using the fluorogenic substrate 4MU-NeuAc as described above. Expression and activity levels for the Janus ASCs are shown in TABLE 26. In TABLE 26, enzymatic activity is indicated as “+++,” which denotes activity >2 fold higher than wild-type Neu2, “++,” which denotes activity comparable to wild-type Neu2, “+,” which denotes activity lower than wild-type Neu2, or “−,” which denotes no detectable activity, and expression is indicated as “++++,” which denotes expression >15 fold higher than wildtype-Neu2, “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 26

| Neu2 | | | |
|---|---|---|---|
| Variant | Mutation(s) | Activity | Expression |
| Neu2-M146 | M1D, V6Y, P62H, I187K, C332A | +++ | +++ |
| Neu2-M147 | M1D, V6Y, P62G, I187K, C332A | ++ | ++ |
| Neu2-M148 | V6Y, P62G, I187K | ++ | +++ |
| Neu2-M149 | M1D, V6Y, P62H, I187K | + | +++ |
| Neu2-M150 | M1D, V6Y, Q108H, I187K | ++ | ++ |
| Neu2-M151 | M1D, V6Y, P62F, I187K, C332A | ++ | +++ |
| Neu2-M152 | M1D, V6Y, P62I, I187K, C332A | ++ | +++ |
| Neu2-M153 | M1D, V6Y, P62N, I187K, C332A | ++ | +++ |
| Neu2-M154 | M1D, V6Y, P62D, I187K, C332A | ++ | ++ |
| Neu2-M155 | M1D, V6Y, P62E, I187K, C332A | ++ | + |
| Neu2-M156 | V6Y, C164G, I187K, T249A | + | + |
| Neu2-M157 | V6Y, C164G, I187K | + | + |
| Neu2-M158 | V6Y, Q126L, I187K D251G | ++ | ++ |
| Neu2-M159 | V6Y, L54M, Q69H, R78K, | + | + |
| | A171G, I187K | | |
| Neu2-M160 | V6Y, P62T, I187K | ++ | ++ |
| Neu2-M161 | V6Y, A150V, I187K | + | ++ |
| Neu2-M162 | P5H, V6Y, P62S, I187K | ++ | ++ |
| Neu2-M163 | V6Y, C164G, I187K | + | + |

Additional Janus ASCs including Neu2 variants described in Example 2 and trastuzumab were made and tested for activity and expression. Janus ASCs were expressed in Expi293F cells in 500 mL cultures and purified using protein A and ion exchange chromatography. Expression was assayed using a ForteBio Octet with anti-human Fc sensors and Western blot and enzymatic activity was assayed using the fluorogenic substrate 4MU-NeuAc as described above. Expression and activity levels for the Janus ASCs are shown in TABLE 27. In TABLE 27, enzymatic activity is indicated as “+++,” which denotes activity >2 fold higher than wild-type Neu2, “++,” which denotes activity comparable to wild-type Neu2, “+,” which denotes activity lower than wild-type Neu2, or “−,” which denotes no detectable activity, and expression is indicated as “++++,” which denotes expression >15 fold higher than wildtype-Neu2, “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 27

| Neu2 | | | |
|---|---|---|---|
| Variant | Mutation(s) | Activity | Expression |
| Neu2-M106 | M1D, V6Y, P62G, A93E, I187K, | ++ | ++++ |
| | C332A | | |
| Neu2-M134 | M1D, V6Y, I187K, Q270A, | +++ | ++++ |
| | S301R, W302K, C332A | | |
| Neu2-M141 | M1D, V6Y, P62S, I187K, Q270A, | ++ | ++++ |
| | S301R, W302K, C332A | | |
| Neu2-M142 | M1D, V6Y, P62T, I187K, Q270A, | ++ | ++++ |
| | S301R, W302K, C332A | | |
| Neu2-M143 | M1D, V6Y, P62N, I187K, Q270A, | ++ | ++++ |
| | S301R, W302K, C332A | | |
| Neu2-M153 | M1D, V6Y, P62N, I187K, C332A | ++ | ++++ |

### Example 4

This example describes the construction of recombinant human sialidases with mutations that increase expression and/or activity of the sialidase.

Unless indicated otherwise, mutant sialidases in this Example were expressed as secreted proteins with a C-terminal human Fc tag in Expi293F cells using the pCEP4 mammalian expression vector. Expression was assayed using a ForteBio Octet with anti-human Fc sensors and Western blot and enzymatic activity was assayed using the fluorogenic substrate 4MU-NeuAc as described above.

Mutant Neu2 sialidases were constructed including rationally designed substitutions at position Q126. Inspection of the Neu2 crystal structure revealed that mutation of Q126 may increase interactions with neighboring amino acid residues.

Additional mutant Neu2 sialidases were constructed including rationally designed substitution at position Q270. Inspection of the Neu2 crystal structure revealed that mutation of Q270 to certain amino acids may stabilize interactions with R237 and stabilize binding in the substrate pocket.

Additional mutant Neu2 sialidases were constructed including a substitution of an amino acid residue in a beta turn with a proline (for example D80P, R189P, and/or H239P substitutions). Substitution with a proline at these positions may, for example, stabilize the protein by influencing local protein folding.

Expression and activity levels for the resulting mutant sialidases are shown in TABLE 28. In TABLE 28, enzymatic activity is indicated as “++,” which denotes activity comparable to wild-type Neu2, “+,” which denotes activity lower than wild-type Neu2, or “−,” which denotes no detectable activity, and expression is indicated as “+++++”, which denotes expression >40 fold higher than wildtype-Neu2, “++++”, which denotes expression >15 fold higher than wildtype-Neu2, “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 28

| Identifier | Mutation(s) | Activity | Expression |
|---|---|---|---|
| Neu2-M164 | M1D, V6Y, P62G, A93E, | ++ | ++++ |
| | Q126E, I187K, C332A | | |
| Neu2-M165 | M1D, V6Y, P62G, A93E, | ++ | ++++ |
| | Q126I, I187K, C332A | | |
| Neu2-M166 | M1D, V6Y, P62G, A93E, | + | +++++ |
| | Q126L, I187K, C332A | | |
| Neu2-M167 | M1D, V6Y, P62G, A93E, | + | +++++ |
| | Q126Y, I187K, C332A | | |
| Neu2-M168 | M1D, V6Y, P62G, A93E, | + | +++++ |
| | Q126F, I187K, C332A | | |
| Neu2-M169 | M1D, V6Y, P62G, A93E, | ++ | ++++ |
| | Q126H, I187K, C332A | | |
| Neu2-M170 | M1D, V6Y, P62G, A93E, | ++ | ++++ |
| | I187K, Q270S, C332A | | |
| Neu2-M171 | M1D, V6Y, P62G, A93E, | ++ | ++++ |
| | I187K, Q270T, C332A | | |
| Neu2-M172 | M1D, V6Y, P62G, A93E, | ++ | +++++ |
| | Q126Y, I187K, Q270T, | | |
| | C332A | | |
| Neu2-M173 | M1D, V6Y, P62G, A93E, | ++ | +++++ |
| | Q126Y, I187K, A242F, | | |
| | Q270T, C332A | | |
| Neu2-M174 | M1D, V6Y, P62G, D80P, | ++ | ++++ |
| | A93E, I187K, C332A | | |
| Neu2-M175 | M1D, V6Y, P62G, A93E, | ++ | +++ |
| | R170P, I187K, C332A | | |
| Neu2-M176 | M1D, V6Y, P62G, A93E, | ++ | ++++ |
| | I187K, Q188P, C332A | | |
| Neu2-M177 | M1D, V6Y, P62G, A93E, | ++ | ++++ |
| | I187K, R189P, C332A | | |
| Neu2-M178 | M1D, V6Y, P62G, A93E, | + | ++++ |
| | I187K, E225P, C332A | | |
| Neu2-M179 | M1D, V6Y, P62G, A93E, | ++ | ++++ |
| | I187K, H239P, C332A | | |
| Neu2-M180 | M1D, V6Y, P62G, A93E, | ++ | +++ |
| | I187K, E257P, C332A | | |

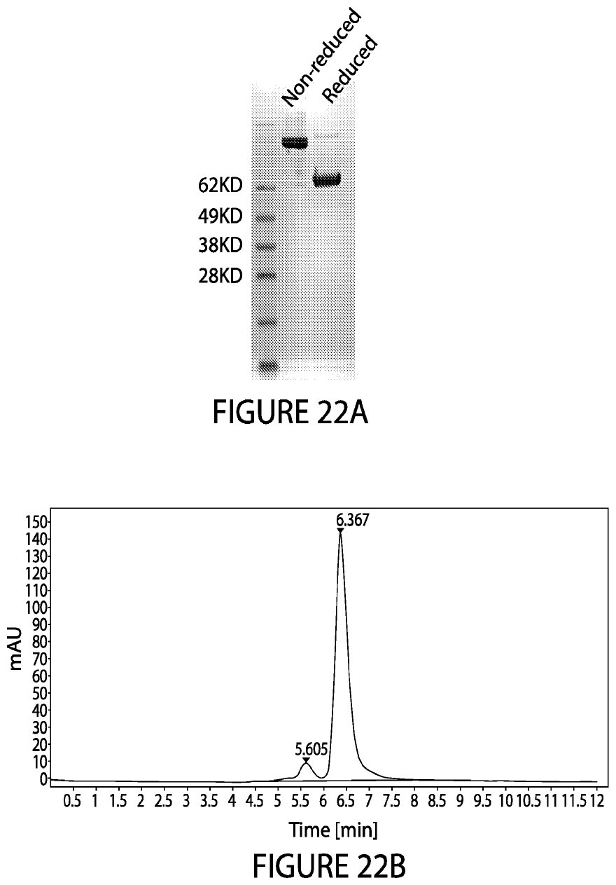

To confirm these results, Neu2-M173 (with amino acid sequence SEQ ID NO: 159, encoded by nucleotide sequence SEQ ID NO: 181) was expressed with a C-terminal human Fc tag and purified with a protein A and a ceramic hydroxyapatite (CHT) column. FIG. 22A is an image of an SDS-PAGE gel showing Neu2-M173-Fc (with a C-terminal human Fc tag) under non-reducing and reducing conditions. FIG. 22B is an SEC-HPLC trace for Neu2-M173-Fc (with a C-terminal human Fc tag). Neu2-M173-Fc had a yield of 120 mg/liter, and a monomer content of 90%.

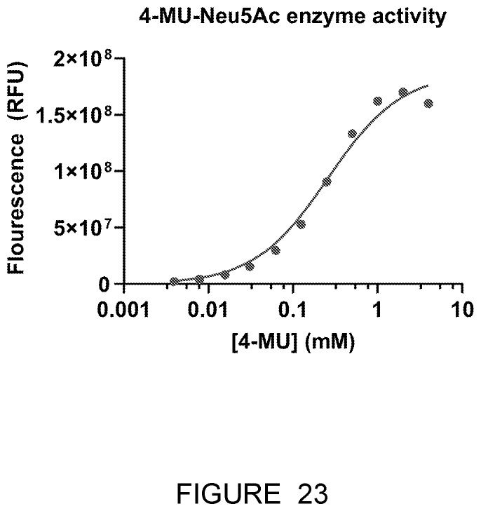

The enzyme kinetics of Neu2-M173-Fc were assayed by measuring the release of sialic acid from the fluorogenic substrate 4-methylumbelliferyl-N-acetylneuraminic acid (4MU-NeuAc) as described above. A fixed concentration of enzyme at 2 μg/well was incubated with fluorogenic substrate 4MU-NeuAc at concentrations ranging from 4 mM to 0.03 μM. FIG. 23 depicts the enzyme activity of Neu2-M173-Fc. Enzymatic activity of Neu2-M173-Fc was comparable to wildtype Neu2, with a K_{M} determined to be 230 μM.

Additional mutant Neu2 sialidases were constructed including rationally designed substitutions at positions S301 and/or W302. Mutations of S301 and/or W302 may influence interactions with neighboring amino acid residues and/or substrate.

Expression and activity levels for the mutant sialidases are shown in TABLE 29. In TABLE 29, enzymatic activity is indicated as “++,” which denotes activity comparable to wild-type Neu2, “+,” which denotes activity lower than wild-type Neu2, or “−,” which denotes no detectable activity, and expression is indicated as “+++++”, which denotes expression >40 fold higher than wildtype-Neu2, “++++”, which denotes expression >15 fold higher than wildtype-Neu2, “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression.

TABLE 29

| Identifier | Mutation(s) | Activity | Expression |
|---|---|---|---|
| Neu2-M182 | M1D, V6Y, P62G, A93E, I187K, | ++ | ++++ |
| | S301A, C332A | | |
| Neu2-M183 | M1D, V6Y, P62G, A93E, I187K, | ++ | ++++ |
| | S301D, C332A | | |
| Neu2-M184 | M1D, V6Y, P62G, A93E, I187K, | ++ | +++ |
| | S301E, C332A | | |
| Neu2-M185 | M1D, V6Y, P62G, A93E, I187K, | + | ++++ |
| | S301F, C332A | | |
| Neu2-M186 | M1D, V6Y, P62G, A93E, I187K, | + | ++++ |
| | S301H, C332A | | |
| Neu2-M187 | M1D, V6Y, P62G, A93E, I187K, | ++ | +++++ |
| | S301K, C332A | | |
| Neu2-M188 | M1D, V6Y, P62G, A93E, I187K, | ++ | ++++ |
| | S301L, C332A | | |
| Neu2-M189 | M1D, V6Y, P62G, A93E, I187K, | ++ | ++++ |
| | S301M, C332A | | |
| Neu2-M190 | M1D, V6Y, P62G, A93E, I187K, | ++ | ++++ |
| | S301N, C332A | | |
| Neu2-M191 | M1D, V6Y, P62G, A93E, I187K, | ++ | ++++ |
| | S301P, C332A | | |
| Neu2-M192 | M1D, V6Y, P62G, A93E, I187K, | ++ | +++ |
| | S301Q, C332A | | |
| Neu2-M193 | M1D, V6Y, P62G, A93E, I187K, | ++ | +++++ |
| | S301R, C332A | | |
| Neu2-M194 | M1D, V6Y, P62G, A93E, I187K, | ++ | ++++ |
| | S301T, C332A | | |
| Neu2-M195 | M1D, V6Y, P62G, A93E, I187K, | ++ | ++++ |
| | S301V, C332A | | |
| Neu2-M196 | M1D, V6Y, P62G, A93E, I187K, | + | ++++ |
| | S301W, C332A | | |
| Neu2-M197 | M1D, V6Y, P62G, A93E, I187K, | ++ | ++++ |
| | S301Y, C332A | | |
| Neu2-M198 | M1D, V6Y, P62G, A93E, I187K, | + | ++++ |
| | W302A, C332A | | |
| Neu2-M199 | M1D, V6Y, P62G, A93E, I187K, | +++ | +++ |
| | W302D, C332A | | |
| Neu2-M200 | M1D, V6Y, P62G, A93E, I187K, | + | ++++ |
| | W302F, C332A | | |
| Neu2-M201 | M1D, V6Y, P62G, A93E, I187K, | ++ | ++++ |
| | W302G, C332A | | |
| Neu2-M202 | M1D, V6Y, P62G, A93E, I187K, | + | ++++ |
| | W302H, C332A | | |
| Neu2-M203 | M1D, V6Y, P62G, A93E, I187K, | + | ++++ |
| | W302I, C332A | | |
| Neu2-M204 | M1D, V6Y, P62G, A93E, I187K, | ++ | ++++ |
| | W302L, C332A | | |
| Neu2-M205 | M1D, V6Y, P62G, A93E, I187K, | + | ++ |
| | W302M, C332A | | |
| Neu2-M206 | M1D, V6Y, P62G, A93E, I187K, | + | ++++ |
| | W302N, C332A | | |
| Neu2-M207 | M1D, V6Y, P62G, A93E, I187K, | − | ++++ |
| | W302P, C332A | | |
| Neu2-M208 | M1D, V6Y, P62G, A93E, I187K, | ++ | ++++ |
| | W302Q, C332A | | |
| Neu2-M209 | M1D, V6Y, P62G, A93E, I187K, | ++ | +++++ |
| | W302R, C332A | | |
| Neu2-M210 | M1D, V6Y, P62G, A93E, I187K, | ++ | ++++ |
| | W302S, C332A | | |
| Neu2-M211 | M1D, V6Y, P62G, A93E, I187K, | ++ | ++++ |
| | W302T, C332A | | |
| Neu2-M212 | M1D, V6Y, P62G, A93E, I187K, | + | ++++ |
| | W302V, C332A | | |
| Neu2-M213 | M1D, V6Y, P62G, A93E, I187K, | + | ++++ |
| | W302Y, C332A | | |
| Neu2-M214 | M1D, V6Y, P62G, A93E, I187K, | ++ | ++++ |
| | S301A, W302A, C332A | | |
| Neu2-M215 | M1D, V6Y, P62G, A93E, I187K, | + | ++++ |
| | S301A, W302R, C332A | | |
| Neu2-M216 | M1D, V6Y, P62G, A93E, I187K, | ++ | ++++ |
| | S301A, W302S, C332A | | |
| Neu2-M217 | M1D, V6Y, P62G, A93E, I187K, | ++ | ++++ |
| | S301A, W302T, C332A | | |
| Neu2-M218 | M1D, V6Y, P62G, A93E, I187K, | + | ++++ |
| | S301K, W302S, C332A | | |
| Neu2-M219 | M1D, V6Y, P62G, A93E, I187K, | ++ | +++++ |
| | S301K, W302R, C332A | | |
| Neu2-M219 | M1D, V6Y, P62G, A93E, I187K, | ++ | ++++ |
| | S301K, W302T, C332A | | |
| Neu2-M220 | M1D, V6Y, P62G, A93E, I187K, | + | ++++ |
| | S301N, W302S, C332A | | |
| Neu2-M221 | M1D, V6Y, P62G, A93E, I187K, | ++ | ++++ |
| | S301N, W302T, C332A | | |
| Neu2-M222 | M1D, V6Y, P62G, A93E, I187K, | + | ++++ |
| | S301T, W302R, C332A | | |

### Example 5

This example describes the construction of recombinant human sialidases with mutations that reduce proteolytic cleavage.

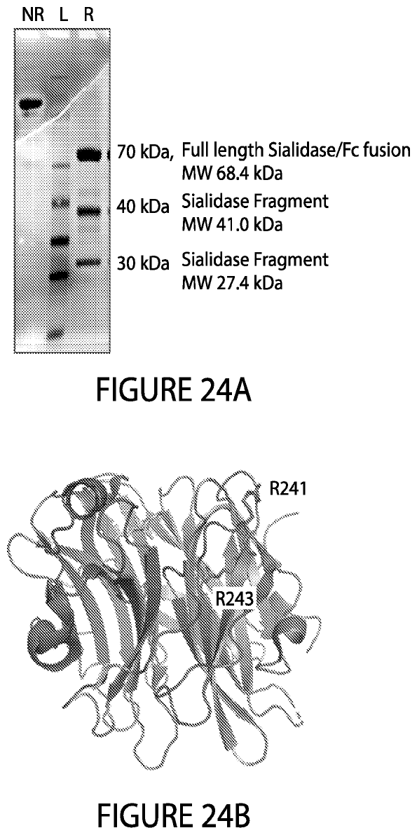

Neu2-M106 (as described in Example 2, and with amino acid sequence SEQ ID NO: 48) was expressed as an Fc-fused single chain protein using a CHO cell expression system in a large scale (10 L) high cell density production and purified with a protein A column. The resulting protein was analyzed by SDS-PAGE. Results are shown in FIG. 24. Under reducing conditions, the protein included a mixture of full length (70 kDa, approx. 50%) and cleaved (40 kDa and 30 kDa, approx. 50%) fractions. However, in non-reducing conditions, there was no cleavage and the protein remained as a single chain (FIG. 24). Additionally, when Neu2-M106 was expressed on a smaller scale (with a shorter duration of cell culture and lower cell density) there was no cleavage and the protein remained as a single chain. A preliminary mass spectrometry analysis showed that the 40 kDa and 30 kDa molecular weight fractions observed under reducing conditions following large scale production are a result of cleavage between amino acid residues R243 and V244 of the sialidase. The enzymatic activity of cleaved Neu2-M106 was similar to that of uncleaved Neu2-M106.

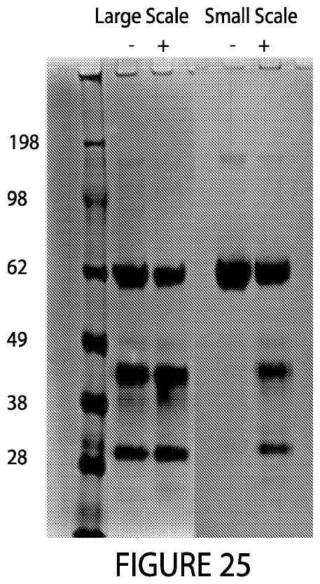

It was hypothesized that the cleavage of Neu2-M106 could be due to the activity of intracellular proteases released as a result of cell lysis during protein production, harvesting, and/or purification. To test this hypothesis, cleaved Neu2-M106 (prepared using the large scale-production described above that results in cleavage) and uncleaved Neu2-M106 (prepared using the smaller scale production described above that does not result in cleavage) were both incubated with trypsin and analyzed by SDS-PAGE under reducing conditions (FIG. 25). Briefly, trypsin digestion reactions were performed by incubation of trypsin (5 μL, 0.005% solution in PBS) with Neu2-M106 (25 μL, 0.25 mg/mL in PBS pH 8.0) for 5 minutes on ice. Reactions were stopped by addition of LDS gel loading buffer (5 μL) and run on a reducing SDS-PAGE gel to observe trypsin mediated cleavage. The SDS-PAGE analysis showed that incubation of the uncleaved Neu2-M106 with trypsin resulted in the same cleavage pattern as that of the cleaved Neu2-M106. Additionally, incubation of the cleaved Neu2-M106 with trypsin resulted in increased intensity of the bands corresponding to the cleavage products.

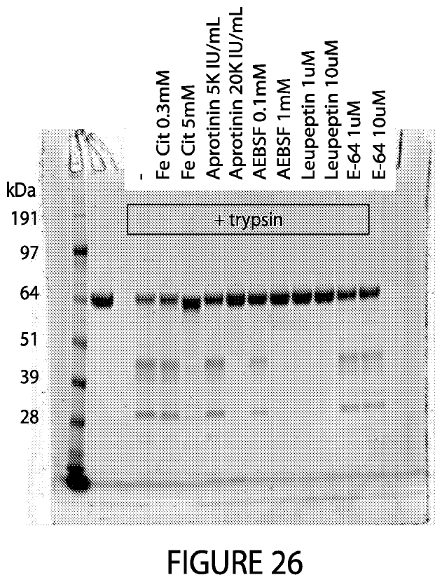

Neu2-M106 was also incubated with trypsin in the presence of various protease inhibitors. Briefly, trypsin digestion reactions were performed by incubation of trypsin (0.005%) with Neu2-M106 (0.5 mg/mL) and protease inhibitor for 5 minutes on ice. Reactions were stopped by addition of LDS gel loading buffer and run on a reducing SDS-PAGE gel to observe trypsin mediated cleavage. Inhibitors used included iron citrate (at 0.3 and 5 mM), aprotinin (at 5,000 and 20,000 U/mL), AEBSF (at 0.1 and 1 mM), leupeptin (at 1 and 10 μM) or E-64 (at 1 and 10 μM). As seen in FIG. 26, protease inhibitors reduced the extent of trypsin cleavage.

Together, these results confirm that cleavage of Neu2-M106 following large-scale production is due to trypsin or a member of a similar class of proteases.

Next, recombinant human sialidases with mutations that increase resistance to trypsin cleavage were rationally designed.

Unless indicated otherwise, in the remainder of this Example mutant sialidases were expressed as secreted proteins with a C-terminal human Fc tag in Expi293F cells (on a 50 mL scale) using the pCEP4 mammalian expression vector. The resulting protein was purified using a Protein A column. Expression was assayed using a ForteBio Octet with anti-human Fc sensors and Western blot and enzymatic activity was assayed using the fluorogenic substrate 4MU-NeuAc as described above. Protease cleavage was assayed by SDS-PAGE as described above.

First, R243 was mutated to different polar/charged amino acids such as K, E, H, N and Q. However, these mutations of R243 resulted in complete loss of activity and reduction in expression yields. As shown in FIG. 27A, which provides a sequence alignment of various human and non-human sialidases, R243 is conserved among sialidases. Next, various amino residues surrounding the cleavage site were mutated and tested for expression, activity and trypsin cleavage resistance. Substitutions and combinations of substitutions that were tested are shown in FIG. 27B. All mutations were tested in a Neu2-M106 background (i.e., including M1D, V6Y, P62G, A93E, I187K, and C332A substitutions).

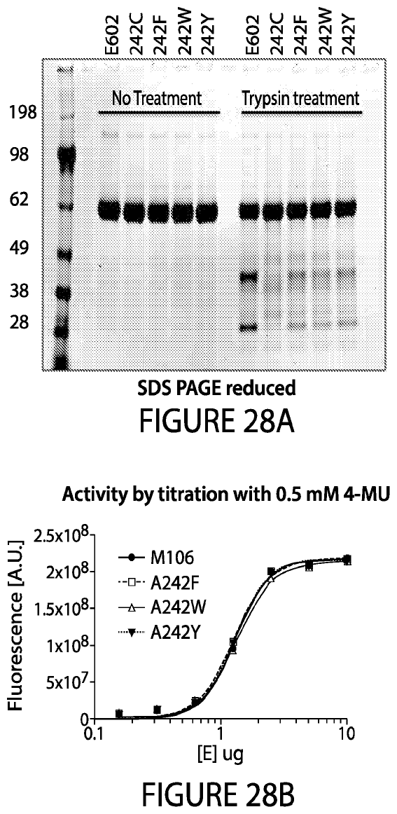

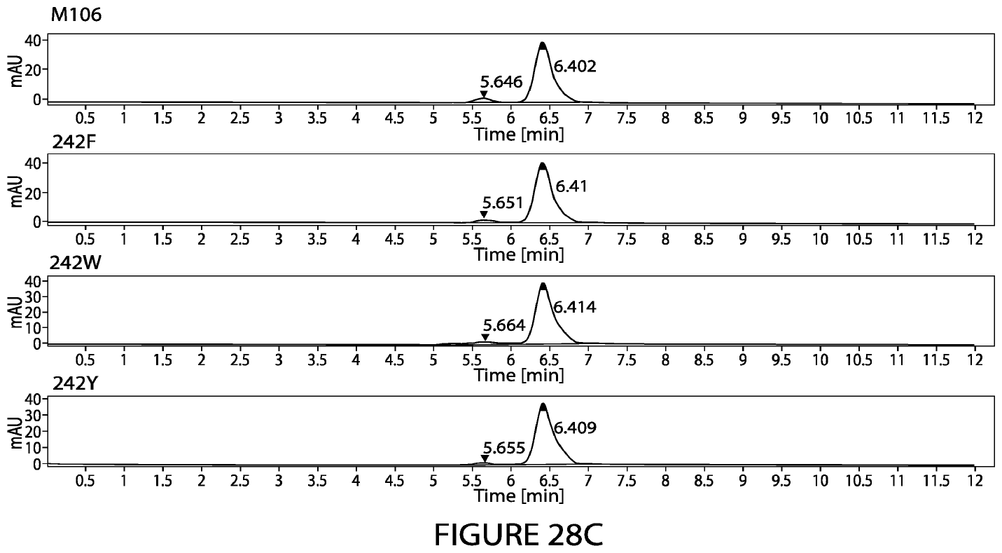

Most of the mutant sialidases depicted in FIG. 27B expressed well, however only two of the mutant sialidases (including V244I or A242C mutations) were active. The A242C mutation resulted in greater than 10 fold improved trypsin resistance and slightly lower activity (both relative to Neu2-M106). However, having an unpaired cysteine could be a potential liability, so, A242 was mutated to all 19 other amino acids and assayed for activity and trypsin resistance. As shown in FIG. 28, mutation of A242 to aromatic amino acids such as F, W and Y resulted in a dramatic improvement in trypsin cleavage resistance compared to Neu2-M106 (FIG. 28A) and similar enzymatic activity to Neu2-M106 (FIG. 28B). SEC analysis showed that proteins containing each of these mutations had a similar pattern to that of Neu2-M106 and more than 95% monomer content (FIG. 28C).

Structural analysis showed that replacing A242 with an aromatic amino acid could provide additional hydrophobic or stacking interactions to L260 and V265 (nonpolar amino acids located in the vicinity of the A242). Therefore, L260 and V265 were also mutated to phenylalanine. Along with these mutations several other rationally designed mutants, which could provide extra stability, for example by increasing stacking interactions, were also tested for expression, activity, and protease resistance.

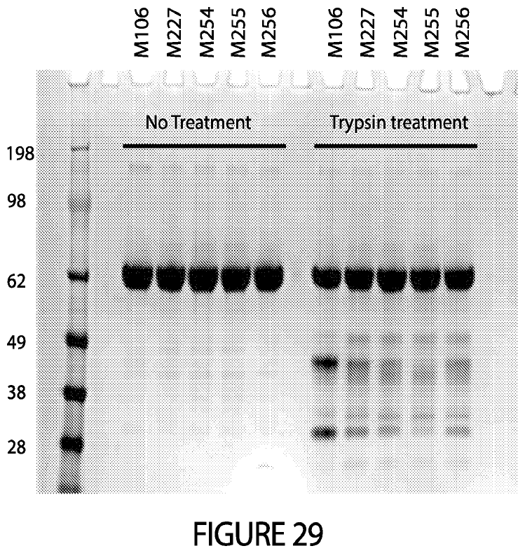

Select results are shown in FIG. 29. As shown in FIG. 29, the combination of R241Y and A242F mutations (Neu2-M255) resulted in the most resistance to trypsin cleavage (a greater than 10 fold improved trypsin resistance relative to Neu2-M106).

Expression, activity, and protease resistance levels for the mutant sialidases are shown in TABLE 30. In TABLE 30, enzymatic activity is indicated as “++,” which denotes activity comparable to wild-type Neu2, “+,” which denotes activity lower than wild-type Neu2, or “−,” which denotes no detectable activity, and expression is indicated as “+++++”, which denotes expression >40 fold higher than wildtype-Neu2, “++++”, which denotes expression >15 fold higher than wildtype-Neu2, “+++,” which denotes expression >6 fold higher than wild-type Neu2, “++,” which denotes expression 2-5 fold higher than wild-type Neu2, “+,” which denotes expression comparable to wild-type Neu2, or “−,” which denotes no detectable expression. Protease/trypsin resistance is indicated as “+++,” which denotes resistance >10 fold higher than Neu2-M106; “++,” which denotes resistance ≥5 fold higher than Neu2-M106, “+,” which denotes resistance comparable to Neu2-M106, or “−,” which denotes resistance lower than Neu2-M106. NT=not tested.

TABLE 30

| | | | | Protease |
|---|---|---|---|---|
| Identifier | Mutation(s) | Activity | Expression | Resistance |
| Neu2-M223 | M1D, V6Y, P62G, | ++ | +++++ | + |
| | A93E, I187K, | | | |
| | L240Y, C332A | | | |
| Neu2-M224 | M1D, V6Y, P62G, | + | +++ | +++ |
| | A93E, I187K, | | | |
| | A242C, C332A | | | |
| Neu2-M225 | M1D, V6Y, P62G, | − | ++++ | − |
| | A93E, I187K, | | | |
| | A242D, C332A | | | |
| Neu2-M226 | M1D, V6Y, P62G, | − | +++ | − |
| | A93E, I187K, | | | |
| | A242E, C332A | | | |
| Neu2-M227 | M1D, V6Y, P62G, | ++ | ++++ | ++ |
| | A93E, I187K, | | | |
| | A242F, C332A | | | |
| Neu2-M228 | M1D, V6Y, P62G, | + | +++ | + |
| | A93E, I187K, | | | |
| | A242G, C332A | | | |
| Neu2-M229 | M1D, V6Y, P62G, | + | +++ | + |
| | A93E, I187K, | | | |
| | A242H, C332A | | | |
| Neu2-M230 | M1D, V6Y, P62G, | + | +++ | + |
| | A93E, I187K, | | | |
| | A242I, C332A | | | |
| Neu2-M231 | M1D, V6Y, P62G, | + | ++++ | − |
| | A93E, I187K, | | | |
| | A242K, C332A | | | |
| Neu2-M232 | M1D, V6Y, P62G, | + | +++ | + |
| | A93E, I187K, | | | |
| | A242L, C332A | | | |
| Neu2-M233 | M1D, V6Y, P62G, | + | +++ | + |
| | A93E, I187K, | | | |
| | A242M, C332A | | | |
| Neu2-M234 | M1D, V6Y, P62G, | + | ++++ | ++ |
| | A93E, I187K, | | | |
| | A242N, C332A | | | |
| Neu2-M235 | M1D, V6Y, P62G, | + | +++ | ++ |
| | A93E, I187K, | | | |
| | A242Q, C332A | | | |
| Neu2-M236 | M1D, V6Y, P62G, | + | ++++ | − |
| | A93E, I187K, | | | |
| | A242R, C332A | | | |
| Neu2-M237 | M1D, V6Y, P62G, | + | +++ | − |
| | A93E, I187K, | | | |
| | A242S, C332A | | | |
| Neu2-M238 | M1D, V6Y, P62G, | − | +++ | + |
| | A93E, I187K, | | | |
| | A242T, C332A | | | |
| Neu2-M239 | M1D, V6Y, P62G, | + | +++ | + |
| | A93E, I187K, | | | |
| | A242V, C332A | | | |
| Neu2-M240 | M1D, V6Y, P62G, | ++ | ++++ | ++ |
| | A93E, I187K, | | | |
| | A242W, C332A | | | |
| Neu2-M241 | M1D, V6Y, P62G, | ++ | ++++ | ++ |
| | A93E, I187K, | | | |
| | A242Y, C332A | | | |
| Neu2-M242 | M1D, V6Y, P62G, | ++ | ++++ | ++ |
| | A93E, I187K, | | | |
| | A242F, L260F, | | | |
| | C332A | | | |
| Neu2-M243 | M1D, V6Y, P62G, | + | ++++ | ++ |
| | A93E, I187K, | | | |
| | A242F, V265F, | | | |
| | C332A | | | |
| Neu2-M244 | M1D, V6Y, P62G, | + | ++++ | ++ |
| | A93E, I187K, | | | |
| | A242F, A213C, | | | |
| | C332A | | | |
| Neu2-M245 | M1D, V6Y, P62G, | + | ++++ | ++ |
| | A93E, I187K, | | | |
| | A242F, A213S, | | | |
| | C332A | | | |
| Neu2-M246 | M1D, V6Y, P62G, | ++ | ++++ | ++ |
| | A93E, I187K, | | | |
| | A242F, A213T, | | | |
| | C332A | | | |
| Neu2-M247 | M1D, V6Y, P62G, | + | ++ | NT |
| | A93E, I187K, | | | |
| | A242F, A213N, | | | |
| | C332A | | | |
| Neu2-M248 | M1D, V6Y, P62G, | ++ | ++++ | ++ |
| | A93E, I187K, | | | |
| | A242F, A213C, | | | |
| | S258C, C332A | | | |
| Neu2-M249 | M1D, V6Y, P62G, | ++ | +++++ | + |
| | A93E, I187K, | | | |
| | L240Y, L260F, | | | |
| | C332A | | | |
| Neu2-M250 | M1D, V6Y, P62G, | + | + | NT |
| | A93E, I187K, | | | |
| | L240D, L260T, | | | |
| | C332A | | | |
| Neu2-M251 | M1D, V6Y, P62G, | + | ++ | NT |
| | A93E, I187K, | | | |
| | L240N, L260D, | | | |
| | C332A | | | |
| Neu2-M252 | M1D, V6Y, P62G, | ++ | ++++ | + |
| | A93E, I187K, | | | |
| | L240N, L260T, | | | |
| | C332A | | | |
| Neu2-M253 | M1D, V6Y, P62G, | ++ | ++++ | + |
| | A93E, I187K, | | | |
| | L240N, L260Q, | | | |
| | C332A | | | |
| Neu2-M254 | M1D, V6Y, P62G, | ++ | +++ | ++ |
| | A93E, I187K, | | | |
| | R241A, A242F, | | | |
| | C332A | | | |
| Neu2-M255 | M1D, V6Y, P62G, | ++ | +++ | +++ |
| | A93E, I187K, | | | |
| | R241Y, A242F, | | | |
| | C332A | | | |
| Neu2-M256 | M1D, V6Y, P62G, | ++ | +++++ | ++ |
| | A93E, Q126Y, | | | |
| | I187K, L240Y, | | | |
| | A242F, Q270T, | | | |
| | C332A | | | |
| Neu2-M257 | M1D, V6Y, P62G, | ++ | ++++ | + |
| | A93E, I187K, | | | |
| | V244I, C332A | | | |
| Neu2-M258 | M1D, V6Y, P62G, | + | +++ | ++ |
| | A93E, I187K, | | | |
| | A242C, V244K, | | | |
| | C332A | | | |

### Example 6

This Example describes the construction and expression of antibody-sialidase genetic fusion proteins, and antibody sialidase conjugates (ASCs) containing the fusion proteins, with mutated human sialidases.

A Janus Antibody Sialidase Conjugate (ASC) was made using Neu2 with M1D, V6Y, P62G, A93E, I187K and C332A substitutions and Trastuzumab, referred to in this Example as “Janus Trastuzumab”. This Janus Trastuzumab (including a first polypeptide chain with amino acid sequence SEQ ID NO: 66, encoded by nucleotide sequence SEQ ID NO: 86, a second polypeptide chain with amino acid sequence SEQ ID NO: 67, encoded by nucleotide sequence SEQ ID NO: 87, and a third polypeptide chain with amino acid sequence SEQ ID NO: 68, encoded by nucleotide sequence SEQ ID NO: 88) was expressed and characterized for purity using SDS-PAGE and enzymatic activity using 4MU-NeuAc as described below.

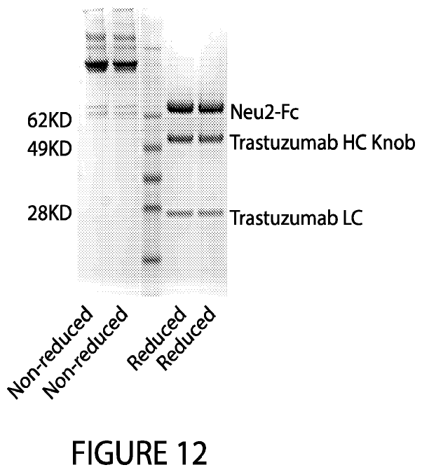

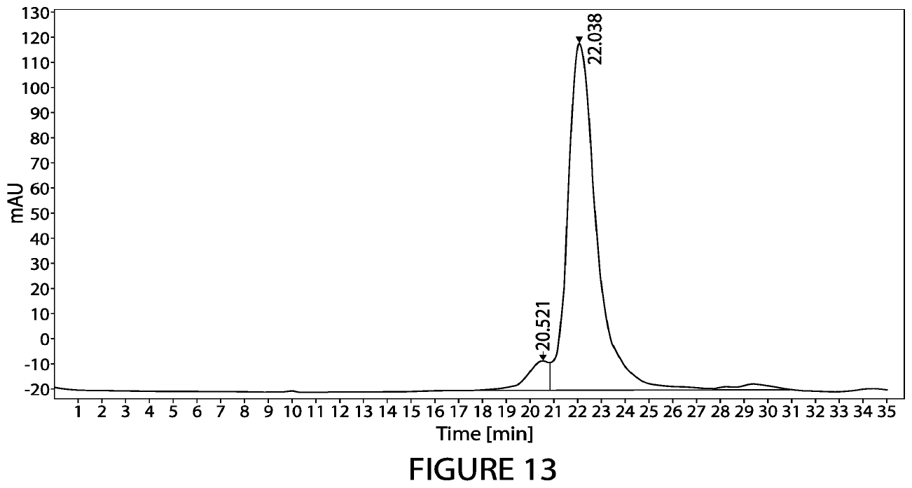

Janus Trastuzumab was expressed in a 1 L transfection of Expi293 human cells using the pCEP4 mammalian expression vector. Janus Trastuzumab was purified using Protein A, followed by cation exchange chromatography (Hitrap SP-HP, GE Lifesciences). Purified proteins were analyzed by SDS-PAGE (FIG. 12), and SEC-HPLC (FIG. 13). Expression yield was 30 mg/L, with 90% monomer purity as determined by SEC-HPLC.

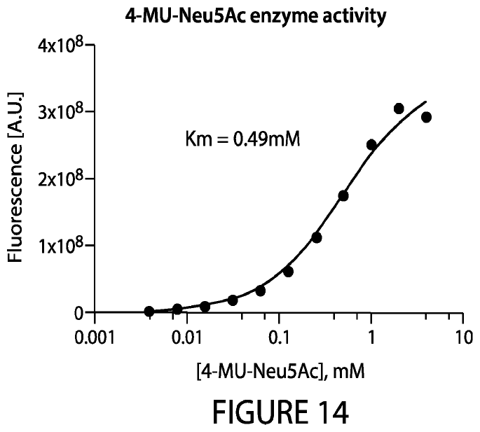

The enzymatic activity of the recombinantly expressed Janus Trastuzumab was assayed by measuring the release of sialic acid from the fluorogenic substrate 4-methylumbelliferyl-N-acetylneuraminic acid (4MU-NeuAc). Specifically, an enzyme kinetics assay was performed using a fixed concentration of enzyme at 2 μg/well, which was incubated with fluorogenic substrate 4MU-NeuAc at concentrations ranging from 4000 μM to 7.8 μM. As shown in FIG. 14, Janus Trastuzumab was enzymatically active, with a Km of 0.48 mM.

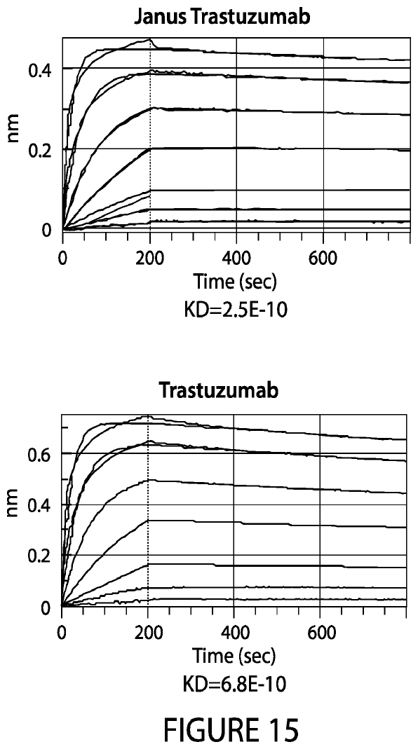

Janus Trastuzumab was tested for antigen (HER2) binding by using ForteBio Octet with the ASC captured on anti-Fc sensors with dipping into serial dilutions of His-tagged HER2 (50 to 0.78 nM at 1:2 dilutions). Janus Trastuzumab bound to HER2 with comparable binding affinity to trastuzumab (FIG. 15).

### Example 7

This Example describes the in vivo administration of antibody sialidase conjugates (ASCs) containing bacterial sialidases.

The following ASCs were made and tested in this Example: (i) a Janus ASC including Salmonella typhimurium sialidase (St-sialidase) and trastuzumab (including a first polypeptide chain with amino acid sequence SEQ ID NO: 66, encoded by nucleotide sequence SEQ ID NO: 86, a second polypeptide chain with amino acid sequence SEQ ID NO: 67, encoded by nucleotide sequence SEQ ID NO: 87, and a third polypeptide chain with amino acid sequence SEQ ID NO: 90, encoded by nucleotide sequence SEQ ID NO: 91); (ii) a Raptor ASC including St-sialidase and trastuzumab (including first and fourth polypeptide chains with amino acid sequence SEQ ID NO: 66, encoded by nucleotide sequence SEQ ID NO: 86, and second and third polypeptide chains with amino acid sequence SEQ ID NO: 92, encoded by nucleotide sequence SEQ ID NO: 93); and (iii) a Lobster ASC including St-sialidase and an scFv derived from trastuzumab (including first and second polypeptide chains with amino acid sequence SEQ ID NO: 94, encoded by nucleotide sequence SEQ ID NO: 95).

These ASCs were compared to trastuzumab in a mouse syngeneic tumor model injected with a murine breast cancer cell line expressing human HER2 (EMT6-hHER2 cells). Female BALB/c mice, 6-8 weeks of age, were inoculated subcutaneously in the right lower flank region with EMT6-HER2 tumor cells (5×105) in 0.1 ml of PBS for tumor development. Mice were randomly allocated to 8 groups when tumors reached 50-100 mm³, mean ˜75-100 mm³. Treatment groups are described in TABLE 31 with dosing schedule indicated post randomization. Anti-mouse NK1.1 (Clone: PK136; BioXcell, 621717N1), anti-mouse CD8α (Clone: 53-6.7; BioXcell, BE0004-1) and liposomal clodronate (FormuMax Scientific, Inc.) were included in treatment groups as indicated.

TABLE 31

| | Animal | | Dose | | Dose | | | Schedule |
|---|---|---|---|---|---|---|---|---|
| Group | No. | Treatment | (mg/kg) | | volume (μL/g) | | Route | (Days) |
| 1 | 8 | Vehicle (PBS) | NA | | 10 | | i.p. | 0, 3, 7, 10, 14, 17 |
| 2 | 8 | Trastuzumab | 10 | | 10 | | i.p | 0, 3, 7, 10, 14, 17 |
| 3 | 8 | Raptor | 10 | | 10 | | i.p | 0, 3, 7, 10, 14, 17 |
| 4 | 8 | Janus | 10 | | 10 | | i.p. | 0, 3, 7, 10, 14, 17 |
| 5 | 8 | Lobster | 10 | | 10 | | i.p. | 0, 3, 7, 10, 14, 17 |
| 6 | 8 | Janus | 10 | | 10 | | i.p. | 0, 3, 7, 10, 14, 17 |
| | | anti-mouse NK1.1 | 10 | | 10 | | i.p. | 0, 3, 7, 10, 14, 17 |
| | | (Clone: PK136) | | | | | | |
| 7 | 8 | Janus | 10 | | 10 | | i.p. | 0, 3, 7, 10, 14, 17 |
| | | anti-mouse CD8α | 10 | | 10 | | i.p. | 0, 3, 7, 10, 14, 17 |
| | | (Clone: 53-6.7) | | | | | | |
| 8 | 8 | Janus | 10 | | 10 | | i.p. | 0, 3, 7, 10, 14, 17 |
| | | liposomal | 0.5 | mg/mouse | 100 | μL/mouse | i.p. | TIW × 2 wks |
| | | clodronate | | | | | | |

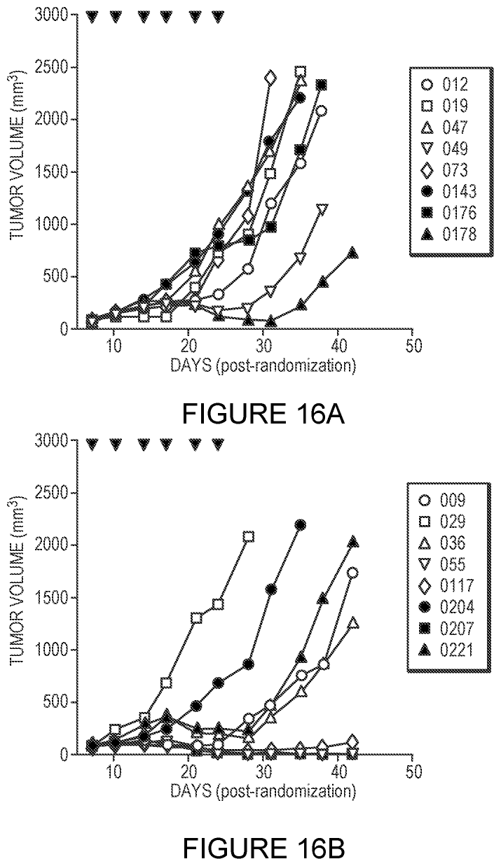

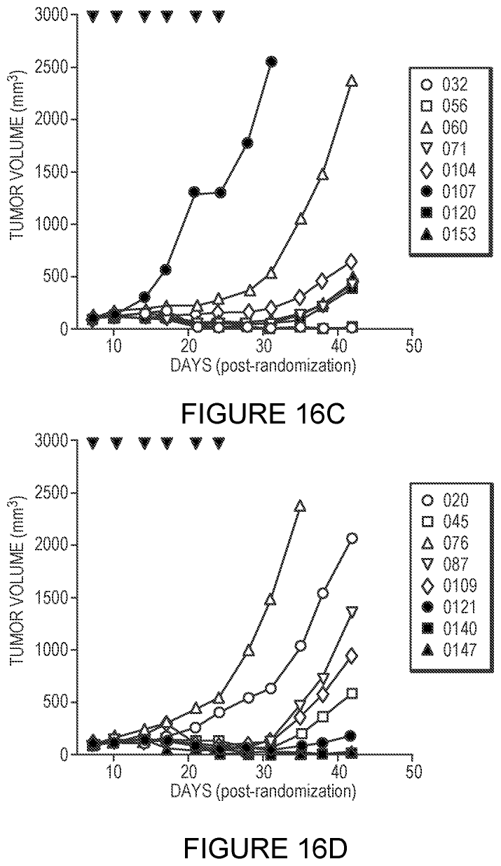

The results from for treatment with trastuzumab, and Raptor, Janus and Lobster ASCs are shown in FIGS. 16A, 16B, 16C and 16D respectively. As can be seen, trastuzumab resulted in no complete responses in eight individual mice as treated (defined as regression below the limit of palpation at any point for the duration of the study, FIG. 16A). This is in contrast to Raptor, which demonstrated 2 out of 8 animals with a complete response (FIG. 16B), Janus which demonstrated 3 out of 8 animals with a complete response (FIG. 16C) and Lobster which demonstrated 2 out of 8 animals with a complete response (FIG. 16D).

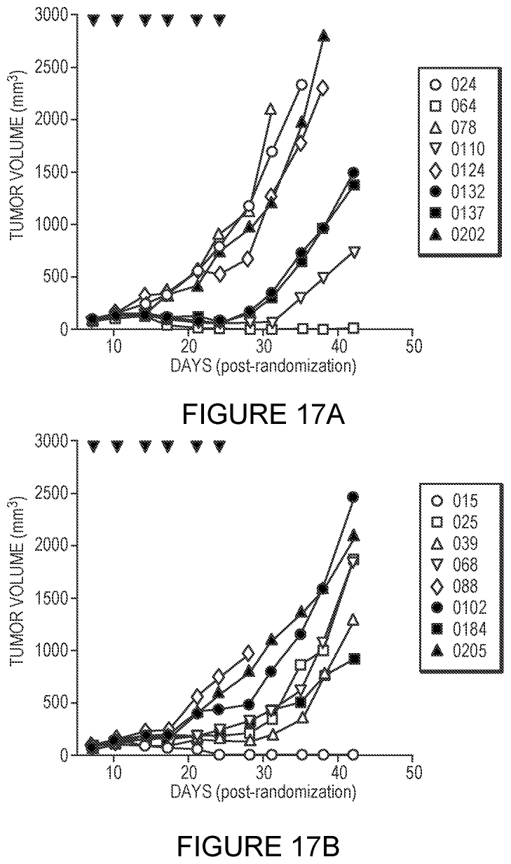

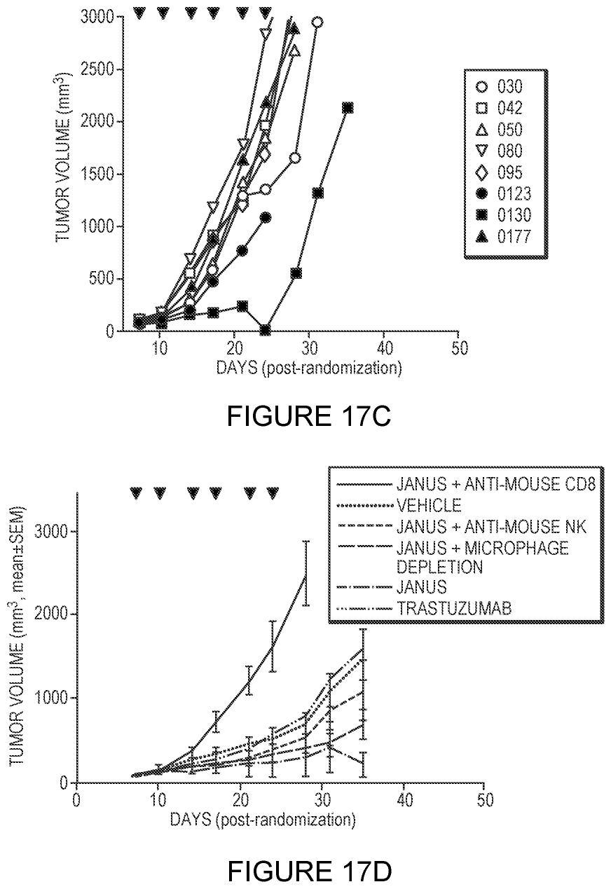

The results of administration of Janus with NK depletion (anti-mouse NK1.1), macrophage depletion (liposomal clodronate) and CD8 T cell depletion (anti-mouse CD8a) are shown in FIG. 17. As can be seen, compared to Janus treatment alone (FIG. 16C), where there was a complete response in 3 out of 8 animals, NK depletion reduced the number of complete responses to 1 out of 8 animals (FIG. 17A). Macrophage depletion also reduced the number of complete responses to 1 out of 8 animals (FIG. 17B). CD8 T cell depletion completely reversed the effects of Janus, with no animals showing a complete response (FIG. 17C). FIG. 17D shows the mean tumor volume for vehicle, Janus alone, trastuzumab alone and Janus with NK, macrophage and CD8 T cell depletions. These results demonstrate that innate immunity (NK and macrophage dependent) as well as adaptive immunity (CD8 T cells) contribute to in vivo ASC activity.

### Example 8

This Example describes the in vivo administration of antibody sialidase conjugates (ASCs) with bacterial sialidases.

The following ASCs were made and tested in this Example: (i) a Janus ASC including Salmonella typhimurium sialidase (St-sialidase) and trastuzumab (including a first polypeptide chain with amino acid sequence SEQ ID NO: 66, encoded by nucleotide sequence SEQ ID NO: 86, a second polypeptide chain with amino acid sequence SEQ ID NO: 67, encoded by nucleotide sequence SEQ ID NO: 87, and a third polypeptide chain with amino acid sequence SEQ ID NO: 90, encoded by nucleotide sequence SEQ ID NO: 91); and (ii) a Janus ASC including St-sialidase with two loss of function mutations, D100V and G231V, and trastuzumab (“Janus-LOF,” including a first polypeptide chain with amino acid sequence SEQ ID NO: 66, encoded by nucleotide sequence SEQ ID NO: 86, a second polypeptide chain with amino acid sequence SEQ ID NO: 67, encoded by nucleotide sequence SEQ ID NO: 87, and a third polypeptide chain with amino acid sequence SEQ ID NO: 96, encoded by nucleotide sequence SEQ ID NO: 97).

These ASCs were tested in a mouse syngeneic orthotopic tumor model injected with an independent EMT6 cell line expressing human HER2 (EMT6-hHER2 cells as described in D'Amico et al. (2016) ANNALS OF ONCOLOGY, Volume 27, Issue suppl_8, 41P. Female BALB/c mice, 6-8 weeks of age, were inoculated via intra mammary implantation with EMT6-HER2 tumor cells (5×10⁶). Mice were randomly allocated to 6 groups when tumors reached approximately 250 mm³. The treatment groups are described in TABLE 32 with dosing schedule indicated post randomization. Anti-mouse PD1 was obtained from BioXcell (RMP1-14, Cat. #665418F1).

TABLE 32

| | Animal | | Dose | Dose volume | | Schedule |
|---|---|---|---|---|---|---|
| Group | No. | Treatment | (mg/kg) | (μL/g) | Route | (Days) |
| 1 | 6 | Vehicle (PBS) | NA | 10 | i.p. | 0, 3, 7, 10 |
| 2 | 6 | Trastuzumab | 10 | 10 | i.p. | 0, 3, 7, 10 |
| 3 | 6 | Janus | 10 | 10 | i.p. | 0, 3, 7, 10 |
| 4 | 6 | Janus Loss of | 10 | 10 | i.p. | 0, 3, 7, 10 |
| | | Function (LOF) | | | | |
| 5 | 6 | anti-mouse PD1 | 10 | 10 | i.p. | 0, 3, 7, 10 |
| 6 | 6 | Janus | 10 | 10 | i.p. | 0, 3, 7, 10 |
| | | anti-mouse PD1 | 10 | 10 | i.p. | 0, 3, 7, 10 |

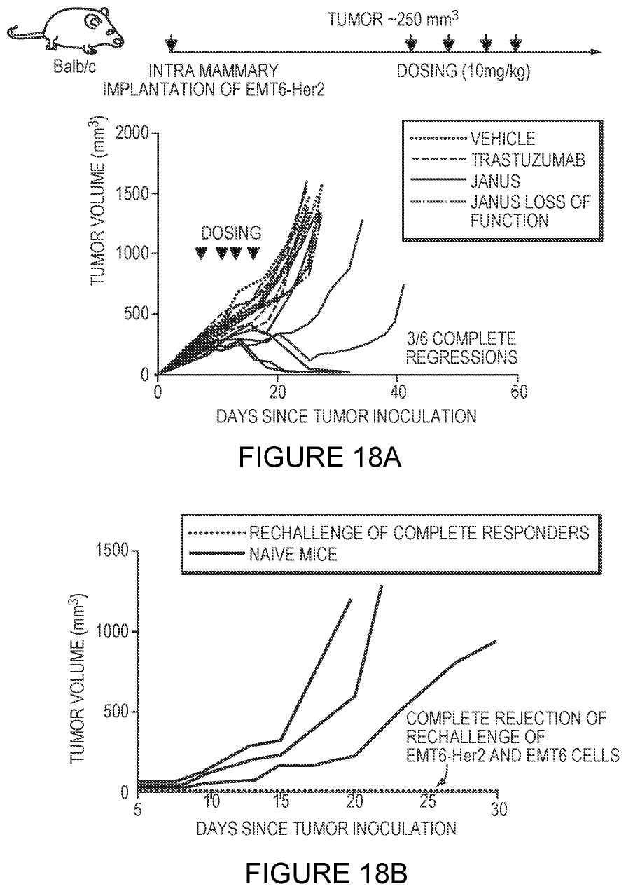

The results for Groups 1 through 4 (vehicle, trastuzumab, Janus and Janus LOF) are shown in FIG. 18A. As can be seen, 3 out of 6 animals treated with Janus had a complete regression of tumor growth. Notably, Janus LOF and trastuzumab were both comparable to vehicle treated animals.

The 3 mice with a complete regression (“cured mice”) were rechallenged with either the same EMT6-HER2 cells used originally or parental EMT6 cells (lacking engineered human HER2 expression). EMT6 cells and EMT6-HER2 cells were inoculated subcutaneously in the right or left lower flank region respectively (5×10⁵) in 0.1 ml of PBS for tumor development of all three cured mice. EMT6-HER2 cells were also inoculated subcutaneously into naïve mice as a control. As can be seen in FIG. 18B, neither EMT6-HER2 cells nor parental EMT6 cells resulted in tumor growth in the cured mice while EMT6-HER2 cells developed into tumors as expected in the naïve mice. These results suggest that the antibody sialidase conjugates of the present invention are capable of inducing long term memory against tumors. In addition, the long term memory is towards the tumor cell and is independent of the originally targeted cancer antigen (HER2 in this case).

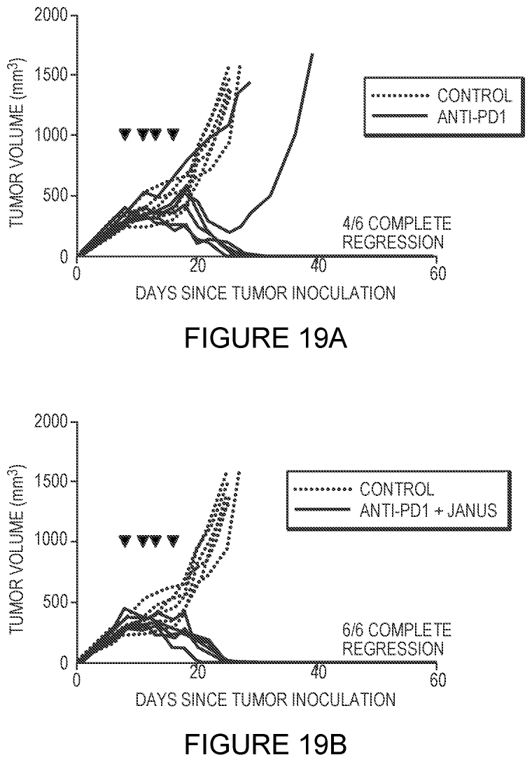

The results for Groups 1, 5 and 6 (vehicle, anti-mouse PD1 and anti-mouse PD1 combined with Janus) are shown in FIG. 19A and FIG. 19B. While anti-mouse PD1 had good activity with 4 out of 6 mice demonstrating complete regressions (similar to Janus alone with 3 out of 6 mice demonstrating complete regression, see FIG. 18A), the combination of anti-mouse PD1 with Janus demonstrated complete regression of tumor growth in all 6 mice (FIG. 19B). There was no body weight loss in any of the animals given this combination.

### Example 9

This Example describes the in vivo administration of antibody sialidase conjugates (ASCs) with bacterial sialidases.

A Janus ASC including Salmonella typhimurium sialidase (St-sialidase) and trastuzumab (including a first polypeptide chain with amino acid sequence SEQ ID NO: 66, encoded by nucleotide sequence SEQ ID NO: 86, a second polypeptide chain with amino acid sequence SEQ ID NO: 67, encoded by nucleotide sequence SEQ ID NO: 87, and a third polypeptide chain with amino acid sequence SEQ ID NO: 90, encoded by nucleotide sequence SEQ ID NO: 91) was made and tested in this Example.

The ASC was tested in a mouse syngeneic tumor model injected with a B16 melanoma cell line expressing human HER2 (B16D5-HER2, Surana et al. CANCER IMMUNOL RES, 2(11): 1103-1112). Female C57BL/6 mice, 6-8 weeks of age, were inoculated subcutaneously in the right lower flank region with B16D5-HER2 tumor cells (5×10⁵). Mice were randomly allocated to 3 groups when tumors reached approximately 50 to 100 mm³. Treatment groups are described in TABLE 33 with dosing schedule indicated post randomization. Anti-mouse PD1, obtained from BioXcell (RMP1-14, Cat. No. 665418F1) and anti-mouse CTLA4, obtained from BioXcell (9D9, Cat. #BE0164), were used in combination.

TABLE 33

| | | | | Dose | | |
|---|---|---|---|---|---|---|
| | No. of | | Dose | volume | | Schedule |
| Group | Animals | Treatment | (mg/kg) | (μL/g) | Route | (Days) |
| 1 | 6 | Janus | NA | 10 | i.p. | 0, 3, 7, 10 |
| 2 | 6 | Trastuzumab | 10 | 10 | i.p. | 0, 3, 7, 10 |
| 3 | 6 | anti-mouse | 10 | 10 | i.p. | 0, 3, 7, 10 |
| | | CTLA4 | | | | |
| | | anti-mouse | 10 | 10 | i.p. | 0, 3, 7, 10 |
| | | PD1 | | | | |

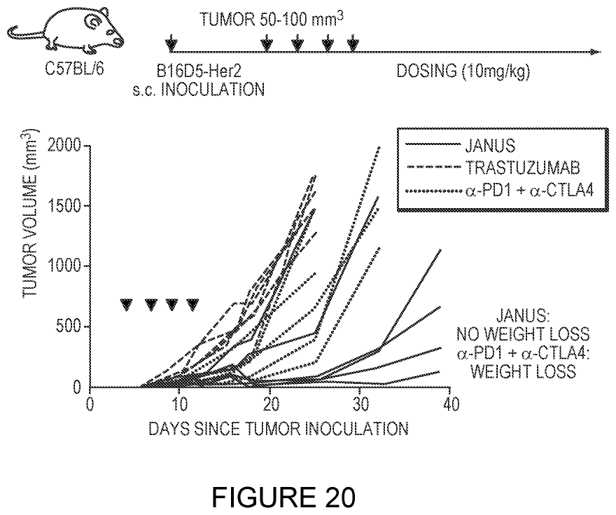

The B16 melanoma mouse model is considered a difficult tumor model to treat with immuno-oncology approaches. A comparison of Janus to a combination of anti-mouse PD1 and anti-mouse CTLA4 was carried out. The results are shown in FIG. 20. Anti-mouse PD1 combined with anti-mouse CTLA4 had an impact on B16D5-HER2 tumor growth, but this combination also demonstrated significant weight loss in the treated animals. By comparison, Janus demonstrated a more robust anti-tumor activity with no significant weight loss. Trastuzumab alone demonstrated marginal activity in this model.

### Example 10

This Example describes the in vivo administration of an antibody sialidase conjugate (ASC) containing a human sialidase.

Janus Trastuzumab, as described in Example 3, including Neu2 with M1D, V6Y, P62G, A93E, I187K and C332A substitutions and Trastuzumab (including a first polypeptide chain with amino acid sequence SEQ ID NO: 66, encoded by nucleotide sequence SEQ ID NO: 86, a second polypeptide chain with amino acid sequence SEQ ID NO: 67, encoded by nucleotide sequence SEQ ID NO: 87, and a third polypeptide chain with amino acid sequence SEQ ID NO: 68, encoded by nucleotide sequence SEQ ID NO: 88) was made and tested in this Example.

Janus Trastuzumab was compared to isotype control antibody in a mouse syngeneic tumor model injected with a murine breast cancer cell line stably expressing human HER2 (EMT6-HER2). Female BALB/c mice, 6-8 weeks of age, were inoculated subcutaneously in the right lower flank region with EMT6-HER2 tumor cells (5×10⁵) for tumor development. Mice were randomly allocated to groups of 8 animals each when tumors reached 50-100 mm³, mean ˜75-100 mm³.

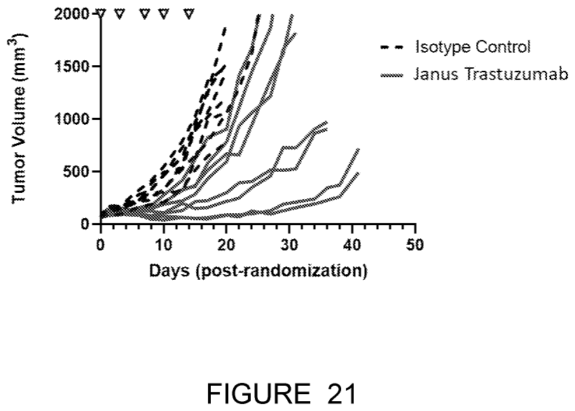

Mice were treated via intraperitoneal injection of 10 mg/kg and tumor volume (mm³) was recorded. FIG. 21 shows individual tumor growth for mice that received treatment with Janus Trastuzumab or control. Significant tumor growth delay was observed following treatment with Janus Trastuzumab in this experiment.

### Example 11

This example describes the construction of additional recombinant human sialidases with mutations that increase expression and/or activity of the sialidase.

Additional mutant Neu2 sialidases were constructed including rationally designed substitutions at position A42. A structural analysis of homologous sialidases revealed that transferring the G147R neuraminidase (sialidase) mutation from influenza A(H1N1)pdm09 onto human Neu2 may have stabilizing effects.

Neu2-M259-Fc (with amino acid sequence SEQ ID NO: 210, encoded by nucleotide sequence SEQ ID NO: 217, and including mutations M1D, V6Y, A42R, P62G, A93E, Q126Y, I187K, A242F, Q270T, and C332A) was expressed in a 1 L transfection of Expi293 human cells using the pCEP4 mammalian expression vector. Neu2-M259-Fc was purified using protein A followed by cation exchange and ceramic hydroxyapatite (CHT) chromatography, quantified with a UV-Vis spectrophotometer (NanoDrop), and examined by SDS-PAGE.

Additional mutant Neu2 sialidases made and tested in this Example include Neu2-M260-Fc (with amino acid sequence SEQ ID NO: 220, encoded by nucleotide sequence SEQ ID NO: 221, and including mutations M1D, V6Y, P62G, A93E, Q112E, Q126Y, I187K, Q270T, A242F, and C332A), Neu2-M261-Fc (with amino acid sequence SEQ ID NO: 222, encoded by nucleotide sequence SEQ ID NO: 223, and including mutations M1D, V6Y, P62G, A93E, Q126Y, I187K, E225C, Q270T, A290C, A242F, and C332A), Neu2-M106-Fc (described in Example 2), and Neu2-173-Fc (described in Example 4).

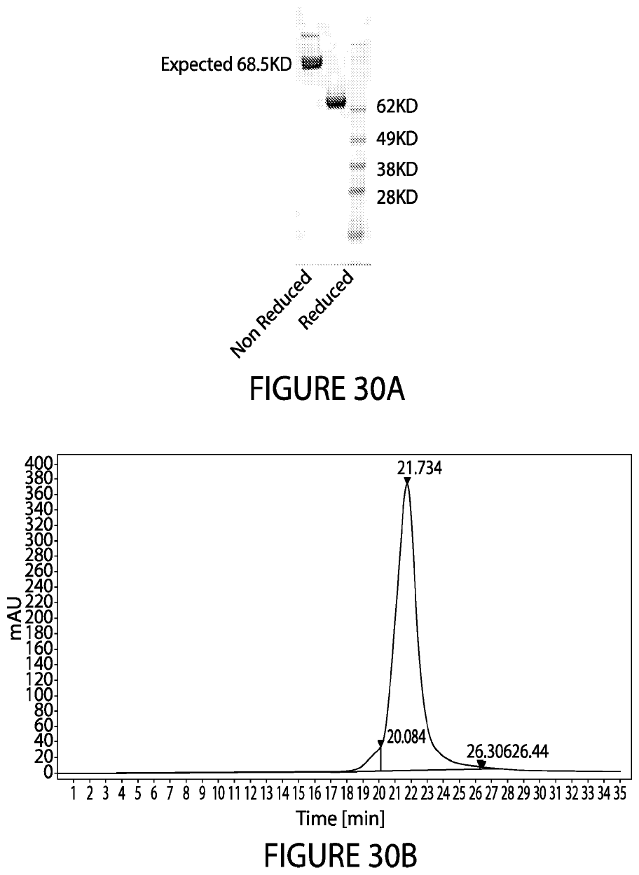

FIG. 30A is an image of an SDS-PAGE gel showing Neu2-M259-Fc under non-reducing and reducing conditions. FIG. 30B is an SEC-HPLC trace for Neu2-M259-Fc, where the monomer species had a retention time of 21.7 minutes, and a monomer content of 96%.

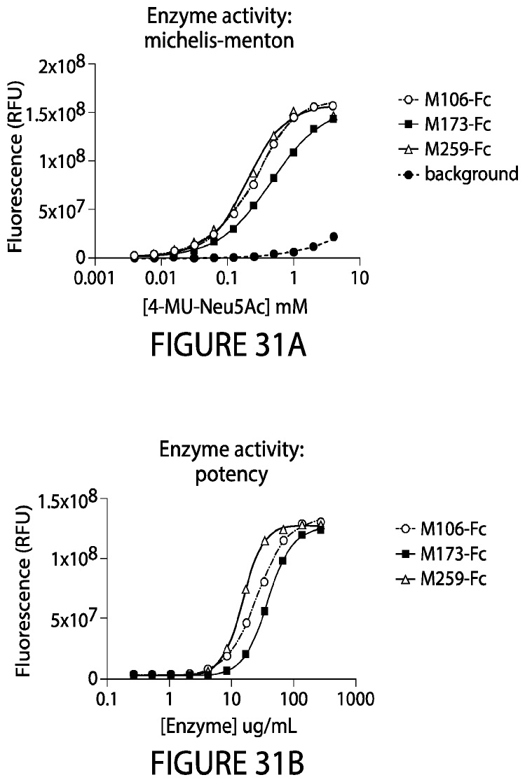

The enzyme kinetics of Neu2-M259-Fc, Neu2-M260-Fc, Neu2-M261-Fc, Neu2-M106-Fc, and Neu2-173-Fc were assayed by measuring the release of sialic acid from the fluorogenic substrate 4-methylumbelliferyl-N-acetylneuraminic acid (4MU-NeuAc) as described above. A Michaelis-Menton kinetics characterization (measured at a variable substrate concentration) of Neu2-M259-Fc, Neu2-M106-Fc, and Neu2-M173-Fc is depicted in FIG. 31A. Estimated K_{M} values were 0.27 mM (Neu2-M106-Fc), 0.46 mM (Neu2-M173-Fc), and 0.20 mM (Neu2-M259-Fc). Enzyme potency (measured at variable enzyme concentration) of Neu2-M259-Fc, Neu2-M106-Fc, and Neu2-M173-Fc is depicted in FIG. 31B. Approximate EC50 values were 20.7 μg/mL (Neu2-M106-Fc), 38.3 μg/mL (Neu2-M173-Fc), and 15.18 μg/mL (Neu2-M259-Fc).

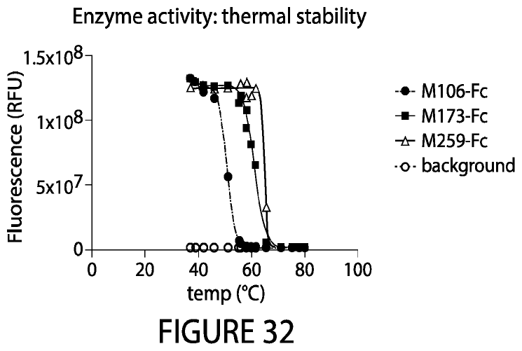

The thermal stability of Neu2-M259-Fc, Neu2-M260-Fc, Neu2-M261-Fc, Neu2-M106-Fc, and Neu2-173-Fc were assayed. Samples were prepared at 0.2 mg/mL, and incubated for 15 minutes across a temperature gradient from 37° C. to 80° C. Enzyme activity was then measured by incubation of 2 μg of enzyme with 0.5 mM of 4-MU-Neu5Ac substrate. Tm was determined by fitting enzyme activity curves versus temperature curves. FIG. 32 depicts a thermal stability characterization of Neu2-M259-Fc, Neu2-M106-Fc, and Neu2-173-Fc.

A summary of certain biochemical attributes of Neu2-Fc variants M106-Fc, M173-Fc, M259-Fc, Neu2-M260-Fc, and Neu2-M261-Fc is depicted in TABLE 34. In TABLE 34, enzymatic activity is indicated as “+++,” which denotes activity >2 fold higher than wild-type Neu2, or “++,” which denotes activity comparable to wild-type Neu2. Together, these results show that Neu2-Fc variants M259-Fc and M260-Fc have improved expression yield and thermal stability relative to the M106-Fc and M173-Fc constructs, and M261-Fc has improved expression yield relative to the M106-Fc and M173-Fc constructs.

TABLE 34

| | | Yield | Tm | Enzyme |
|---|---|---|---|---|
| Name | Mutations | (mg/L) | (° C.) | activity |
| M106-Fc | M1D, V6Y, P62G, A93E, I187K, | 13 | 49.0 | +++ |
| | C332A | | | |
| M173-Fc | M1D, V6Y, P62G, A93E, Q126Y, | 27.2 | 57.1 | ++ |
| | I187K, Q270T, A242F, C332A | | | |
| M259-Fc | M1D, V6Y, A42R, P62G, A93E, | 32 | 61.4 | +++ |
| | Q126Y, I187K, Q270T, A242F, | | | |
| | C332A | | | |
| M260-Fc | M1D, V6Y, P62G, A93E, Q112E, | 34 | 58.7 | +++ |
| | Q126Y, I187K, Q270T, A242F, | | | |
| | C332A | | | |
| M261-Fc | M1D, V6Y, P62G, A93E, Q126Y, | 66 | 55.7 | ++ |
| | I187K, E225C, Q270T, A290C, | | | |
| | A242F, C332A | | | |

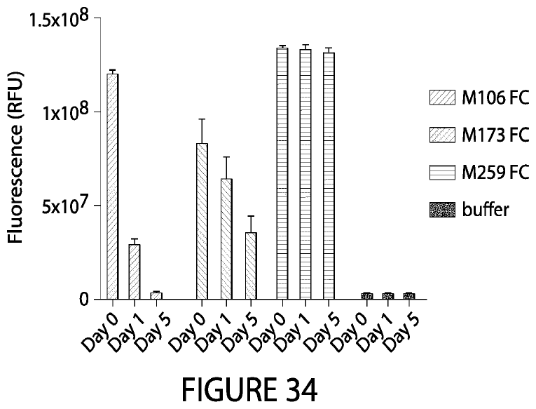

The thermal stability of Neu2-M259-Fc, Neu2-M106-Fc, and Neu2-173-Fc was further assayed by incubating samples at 1 mg/mL at 37° C. for 4 hours, 24 hours, or 5 days. Enzyme activity was then measured using the 4-MU-Neu5Ac assay. Results are depicted in FIG. 34, and show increased thermal stability for Neu2-M259-Fc.

A systematic analysis of the ten mutations in Neu2 variant M-259-Fc was conducted to determine the contribution of each substitution. Each of the ten mutations in Neu2 variant M-259-Fc was back-mutated to the corresponding residue in wildtype Neu2, resulting in ten variants (M262-Fc through M271-Fc) each having nine mutations. The resulting variants are described in TABLE 35.

TABLE 35

| | Backmutation | | | | AA | Nuc |
|---|---|---|---|---|---|---|
| | Relative to M259-Fc | | | | SEQ | SEQ |
| Name | AA # | Mut | WT | Total Mutations | ID NO | ID NO |
| M259-Fc | | — | — | M1D, V6Y, A42R, P62G, A93E, | 210 | 217 |
| | | | | Q126Y, I187K, Q270T, A242F, C332A | | |
| M262-Fc | 1 | D | M | ΔD, V6Y, A42R, P62G, A93E, Q126Y, | 224 | 225 |
| | | | | I187K, Q270T, A242F, C332A | | |
| M263-Fc | 6 | Y | V | M1D, A42R, P62G, A93E, Q126Y, | 226 | 227 |
| | | | | I187K, Q270T, A242F, C332A | | |
| M173-Fc | 42 | R | A | M1D, V6Y, P62G, A93E, Q126Y, | 228 | 229 |
| | | | | I187K, Q270T, A242F, C332A | | |
| M264-Fc | 62 | G | P | M1D, V6Y, A42R, A93E, Q126Y, | 230 | 231 |
| | | | | I187K, Q270T, A242F, C332A | | |
| M265-Fc | 93 | E | A | M1D, V6Y, A42R, P62G, Q126Y, | 232 | 233 |
| | | | | I187K, Q270T, A242F, C332A | | |
| M266-Fc | 126 | Y | Q | M1D, V6Y, A42R, P62G, A93E, | 234 | 235 |
| | | | | I187K, Q270T, A242F, C332A | | |
| M267-Fc | 187 | K | I | M1D, V6Y, A42R, P62G, A93E, | 236 | 237 |
| | | | | Q126Y, Q270T, A242F, C332A | | |
| M268-Fc | 242 | F | A | M1D, V6Y, A42R, P62G, A93E, | 238 | 239 |
| | | | | Q126Y, I187K, Q270T, C332A | | |
| M269-Fc | 270 | T | Q | M1D, V6Y, A42R, P62G, A93E, | 240 | 241 |
| | | | | Q126Y, I187K, A242F, C332A | | |
| M270-Fc | 332 | A | C | M1D, V6Y, A42R, P62G, A93E, | 242 | 243 |
| | | | | Q126Y, I187K, Q270T, A242F | | |

M259-Fc, M262-Fc through M270-Fc, and M173-Fc were expressed in Expi293 human cells using the pCEP4 mammalian expression vector, purified using a single step Protein-A purification, quantified with a UV-Vis spectrophotometer (NanoDrop), and examined by SEC-HPLC. Enzyme kinetics were assayed by measuring the release of sialic acid from the fluorogenic substrate 4-methylumbelliferyl-N-acetylneuraminic acid (4MU-NeuAc) as described above. Thermal stability was assayed by incubating samples across a temperature gradient from 37° C. to 80° C., followed by measurement of enzyme activity, as described above. Tm was determined by fitting enzyme activity curves versus temperature curves. Results are depicted in TABLE 36. In TABLE 36, enzymatic activity is indicated as “+++,” which denotes activity >2 fold higher than wild-type Neu2, “++,” which denotes activity comparable to wild-type Neu2, or “+,” which denotes activity lower than wild-type Neu2.

TABLE 36

| | SEC-HPLC | Yield | Tm | Enzyme |
|---|---|---|---|---|
| Name | Monomer % | (mg/L) | (° C.) | activity |
| M259-Fc | 84% | 37.8 | 60 | +++ |
| M262-Fc | 79% | 32.1 | 57.7 | + |
| M263-Fc | 88% | 17.1 | 62 | +++ |
| M173-Fc | 82% | 35.3 | 56 | + |
| M264-Fc | 92% | 22 | 62 | +++ |
| M265-Fc | 81% | 24.9 | 60 | +++ |
| M266-Fc | 90% | 9.6 | 64 | +++ |
| M267-Fc | 70% | 14 | 61 | +++ |
| M268-Fc | 76% | 38 | 57 | +++ |
| M269-Fc | 79% | 42.9 | 53 | ++ |
| M270-Fc | 87% | 30 | 59 | +++ |

Together, the results showed that removal of each of the individual mutations in M259-Fc negatively impacted at least one of monomer percentage, yield, thermal stability, or enzyme activity. In other words, each of the mutations were found to contribute to at least one of the monomer percentage, yield, stability and activity of the M259-Fc construct.

### Example 12

This Example describes the in vivo administration of a sialidase-Fc fusion protein containing a human sialidase.

A Neu2-Fc fusion protein, including Neu2 with M1D, V6Y, A42R, P62G, A93E, Q126Y, I187K, Q270T, A242F, and C332A substitutions and a human IgG1 with a N297A mutation (having an amino acid sequence depicted in SEQ ID NO: 218, and encoded by the nucleotide sequence depicted in SEQ ID NO: 219) was made and tested in this Example. The Neu2-Fc fusion protein was the same as M259-Fc described in Example 11 but with an additional N297A mutation, and is referred to as M259-Fc-N297A.

M259-Fc-N297A was tested in a transgenic mouse engineered to express human PD-L1 and human PD-1 and where mouse PD-L1 and mouse PD-1 have been disrupted (Biocytogen Inc.). Mice, 6-8 weeks of age, were inoculated subcutaneously in the right lower flank region with a human PD-L1 expressing MC38 tumor cell line for tumor development. Mice were randomly allocated to 3 groups of 8 animals each when tumors reached 100-130 mm³ (mean ˜111 mm³) and treated as shown in TABLE 37.

TABLE 37

| Group | Treatment | Dose | Route | Schedule |
|---|---|---|---|---|
| 1 | Isotype Control | 10 mg/kg | IP | Every |
| 2 | M259-Fc-N297A | 5 mg/kg | | other day; |
| 3 | Atezolizumab | 5 mg/kg | | 8 doses |

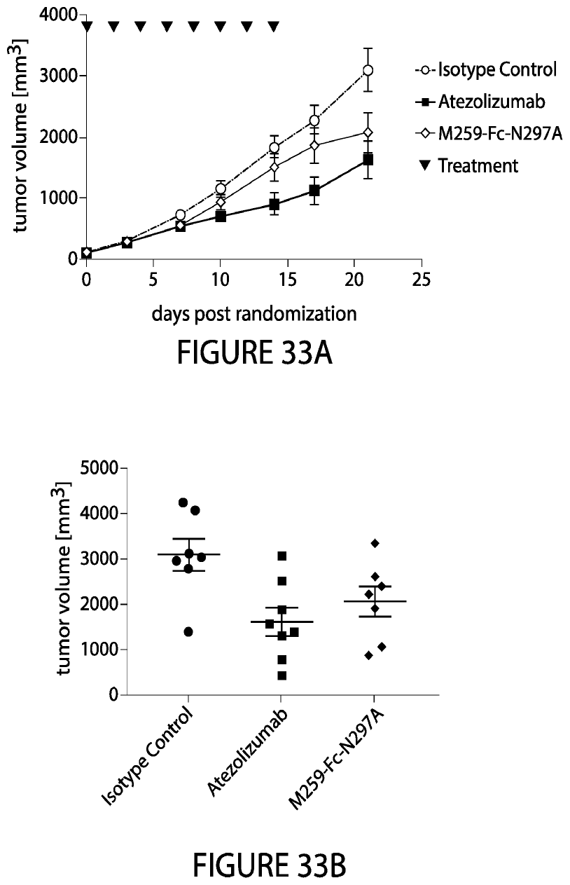

Mice were treated with intraperitoneal injections of 5 mg/kg of M259-Fc-N297A, 5 mg/kg of anti-PD-L1 antibody atezolizumab, or 10 mg/kg of isotype control every other day for 8 doses, and tumor volume (mm³) was recorded. As shown in FIG. 33A (average tumor volume throughout the course of the experiment) and FIG. 33B (tumor volume of individual mice on day 21), mice treated with M259-Fc-N297A exhibited reduced tumor volume compared to mice treated with the isotype control antibody.

### Example 13

This Example describes the construction and expression of anti-HER2 antibody-sialidase genetic fusion proteins, and anti-HER2 antibody sialidase conjugates (ASCs) containing the fusion proteins, with mutated human sialidases.

A Janus Antibody Sialidase Conjugate (ASC) was made using Neu2 with M1D, V6Y, A42R, P62G, A93E, Q126Y, I187K, A242F, Q270T, and C332A substitutions and trastuzumab. This ASC, referred to as “Janus Trastuzumab 2,” included a first polypeptide chain with amino acid sequence SEQ ID NO: 66, encoded by nucleotide sequence SEQ ID NO: 86, a second polypeptide chain with amino acid sequence SEQ ID NO: 189, encoded by nucleotide sequence SEQ ID NO: 245, and a third polypeptide chain with amino acid sequence SEQ ID NO: 205, encoded by nucleotide sequence SEQ ID NO: 246.

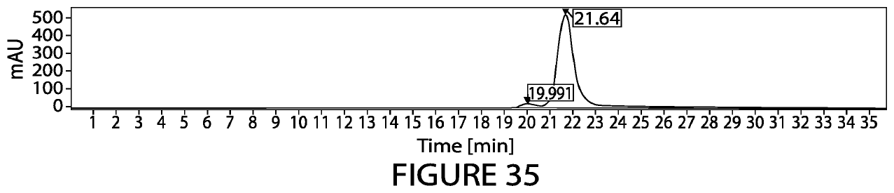

Janus Trastuzumab 2 was expressed in a 1,000 mL transfection of Expi293 human cells using the pCEP4 mammalian expression vector. The ASC was purified using protein A, cation exchange and ceramic hydroxyapatite (CHT) chromatography, quantified with a UV-Vis spectrophotometer (NanoDrop), and examined by SDS-PAGE. Janus Trastuzumab 2 expressed well with 95% monomer purity as determined by SEC-HPLC (FIG. 35).

The enzymatic activity of the recombinantly expressed Janus Trastuzumab 2 was assayed by measuring the release of sialic acid from the fluorogenic substrate 4-methylumbelliferyl-N-acetylneuraminic acid (4MU-NeuAc), as described above. Janus Trastuzumab 2 was enzymatically active, with a Vmax of 2.2×108.

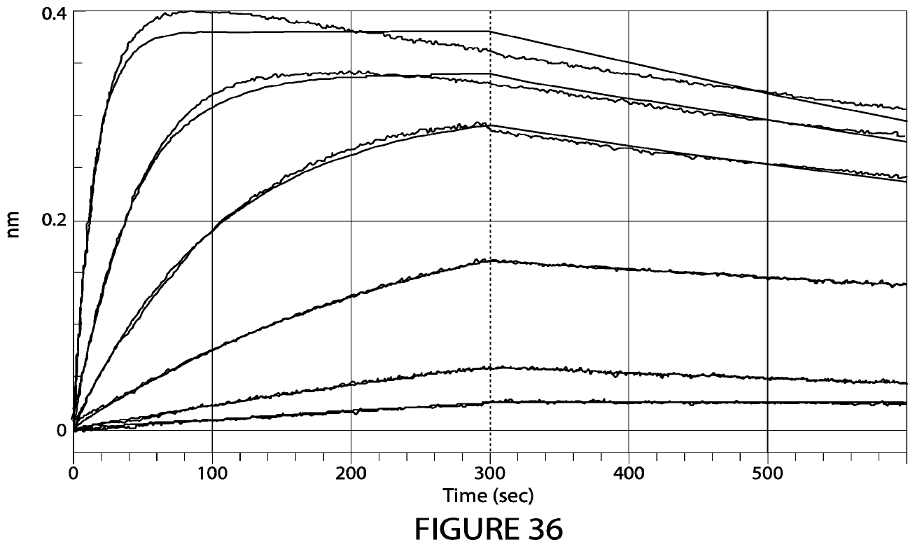

Janus Trastuzumab 2 was tested for antigen (HER2) binding by using ForteBio Octet with the ASC captured on anti-Fc sensors with dipping into serial dilutions of HER2 (titrated from 100 nM in a 2× series dilution). The buffer reference was subtracted from the signal and aligned to the baseline. KD, Kon and Koff values were generated using 1:1 fitting model. Janus Trastuzumab 2 bound to HER2 with a KD of 5.5E-10, Kon of 1.24E06 (1/Ms), and a Koff of 6.91E-04 (1/s) as shown in FIG. 36.

### Example 14

This Example describes the in vivo administration of anti-HER2 antibody sialidase conjugates (ASCs) containing human sialidases.

Janus Trastuzumab 2, as described above in Example 13, and including a first polypeptide chain with amino acid sequence SEQ ID NO: 66, encoded by nucleotide sequence SEQ ID NO: 86, a second polypeptide chain with amino acid sequence SEQ ID NO: 189, encoded by nucleotide sequence SEQ ID NO: 245, and a third polypeptide chain with amino acid sequence SEQ ID NO: 205, encoded by nucleotide sequence SEQ ID NO: 246, was made and tested in this Example.

Janus Trastuzumab 2 was compared to isotype control antibody and trastuzumab in a mouse syngeneic tumor model injected with a murine breast cancer cell line stably expressing human HER2 (EMT6-HER2). Mice, 6-8 weeks of age, were inoculated subcutaneously in the right lower flank region with HER2-expressing cells for tumor development. Mice were randomly allocated to 5 groups of 8 animals each when tumors reached a mean ˜75-100 mm³ and treated as shown in TABLE 38.

TABLE 38

| Group | Treatment | Dose | | Route | Schedule |
|---|---|---|---|---|---|
| 1 | Isotype Control | 10 | mg/kg | IP | Every |
| 2 | Trastuzumab | 1 | mg/kg | | other day; |
| 3 | Trastuzumab | 10 | mg/kg | | 8 doses |
| 4 | Janus Trastuzumab 2 | 1 | mg/kg | | |
| 5 | Janus Trastuzumab 2 | 10 | mg/kg | | |

Mice were treated with intraperitoneal (IP) injections of 10 mg/kg of trastuzumab, Janus Trastuzumab 2, or isotype control every other day, and tumor volume (mm³) was recorded. Complete Responses (CR) were defined as regression below the limit of palpitation at any point during the study and Partial Responses (PR) were defined as palpable tumors which were not.

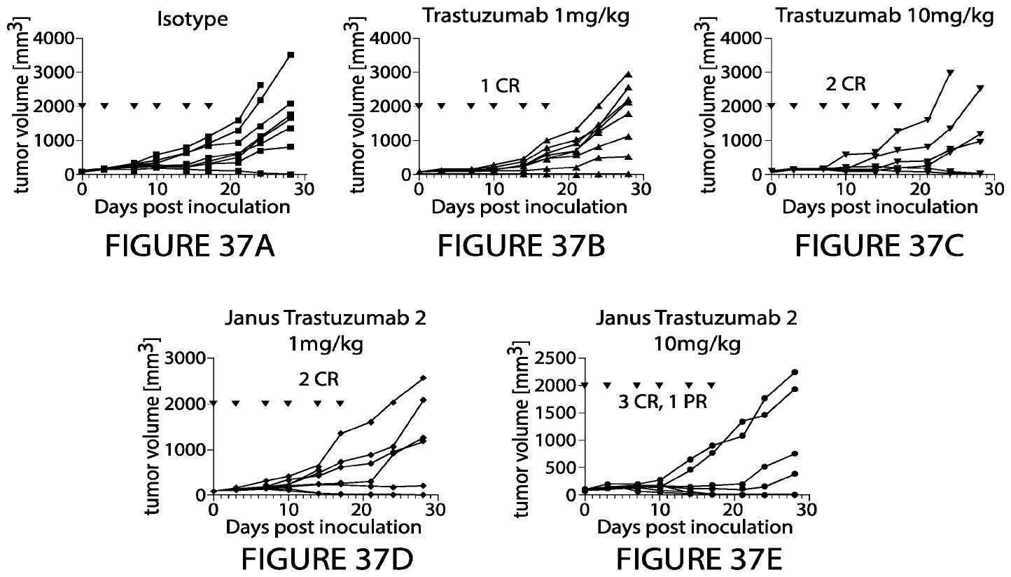

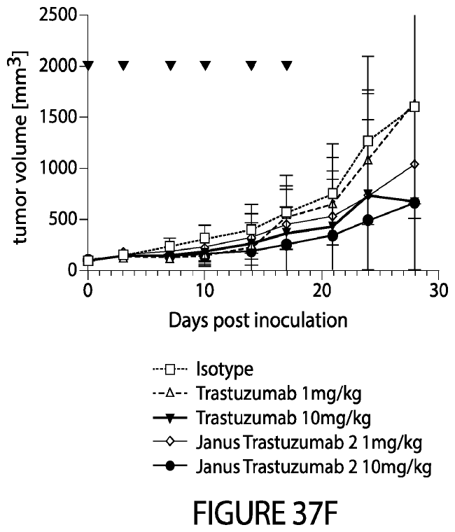

Tumor volumes for individual mice are shown in FIG. 37A-37E. As depicted, Janus Trastuzumab 2 exhibited increased anti-tumor activity based on CRs and PRs as compared to equivalent doses of trastuzumab. Mean tumor volumes are shown in FIG. 37F. Collectively, these results show that the Janus Trastuzumab 2 construct showed comparable or greater activity than trastuzumab.

### INCORPORATION BY REFERENCE

The entire disclosure of each of the patent and scientific documents referred to herein is incorporated by reference for all purposes. Additionally, the entire disclosure of each of U.S. Provisional Patent Application No. 62/870,354, filed Jul. 3, 2019, U.S. Provisional Patent Application No. 62/956,957, filed Jan. 3, 2020, International (PCT) Patent Application No. PCT/US20/40815, filed Jul. 3, 2020, U.S. Provisional Patent Application No. 62/870,348, filed Jul. 3, 2019, U.S. Provisional Patent Application No. 62/956,977, filed Jan. 3, 2020, International (PCT) Patent Application No. PCT/US20/40814, filed Jul. 3, 2020, U.S. Provisional Patent Application No. 62/870,341, filed Jul. 3, 2019, U.S. Provisional Patent Application No. 62/957,041, filed Jan. 3, 2020, and International (PCT) Patent Application No. PCT/US20/40816, filed Jul. 3, 2020, are incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A recombinant mutant sialidase enzyme (e.g., human sialidase enzyme), wherein the sialidase comprises a mutation that increases resistance to cleavage by a protease.

2. The enzyme of claim 1, wherein incubation of the sialidase with the protease results in less than 50% (e.g., less than 40%, less than 30%, less than 10%, less than 5%, less than 3%, less than 1%, or less than 0.5%) of the proteolytic cleavage of a corresponding wild-type sialidase when incubated with the protease under the same conditions.

3. The enzyme of claim 1 or claim 2, wherein the protease is trypsin.

4. The enzyme of any one of claims 1-3, wherein the sialidase comprises:
(a) a substitution of an alanine residue at a position corresponding to position 213 of wild-type human Neu2 (A213);
(b) a substitution of a leucine residue at a position corresponding to position 240 of wild-type human Neu2 (L240);
(c) a substitution of an arginine residue at a position corresponding to position 241 of wild-type human Neu2 (R241);
(d) a substitution of an alanine residue at a position corresponding to position 242 of wild-type human Neu2 (A242);
(e) a substitution of an arginine residue at a position corresponding to position 243 of wild-type human Neu2 (R243);
(f) a substitution of a valine residue at a position corresponding to position 244 of wild-type human Neu2 (V244);
(g) a substitution of a serine residue at a position corresponding to position 258 of wild-type human Neu2 (S258);
(h) a substitution of a leucine residue at a position corresponding to position 260 of wild-type human Neu2 (L260);
(i) a substitution of a valine residue at a position corresponding to position 265 of wild-type human Neu2 (V265);
or a combination of any of the foregoing substitutions.

5. The enzyme of claim 4, wherein, in the sialidase:
(a) the alanine residue at a position corresponding to position 213 of wild-type human Neu2 is substituted by cysteine (A213C), asparagine (A213N), serine (A213S), or threonine (A213T);
(b) the leucine residue at a position corresponding to position 240 of wild-type human Neu2 (L240) is substituted by aspartic acid (L240D), asparagine (L240N), or tyrosine (L240Y);
(c) the arginine residue at a position corresponding to position 241 of wild-type human Neu2 (R241) is substituted by alanine (R241A), aspartic acid (R241D), leucine (R241L), glutamine (R241Q). or tyrosine (R241Y);
(d) the alanine residue at a position corresponding to position 242 of wild-type human Neu2 (A242) is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y);
(e) the valine residue at a position corresponding to position 244 of wild-type human Neu2 (V244) is substituted by isoleucine (V244I), lysine (V244K), or proline (V244P);
(f) the serine residue at a position corresponding to position 258 of wild-type human Neu2 (S258) is substituted by cysteine (S258C);
(g) the leucine residue at a position corresponding to position 260 of wild-type human Neu2 (L260) is substituted by aspartic acid (L260D), phenylalanine (L260F), glutamine (L260Q), or threonine (L260T);
(h) the valine residue at a position corresponding to position 265 of wild-type human Neu2 (V265) is substituted by phenylalanine (V265F);
or a combination of any of the foregoing substitutions.

6. The enzyme of claim 5, wherein, the sialidase comprises a combination of substitutions as set forth in TABLE 2.

7. A recombinant mutant human sialidase enzyme, wherein the sialidase comprises:
(a) a substitution of a proline residue at a position corresponding to position 5 of wild-type human Neu2 (P5);
(b) a substitution of a lysine residue at a position corresponding to position 9 of wild-type human Neu2 (K9);
(c) a substitution of an alanine residue at a position corresponding to position 42 of wild-type human Neu2 (A42);
(d) a substitution of a lysine residue at a position corresponding to position 44 of wild-type human Neu2 (K44);
(e) a substitution of a lysine residue at a position corresponding to position 45 of wild-type human Neu2 (K45);
(f) a substitution of a leucine residue at a position corresponding to position 54 of wild-type human Neu2 (L54);
(g) a substitution of a proline residue at a position corresponding to position 62 of wild-type human Neu2 (P62);
(h) a substitution of a glutamine residue at a position corresponding to position 69 of wild-type human Neu2 (Q69);
(i) a substitution of an arginine residue at a position corresponding to position 78 of wild-type human Neu2 (R78);
(j) a substitution of an aspartic acid residue at a position corresponding to position 80 of wild-type human Neu2 (D80);
(k) a substitution of an alanine residue at a position corresponding to position 93 of wild-type human Neu2 (A93);
(l) a substitution of a glycine residue at a position corresponding to position 107 of wild-type human Neu2 (G107);
(m) a substitution of a glutamine residue at a position corresponding to position 108 of wild-type human Neu2 (Q108);
(n) a substitution of a glutamine residue at a position corresponding to position 112 of wild-type human Neu2 (Q112);
(o) a substitution of a cysteine residue at a position corresponding to position 125 of wild-type human Neu2 (C125);
(p) a substitution of a glutamine residue at a position corresponding to position 126 of wild-type human Neu2 (Q126);
(q) a substitution of an alanine residue at a position corresponding to position 150 of wild-type human Neu2 (A150);
(r) a substitution of a cysteine residue at a position corresponding to position 164 of wild-type human Neu2 (C164);
(s) a substitution of an arginine residue at a position corresponding to position 170 of wild-type human Neu2 (R170);
(t) a substitution of an alanine residue at a position corresponding to position 171 of wild-type human Neu2 (A171);
(u) a substitution of a glutamine residue at a position corresponding to position 188 of wild-type human Neu2 (Q188);
(v) a substitution of an arginine residue at a position corresponding to position 189 of wild-type human Neu2 (R189);
(w) a substitution of an alanine residue at a position corresponding to position 213 of wild-type human Neu2 (A213);
(x) a substitution of a leucine residue at a position corresponding to position 217 of wild-type human Neu2 (L217);
(y) a substitution of a glutamic acid residue at a position corresponding to position 225 of wild-type human Neu2 (E225);
(z) a substitution of a histidine residue at a position corresponding to position 239 of wild-type human Neu2 (H239);
(aa) a substitution of a leucine residue at a position corresponding to position 240 of wild-type human Neu2 (L240);
(bb) a substitution of an arginine residue at a position corresponding to position 241 of wild-type human Neu2 (R241);
(cc) a substitution of an alanine residue at a position corresponding to position 242 of wild-type human Neu2 (A242);
(dd) a substitution of a valine residue at a position corresponding to position 244 of wild-type human Neu2 (V244);
(ee) a substitution of a threonine residue at a position corresponding to position 249 of wild-type human Neu2 (T249);
(ff) a substitution of an aspartic acid residue at a position corresponding to position 251 of wild-type human Neu2 (D251);
(gg) a substitution of a glutamic acid residue at a position corresponding to position 257 of wild-type human Neu2 (E257);
(hh) a substitution of a serine residue at a position corresponding to position 258 of wild-type human Neu2 (S258);
(ii) a substitution of a leucine residue at a position corresponding to position 260 of wild-type human Neu2 (L260);
(jj) a substitution of a valine residue at a position corresponding to position 265 of wild-type human Neu2 (V265);
(kk) a substitution of a glutamine residue at a position corresponding to position 270 of wild-type human Neu2 (Q270);
(ll) a substitution of a tryptophan residue at a position corresponding to position 292 of wild-type human Neu2 (W292);
(mm) a substitution of a serine residue at a position corresponding to position 301 of wild-type human Neu2 (S301);
(nn) a substitution of a tryptophan residue at a position corresponding to position 302 of wild-type human Neu2 (W302);
(oo) a substitution of a valine residue at a position corresponding to position 363 of wild-type human Neu2 (V363); or
(pp) a substitution of a leucine residue at a position corresponding to position 365 of wild-type human Neu2 (L365);
or a combination of any of the foregoing substitutions.

8. A recombinant mutant human sialidase enzyme, wherein the sialidase comprises:
(a) a substitution of a proline residue at a position corresponding to position 5 of wild-type human Neu2 (P5);
(b) a substitution of a lysine residue at a position corresponding to position 9 of wild-type human Neu2 (K9);
(c) a substitution of a lysine residue at a position corresponding to position 44 of wild-type human Neu2 (K44);
(d) a substitution of a lysine residue at a position corresponding to position 45 of wild-type human Neu2 (K45);
(e) a substitution of a leucine residue at a position corresponding to position 54 of wild-type human Neu2 (L54);
(f) a substitution of a proline residue at a position corresponding to position 62 of wild-type human Neu2 (P62);
(g) a substitution of a glutamine residue at a position corresponding to position 69 of wild-type human Neu2 (Q69);
(h) a substitution of an arginine residue at a position corresponding to position 78 of wild-type human Neu2 (R78);
(i) a substitution of an aspartic acid residue at a position corresponding to position 80 of wild-type human Neu2 (D80);
(j) a substitution of an alanine residue at a position corresponding to position 93 of wild-type human Neu2 (A93);
(k) a substitution of a glycine residue at a position corresponding to position 107 of wild-type human Neu2 (G107);
(l) a substitution of a glutamine residue at a position corresponding to position 108 of wild-type human Neu2 (Q108);
(m) a substitution of a glutamine residue at a position corresponding to position 112 of wild-type human Neu2 (Q112);
(n) a substitution of a cysteine residue at a position corresponding to position 125 of wild-type human Neu2 (C125);
(o) a substitution of a glutamine residue at a position corresponding to position 126 of wild-type human Neu2 (Q126);
(p) a substitution of an alanine residue at a position corresponding to position 150 of wild-type human Neu2 (A150);
(q) a substitution of a cysteine residue at a position corresponding to position 164 of wild-type human Neu2 (C164);
(r) a substitution of an arginine residue at a position corresponding to position 170 of wild-type human Neu2 (R170);
(s) a substitution of an alanine residue at a position corresponding to position 171 of wild-type human Neu2 (A171);
(t) a substitution of a glutamine residue at a position corresponding to position 188 of wild-type human Neu2 (Q188);
(u) a substitution of an arginine residue at a position corresponding to position 189 of wild-type human Neu2 (R189);
(v) a substitution of an alanine residue at a position corresponding to position 213 of wild-type human Neu2 (A213);
(w) a substitution of a leucine residue at a position corresponding to position 217 of wild-type human Neu2 (L217);
(x) a substitution of a glutamic acid residue at a position corresponding to position 225 of wild-type human Neu2 (E225);
(y) a substitution of a histidine residue at a position corresponding to position 239 of wild-type human Neu2 (H239);
(z) a substitution of a leucine residue at a position corresponding to position 240 of wild-type human Neu2 (L240);
(aa) a substitution of an arginine residue at a position corresponding to position 241 of wild-type human Neu2 (R241);
(bb) a substitution of an alanine residue at a position corresponding to position 242 of wild-type human Neu2 (A242);
(cc) a substitution of a valine residue at a position corresponding to position 244 of wild-type human Neu2 (V244);
(dd) a substitution of a threonine residue at a position corresponding to position 249 of wild-type human Neu2 (T249);
(ee) a substitution of an aspartic acid residue at a position corresponding to position 251 of wild-type human Neu2 (D251);
(ff) a substitution of a glutamic acid residue at a position corresponding to position 257 of wild-type human Neu2 (E257);
(gg) a substitution of a serine residue at a position corresponding to position 258 of wild-type human Neu2 (S258);
(hh) a substitution of a leucine residue at a position corresponding to position 260 of wild-type human Neu2 (L260);
(ii) a substitution of a valine residue at a position corresponding to position 265 of wild-type human Neu2 (V265);
(jj) a substitution of a glutamine residue at a position corresponding to position 270 of wild-type human Neu2 (Q270);
(kk) a substitution of a tryptophan residue at a position corresponding to position 292 of wild-type human Neu2 (W292);
(ll) a substitution of a serine residue at a position corresponding to position 301 of wild-type human Neu2 (S301);
(mm) a substitution of a tryptophan residue at a position corresponding to position 302 of wild-type human Neu2 (W302);
(nn) a substitution of a valine residue at a position corresponding to position 363 of wild-type human Neu2 (V363); or
(oo) a substitution of a leucine residue at a position corresponding to position 365 of wild-type human Neu2 (L365);
or a combination of any of the foregoing substitutions.

9. The enzyme of claim 7 or 8, wherein the sialidase comprises a substitution of K9, A42, P62, A93, Q126, A242, Q270, S301, W302, V363, or L365, or a combination of any of the foregoing substitutions.

10. The enzyme of claim 7 or 8, wherein the sialidase comprises a substitution of K9, P62, A93, Q126, A242, Q270, S301, W302, V363, or L365, or a combination of any of the foregoing substitutions.

11. The enzyme of any one of claims 7-10, wherein, in the sialidase:
(a) the proline residue at a position corresponding to position 5 of wild-type human Neu2 is substituted by histidine (P5H);
(b) the lysine residue at a position corresponding to position 9 of wild-type human Neu2 is substituted by aspartic acid (K9D);
(c) the alanine residue at a position corresponding to position 42 of wild-type human Neu2 is substituted by arginine (A42R);
(d) the lysine residue at a position corresponding to position 44 of wild-type human Neu2 is substituted by arginine (K44R) or glutamic acid (K44E);
(e) the lysine residue at a position corresponding to position 45 of wild-type human Neu2 is substituted by alanine (K45A), arginine (K45R), or glutamic acid (K45E);
(f) the leucine residue at a position corresponding to position 54 of wild-type human Neu2 is substituted by methionine (L54M);
(g) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T);
(h) the glutamine residue at a position corresponding to position 69 of wild-type human Neu2 is substituted by histidine (Q69H);
(i) the arginine residue at a position corresponding to position 78 of wild-type human Neu2 is substituted by lysine (R78K);
(j) the aspartic acid residue at a position corresponding to position 80 of wild-type human Neu2 is substituted by proline (D80P);
(k) the alanine residue at a position corresponding to position 93 of wild-type human Neu2 is substituted by glutamic acid (A93E) or lysine (A93K);
(l) the glycine residue at a position corresponding to position 107 of wild-type human Neu2 is substituted by aspartic acid (G107D);
(m) the glutamine residue at a position corresponding to position 108 of wild-type human Neu2 is substituted by histidine (Q108H);
(n) the glutamine residue at a position corresponding to position 112 of wild-type human Neu2 is substituted by arginine (Q112R) or lysine (Q112K);
(o) the cysteine residue at a position corresponding to position 125 of wild-type human Neu2 is substituted by leucine (C125L);
(p) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y);
(q) the alanine residue at a position corresponding to position 150 of wild-type human Neu2 is substituted by valine (A150V);
(r) the cysteine residue at a position corresponding to position 164 of wild-type human Neu2 is substituted by glycine (C164G);
(s) the arginine residue at a position corresponding to position 170 of wild-type human Neu2 is substituted by proline (R170P);
(t) the alanine residue at a position corresponding to position 171 of wild-type human Neu2 is substituted by glycine (A171G);
(u) the glutamine residue at a position corresponding to position 188 of wild-type human Neu2 is substituted by proline (Q188P);
(v) the arginine residue at a position corresponding to position 189 of wild-type human Neu2 is substituted by proline (R189P);
(w) the alanine residue at a position corresponding to position 213 of wild-type human Neu2 is substituted by cysteine (A213C), asparagine (A213N), serine (A213S) or threonine (A213 T);
(x) the leucine residue at a position corresponding to position 217 of wild-type human Neu2 is substituted by alanine (L217A) or valine (L217V);
(y) the glutamic acid residue at a position corresponding to position 225 of wild-type human Neu2 is substituted by proline (E225P);
(z) the histidine residue at a position corresponding to position 239 of wild-type human Neu2 is substituted by proline (H239P);
(aa) the leucine residue at a position corresponding to position 240 of wild-type human Neu2 is substituted by aspartic acid (L240D), asparagine (L240N), or tyrosine (L240Y);
(bb) the arginine residue at a position corresponding to position 241 of wild-type human Neu2 is substituted by alanine (R241A), aspartic acid (R241D), leucine (R241L), glutamine (R241Q). or tyrosine (R241Y);
(cc) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y);
(dd) the valine residue at a position corresponding to position 244 of wild-type human Neu2 is substituted by isoleucine (V244I) or proline (V244P);
(ee) the threonine residue at a position corresponding to position 249 of wild-type human Neu2 is substituted by alanine (T249A);
(ff) the aspartic acid residue at a position corresponding to position 251 of wild-type human Neu2 is substituted by glycine (D251G);
(gg) the glutamic acid residue at a position corresponding to position 257 of wild-type human Neu2 is substituted by proline (E257P);
(hh) the serine residue at a position corresponding to position 258 is substituted by cysteine (S258C);
(ii) the leucine residue at a position corresponding to position 260 of wild-type human Neu2 is substituted by aspartic acid (L260D), phenylalanine (L260F), glutamine (L260Q), or threonine (L260T);
(jj) the valine residue at a position corresponding to position 265 of wild-type human Neu2 is substituted by phenylalanine (V265F);
(kk) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T);
(ll) the tryptophan residue at a position corresponding to position 292 of wild-type human Neu2 is substituted by arginine (W292R);
(mm) the serine residue at a position corresponding to position 301 of wild-type human Neu2 is substituted by alanine (S301A), aspartic acid (S301D), glutamic acid (S301E), phenylalanine (S301F), histidine (S301H), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y);
(nn) the tryptophan residue at a position corresponding to position 302 of wild-type human Neu2 is substituted by alanine (W302A), aspartic acid (W302D), phenylalanine (W302F), glycine (W302G), histidine (W302H), isoleucine (W302I), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y);
(oo) the valine residue at a position corresponding to position 363 of wild-type human Neu2 is substituted by arginine (V363R); or
(pp) the leucine residue at a position corresponding to position 365 of wild-type human Neu2 is substituted by glutamine (L365Q), histidine (L365H), isoleucine (L365I), lysine (L365K) or serine (L365S);
or the sialidase comprises a combination of any of the foregoing substitutions.

12. The enzyme of any one of claims 7-10, wherein, in the sialidase:
(a) the proline residue at a position corresponding to position 5 of wild-type human Neu2 is substituted by histidine (P5H);
(b) the lysine residue at a position corresponding to position 9 of wild-type human Neu2 is substituted by aspartic acid (K9D);
(c) the lysine residue at a position corresponding to position 44 of wild-type human Neu2 is substituted by arginine (K44R) or glutamic acid (K44E);
(d) the lysine residue at a position corresponding to position 45 of wild-type human Neu2 is substituted by alanine (K45A), arginine (K45R), or glutamic acid (K45E);
(e) the leucine residue at a position corresponding to position 54 of wild-type human Neu2 is substituted by methionine (L54M);
(f) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T);
(g) the glutamine residue at a position corresponding to position 69 of wild-type human Neu2 is substituted by histidine (Q69H);
(h) the arginine residue at a position corresponding to position 78 of wild-type human Neu2 is substituted by lysine (R78K);
(i) the aspartic acid residue at a position corresponding to position 80 of wild-type human Neu2 is substituted by proline (D80P);
(j) the alanine residue at a position corresponding to position 93 of wild-type human Neu2 is substituted by glutamic acid (A93E) or lysine (A93K);
(k) the glycine residue at a position corresponding to position 107 of wild-type human Neu2 is substituted by aspartic acid (G107D);
(l) the glutamine residue at a position corresponding to position 108 of wild-type human Neu2 is substituted by histidine (Q108H);
(m) the glutamine residue at a position corresponding to position 112 of wild-type human Neu2 is substituted by arginine (Q112R) or lysine (Q112K);
(n) the cysteine residue at a position corresponding to position 125 of wild-type human Neu2 is substituted by leucine (C125L);
(o) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y);
(p) the alanine residue at a position corresponding to position 150 of wild-type human Neu2 is substituted by valine (A150V);
(q) the cysteine residue at a position corresponding to position 164 of wild-type human Neu2 is substituted by glycine (C164G);
(r) the arginine residue at a position corresponding to position 170 of wild-type human Neu2 is substituted by proline (R170P);
(s) the alanine residue at a position corresponding to position 171 of wild-type human Neu2 is substituted by glycine (A171G);
(t) the glutamine residue at a position corresponding to position 188 of wild-type human Neu2 is substituted by proline (Q188P);
(u) the arginine residue at a position corresponding to position 189 of wild-type human Neu2 is substituted by proline (R189P);
(v) the alanine residue at a position corresponding to position 213 of wild-type human Neu2 is substituted by cysteine (A213C), asparagine (A213N), serine (A213S) or threonine (A213 T);
(w) the leucine residue at a position corresponding to position 217 of wild-type human Neu2 is substituted by alanine (L217A) or valine (L217V);
(x) the glutamic acid residue at a position corresponding to position 225 of wild-type human Neu2 is substituted by proline (E225P);
(y) the histidine residue at a position corresponding to position 239 of wild-type human Neu2 is substituted by proline (H239P);
(z) the leucine residue at a position corresponding to position 240 of wild-type human Neu2 is substituted by aspartic acid (L240D), asparagine (L240N), or tyrosine (L240Y);
(aa) the arginine residue at a position corresponding to position 241 of wild-type human Neu2 is substituted by alanine (R241A), aspartic acid (R241D), leucine (R241L), glutamine (R241Q). or tyrosine (R241Y);
(bb) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y);
(cc) the valine residue at a position corresponding to position 244 of wild-type human Neu2 is substituted by isoleucine (V244I) or proline (V244P);
(dd) the threonine residue at a position corresponding to position 249 of wild-type human Neu2 is substituted by alanine (T249A);
(ee) the aspartic acid residue at a position corresponding to position 251 of wild-type human Neu2 is substituted by glycine (D251G);
(ff) the glutamic acid residue at a position corresponding to position 257 of wild-type human Neu2 is substituted by proline (E257P);
(gg) the serine residue at a position corresponding to position 258 is substituted by cysteine (S258C);
(hh) the leucine residue at a position corresponding to position 260 of wild-type human Neu2 is substituted by aspartic acid (L260D), phenylalanine (L260F), glutamine (L260Q), or threonine (L260T);
(ii) the valine residue at a position corresponding to position 265 of wild-type human Neu2 is substituted by phenylalanine (V265F);
(jj) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T);
(kk) the tryptophan residue at a position corresponding to position 292 of wild-type human Neu2 is substituted by arginine (W292R);
(ll) the serine residue at a position corresponding to position 301 of wild-type human Neu2 is substituted by alanine (S301A), aspartic acid (S301D), glutamic acid (S301E), phenylalanine (S301F), histidine (S301H), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y);
(mm) the tryptophan residue at a position corresponding to position 302 of wild-type human Neu2 is substituted by alanine (W302A), aspartic acid (W302D), phenylalanine (W302F), glycine (W302G), histidine (W302H), isoleucine (W302I), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y);
(nn) the valine residue at a position corresponding to position 363 of wild-type human Neu2 is substituted by arginine (V363R); or
(oo) the leucine residue at a position corresponding to position 365 of wild-type human Neu2 is substituted by glutamine (L365Q), histidine (L365H), isoleucine (L365I), lysine (L365K) or serine (L365S);
or the sialidase comprises a combination of any of the foregoing substitutions.

13. The enzyme of claim 11 or 12, wherein the sialidase comprises a substitution selected from the K9D, A42R, P62G, P62N, P62S, P62T, A93E, Q126Y, A242F, A242W, A242Y, Q270A, Q270T, S301A, S301R, W302K, W302R, V363R, and L365I substitutions, or a combination of any of the foregoing substitutions.

14. The enzyme of claim 11 or 12, wherein the sialidase comprises a substitution selected from the K9D, P62G, P62N, P62S, P62T, A93E, Q126Y, A242F, A242W, A242Y, Q270A, Q270T, S301A, S301R, W302K, W302R, V363R, and L365I substitutions, or a combination of any of the foregoing substitutions.

15. The enzyme of any one of claims 1-14, wherein the sialidase further comprises:
(a) a substitution or deletion of a methionine residue at a position corresponding to position 1 of wild-type human Neu2 (M1);
(b) a substitution of a valine residue at a position corresponding to position 6 of wild-type human Neu2 (V6);
(c) a substitution of an isoleucine residue at a position corresponding to position 187 of wild-type human Neu2 (I187); or
(d) a substitution of a cysteine residue at a position corresponding to position 332 of wild-type human Neu2 (C332);
or a combination of any of the foregoing substitutions.

16. The enzyme of claim 15, wherein, in the sialidase:
(a) the methionine residue at a position corresponding to position 1 of wild-type human Neu2 is deleted (ΔM1), is substituted by alanine (M1A), or is substituted by aspartic acid (M1D);
(b) the valine residue at a position corresponding to position 6 of wild-type human Neu2 is substituted by tyrosine (V6Y);
(c) the isoleucine residue at a position corresponding to position 187 of wild-type human Neu2 is substituted by lysine (I187K); or
(d) the cysteine residue at a position corresponding to position 332 of wild-type human Neu2 is substituted by alanine (C332A);
or the sialidase comprises a combination of any of the foregoing substitutions.

17. The enzyme of claim 16, wherein the sialidase comprises:
(a) the M1D, V6Y, P62G, A93E, I187K, and C332A substitutions;
(b) the M1D, V6Y, K9D, A93E, I187K, C332A, V363R, and L365I substitutions;
(c) the M1D, V6Y, P62N, I187K, and C332A substitutions;
(d) the M1D, V6Y, I187K, Q270A, S301R, W302K, and C332A substitutions;
(e) the M1D, V6Y, P62S, I187K, Q270A, S301R, W302K, and C332A substitutions;
(f) the M1D, V6Y, P62T, I187K, Q270A, S301R, W302K, and C332A substitutions;
(g) the M1D, V6Y, P62N, I187K, Q270A, S301R, W302K, and C332A substitutions;
(h) the M1D, V6Y, P62G, A93E, I187K, S301A, W302R, and C332A substitutions;
(i) the M1D, V6Y, P62G, A93E, Q126Y, I187K, Q270T, and C332A substitutions;
(j) the M1D, V6Y, P62G, A93E, Q126Y, I187K, and C332A substitutions;
(k) the M1D, V6Y, P62G, A93E, Q126Y, I187K, A242F, Q270T, and C332A substitutions; or
(l) the M1D, V6Y, A42R, P62G, A93E, Q126Y, I187K, A242F, Q270T, and C332A substitutions.

18. The enzyme of claim 16, wherein the sialidase comprises:
(a) the M1D, V6Y, P62G, A93E, I187K, and C332A substitutions;
(b) the M1D, V6Y, K9D, A93E, I187K, C332A, V363R, and L365I substitutions;
(c) the M1D, V6Y, P62N, I187K, and C332A substitutions;
(d) the M1D, V6Y, I187K, Q270A, S301R, W302K, and C332A substitutions;
(e) the M1D, V6Y, P62S, I187K, Q270A, S301R, W302K, and C332A substitutions;
(f) the M1D, V6Y, P62T, I187K, Q270A, S301R, W302K, and C332A substitutions;
(g) the M1D, V6Y, P62N, I187K, Q270A, S301R, W302K, and C332A substitutions;
(h) the M1D, V6Y, P62G, A93E, I187K, S301A, W302R, and C332A substitutions;
(i) the M1D, V6Y, P62G, A93E, Q126Y, I187K, Q270T, and C332A substitutions; or
(j) the M1D, V6Y, P62G, A93E, Q126Y, I187K, A242F, Q270T, and C332A substitutions.

19. The enzyme of any one of claims 1-18, wherein the sialidase is selected from Neu1, Neu2, Neu3, and Neu4.

20. The enzyme of claim 19, wherein the sialidase is Neu2.

21. The enzyme of any one of claims 1-20, wherein the sialidase has a different substrate specificity than the corresponding wild-type sialidase.

22. The enzyme of claim 21, wherein the sialidase can cleave α2,3, α2,6, and/or α2,8 linkages.

23. The enzyme of claim 22, wherein the sialidase can cleave α2,3 and α2,8 linkages.

24. The enzyme of any one of claims 1-23, wherein the sialidase comprises any one of SEQ ID NOs: 48-54, 149, 154, 159, 191, or 198.

25. The enzyme of any one of claims 1-23, wherein the sialidase comprises any one of SEQ ID NOs: 48-54, 149, 154, or 159.

26. A recombinant mutant human sialidase comprising a mutation or combination of mutations set forth in any one of TABLES 1, 2, 7-9, 11-13, 15-30, 34, or 35, and optionally further comprising a mutation or combination of mutations set forth in any one of TABLES 3-6.

27. A fusion protein comprising:
(a) the recombinant enzyme of any one of claims 1-26; and
(b) an immunoglobulin Fc domain and/or an immunoglobulin antigen-binding domain;
wherein the enzyme and the immunoglobulin Fc domain and/or the immunoglobulin antigen-binding domain are linked by a peptide bond or an amino acid linker.

28. The fusion protein of claim 27, wherein the fusion protein comprises an immunoglobulin Fc domain.

29. The fusion protein of claim 28, wherein the immunoglobulin Fc domain is derived from a human IgG1, IgG2, IgG3, IgG4, IgA1, IgA2, IgD, IgE, or IgM Fc domain.

30. The fusion protein of claim 29, wherein the immunoglobulin Fc domain is derived from a human IgG1, IgG2, IgG3, or IgG4 Fc domain.

31. The fusion protein of claim 30, wherein the immunoglobulin Fc domain is derived from a human IgG1 Fc domain.

32. The fusion protein of any one of claims 27-31, wherein the fusion protein comprises an immunoglobulin antigen-binding domain.

33. The fusion protein of claim 32, wherein the immunoglobulin antigen-binding domain is associated with a second immunoglobulin antigen-binding domain to produce an antigen-binding site.

34. The fusion protein of claim 32 or 33, wherein the immunoglobulin antigen-binding domain is derived from an antibody selected from trastuzumab, daratumumab, girentuximab, ofatumumab, avelumab, and rituximab.

35. The fusion protein of any one of claims 27-34, wherein the fusion protein comprises any one of SEQ ID NOs: 203-210, 218, 220, 222, 224, 226, 228, 230, 232, 234, 236, 238, 240, or 242

36. An antibody conjugate comprising the fusion protein of any one of claims 27-35.

37. The antibody conjugate of claim 36, wherein the antibody conjugate comprises a single sialidase.

38. The antibody conjugate of claim 36, wherein the antibody conjugate comprises two sialidases.

39. The antibody conjugate of claim 37, wherein the two sialidases are identical.

40. The antibody conjugate of any one of claims 36-39, wherein the antibody conjugate comprises a single antigen-binding site.

41. The antibody conjugate of any one of claims 36-39, wherein the antibody conjugate comprises two antigen-binding sites.

42. The antibody conjugate of claim 41, wherein the two antigen-binding sites are identical.

43. The antibody conjugate of any one of claims 36-42, wherein the antibody conjugate has a molecular weight from about 135 kDa to about 165 kDa.

44. The antibody conjugate of any one of claims 36-42, wherein the antibody conjugate has a molecular weight from about 215 kDa to about 245 kDa.

45. The antibody conjugate of any one of claims 36-44, wherein the antibody conjugate comprises:
(a) a first polypeptide comprising an immunoglobulin light chain;
(b) a second polypeptide comprising an immunoglobulin heavy chain; and
(c) a third polypeptide comprising an immunoglobulin Fc domain and a sialidase;
wherein the first and second polypeptides are covalently linked together and the second and third polypeptides are linked together, and wherein the first polypeptide and the second polypeptide together define an antigen-binding site.

46. The antibody conjugate of claim 45, wherein the third polypeptide comprises the sialidase and the immunoglobulin Fc domain in an N- to C-terminal orientation.

47. The antibody conjugate of any one of claims 36-44, wherein the fusion protein comprises:
(a) a first polypeptide comprising a first immunoglobulin light chain;
(b) a second polypeptide comprising a first immunoglobulin heavy chain and a first sialidase;
(c) a third polypeptide comprising a second immunoglobulin heavy chain and a second sialidase; and
(d) a fourth polypeptide comprising a second immunoglobulin light chain;
wherein the first and second polypeptides are covalently linked together, the third and fourth polypeptides are covalently linked together, and the second and third polypeptides are covalently linked together, and wherein the first polypeptide and the second polypeptide together define a first antigen-binding site, and the third polypeptide and the fourth polypeptide together define a second antigen-binding site.

48. The antibody conjugate of claim 47, wherein the second and third polypeptides comprise the first and second immunoglobulin heavy chain and the first and second sialidase, respectively, in an N- to C-terminal orientation.

49. The antibody conjugate of any one of claims 36-44, wherein the antibody conjugate comprises:
(a) a first polypeptide comprising a first sialidase, a first immunoglobulin Fc domain, and a first single chain variable fragment (scFv); and
(b) a second polypeptide comprising a second sialidase, a second immunoglobulin Fc domain, and a second single chain variable fragment (scFv);
wherein the first and second polypeptides are covalently linked together, and wherein the first scFv defines a first antigen-binding site, and the second scFv defines a second antigen-binding site.

50. The antibody conjugate of claim 49, wherein the first polypeptide comprises the first sialidase, the first immunoglobulin Fc domain, and the first scFv in an N- to C-terminal orientation, and the second polypeptide comprises the second sialidase, the second immunoglobulin Fc domain, and the second scFv in an N- to C-terminal orientation.

51. The antibody conjugate of any one of claims 36-44, wherein the antibody conjugate comprises:
(a) a first polypeptide comprising an immunoglobulin light chain;
(b) a second polypeptide comprising an immunoglobulin heavy chain and a single chain variable fragment (scFv); and
(c) a third polypeptide comprising an immunoglobulin Fc domain and a sialidase;
wherein the first and second polypeptides are covalently linked together and the second and third polypeptides are covalently linked together, and wherein the immunoglobulin light chain and immunoglobulin heavy chain together define a first antigen-binding site and the scFv defines a second antigen-binding site.

52. The antibody conjugate of claim 51, wherein the second polypeptide comprises the immunoglobulin heavy chain and the scFv in an N- to C-terminal orientation, and the third polypeptide comprises the sialidase and the immunoglobulin Fc domain in an N- to C-terminal orientation.

53. An isolated nucleic acid comprising a nucleotide sequence encoding the recombinant enzyme of any one of claims 1-26, the fusion protein of any one of claims 27-35, or at least a portion of the antibody conjugate of any one of claims 36-52.

54. An expression vector comprising the nucleic acid of claim 53.

55. A host cell comprising the expression vector of claim 54.

56. A pharmaceutical composition comprising the recombinant enzyme of any one of claims 1-26, the fusion protein of any one of claims 27-35, or the antibody conjugate of any one of claims 36-52.

57. A method of treating cancer in a subject in need thereof, the method comprising administering to the subject an effective amount of the recombinant enzyme of any one of claims 1-26, the fusion protein of any one of claims 27-35, or at least a portion of the antibody conjugate of any one of claims 36-52, or the pharmaceutical composition of claim 56.

58. The method of claim 57, wherein the cancer is a solid tumor, soft tissue tumor, hematopoietic tumor or metastatic lesion.

59. The method of claim 58, wherein the solid tumor is a sarcoma, adenocarcinoma, or carcinoma.

60. The method of claim 58 or 59, wherein the solid tumor is a head and neck (e.g., pharynx), thyroid, lung (e.g., small cell or non-small cell lung carcinoma (NSCLC)), breast, lymphoid, gastrointestinal (e.g., oral, esophageal, stomach, liver, pancreas, small intestine, colon and rectum, anal canal), genital or genitourinary tract (e.g., renal, urothelial, bladder, ovarian, uterine, cervical, endometrial, prostate, testicular), CNS (e.g., neural or glial cell, e.g., neuroblastoma or glioma), or skin (e.g., melanoma) tumor.

61. The method of claim 58, wherein the hematopoietic tumor is a leukemia, acute leukemia, acute lymphoblastic leukemia (ALL), B-cell, T-cell or FAB ALL, acute myeloid leukemia (AML), chronic myelocytic leukemia (CIVIL), chronic lymphocytic leukemia (CLL), e.g., transformed CLL, diffuse large B-cell lymphoma (DLBCL), follicular lymphoma, hairy cell leukemia, myelodyplastic syndrome (MDS), lymphoma, Hodgkin's disease, malignant lymphoma, non-Hodgkin's lymphoma, Burkitt's lymphoma, multiple myeloma, or Richter's Syndrome (Richter's Transformation).
